SEQUENCE LISTING (173 sequences)

SEQ ID NO: 1 - protein, 11 aa, Hepatitis B Virus
  VARIANT (1)..(11): Xaa = any amino acid
XXXTCXXXAXG

SEQ ID NO: 2 - protein, 11 aa, Hepatitis B Virus
  VARIANT (1)..(1): Xaa = P, T or S
  VARIANT (2)..(2): Xaa = C or S
  VARIANT (3)..(3): Xaa = R, K, D or I
  VARIANT (6)..(6): Xaa = M or T
  VARIANT (7)..(7): Xaa = T, A or I
  VARIANT (8)..(8): Xaa = T, P or L
  VARIANT (10)..(10): Xaa = Q, H or L
XXXTCXXXAXG

SEQ ID NO: 3 - protein, 226 aa, Hepatitis B Virus
  MISC_FEATURE (1)..(226): S domain of HBsAg (GenBank acc. no. J02203)
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 4 - protein, 226 aa, Hepatitis B Virus
  MISC_FEATURE (1)..(226): S domain of HBsAg (GenBank acc. no. FJ899792)
MENVTSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTGTGPCRTCTTPAQGTSMYPSCCCTKPSDGNCTCIPIPSSWAFGKFLWEWASARFSWLSLLVPFVQWFVGLSPTVWLSVIWMMWYWGPSLYSTLSPFLPLLPIFFCLWVYI

SEQ ID NO: 5 - protein, 72 aa, Hepatitis B Virus
  MISC_FEATURE (1)..(72): J02203 (D, ayw3)
QGMLPVCPLIPGSSTTSTGPCRTCMTTAQGTSMYPSCCCTKPSDGNCTCIPIPSSWAFGKFLWEWASARFSW

SEQ ID NO: 6 - protein, 72 aa, Hepatitis B Virus
  MISC_FEATURE (1)..(72): FJ899792 (D, adw2)
QGMLPVCPLIPGSSTTGTGPCRTCTTPAQGTSMYPSCCCTKPSDGNCTCIPIPSSWAFGKFLWEWASARFSW

SEQ ID NO: 7 - protein, 72 aa, Hepatitis B Virus
  MISC_FEATURE (1)..(72): AM282986 (A)
QGMLPVCPLIPGTTTTSTGPCKTCTTPAQGNSMFPSCCCTKPSDGNCTCIPIPSSWAFAKYLWEWASVRFSW

SEQ ID NO: 8 - protein, 72 aa, Hepatitis B Virus
  MISC_FEATURE (1)..(72): D23678 (B1)
QGMLPVCPLIPGSSTTSTGPCKTCTTPAQGTSMFPSCCCTKPTDGNCTCIPIPSSWAFAKYLWEWASVRFSW

SEQ ID NO: 9 - protein, 72 aa, Hepatitis B Virus
  MISC_FEATURE (1)..(72): AB117758 (C1)
QGMLPVCPLLPGTSTTSTGPCKTCTIPAQGTSMFPSCCCTKPSDGNCTCIPIPSSWAFARFLWEWASVRFSW

SEQ ID NO: 10 - protein, 72 aa, Hepatitis B Virus
  MISC_FEATURE (1)..(72): AB205192 (E)
QGMLPVCPLIPGSSTTSTGPCRTCTTLAQGTSMFPSCCCSKPSDGNCTCIPIPSSWAFGKFLWEWASARFSW

SEQ ID NO: 11 - protein, 72 aa, Hepatitis B Virus
  MISC_FEATURE (1)..(72): X69798 (F4)
QGMLPVCPLLPGSTTTSTGPCKTCTTLAQGTSMFPSCCCSKPSDGNCTCIPIPSSWALGKYLWEWASARFSW

SEQ ID NO: 12 - protein, 72 aa, Hepatitis B Virus
  MISC_FEATURE (1)..(72): AF160501 (G)
QGMLPVCPLIPGSSTTSTGPCKTCTTPAQGNSMYPSCCCTKPSDGNCTCIPIPSSWAFAKYLWEWASVRFSW

SEQ ID NO: 13 - protein, 72 aa, Hepatitis B Virus
  MISC_FEATURE (1)..(72): AY090454 (H)
QGMLPVCPLLPGSTTTSTGPCKTCTTLAQGTSMFPSCCCTKPSDGNCTCIPIPSSWAFGKYLWEWASARFSW

SEQ ID NO: 14 - protein, 72 aa, Hepatitis B Virus
  MISC_FEATURE (1)..(72): AF241409 (I)
QGMLPVCPLIPGSSTTSTGPCKTCTTPAQGNSMYPSCCCTKPSDGNCTCIPIPSSWAFAKYLWEWASARFSW

SEQ ID NO: 15 - protein, 72 aa, Hepatitis B Virus
  MISC_FEATURE (1)..(72): AB486012 (J)
QGMLPVCPLLPGSTTTSTGPCRTCTITAQGTSMFPSCCCTKPSDGNCTCIPIPSSWAFAKFLWEWASVRFSW

SEQ ID NO: 16 - protein, 73 aa, Hepatitis B Virus
  MISC_FEATURE (1)..(73): HBsAg Y100C/P120T
CQGMLPVCPLIPGSSTTGTGTCRTCTTPAQGTSMYPSCCCTKPSDGNCTCIPIPSSWAFGKFLWEWASARFSW

SEQ ID NO: 17 - protein, 72 aa, Hepatitis B Virus
  MISC_FEATURE (1)..(72): HBsAg P120T
QGMLPVCPLIPGSSTTGTGTCRTCTTPAQGTSMYPSCCCTKPSDGNCTCIPIPSSWAFGKFLWEWASARFSW

SEQ ID NO: 18 - protein, 72 aa, Hepatitis B Virus
  MISC_FEATURE (1)..(72): HBsAg P120T/S143L
QGMLPVCPLIPGSSTTGTGTCRTCTTPAQGTSMYPSCCCTKPLDGNCTCIPIPSSWAFGKFLWEWASARFSW

SEQ ID NO: 19 - protein, 72 aa, Hepatitis B Virus
  MISC_FEATURE (1)..(72): HBsAg C121S
QGMLPVCPLIPGSSTTGTGPSRTCTTPAQGTSMYPSCCCTKPSDGNCTCIPIPSSWAFGKFLWEWASARFSW

SEQ ID NO: 20 - protein, 72 aa, Hepatitis B Virus
  MISC_FEATURE (1)..(72): HBsAg R122D
QGMLPVCPLIPGSSTTGTGPCDTCTTPAQGTSMYPSCCCTKPSDGNCTCIPIPSSWAFGKFLWEWASARFSW

SEQ ID NO: 21 - protein, 72 aa, Hepatitis B Virus
  MISC_FEATURE (1)..(72): HBsAg R122I
QGMLPVCPLIPGSSTTGTGPCITCTTPAQGTSMYPSCCCTKPSDGNCTCIPIPSSWAFGKFLWEWASARFSW

SEQ ID NO: 22 - protein, 72 aa, Hepatitis B Virus
  MISC_FEATURE (1)..(72): HBsAg T123N
QGMLPVCPLIPGSSTTGTGPCRNCTTPAQGTSMYPSCCCTKPSDGNCTCIPIPSSWAFGKFLWEWASARFSW

SEQ ID NO: 23 - protein, 72 aa, Hepatitis B Virus
  MISC_FEATURE (1)..(72): HBsAg Q129H
QGMLPVCPLIPGSSTTGTGPCRTCTTPAHGTSMYPSCCCTKPSDGNCTCIPIPSSWAFGKFLWEWASARFSW

SEQ ID NO: 24 - protein, 72 aa, Hepatitis B Virus
  MISC_FEATURE (1)..(72): HBsAg Q129L
QGMLPVCPLIPGSSTTGTGPCRTCTTPALGTSMYPSCCCTKPSDGNCTCIPIPSSWAFGKFLWEWASARFSW

SEQ ID NO: 25 - protein, 72 aa, Hepatitis B Virus
  MISC_FEATURE (1)..(72): HBsAg M133H
QGMLPVCPLIPGSSTTGTGPCRTCTTPAQGTSHYPSCCCTKPSDGNCTCIPIPSSWAFGKFLWEWASARFSW

SEQ ID NO: 26 - protein, 72 aa, Hepatitis B Virus
  MISC_FEATURE (1)..(72): HBsAg M133L
QGMLPVCPLIPGSSTTGTGPCRTCTTPAQGTSLYPSCCCTKPSDGNCTCIPIPSSWAFGKFLWEWASARFSW

SEQ ID NO: 27 - protein, 72 aa, Hepatitis B Virus
  MISC_FEATURE (1)..(72): HBsAg M133T
QGMLPVCPLIPGSSTTGTGPCRTCTTPAQGTSTYPSCCCTKPSDGNCTCIPIPSSWAFGKFLWEWASARFSW

SEQ ID NO: 28 - protein, 72 aa, Hepatitis B Virus
  MISC_FEATURE (1)..(72): HBsAg K141E
QGMLPVCPLIPGSSTTGTGPCRTCTTPAQGTSMYPSCCCTEPSDGNCTCIPIPSSWAFGKFLWEWASARFSW

SEQ ID NO: 29 - protein, 72 aa, Hepatitis B Virus
  MISC_FEATURE (1)..(72): HBsAg P142S
QGMLPVCPLIPGSSTTGTGPCRTCTTPAQGTSMYPSCCCTKSSDGNCTCIPIPSSWAFGKFLWEWASARFSW

SEQ ID NO: 30 - protein, 72 aa, Hepatitis B Virus
  MISC_FEATURE (1)..(72): HBsAg S143K
QGMLPVCPLIPGSSTTGTGPCRTCTTPAQGTSMYPSCCCTKPKDGNCTCIPIPSSWAFGKFLWEWASARFSW

SEQ ID NO: 31 - protein, 72 aa, Hepatitis B Virus
  MISC_FEATURE (1)..(72): HBsAg D144A
QGMLPVCPLIPGSSTTGTGPCRTCTTPAQGTSMYPSCCCTKPSAGNCTCIPIPSSWAFGKFLWEWASARFSW

SEQ ID NO: 32 - protein, 72 aa, Hepatitis B Virus
  MISC_FEATURE (1)..(72): HBsAg G145R
QGMLPVCPLIPGSSTTGTGPCRTCTTPAQGTSMYPSCCCTKPSDRNCTCIPIPSSWAFGKFLWEWASARFSW

SEQ ID NO: 33 - protein, 72 aa, Hepatitis B Virus
  MISC_FEATURE (1)..(72): HBsAg N146A
QGMLPVCPLIPGSSTTGTGPCRTCTTPAQGTSMYPSCCCTKPSDGACTCIPIPSSWAFGKFLWEWASARFSW

SEQ ID NO: 34 - protein, 10 aa, Artificial Sequence
  Synthetic sequence HBC34 Ab CDRH1 aa
GRIFRSFYMS

SEQ ID NO: 35 - protein, 17 aa, Artificial Sequence
  Synthetic sequence HBC34 Ab CDRH2 aa
TINQDGSEKLYVDSVKG

SEQ ID NO: 36 - protein, 17 aa, Artificial Sequence
  Synthetic sequence HBC34 Ab CDRH2_2 aa
NINQDGSEKLYVDSVKG

SEQ ID NO: 37 - protein, 10 aa, Artificial Sequence
  Synthetic sequence HBC34 Ab CDRH3 aa
WSGNSGGMDV

SEQ ID NO: 38 - protein, 119 aa, Artificial Sequence
  Synthetic sequence HBC34 VH_1
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSS

SEQ ID NO: 39 - protein, 119 aa, Artificial Sequence
  Synthetic sequence HBC34 VH_2
EVQLVESGGGLVQPGGSLRLSCAASGRIFRSFYMSWVRQAPGKGLEWVANINQDGSEKLYVDSVKGRFTISRDNAKNSLFLQMNNLRVEDTAVYYCAAWSGNSGGMDVWGQGTTVTVSS

SEQ ID NO: 40 - protein, 11 aa, Artificial Sequence
  Synthetic sequence HBC34, -v7, -v31, -v32 CDRL1 aa
SGDKLGNKNVC

SEQ ID NO: 41 - protein, 11 aa, Artificial Sequence
  Synthetic sequence HBC34-v35 - v50 CDRL1 aa
SGDKLGNKNVA

SEQ ID NO: 42 - protein, 11 aa, Artificial Sequence
  Synthetic sequence HBC34-v34 CDRL1 aa
SGDKLGNKNVS

SEQ ID NO: 43 - protein, 11 aa, Artificial Sequence
  Synthetic sequence HBC34-v23, -v33 CDRL1 aa
SGDKLGNKNAC

SEQ ID NO: 44 - protein, 7 aa, Artificial Sequence
  Synthetic sequence HBC34, -v7, -v23, -v31-v33, -v35, -v45, -v46, -v48 CDRL2 aa
EVKYRPS

SEQ ID NO: 45 - protein, 7 aa, Artificial Sequence
  Synthetic sequence HBC34-v36 CDRL2 aa
QDSKRPS

SEQ ID NO: 46 - protein, 7 aa, Artificial Sequence
  Synthetic sequence HBC34-v37 CDRL2 aa
EDSKRPS

SEQ ID NO: 47 - protein, 7 aa, Artificial Sequence
  Synthetic sequence HBC34-v38 CDRL2 aa
QVSKRPS

SEQ ID NO: 48 - protein, 7 aa, Artificial Sequence
  Synthetic sequence HBC34-v39 CDRL2 aa
QDDKRPS

SEQ ID NO: 49 - protein, 7 aa, Artificial Sequence
  Synthetic sequence HBC34-v40 CDRL2 aa
QDSYRPS

SEQ ID NO: 50 - protein, 7 aa, Artificial Sequence
  Synthetic sequence HBC34-v41 CDRL2 aa
QVSYRPS

SEQ ID NO: 51 - protein, 7 aa, Artificial Sequence
  Synthetic sequence HBC34-v42, -v47, -v49, -v50 CDRL2 aa
EVSYRPS

SEQ ID NO: 52 - protein, 7 aa, Artificial Sequence
  Synthetic sequence HBC34-v43 CDRL2 aa
QVKYRPS

SEQ ID NO: 53 - protein, 7 aa, Artificial Sequence
  Synthetic sequence HBC34-v44 CDRL2 aa
AVKYRPS

SEQ ID NO: 54 - skipped

SEQ ID NO: 55 - protein, 9 aa, Artificial Sequence
  Synthetic sequence HBC34-v7, -v23, -v32, -v33, -v35-v50 CDRL3 aa
QTFDSTTVV

SEQ ID NO: 56 - protein, 9 aa, Artificial Sequence
  Synthetic sequence HBC34, -v31 CDRL3 aa
QTWDSTTVV

SEQ ID NO: 57 - protein, 106 aa, Artificial Sequence
  Synthetic sequence HBC34-v35 VL aa
SYELTQPPSVSVSPGQTVSIPCSGDKLGNKNVAWFQHKPGQSPVLVIYEVKYRPSGIPERFSGSNSGNTATLTISGTQAMDEAAYFCQTFDSTTVVFGGGTRLTVL

SEQ ID NO: 58 - protein, 106 aa, Artificial Sequence
  Synthetic sequence HBC34-v36 VL aa
SYELTQPPSVSVSPGQTVSIPCSGDKLGNKNVAWFQHKPGQSPVLVIYQDSKRPSGIPERFSGSNSGNTATLTISGTQAMDEAAYFCQTFDSTTVVFGGGTRLTVL

SEQ ID NO: 59 - protein, 106 aa, Artificial Sequence
  Synthetic sequence HBC34-v37 VL aa
SYELTQPPSVSVSPGQTVSIPCSGDKLGNKNVAWFQHKPGQSPVLVIYEDSKRPSGIPERFSGSNSGNTATLTISGTQAMDEAAYFCQTFDSTTVVFGGGTRLTVL

SEQ ID NO: 60 - protein, 106 aa, Artificial Sequence
  Synthetic sequence HBC34-v38 VL aa
SYELTQPPSVSVSPGQTVSIPCSGDKLGNKNVAWFQHKPGQSPVLVIYQVSKRPSGIPERFSGSNSGNTATLTISGTQAMDEAAYFCQTFDSTTVVFGGGTRLTVL

SEQ ID NO: 61 - protein, 106 aa, Artificial Sequence
  Synthetic sequence HBC34-v39 VL aa
SYELTQPPSVSVSPGQTVSIPCSGDKLGNKNVAWFQHKPGQSPVLVIYQDKKRPSGIPERFSGSNSGNTATLTISGTQAMDEAAYFCQTFDSTTVVFGGGTRLTVL

SEQ ID NO: 62 - protein, 106 aa, Artificial Sequence
  Synthetic sequence HBC34-v40 VL aa
SYELTQPPSVSVSPGQTVSIPCSGDKLGNKNVAWFQHKPGQSPVLVIYQDSYRPSGIPERFSGSNSGNTATLTISGTQAMDEAAYFCQTFDSTTVVFGGGTRLTVL

SEQ ID NO: 63 - protein, 106 aa, Artificial Sequence
  Synthetic sequence HBC34-v41 VL aa
SYELTQPPSVSVSPGQTVSIPCSGDKLGNKNVAWFQHKPGQSPVLVIYQVSYRPSGIPERFSGSNSGNTATLTISGTQAMDEAAYFCQTFDSTTVVFGGGTRLTVL

SEQ ID NO: 64 - protein, 106 aa, Artificial Sequence
  Synthetic sequence HBC34-v42 VL aa
SYELTQPPSVSVSPGQTVSIPCSGDKLGNKNVAWFQHKPGQSPVLVIYEVSYRPSGIPERFSGANSGNTATLTISGTQAMDEAAYFCQTFDSTTVVFGGGTRLTVL

SEQ ID NO: 65 - protein, 106 aa, Artificial Sequence
  Synthetic sequence HBC34-v43 VL aa
SYELTQPPSVSVSPGQTVSIPCSGDKLGNKNVAWFQHKPGQSPVLVIYQVKYRPSGIPERFSGSNSGNTATLTISGTQAMDEAAYFCQTFDSTTVVFGGGTRLTVL

SEQ ID NO: 66 - protein, 106 aa, Artificial Sequence
  Synthetic sequence HBC34-v44 VL aa
SYELTQPPSVSVSPGQTVSIPCSGDKLGNKNVAWFQHKPGQSPVLVIYAVKYRPSGIPERFSGSNSGNTATLTISGTQAMDEAAYFCQTFDSTTVVFGGGTRLTVL

SEQ ID NO: 67 - protein, 106 aa, Artificial Sequence
  Synthetic sequence HBC34-v45 VL aa
SYELTQPPSVSVSPGQTVSIPCSGDKLGNKNVAWFQHKPGQSPVLVIYEVKYRPSGIPENFSGSNSGNTATLTISGTQAMDEAAYFCQTFDSTTVVFGGGTRLTVL

SEQ ID NO: 68 - protein, 106 aa, Artificial Sequence
  Synthetic sequence HBC34-v46 VL aa
SYELTQPPSVSVSPGQTVSIPCSGDKLGNKNVAWFQHKPGQSPVLVIYEVKYRPSGIPEAFSGSNSGNTATLTISGTQAMDEAAYFCQTFDSTTVVFGGGTRLTVL

SEQ ID NO: 69 - protein, 106 aa, Artificial Sequence
  Synthetic sequence HBC34-v47 VL aa
SYELTQPPSVSVSPGQTVSIPCSGDKLGNKNVAWFQHKPGQSPVLVIYEVSYRPSGIPENFSGANSGNTATLTASGTQAMDEAAYFCQTFDSTTVVFGGGTRLTVL

SEQ ID NO: 70 - protein, 106 aa, Artificial Sequence
  Synthetic sequence HBC34-v48 VL aa
SYELTQPPSVSVSPGQTVSIPCSGDKLGNKNVAWFQHKPGQSPVLVIYEVKYRPSGIPENFSGANSGNTATLTASGTQAMDEAAYFCQTFDSTTVVFGGGTRLTVL

SEQ ID NO: 71 - protein, 106 aa, Artificial Sequence
  Synthetic sequence HBC34-v49 VL aa
SYELTQPPSVSVSPGQTVSIPCSGDKLGNKNVAWFQHKPGQSPVLVIYEVSYRPSGIPERFSGSNSGNTATLTISGTQAMDEAAYFCQTFDSTTVVFGGGTRLTVL

SEQ ID NO: 72 - protein, 106 aa, Artificial Sequence
  Synthetic sequence HBC34-v50 VL aa
SYELTQPPSVSVSPGQTVSIPCSGDKLGNKNVAWFQHKPGQSPVLVIYEVKYRPSGIPEKFSGSNSGNTATLTISGTQAMDEAAYFCQTFDSTTVVFGGGTRLTVL

SEQ ID NO: 73 - protein, 217 aa, Homo sapiens
  MISC_FEATURE (1)..(217): IgGI - WT hIgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 19 aa, Artificial Sequence
  Synthetic Chimeric hinge sequence
ESKYGPPCPPCPAPPVAGP

SEQ ID NO: 75 - protein, 449 aa, Artificial Sequence
  Synthetic sequence HC of HBC34-v35-50 MLNS-GAALIE (g1M17, 1)
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 76 - protein, 449 aa, Artificial Sequence
  Synthetic sequence HC of HBC34-v35-50 MLNS (g1M17, 1)
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 77 - protein, 449 aa, Artificial Sequence
  Synthetic sequence HBC34-v35-50 HC with GAALIE mutation in hIgG1 Fc
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 449 aa, Artificial Sequence
  Synthetic sequence HBC34-V35-50 HC (wild-type)
ELQLVESGGGWVQPGGSQRLSCAASGRIFRSFYMSWVRQAPGKGLEWVATINQDGSEKLYVDSVKGRFTISRDNAKNSLFLQMNNLRVEDTAVYYCAAWSGNSGGMDVWGQGTTVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 106 aa, Artificial Sequence
  Synthetic sequence HBC antibody light chain constant region
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 80 - DNA, 357 bp, Artificial Sequence
  Synthetic sequence HBC34 VH nuc
gaactgcagctggtggagtctgggggaggctgggtccagccgggggggtcccagagactgtcctgtgcagcctctggacgcatctttagaagtttttacatgagctgggtccgccaggccccagggaaggggctggagtgggtggccactataaaccaagatggaagtgagaaattatatgtggactctgtgaagggccgattcaccatctccagagacaacgccaagaactcactatttctgcaaatgaacaacctgagagtcgaggacacggccgtttattactgcgcggcttggagcggcaatagtgggggtatggacgtctggggccaggggaccacggtctccgtctcctca

SEQ ID NO: 81 - DNA, 357 bp, Artificial Sequence
  Synthetic sequence HBC34-v35-v50 VH (nuc)
gaggtgcagctggtggaatccggcgggggactggtgcagcctggcggctcactgagactgagctgtgcagcttctggaagaatcttcagatctttttacatgagttgggtgagacaggctcctgggaagggactggagtgggtcgcaaacatcaatcaggacggatcagaaaagctgtatgtggatagcgtcaaaggcaggttcactatttcccgcgacaacgccaaaaattctctgtttctgcagatgaacaatctgcgggtggaggataccgctgtctactattgtgcagcctggtctggcaacagtggaggcatggacgtgtggggacagggaaccacagtgacagtcagctcc

SEQ ID NO: 82 - DNA, 357 bp, Artificial Sequence
  Synthetic sequence HBC34 wt VH codon optimized
gaactgcagctggtcgaatcaggaggagggtgggtccagcccggagggagccagagactgtcttgtgccgcatcagggaggatcttcaggagcttctacatgtcctgggtgcgccaggcaccaggcaagggactggagtgggtcgccaccatcaaccaggacggatctgaaaagctgtatgtggatagtgtcaaaggccggttcacaattagcagagacaacgctaaaaattctctgtttctgcagatgaacaatctgcgagtggaggataccgccgtctactattgcgccgcttggtctggcaacagcggcgggatggatgtctgggggcagggcacaacagtgagcgtctcttcc

SEQ ID NO: 83 - DNA, 990 bp, Artificial Sequence
  Synthetic sequence HBC34-V35-50 CH1-hinge-CH2-CH3 (codon-optimized)
gcctccacaaagggcccaagcgtgtttccactggctccctcttccaagtctacctccggcggcacagccgctctgggatgtctggtgaaggattacttcccagagcccgtgaccgtgtcttggaactccggcgccctgaccagcggagtgcatacatttccagctgtgctgcagagctctggcctgtactctctgtccagcgtggtgaccgtgccctcttccagcctgggcacccagacatatatctgcaacgtgaatcacaagccaagcaatacaaaggtggacaagaaggtggagcccaagtcttgtgataagacccatacatgccctccatgtccagctccagagctgctgggcggcccaagcgtgttcctgtttccacccaagcctaaggataccctgatgatctccagaacccccgaggtgacatgcgtggtggtggacgtgagccacgaggatcctgaggtgaagttcaactggtacgtggacggcgtggaggtgcataatgctaagaccaagcccagggaggagcagtacaactctacctatcgggtggtgtccgtgctgacagtgctgcaccaggattggctgaacggcaaggagtataagtgcaaggtgtctaataaggccctgcccgctcctatcgagaagaccatctccaaggccaagggccagcctagagagccacaggtgtacacactgcctccatctcgcgatgagctgaccaagaaccaggtgtccctgacatgtctggtgaagggcttctatccttccgacatcgctgtggagtgggagagcaatggccagccagagaacaattacaagaccacaccccctgtgctggacagcgatggctctttctttctgtatagcaagctgaccgtggacaagtctcgctggcagcagggcaacgtgtttagctgttctgtgatgcatgaggccctgcacaatcattatacacagaagtccctgagcctgtctcctggcaag

SEQ ID NO: 84 - DNA, 1417 bp, Artificial Sequence
  Synthetic sequence HBC34-v35-50 VH-CH1-hinge-CH2-CH3 (codon-optimized)
gagctgcagctggtggagtccggcggcggctgggtgcagcctggcggctcccagaggctgagctgtgccgcttctggcaggatcttccggtccttttacatgtcttgggtgcggcaggctccaggcaagggcctggagtgggtggctaccatcaaccaggacggctccgagaagctgtatgtggatagcgtgaagggcagattcacaatctctcgcgacaacgccaagaactccctgtttctgcagatgaacaatctgagggtggaggataccgccgtgtactattgcgccgcttggtctggcaatagcggcggcatggacgtgtggggacagggcaccaccgtgtccgtgtccagcgcctccacaaagggcccaagcgtgtttccactggctccctcttccaagtctacctccggcggcacagccgctctgggatgtctggtgaaggattacttcccagagcccgtgaccgtgtcttggaactccggcgccctgaccagcggagtgcatacatttccagctgtgctgcagagctctggcctgtactctctgtccagcgtggtgaccgtgccctcttccagcctgggcacccagacatatatctgcaacgtgaatcacaagccaagcaatacaaaggtggacaagaaggtggagcccaagtcttgtgataagacccatacatgccctccatgtccagctccagagctgctgggcggcccaagcgtgttcctgtttccacccaagcctaaggataccctgatgatctccagaacccccgaggtgacatgcgtggtggtggacgtgagccacgaggatcctgaggtgaagttcaactggtacgtggacggcgtggaggtgcataatgctaagaccaagcccagggaggagcagtacaactctacctatcgggtggtgtccgtgctgacagtgctgcaccaggattggctgaacggcaaggagtataagtgcaaggtgtctaataaggccctgcccgctcctatcgagaagaccatctccaaggccaagggccagcctagagagccacaggtgtacacactgcctccatctcgcgatgagctgaccaagaaccaggtgtccctgacatgtctggtgaagggcttctatccttccgacatcgctgtggagtgggagagcaatggccagccagagaacaattacaagaccacaccccctgtgctggacagcgatggctctttctttctgtatagcaagctgaccgtggacaagtctcgctggcagcagggcaacgtgtttagctgttctgtgatgcatgaggccctgcacaatcattatacacagaagtccctgagcctgtctcctggcaagtgatgaggtaccgtgcgacggccggcaagcccccgctccccgggctctcgcggtcgtacgaggaaagctt

SEQ ID NO: 85 - DNA, 344 bp, Artificial Sequence
  Synthetic sequence HBC34-v36 VL nt
agctgacacagcccccttccgtgtccgtgtcccctggacagaccgtgtccatcccatgcagcggcgacaagctgggcaacaagaacgtggcctggtttcagcataagcctggccagtcccccgtgctggtcatctaccaggactccaagaggcccagcggcatccctgagcggttctctggctccaacagcggcaatacagccaccctgacaatctctggcacacaggctatggacgaggccgcttatttctgccagacctttgattccaccacagtggtgttcggcggcggcaccagactgacagtgctgggtcagcccaaggctgccccctcggtcactctg

SEQ ID NO: 86 - DNA, 318 bp, Artificial Sequence
  Synthetic sequence HBC34-v37 VL nt
tcctacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctacgaggattctaagaggccttccggcatcccagagcggttttccggcagcaactctggcaataccgccacactgaccatcagcggcacacaggctatggacgaggccgcttatttctgtcagacctttgattctaccacagtggtgttcggcggcggcacaaggctgaccgtgctg

SEQ ID NO: 87 - DNA, 318 bp, Artificial Sequence
  Synthetic sequence HBC34-v38 VL nt
tcctacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctaccaggtgtctaagaggccttccggcatcccagagcggttttccggcagcaactctggcaataccgccacactgaccatcagcggcacacaggctatggacgaggccgcttatttctgtcagacctttgattctaccacagtggtgttcggcggcggcacaaggctgaccgtgctg

SEQ ID NO: 88 - DNA, 318 bp, Artificial Sequence
  Synthetic sequence HBC34-v39 VL nt
tcctacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctaccaggataagaagaggccttccggcatcccagagcggttttccggcagcaactctggcaataccgccacactgaccatcagcggcacacaggctatggacgaggccgcttatttctgtcagacctttgattctaccacagtggtgttcggcggcggcacaaggctgaccgtgctg

SEQ ID NO: 89 - DNA, 318 bp, Artificial Sequence
  Synthetic sequence HBC34-v40 VL nt
tcctacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctaccaggattcttataggccttccggcatcccagagcggttttccggcagcaactctggcaataccgccacactgaccatcagcggcacacaggctatggacgaggccgcttatttctgtcagacctttgattctaccacagtggtgttcggcggcggcacaaggctgaccgtgctg

SEQ ID NO: 90 - DNA, 318 bp, Artificial Sequence
  Synthetic sequence HBC34-v41 VL nt
tcctacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctaccaggtgtcttataggccttccggcatcccagagcggttttccggcagcaactctggcaataccgccacactgaccatcagcggcacacaggctatggacgaggccgcttatttctgtcagacctttgattctaccacagtggtgttcggcggcggcacaaggctgaccgtgctg

SEQ ID NO: 91 - DNA, 318 bp, Artificial Sequence
  Synthetic sequence HBC34-v42 VL nt
tcttacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctacgaggtgtcttataggccttccggcatcccagagcggtttagcggcgccaactctggcaataccgctacactgaccatctccggcacacaggctatggacgaggccgcttatttctgtcagacctttgatagcaccacagtggtgttcggcggcggcacaaggctgaccgtgctg

SEQ ID NO: 92 - DNA, 318 bp, Artificial Sequence
  Synthetic sequence HBC34-v43 VL nt
tcctacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctaccaggtgaagtataggccttccggcatcccagagcggttttccggcagcaactctggcaataccgccacactgaccatcagcggcacacaggctatggacgaggccgcttatttctgtcagacctttgattctaccacagtggtgttcggcggcggcacaaggctgaccgtgctg

SEQ ID NO: 93 - DNA, 318 bp, Artificial Sequence
  Synthetic sequence HBC34-v44 VL nt
tcctacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctacgctgtgaagtataggccttccggcatcccagagcggttttccggcagcaactctggcaataccgccacactgaccatcagcggcacacaggctatggacgaggccgcttatttctgtcagacctttgattctaccacagtggtgttcggcggcggcacaaggctgaccgtgctg

SEQ ID NO: 94 - DNA, 318 bp, Artificial Sequence
  Synthetic sequence HBC34-v45 VL nt
tcctacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctacgaggtgaagtataggccttccggcatcccagagaacttttccggcagcaactctggcaataccgccacactgaccatcagcggcacacaggctatggacgaggccgcttatttctgtcagacctttgattctaccacagtggtgttcggaggaggaacaaggctgaccgtgctg

SEQ ID NO: 95 - DNA, 318 bp, Artificial Sequence
  Synthetic sequence HBC34-v46 VL nt
tcctacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctacgaggtgaagtataggccttccggcatcccagaggctttttccggcagcaactctggcaataccgccacactgaccatcagcggcacacaggctatggacgaggccgcttatttctgtcagacctttgattctaccacagtggtgttcggaggaggaacaaggctgaccgtgctg

SEQ ID NO: 96 - DNA, 318 bp, Artificial Sequence
  Synthetic sequence HBC34-v49 VL nt
tcctacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctacgaggtgtcttataggccttccggcatcccagagcggttttccggcagcaactctggcaataccgccacactgaccatcagcggcacacaggctatggacgaggccgcttatttctgtcagacctttgattctaccacagtggtgttcggcggcggcacaaggctgaccgtgctg

SEQ ID NO: 97 - DNA, 318 bp, Artificial Sequence
  Synthetic sequence HBC34-v50 VL nt
tcctacgagctgacacagccaccttccgtgagcgtgtctccaggacagaccgtgtccatcccttgcagcggcgacaagctgggcaacaagaatgtggcctggttccagcacaagccaggccagtcccccgtgctggtcatctacgaggtgaagtataggccttccggcatcccagagaagttttccggcagcaactctggcaataccgccacactgaccatcagcggcacacaggctatggacgaggccgcttatttctgtcagacctttgattctaccacagtggtgttcggaggaggaacaaggctgaccgtgctg

SEQ ID NO: 98 - DNA, 318 bp, Artificial Sequence
  Synthetic sequence HBC34-V35-50 CL (codon-optimized)_1
ggacagccaaaggctgctccatctgtgaccctgtttccaccctcttccgaggagctgcaggccaacaaggccaccctggtgtgcctgatctctgacttctaccctggagctgtgacagtggcttggaaggctgatagctctcccgtgaaggctggcgtggagacaacaacccctagcaagcagtctaacaataagtacgccgcttccagctatctgtctctgacaccagagcagtggaagtcccaccgctcttattcctgccaggtgacccatgagggcagcaccgtggagaagacagtggcccccaccgagtgttct

SEQ ID NO: 99 - DNA, 318 bp, Artificial Sequence; identical to SEQ ID NO: 98
  Synthetic sequence HBC34-V35-50 CL (codon-optimized)_2

SEQ ID NO: 100 - protein, 33 aa, Artificial Sequence
  Synthetic peptide
  VARIANT (1)..(33): Xaa = Cys
XGSSTTSTGPCRTCMTXPSDGNATAIPIPSSWX

SEQ ID NO: 101 - protein, 15 aa, Artificial Sequence
  Synthetic peptide
TSTGPCRTCMTTAQG

SEQ ID NO: 102 - protein, 26 aa, Artificial Sequence
  Synthetic peptide
GMLPVCPLIPGSSTTSTGPCRTCMTT

SEQ ID NO: 103 - protein, 33 aa, Artificial Sequence
  Synthetic peptide
  VARIANT (1)..(33): Xaa = Cys
XSMYPSASATKPSDGNXTGPCRTCMTTAQGTSX

SEQ ID NO: 104 - protein, 11 aa, Heptatits B Virus
  MISC_FEATURE (1)..(11): amino acids 120 - 130 of the S domain of HBsAg (HBV-D J02203
PCRTCMTTAQG

SEQ ID NO: 105 - protein, 11 aa, Hepatitis B Virus
  VARIANT (3)..(3): Xaa = R or K
  VARIANT (6)..(6): Xaa = M or T
  VARIANT (7)..(7): Xaa = T or I
  VARIANT (8)..(8): Xaa = T, P, or L
PCXTCXXXAQG

SEQ ID NO: 106 - protein, 5 aa, Artificial Sequence
  Synthetic linker
GGSGG

SEQ ID NO: 107 - protein, 7 aa, Hepatitis B Virus
TGPCRTC

SEQ ID NO: 108 - protein, 7 aa, Heptatits B Virus
GNCTCIP

SEQ ID NO: 109 - protein, 25 aa, Hepatitis B Virus
  MOD_RES (2)..(2): coupled to acetamidomethyl
  MOD_RES (21)..(21): coupled to acetamidomethyl
  MOD_RES (24)..(24): coupled to acetamidomethyl
CCIPIPSSWAFGCSTTSTGPCRTCC

SEQ ID NO: 110 - protein, 28 aa, Hepatitis B Virus
  MOD_RES (4)..(4): coupled to acetamidomethyl
  MOD_RES (6)..(6): coupled to acetamidomethyl
  MOD_RES (24)..(24): coupled to acetamidomethyl
  MOD_RES (27)..(27): coupled to acetamidomethyl
CGNCTCIPIPSSWAFCSTTSTGPCRTCC

SEQ ID NO: 111 - protein, 17 aa, Hepatitis B Virus
  MOD_RES (13)..(16): coupled to acetamidomethyl
CGGGCSTTSTGPCRTCC

SEQ ID NO: 112 - protein, 11 aa, Hepatitis B Virus
STTSTGPCRTC

SEQ ID NO: 113 - protein, 15 aa, Hepatitis B Virus
GNCTCIPIPSSWAFC

SEQ ID NO: 114 - protein, 14 aa, Hepatitis B Virus
GNCTCIPIPSSWAF

SEQ ID NO: 115 - protein, 5 aa, Hepatitis B Virus
  VARIANT (1)..(5): Xaa = Any Amino Acid
PCRXC

SEQ ID NO: 116 - DNA, 3182 bp, Hepatits B Virus
aattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttagtattccttggactcataaggtggggaactttactgggctttattcttctactgtacctgtctttaatcctcattggaaaacaccatcttttcctaatatacatttacaccaagacattatcaaaaaatgtgaacagtttgtaggcccactcacagttaatgagaaaagaagattgcaattgattatgcctgccaggttttatccaaaggttaccaaatatttaccattggataagggtattaaaccttattatccagaacatctagttaatcattacttccaaactagacactatttacacactctatggaaggcgggtatattatataagagagaaacaacacatagcgcctcattttgtgggtcaccatattcttgggaacaagatctacagcatggggcagaatctttccaccagcaatcctctgggattctttcccgaccaccagttggatccagccttcagagcaaacaccgcaaatccagattgggacttcaatcccaacaaggacacctggccagacgccaacaaggtaggagctggagcattcgggctgggtttcaccccaccgcacggaggccttttggggtggagccctcaggctcagggcatactacaaactttgccagcaaatccgcctcctgcctccaccaatcgccagtcaggaaggcagcctaccccgctgtctccacctttgagaaacactcatcctcaggccatgcagtgg

SEQ ID NO: 117 - DNA, 3215 bp, Hepatitis B Virus
ctccacaacattccaccaagctctgctagatcccagagtgaggggcctatattttcctgctggtggctccagttccggaacagtaaaccctgttccgactactgcctcacccatatcgtcaatcttctcgaggactggggaccctgcaccgaacatggagagcacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaacttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaactaccagcacgggaccatgcagaacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagctatatggatgatgtggtattgggggccaagtctgtacaacatcttgagtccctttttacctctattaccaattttcttttgtctttgggtatacatttgaaccctaataaaaccaaacgttggggctactcccttaacttcatgggatatgtaattggaagttggggtactttaccgcaggaacatattgtacaaaaactcaagcaatgttttcgaaaattgcctgtaaatagacctattgattggaaagtatgtcaaagaattgtgggtcttttgggctttgctgccccttttacacaatgtggctatcctgccttgatgcctttatatgcatgtatacaatctaagcaggctttcactttctcgccaacttacaaggcctttctgtgtaaacaatatctaaacctttaccccgttgcccggcaacggtcaggtctctgccaagtgtttgctgacgcaacccccacgggttggggcttggccataggccatcggcgcatgcgtggaacctttgtggctcctctgccgatccatactgcggaactcctagcagcttgttttgctcgcagccggtctggagcgaaacttatcggaaccgacaactcagttgtcctctctcggaaatacacctcctttccatggctgctaggctgtgctgccaactggatcctgcgcgggacgtcctttgtctacgtcccgtcggcgctgaatcccgcggacgacccgtctcggggccgtttgggcctctaccgtccccttcttcatctgccgttccggccgaccacggggcgcacctctctttacgcggtctccccgtctgtgccttctcatctgccggaccgtgtgcacttcgcttcacctctgcacgtagcatggagaccaccgtgaacgcccaccaggtcttgcccaaggtcttacacaagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgttcaccagcaccatgcaactttttcccctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacagcactcaggcaagctattctgtgttggggtgagttgatgaatctggccacctgggtgggaagtaatttggaagacccagcatccagggaattagtagtcagctatgtcaatgttaatatgggcctaaaaattagacaactattgtggtttcacatttcctgccttacttttggaagagaaactgtccttgagtatttggtgtcttttggagtgtggattcgcactcctcccgcttacagaccaccaaatgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttagtatcccttggactcataaggtgggaaactttactgggctttattcttctactgtacctgtctttaatcctgattggaaaactccctcctttcctcacattcatttacaggaggacattattaatagatgtcaacaatatgtgggccctctgacagttaatgaaaaaaggagattaaaattaattatgcctgctaggttctatcctaaccttaccaaatatttgcccttggacaaaggcattaaaccgtattatcctgaatatgcagttaatcattacttcaaaactaggcattatttacatactctgtggaaggctggcattctatataagagagaaactacacgcagcgcctcattttgtgggtcaccatattcttgggaacaagagctacagcatgggaggttggtcttccaaacctcgacaaggcatggggacgaatctttctgttcccaatcctctgggattctttcccgatcaccagttggaccctgcgttcggagccaactcaaacaatccagattgggacttcaaccccaacaaggatcactggccagaggcaaatcaggtaggagcgggagcatttggtccagggttcaccccaccacacggaggccttttggggtggagccctcaggctcagggcatattgacaacactgccagcagcacctcctcctgcctccaccaatcggcagtcaggaagacagcctactcccatctctccacctctaagagacagtcatcctcaggccatgcagtggaa

SEQ ID NO: 118 - RNA, 19 nt, Artificial Sequence
  Synthetic sequence HBV001 sense
gugugcacuucgcuucaca

SEQ ID NO: 119 - RNA, 21 nt, Artificial Sequence
  Synthetic sequence HBV001 antisense
ugugaagcgaagugcacacuu

SEQ ID NO: 120 - RNA, 21 nt, Artificial Sequence
  Synthetic sequence HBV003 sense
gguggacuucucucaauuuua

SEQ ID NO: 121 - RNA, 23 nt, Artificial Sequence
  Synthetic sequence HBV003 antisense
uaaaauugagagaaguccaccac

SEQ ID NO: 122 - RNA, 19 nt, Artificial Sequence
  Synthetic sequence HBV002v2 sense
gugugcacuucgcuucaca

SEQ ID NO: 123 - RNA, 21 nt, Artificial Sequence
  Synthetic sequence HBV002v2 antisense
ugugaagcgaagugcacacuu

SEQ ID NO: 124 - RNA, 19 nt, Artificial Sequence
  Synthetic sequence HBV002v1 sense
gugugcacuucgcuucaca

SEQ ID NO: 125 - RNA, 21 nt, Artificial Sequence
  Synthetic sequence HBV002v1 antisense
ugugaagcgaagugcacacuu

SEQ ID NO: 126 - RNA, 21 nt, Artificial Sequence
  Synthetic sequence No. 126 sense
gguggacuucucucaauuuua

SEQ ID NO: 127 - RNA, 23 nt, Artificial Sequence
  Synthetic sequence No. 127 antisense
uaaaauugagagaaguccaccac

SEQ ID NO: 128 - protein, 16 aa, Artificial Sequence
  Synthetic sequence RFGF
AAVALLPAVLLALLAP

SEQ ID NO: 129 - protein, 11 aa, Artificial Sequence
  Synthetic sequence RFGF analogue
AALLPVLLAAP

SEQ ID NO: 130 - protein, 13 aa, Artificial Sequence
  Synthetic sequence HIV Tat protein
GRKKRRQRRRPPQ

SEQ ID NO: 131 - protein, 15 aa, Artificial Sequence
  Synthetic sequence Drosophila Antennapedia protein
RQIKIWFQNRRMKWK

SEQ ID NO: 132 - protein, 26 aa, Artificial Sequence
  Synthetic sequence CDR framework
GQSPVLVIYEVKYRPSGIPERFSGSN

SEQ ID NO: 133 - protein, 31 aa, Artificial Sequence
  Synthetic sequence CDR framework
PSGIPERFSGSNSGNTATLTISGTQAMDEAA

SEQ ID NO: 134 - protein, 48 aa, Artificial Sequence
  Synthetic sequence HBC34
GQSPVLVIYEVKYRPSGIPERFSGSNSGNTATLTISGTQAMDEAAYFC

SEQ ID NO: 135 - protein, 48 aa, Artificial Sequence
  Synthetic sequence GL L2
GQSPVLVIYQDSKRPSGIPERFSGSNSGNTATLTISGTQAMDEAAYFC

SEQ ID NO: 136 - protein, 48 aa, Artificial Sequence
  Synthetic sequence v36 + Q49E
GQSPVLVIYEDSKRPSGIPERFSGSNSGNTATLTISGTQAMDEAAYFC

SEQ ID NO: 137 - protein, 48 aa, Artificial Sequence
  Synthetic sequence v36 + D50V
GQSPVLVIYQVSKRPSGIPERFSGSNSGNTATLTISGTQAMDEAAYFC

SEQ ID NO: 138 - protein, 48 aa, Artificial Sequence
  Synthetic sequence v36 + S51K
GQSPVLVIYQDKKRPSGIPERFSGSNSGNTATLTISGTQAMDEAAYFC

SEQ ID NO: 139 - protein, 48 aa, Artificial Sequence
  Synthetic sequence v36 + K52Y
GQSPVLVIYQDSYRPSGIPERFSGSNSGNTATLTISGTQAMDEAAYFC

SEQ ID NO: 140 - protein, 48 aa, Artificial Sequence
  Synthetic sequence v36 + D50V + K52Y
GQSPVLVIYQVSYRPSGIPERFSGSNSGNTATLTISGTQAMDEAAYFC

SEQ ID NO: 141 - protein, 48 aa, Artificial Sequence
  Synthetic sequence HBC34 +K51S + S64A
GQSPVLVIYEVSYRPSGIPERFSGANSGNTATLTISGTQAMDEAAYFC

SEQ ID NO: 142 - protein, 48 aa, Artificial Sequence
  Synthetic sequence HBC34 +E49Q
GQSPVLVIYQVKYRPSGIPERFSGSNSGNTATLTISGTQAMDEAAYFC

SEQ ID NO: 143 - protein, 48 aa, Artificial Sequence
  Synthetic sequence HBC34 +E49A
GQSPVLVIYAVKYRPSGIPERFSGSNSGNTATLTISGTQAMDEAAYFC

SEQ ID NO: 144 - protein, 48 aa, Artificial Sequence
  Synthetic sequence HBC34 +R60N
GQSPVLVIYEVKYRPSGIPENFSGSNSGNTATLTISGTQAMDEAAYFC

SEQ ID NO: 145 - protein, 48 aa, Artificial Sequence
  Synthetic sequence HBC34 +R60A
GQSPVLVIYEVKYRPSGIPEAFSGSNSGNTATLTISGTQAMDEAAYFC

SEQ ID NO: 146 - protein, 48 aa, Artificial Sequence
  Synthetic sequence HBC34 +K51S + S64A + R60N + I74A
GQSPVLVIYEVSYRPSGIPENFSGANSGNTATLTASGTQAMDEAAYFC

SEQ ID NO: 147 - protein, 48 aa, Artificial Sequence
  Synthetic sequence HBC34 +R60N + S64A + I74A
GQSPVLVIYEVKYRPSGIPENFSGANSGNTATLTASGTQAMDEAAYFC

SEQ ID NO: 148 - protein, 48 aa, Artificial Sequence
  Synthetic sequence HBC34 +K51S
GQSPVLVIYEVSYRPSGIPERFSGSNSGNTATLTISGTQAMDEAAYFC

SEQ ID NO: 149 - protein, 48 aa, Artificial Sequence
  Synthetic sequence HBC34 + R60K
GQSPVLVIYEVKYRPSGIPEKFSGSNSGNTATLTISGTQAMDEAAYFC

SEQ ID NO: 150 - protein, 8 aa, Artificial Sequence
  Synthetic sequence CDRH1 (IMGT)
GRIFRSFY

SEQ ID NO: 151 - protein, 6 aa, Artificial Sequence
  Synthetic sequence CDRH2 - short (IMGT)
QDGSEK

SEQ ID NO: 152 - protein, 8 aa, Artificial Sequence
  Synthetic sequence CDRH2 - long (IMGT)
INQDGSEK

SEQ ID NO: 153 - protein, 12 aa, Artificial Sequence
  Synthetic sequence CDRH3 (IMGT)
AAWSGNSGGMDV

SEQ ID NO: 154 - protein, 6 aa, Artificial Sequence
  Synthetic sequence CDRL1 (IMGT)
KLGNKN

SEQ ID NO: 155 - protein, 3 aa, Artificial Sequence
  Synthetic sequence HBC34-v35, -v45, -v46, -v48 CDRL2 - short
EVK

SEQ ID NO: 156 - protein, 3 aa, Artificial Sequence
  Synthetic sequence HBC34-v36, -v40 CDRL2 - short (IMGT)
QDS

SEQ ID NO: 157 - protein, 3 aa, Artificial Sequence
  Synthetic sequence HBC34-v37 CDRL2 - short (IMGT)
EDS

SEQ ID NO: 158 - protein, 3 aa, Artificial Sequence
  Synthetic sequence HBC34-v38, -v41,  CDRL2 - short (IMGT)
QVS

SEQ ID NO: 159 - protein, 3 aa, Artificial Sequence
  Synthetic sequence HBC34-v39 CDRL2 - short (IMGT)
QDK

SEQ ID NO: 160 - protein, 3 aa, Artificial Sequence
  Synthetic sequence HBC34-v42, -v47,  -v49, -v50 CDRL2 - short (IMGT)
EVS

SEQ ID NO: 161 - protein, 3 aa, Artificial Sequence
  Synthetic sequence HBC34-v43 CDRL2 - short (IMGT)
QVK

SEQ ID NO: 162 - protein, 3 aa, Artificial Sequence
  Synthetic sequence HBC34-v44 CDRL2 - short (IMGT)
AVK

SEQ ID NO: 163 - protein, 10 aa, Artificial Sequence
  Synthetic sequence HBC34-v35, -v45, -v46, -v48 CDRL2 - long
VIYEVKYRPS

SEQ ID NO: 164 - protein, 10 aa, Artificial Sequence
  Synthetic sequence HBC34-v36 CDRL2 - long (IMGT)
VIYQDSKRPS

SEQ ID NO: 165 - protein, 10 aa, Artificial Sequence
  Synthetic sequence HBC34-v37 CDRL2 - long (IMGT)
VIYEDSKRPS

SEQ ID NO: 166 - protein, 10 aa, Artificial Sequence
  Synthetic sequence HBC34-v38 CDRL2 - long (IMGT)
VIYQVSKRPS

SEQ ID NO: 167 - protein, 10 aa, Artificial Sequence
  Synthetic sequence HBC34-v39 CDRL2 - long (IMGT)
VIYQDKKRPS

SEQ ID NO: 168 - protein, 10 aa, Artificial Sequence
  Synthetic sequence HBC34-v40 CDRL2 - long (IMGT)
VIYQDSYRPS

SEQ ID NO: 169 - protein, 10 aa, Artificial Sequence
  Synthetic sequence HBC34-v41 CDRL2 - long (IMGT)
VIYQVSYRPS

SEQ ID NO: 170 - protein, 10 aa, Artificial Sequence
  Synthetic sequence HBC34-v42, -v47, -v49, -v50 CDRL2 - long
VIYEVSYRPS

SEQ ID NO: 171 - protein, 10 aa, Artificial Sequence
  Synthetic sequence HBC34-v43 CDRL2 - long (IMGT)
VIYQVKYRPS

SEQ ID NO: 172 - protein, 10 aa, Artificial Sequence
  Synthetic sequence HBC34-v44 CDRL2 - long (IMGT)
VIYAVKYRPS

SEQ ID NO: 173 - protein, 9 aa, Artificial Sequence
  Synthetic sequence HBC34-v35-v50 CDRL3 (IMGT)
QTFDSTTVV

(19) United States
(12) Patent Application Publication
(10) Pub. No.: US 2023/0242621 A1
(43) Pub. Date: Aug. 3, 2023

(54) ENGINEERED HEPATITIS B VIRUS NEUTRALIZING ANTIBODIES AND USES THEREOF

(71) Applicants: VIR BIOTECHNOLOGY, INC., San Francisco, CA (US); HUMABS BIOMED SA, Bellinzona (CH)

(72) Inventors: Elisabetta CAMERONI, Bellinzona (CH); Davide CORTI, Bellinzona (CH); Nadine CZUDNOCHOWSKI, San Francisco, CA (US); Florian A. LEMPP, San Francisco, CA (US); Laura ROSEN, San Francisco, CA (US); Gyorgy SNELL, San Francisco, CA (US)

(21) Appl. No.: 18/002,844

(22) PCT Filed: Jun. 23, 2021
(86) PCT No.: PCT/US2021/038667

§ 371 Date: Dec. 21, 2022

Related U.S. Application Data
(60) Provisional application No. 63/043,692, filed on Jun. 24, 2020.

(51) Int. Cl. C07K 16/08; A61P 31/20; C12N 15/86; A61K 39/00

(52) U.S. Cl. CPC C07K 16/082; A61P 31/20; C12N 15/86; A61K 2039/545; C07K 2317/55; C07K 2317/565; C12N 2740/15041; C07K 2317/31; C07K 2317/52; C07K 2317/622

(57) ABSTRACT

The present disclosure relates, in part, to antibodies, and antigen-binding fragments thereof, that can bind to the antigenic loop region of hepatitis B surface antigen (HBsAg) and, optionally, can neutralize infection hepatitis B virus (HBV), and further optionally, of hepatitis delta virus (HDV). Presently disclosed antibodies and antigen-binding fragments have advantageous production characteristics, such as reduced formation of aggregates and/or improved production titer in transformed host cells, as compared to a reference antibody or antigen-binding fragment. The present disclosure also relates to fusion proteins that comprise an antigen-binding fragment, and to nucleic acids that encode and cells that produce such antibodies, antigen-binding fragments, and fusion proteins. In addition, the present disclosure relates to the use of the antibodies, antigen-binding fragments, fusion proteins, and related polynucleotides, vectors, host cells, and compositions of the present disclosure in the diagnosis, prophylaxis and treatment of hepatitis B and hepatitis D. Also provided are combination therapies comprising (i) an antibody or antigen-binding fragment and (ii) an agent that is an inhibitor of HBV gene expression and/or that reduces HBV antigenic load.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930485.414WO_SEQUENCE_LISTING.txt. The text file is 101 KB, was created on Jun. 22, 2021, and is being submitted electronically via EFS-Web.

### BACKGROUND

Hepatitis B virus causes potentially life-threatening acute and chronic liver infections. Acute hepatitis B is characterized by viremia, with or without symptoms, with the risk of fulminant hepatitis occurrence (Liang T J, Block T M, McMahon B J, Ghany M G, Urban S, Guo J T, Locarnini S, Zoulim F, Chang K M, Lok A S. Present and future therapies of hepatitis B: From discovery to cure. Hepatology. 2015 Aug. 3. doi: 10.1002/hep.28025. [Epub ahead of print]). Despite an efficacious vaccine against hepatitis B being available since 1982, WHO reports that 240 million people are chronically infected with hepatitis B and more than 780,000 people die every year due to hepatitis B complications. Approximately one third of chronic hepatitis B (CHB) patients develop cirrhosis, liver failure and hepatocellular carcinoma, accounting for 600,000 deaths per year (Liang T J, Block T M, McMahon B J, Ghany M G, Urban S, Guo J T, Locarnini S, Zoulim F, Chang K M, Lok A S. Present and future therapies of hepatitis B: From discovery to cure. Hepatology. 2015 Aug. 3. doi: 10.1002/hep.28025. [Epub ahead of print]).

For patients infected with HBV, severe complications can develop as a result of coinfection or superinfection with HDV. According to the WHO, hepatitis D infects about 15 million people worldwide. HDV is considered a subviral satellite because it can propagate only in the presence of HBV. HDV is one of the smallest known animal viruses (40 nm), whereby its genome is only 1.6 kb and encodes for S and L HDAg. All other proteins needed for genome replication of HDV, including the RNA polymerase, are provided by the host cell, and the HDV envelope is provided by HBV. When introduced into permissive cells, the HDV RNA genome replicates and associates with multiple copies of the HDV-encoded proteins to assemble a ribonucleoprotein (RNP) complex. The RNP is exported from the cell by the HBV envelope proteins, which are able to assemble lipoprotein vesicles that bud into the lumen of a pre-Golgi compartment before being secreted. Moreover, the HBV envelope proteins also provide a mechanism for the targeting of HDV to an uninfected cell, thereby ensuring the spread of HDV.

Complications caused by HDV include a greater likelihood of experiencing liver failure in acute infections and a rapid progression to liver cirrhosis, with an increased chance of developing liver cancer in chronic infections. In combination with hepatitis B virus, hepatitis D has the highest fatality rate of all the hepatitis infections, at 20% (Fattovich G, Giustina G, Christensen E, Pantalena M, Zagni I, Realdi G, Schalm S W. Influence of hepatitis delta virus infection on morbidity and mortality in compensated cirrhosis type B. Gut. 2000 March; 46(3):420-6). The only approved therapy for chronic HDV infection is interferon-alpha. However, treatment of HDV with interferon-alpha is relatively inefficient and is not well-tolerated. Treatment with interferon-alpha results in sustained virological response six months post-treatment in one-fourth of the patients. Also, nucleos(t)ide analogs (NAs) have been widely tested in hepatitis delta, but they appear to be ineffective. Combination treatment using NAs with interferon also proved to be disappointing (Zaigham Abbas, Minaam Abbas Management of hepatitis delta: Need for novel therapeutic Options. World J Gastroenterol 2015 August 28; 21(32): 9461-9465).

Accordingly, new therapeutic options, such as new therapies with neutralizing activity against Hepatitis B and/or D infection are needed.

### BRIEF DESCRIPTION OF THE DRAWINGS

The figures provided herein are intended to illustrate subject matter included in the present disclosure in more detail. The figures are not intended to limit the disclosure in any way.

FIG. 1 shows binding to HBsAg (left) and neutralization of HBV infection (right) by anti-HBV antibody “HBC34-v35-GAALIE-MLNS” (rIgG1m17, 1), comprising a VH according to SEQ ID NO.:38 and a VL according to SEQ ID NO.:57, and, in the Fc, the mutations G236A, A330L, I332E, M428L, and N434S. Binding of varying concentrations of antibody to HBsAg of ten ((A)-(J)) genotypes is shown at left. Neutralization was measured by HBsAg concentration (IU/ml) and HBeAg Index, as indicated. HBC34-v35-GAALIE-MLNS binds to a conserved conformational epitope of HBsAg with picomolar affinity and potently neutralizes 10 HBV genotypes.

FIG. 2 provides Size Exclusion Chromatography (SEC) data showing high molecular weight species present in purified HBC34-v35-GAALIE-MLNS and HBC34-v35-MLNS (which differs from HBC34-v35-GAALIE-MLNS by not comprising the G236A, A330L, and I332E Fc mutations) IgG after one week RT incubation (approx. 75 mg/mL). The maximum peak is shown in the inset image at the upper right corner of the graph.

FIG. 3 provides Size Exclusion Chromatography (SEC) data showing high molecular weight species in purified HBC34-v35 Fab over time. The maximum peak is shown in the inset image at the upper right corner of the graph.

FIG. 4 shows binding of HBC34-v35 IgG (upper row of panels) and Fab (lower row of panels) monomers (center panels) and enriched dimers (right panels) to HBsAg, as measured by surface plasmon resonance (SPR). At left are schematic illustrations showing binding by IgG and Fab to HBsAg. The HBsAg concentrations used were as indicated in the figure key.

FIGS. 5A and 5B show (A) preparative SEC data illustrating isolated HBC34-v35 recombinant Fab dimer (left peak) and (B) crystallization of Fab dimer.

FIGS. 6A and 6B show (A) preparative SEC data of isolated HBC34-v35 recombinant Fab monomer and (B) crystallization of Fab monomer.

FIG. 7 provides (left) a schematic illustration of dimer formation involving antibody CDRs and (right) ribbon models showing HBC34-v35 Fab dimer.

FIG. 8 illustrates (right) VL-VL interactions are involved in dimer formation in HBC34-v35, and (left) summarizes interactions within L-CDR2.

FIG. 9 provides another illustration of HBC34-v35 Fab-Fab interactions in L-CDR2 and light chain framework regions.

FIG. 10 provides illustrations of (left) HBC34-v35 Fabs in a dimer and (right) conformation of a Fab monomer.

FIGS. 11A-11C show reduced dimerization by a variant Fab engineered from HBC34-v35 (the variant is shown in FIG. 11A as “L-CDR2 GL Fab” and is also referred-to herein as HBC34-v36) in which three L-CDR2 residues were back-mutated to the germline sequence, as compared to HBC34-v35 Fab. (A) Percent dimer in the purified Fabs by absolute size-exclusion chromatography (aSEC) at 0 and 5-7 days. (B) SEC analysis of stressed L-CDR2 GL Fab sample at 0 days. (C) SEC analysis of stressed L-CDR2 GL Fab sample at 5 days.

FIG. 12 shows HBC34-v35 and HBC34-v36 binding to HBsAg, determined by ELISA. Antibodies were expressed as IgG1 with wild-type Fc (allotype G1m17, 1).

FIG. 13 shows in vitro neutralization of HBV genotype D infection by HBC34-v35 and HBC34-v36. Antibodies were expressed as IgG1 with wild-type Fc (allotype G1m17, 1). Neutralization was as measured by percentage of target cells expressing HBsAg (left) or HBeAg (right). N=1 experiment.

FIGS. 14A-14E show binding of additional antibodies HBC34-v37-HBC34-v50 (unpurified supernatants from CHO cells) to HBsAg, as determined by ELISA. Purified HBC34-v35 was included as a control. Antibodies were expressed as IgG1 with wild-type Fc (allotype G1m17, 1). Calculated EC50 values are shown at the bottom of each graph.

FIG. 15 shows neutralization of HBV genotype D by HBC34-v35 and certain antibodies of the present disclosure, with HBeAg as the viral readout. Calculated EC50 values of each mAb are shown at right. HBC34-v35 (purified IgG and supernatant) and HBC34-v36 (purified IgG) were used as controls.

FIGS. 16A-16D provide Size Exclusion Chromatography (SEC) data showing high molecular weight species (HMWS) present in HBC34-v35 and nine purified variant antibodies of the present disclosure over the course of 32 days. HBC34-v35 and variant antibodies were concentrated to approximately 25 mg/mL and incubated at different temperatures. HMWS were evaluated by SEC at day −1, day 0, day 5, day 15, and day 32. Day −1 samples were evaluated prior to concentration. Antibody compositions were incubated at 4° C. (FIG. 16A), 25° C. (FIG. 16B), or 40° C. (FIG. 16C) over the course of the experiment. The frequency of HMWS following a 32-day incubation at 40° C. is summarized in FIG. 16D.

FIGS. 17A-17J show binding to HBsAg of ten ((A)-(J)) genotypes by HBC34-v35, HBC34-v40, HBC34-v44, HBC34-v45, and HBC34-v50, as determined by FACS. Data are reported as Mean Fluorescence Intensity (MFI) relative to antibody concentration (ng/ml). Mock-staining is included as a negative control.

FIGS. 18A-18K show binding to HBsAg-genotype D and ten HBsAg-genotype D mutants by HBC34-v35, HBC34-v40, HBC34-v44, HBC34-v45, and HBC34-v50, as determined by FACS. Data are reported as Mean Fluorescence Intensity (MFI) relative to antibody concentration (ng/ml). Mock-staining is included as a negative control.

FIG. 19 shows antibody titers generated via transfection for HBC34-v35, HBC34-v40, HBC34-v44, HBC34-v45, and HBC34-v50. Both 5 ml- and 100 ml-scale transfection systems were evaluated, with the 100 ml system tested in duplicate or triplicate. Antibody titers from individual 5 ml- and 100 ml-scale tests, as well as average titer from 100 ml-scale tests, are shown (reported as mg/L).

FIG. 20 shows SEC data reflecting thermostability of HBC34-v35 (“HBC35” in the figure key), HBC34-v40 (“HBC40”), HBC34-v44 (“HBC44”), HBC34-v45 (“HBC45”), and HBC34-v50 (“HBC50”). Antibodies were concentrated to 25 mg/ml and incubated at 40° C. for four days prior to quantification of HMWS.

FIGS. 21A-21C, 22A-22C, and 23A-23C show light chain amino acid residues that were selected for engineering to reduce aggregation seen in HBC34-v35. FIGS. 21A, 22A, and 23A show light chain CDR2 residues of HBC34-v35 that were selected for engineering. FIGS. 21B, 22B, and 23B show framework residues that that were selected for engineering. FIGS. 21C, 22C, and 23C show sequence alignments of partial VL sequences of variant antibodies.

### DETAILED DESCRIPTION

The present disclosure relates to the field of immunotherapy for hepatitis B virus (HBV) and hepatitis delta virus (HDV). Disclosed binding proteins, e.g., antibodies, antigen-binding fragments, and fusion proteins, are capable of binding to an epitope located in the antigenic loop region of the S domain of the HBV envelope protein (HBsAg), are capable of neutralizing a HBV infection and, in some embodiments, a HDV infection.

Presently disclosed binding proteins possess advantageous production properties (e.g., reduced formation of antibody dimers and/or increased production in a host cell), such as compared to a reference anti-HBV antibody comprising the CDRs and, optionally, the VH and VL of “HBC34-v35”, disclosed in PCT Publication No. WO 2020/132091). Briefly, HBC34-v35 antibody has favorable binding and neutralization properties, but, as disclosed herein, can form antibody dimers through inter-light chain interactions during antibody production/purification. HBC34-v35 dimers have reduced ability to bind to HBsAg as compared to HBC34-v35 antibody monomers. Reducing dimer formation may improve, e.g., efficiency of antibody (or antigen-binding fragment) production and potency of a dose of the antibody (or antigen-binding fragment).

In certain embodiments, presently disclosed binding proteins can bind to any or all of the known HBsAg genotypes, as well as HBsAg variants, and can neutralize HBV infection, as well as HDV infection. In certain embodiments, a presently disclosed binding protein can bind to and/or can neutralize HBV and/or HDV with similar or even increased potency as compared to HBC34-v35.

Nucleic acids that encode, and host cells that express, such binding proteins are also provided herein. In addition, the present disclosure provides methods of using the binding proteins described herein in the diagnosis, prophylaxis, and treatment of diseases, as well as in methods of screening.

For example, embodiments of the antibodies, antigen-binding fragments, and fusion proteins according to the present description may be used in methods of preventing, treating, or attenuating, or diagnosing HBV and HDV. In particular embodiments, the antibodies, antigen-binding fragments, and fusion proteins described herein bind to two or more different genotypes of hepatitis B virus surface antigen and to two or more different infectious mutants of hepatitis B virus surface antigen. In specific embodiments, the antibodies, antigen-binding fragments, and fusion proteins described herein bind to all known genotypes of hepatitis B virus surface antigen and to all known infectious mutants of hepatitis B virus surface antigen.

The present disclosure also provides a method of treating chronic HBV infection in a subject in need thereof, comprising: administering to the subject an anti-HBV antibody or antigen-binding fragment in combination with an agent that reduces HBV antigenic load. The present disclosure also provides a method of treating chronic HBV infection in a subject in need thereof, comprising: administering to the subject an anti-HBV antibody or antigen-binding fragment in combination with an inhibitor of HBV gene expression.

In some methods, compositions for use, or uses described herein, the agent that reduces HBV antigenic load or the inhibitor of HBV gene expression is an RNAi agent (e.g., an siRNA, such as HBV001 or HBV002 or HBV003).

Prior to setting forth this disclosure in more detail, it may be helpful to an understanding thereof to provide definitions of certain terms to be used herein. Additional definitions are set forth throughout this disclosure.

Unless defined otherwise, all technical and scientific terms used herein have the same meaning as commonly understood by one of ordinary skill in the art.

Throughout this disclosure, unless the context requires otherwise, the term “comprise,” and variations thereof, such as “comprises,” and “comprising,” is used synonymously with, e.g. “having,” “has,” “including,” “includes,” or the like, and will be understood to imply the inclusion of a stated member, ratio, integer (including, where appropriate, a fraction thereof; e.g., one tenth and one hundredth of an integer), concentration, or step but not the exclusion of any other non-stated member, ratio, integer, concentration, or step. Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The terms “a” and “an” and “the” and similar reference used in the context of describing the disclosure (including in the context of the claims) are to be construed to cover both the singular and the plural, unless otherwise indicated herein or clearly contradicted by context. The use of the alternative (e.g., “or”) should be understood to mean either one, both, or any combination of the alternatives. Recitation of ranges of values herein is intended to serve as a shorthand method of referring individually to each separate value falling within the range. Unless otherwise indicated herein, each individual value is incorporated into the disclosure as if it were individually recited herein. No language in the specification should be construed as indicating any non-claimed element as essential to the practice of the subject matter disclosed herein.

The word “substantially” does not exclude “completely”; e.g., a composition which is “substantially free” from Y may be completely free from Y. In certain embodiments, “substantially” refers to a given amount, effect, or activity of a composition, method, or use of the present disclosure as compared to that of a reference composition, method, or use, and describes a reduction in the amount, effect, or activity of no more than 50%, such as no more than 40%, 30%, 25%, 20%, 15%, 10%, 5%, or 1%, or less, of the amount, effect, or activity of the reference composition, method, or use.

As used herein, the term “about” means±20% of the indicated range, value, or structure, unless otherwise indicated. In certain embodiments, “about” includes ±15%, ±10%, or ±5%.

“Optional” or “optionally” means that the subsequently described element, component, event, or circumstance may or may not occur, and that the description includes instances in which the element, component, event, or circumstance occurs and instances in which they do not.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

As used herein, the terms “peptide,” “polypeptide,” and “protein,” and variations of these terms, refer to a molecule that comprises at least two amino acids joined to each other by a (normal or modified) peptide bond. Accordingly, a protein or polypeptide comprises a polymer of amino acid residues. For example, a peptide, polypeptide or protein may comprise or be composed of a plurality of amino acids selected from the 20 amino acids defined by the genetic code or an amino acid analog or mimetic, each being linked to at least one other by a peptide bond. A peptide, polypeptide or protein can comprise or be composed of L-amino acids and/or D-amino acids (or analogs or mimetics thereof). The terms “peptide”, “polypeptide,” “protein” also include “peptidomimetics” which are defined as peptide analogs containing non-peptidic structural elements, which peptides are capable of mimicking or antagonizing the biological action(s) of a natural parent peptide. In certain embodiments, a peptidomimetic lacks characteristics such as enzymatically scissile peptide bonds.

A peptide, polypeptide or protein may comprise amino acids other than the 20 amino acids defined by the genetic code in addition to these amino acids, or it can be composed of amino acids other than the 20 amino acids defined by the genetic code. In certain embodiments, a peptide, polypeptide or protein in the context of the present disclosure can comprise amino acids that are modified by natural processes, such as post-translational maturation processes, or by chemical processes (e.g., synthetic processes), which are known in the art and include those described herein. Such modifications can appear anywhere in the polypeptide; e.g., in the peptide skeleton; in the amino acid chain; or at the carboxy- or amino-terminal ends. A peptide or polypeptide can be branched, such as following an ubiquitination, or may be cyclic, with or without branching. The terms “peptide”, “polypeptide”, and “protein” also include modified peptides, polypeptides and proteins. For example, peptide, polypeptide or protein modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications have been described in the literature (see Proteins Structure and Molecular Properties (1993) 2nd Ed., T. E. Creighton, New York; Post-translational Covalent Modifications of Proteins (1983) B. C. Johnson, Ed., Academic Press, New York; Seifter et al. (1990) Analysis for protein modifications and nonprotein cofactors, Meth. Enzymol. 182: 626-646 and Rattan et al., (1992) Protein Synthesis: Post-translational Modifications and Aging, Ann NY Acad Sci, 663: 48-62). Accordingly, the terms “peptide”, “polypeptide”, “protein” can include for example lipopeptides, lipoproteins, glycopeptides, glycoproteins and the like. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

As used herein, “(poly)peptide” and “protein” may be used interchangeably in reference to a polymer of amino acid residues, such as a plurality of amino acid monomers linked by peptide bonds.

“Nucleic acid molecule” or “polynucleotide” or “nucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, or the like.

Nucleic acid molecules include polyribonucleic acid (RNA), polydeoxyribonucleic acid (DNA), which includes cDNA, genomic DNA, and synthetic DNA, any of which may be single or double-stranded. If single-stranded, the nucleic acid molecule may be the coding strand or non-coding (anti-sense strand). Also contemplated are microRNA, siRNA, viral genomic RNA, and synthetic RNA. Polynucleotides (including oligonucleotides), and fragments thereof may be generated, for example, by polymerase chain reaction (PCR) or by in vitro translation, or generated by any of ligation, scission, endonuclease action, or exonuclease action.

A nucleic acid molecule encoding an amino acid sequence includes all nucleotide sequences that encode the same amino acid sequence. Some versions of the nucleotide sequences may also include intron(s) to the extent that the intron(s) may be removed through co- or post-transcriptional mechanisms. Different nucleotide sequences may encode the same amino acid sequence as the result of the redundancy or degeneracy of the genetic code, or by splicing, or both.

Variants of nucleic acid molecules of this disclosure are also contemplated. Variant nucleic acid molecules are at least 70%, 75%, 80%, 85%, 90%, 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a fusion protein or a binding domain thereof having a functionality described herein, such as specifically binding a target molecule.

As used herein, the term “sequence variant” refers to any sequence having one or more alterations in comparison to a reference sequence, whereby a reference sequence is any published sequence and/or of the sequences listed in the “Table of Sequences and SEQ ID Numbers” (sequence listing) herein. Thus, the term “sequence variant” includes nucleotide sequence variants and amino acid sequence variants. In certain embodiments, a sequence variant in the context of a nucleotide sequence, the reference sequence is also a nucleotide sequence, whereas in certain embodiments for a sequence variant in the context of an amino acid sequence, the reference sequence is also an amino acid sequence. A “sequence variant” as used herein can be at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the reference sequence.

“Percent sequence identity” refers to a relationship between two or more sequences, as determined by comparing the sequences. Methods to determine sequence identity can be designed to give the best match between the sequences being compared. For example, the sequences may be aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

A “sequence variant” in the context of a nucleic acid (nucleotide) sequence has an altered sequence in which one or more of the nucleotides in the reference sequence is deleted, or substituted, or one or more nucleotides are inserted into the sequence of the reference nucleotide sequence. Nucleotides are referred to herein by the standard one-letter designation (A, C, G, or T). Due to the degeneracy of the genetic code, a “sequence variant” of a nucleotide sequence can either result in a change in the respective reference amino acid sequence, i.e. in an amino acid “sequence variant” or not. In certain embodiments, a nucleotide sequence variant does not result in an amino acid sequence variant (e.g., a silent mutation). In some embodiments, a nucleotide sequence variant that results in one or more “non-silent” mutation is contemplated. In some embodiments, a nucleotide sequence variant of the present disclosure encodes an amino acid sequence that is at least 80%, at least 85%, at least 90%, at least 91%, at least 92%, at least 93%, at least 94%, at least 95%, at least 96%, at least 97%, at least 98%, or at least 99% identical to a reference amino acid sequence. Nucleotide and amino sequences as disclosed herein refer also to codon-optimized versions of a reference or wild-type nucleotide or amino acid sequence. In any of the embodiments described herein, a polynucleotide of the present disclosure may be codon-optimized for a host cell containing the polynucleotide. Codon optimization can be performed using known techniques and tools, e.g., using the GenScript® OptimumGene™ tool, or the GeneArt Gene Synthesis Tool (Thermo Fisher Scientific). Codon-optimized sequences include sequences that are partially codon-optimized (i.e., at least one codon is optimized for expression in the host cell) and those that are fully codon-optimized.

A “sequence variant” in the context of an amino acid sequence has an altered sequence in which one or more of the amino acids is deleted, substituted, or inserted in comparison to a reference amino acid sequence. As a result of the alterations, such a sequence variant has an amino acid sequence which is at least 80%, at least 85%, at least 90%, at least 91%, at least 92%, at least 93%, at least 94%, at least 95%, at least 96%, at least 97%, at least 98%, or at least 99% identical to the reference amino acid sequence. For example, per 100 amino acids of the reference sequence a variant sequence that has no more than 10 alterations, i.e. any combination of deletions, insertions or substitutions, is “at least 90% identical” to the reference sequence.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

Amino acid sequence insertions can include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

In general, alterations in the sequence variants do not abolish or significantly reduce a desired functionality of the respective reference sequence. For example, it is preferred that a variant sequence of the present disclosure does not significantly reduce or abrogate the functionality of a sequence of an antibody, or antigen-binding fragment thereof, to bind to the same epitope, to sufficiently neutralize infection of HBV and HDV, and/or does not cause or increase formation of antibody dimer, and/or is not produced at a lower titer in a host cell, as compared to antibody or antigen binding fragment having (or encoded by) the reference sequence.

As used herein, a nucleic acid sequence or an amino acid sequence “derived from” a specified nucleic acid, peptide, polypeptide or protein refers to the origin of the nucleic acid, peptide, polypeptide or protein. A nucleic acid sequence or amino acid sequence which is derived from a particular sequence may have an amino acid sequence that is essentially identical to that sequence or a portion thereof, from which it is derived, whereby “essentially identical” includes sequence variants as defined above. A nucleic acid sequence or amino acid sequence which is derived from a particular peptide or protein may be derived from the corresponding domain in the particular peptide or protein. In this context, “corresponding” refers to possession of a same functionality or characteristic of interest. For example, an “extracellular domain” corresponds to another “extracellular domain” (of another protein), or a “transmembrane domain” corresponds to another “transmembrane domain” (of another protein). “Corresponding” parts of peptides, proteins and nucleic acids are thus easily identifiable to one of ordinary skill in the art. Likewise, a sequence “derived from” another (e.g., “source”) sequence can be identified by one of ordinary skill in the art as having its origin in the source sequence.

A nucleic acid sequence or an amino acid sequence derived from another nucleic acid, peptide, polypeptide or protein may be identical to the starting nucleic acid, peptide, polypeptide or protein (from which it is derived). However, a nucleic acid sequence or an amino acid sequence derived from another nucleic acid, peptide, polypeptide or protein may also have one or more mutations relative to the starting nucleic acid, peptide, polypeptide or protein (from which it is derived), in particular a nucleic acid sequence or an amino acid sequence derived from another nucleic acid, peptide, polypeptide or protein may be a functional sequence variant as described above of the starting nucleic acid, peptide, polypeptide or protein (from which it is derived). For example, in a peptide/protein, one or more amino acid residues may be substituted with other amino acid residues, or one or more amino acid residue insertions or deletions may occur.

As used herein, the term “mutation” relates to a change in a nucleic acid sequence and/or in an amino acid sequence in comparison to a reference sequence, e.g. a corresponding genomic, wild type, or reference sequence. A mutation, e.g. in comparison to a reference genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making or inducing a mutation, e.g. in a nucleic acid sequence or in an amino acid sequence. A mutation includes substitution, deletion and/or insertion of one or more nucleotides or amino acids, as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved, for example, by altering (e.g., by site-directed mutagenesis) a codon (e.g., by alterning one, two, or three nucleotide bases therein) of a nucleic acid molecule encoding one amino acid to provide a codon that encodes a different amino acid, or that encodes a same amino acid, or by synthesizing a sequence variant.

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (KD) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. “Isolated” can, in some embodiments, describe an antibody, antigen-binding fragment, fusion protein, polynucleotide, vector, host cell, or composition that is outside of a human body.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

The term “recombinant”, as used herein (e.g. a recombinant antibody, a recombinant protein, a recombinant nucleic acid, or the like), refers to any molecule (antibody, protein, nucleic acid, or the like) which is prepared, expressed, created or isolated by recombinant means, and which is not naturally occurring. “Recombinant” can be used synonymously with “engineered” or “non-natural” and can refer to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof A non-endogenous polynucleotide or gene, as well as the encoded polypeptide or activity, may be from the same species, a different species, or a combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the terms “cell,” “cell line,” and “cell culture” are used interchangeably and all such designations include progeny. Thus, the terms “transformants” and “transformed cells” and “host cells” include the primary subject cell and cultures derived therefrom without regard for the number of transfers. It is also understood that all progeny may not be precisely identical in DNA content, due to deliberate or inadvertent mutations. Variant progeny that have the same or substantially the same function, phenotype, or biological activity as screened for in the originally transformed cell are included. Where distinct designations are intended, it will be clear from the context.

The term “construct” refers to any polynucleotide that contains a recombinant nucleic acid molecule (or, if the context clearly indicates, a fusion protein of the present disclosure).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

### Antibodies and Antigen-Binding Fragments

Presently disclosed embodiments include antibodies, and antigen-binding fragments thereof, that are capable of binding to the antigenic loop region of HBsAg (HBsAg and the antigenic loop region are described in further detail here) and, optionally, neutralizing infection by a hepatitis B virus (HBV) of genotype D, A, B, C, E, F, G, H, I, or J, or any combination thereof; i.e., any one, any two, any three, any four, any five, any six, any seven, any eight, any nine, or all ten of these genotypes. As discussed further herein, presently disclosed antibodies and antigen-binding fragments possess other advantages, including, for example and not limited to, characteristics that favor production in a host cell, and reduced propensity to form undesirable aggregates, such as dimers.

As used herein, and unless the context clearly indicates otherwise, “antibody” refers to an intact antibody comprising at least two heavy (H) chains and two light (L) chains inter-connected by disulfide bonds (though it will be understood that heavy chain antibodies, which lack light chains, are still generally encompassed by the term “antibody”, though preferred embodiments of the present disclosure comprise both of a VH and a VL, and in some embodiments, both of a heavy chain and a light chain), as well as any antigen-binding portion or fragment of an intact antibody that has or retains the ability to bind to the antigen target molecule recognized by the intact antibody, such as, for example, a scFv, Fab, or F(ab′)2 fragment. Thus, the term “antibody” herein is used in the broadest sense and includes polyclonal and monoclonal antibodies, including intact antibodies and functional (antigen-binding) antibody fragments thereof, including fragment antigen-binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv, and other antibody formats known in the art. Unless otherwise stated, the term “antibody” should be understood to encompass functional antibody fragments thereof. The term “antibody” also encompasses intact or full-length antibodies, including antibodies of any class or subclass thereof, including IgG and sub-classes thereof (IgG1, IgG2, IgG2, IgG4), IgM, IgE, IgA, and IgD.

As used herein, in the context of an antibody, the terms “antigen-binding fragment,” “fragment,” and “antibody fragment” are used interchangeably to refer to any fragment of an antibody of the disclosure that retains the antigen-binding activity of the antibody. Examples of antibody fragments include, but are not limited to, a single chain antibody, Fab, Fab′, F(ab′)2, Fv or scFv.

Human antibodies are known (e.g., van Dijk, M. A., and van de Winkel, J. G., Curr. Opin. Chem. Biol. 5 (2001) 368-374). Human antibodies can be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). Human monoclonal antibodies may be prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5. The term “human antibody” as used herein also comprises such antibodies which are modified, e.g., in the variable region or the constant region, to generate properties according to the antibodies and antibody fragments of the present disclosure.

Antibodies according to the present disclosure can be of any isotype (e.g., IgA, IgG, IgM, IgE, IgD; i.e., comprising an α, γ, μ, ε, or δ heavy chain). Within the IgG isotype, for example, antibodies may be IgG1, IgG2, IgG3 or IgG4 subclass. In specific embodiments, an antibody of the present disclosure is an IgG1 antibody. Antibodies or antigen binding fragments provided herein may include a κ or a λ light chain. Preferably, an antibody or antigen-binding fragment can comprise a λ light chain. In certain embodiments, HBsAg-specific antibodies described herein are of the IgG isotype (e.g., IgG1M,17 1 allotype) and may block the release of HBV and HBsAg from infected cells. Accordingly, in certain embodiments, an antibody according to the present description can bind intracellularly and thereby block the release of HBV virions and HBsAg.

The terms “V_{L}” or “VL” and “V_{H}” or “VH” refer to the variable region (also called variable domain) from an antibody light chain and an antibody heavy chain, respectively; typically, these regions are involved directly in the binding of an antibody or antigen-binding fragment to an antigen. A VL (as well as a CL or a light chain) can be a kappa (κ) class (also “VK” herein) or a lambda (λ) class. The variable binding regions comprise discrete sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary amino acid sequence by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs, though it will be understood that in some cases, a binding site can be formed by or can comprise one, two, three, four, or five of the CDRs which CDR(s) may disposed in the VH, in the VL, or in both.

In certain embodiments, antibody CDRs and amino acid numbering of variable regions are according to the system developed by the Chemical Computing Group (“CCG”); e.g., using Molecular Operating Environment (MOE) software (www.chemcomp.com).

In certain embodiments, antibody CDRs and amino acid numbering of variable regions are according to the IMGT numbering scheme (see, e.g., Lefranc et al., Dev. Comp. Immunol. 27:55, 2003).

Equivalent residue positions can be annotated and for different molecules to be compared using Antigen receptor Numbering And Receptor Classification (ANARCI) software tool (2016, Bioinformatics 15:298-300).

As used herein, a “variant” of a CDR refers to a functional variant (as provided herein) of a CDR sequence having up to 1-3 amino acid substitutions, deletions, or combinations thereof.

In certain embodiments, the present disclosure provides an antibody, or an antigen-binding fragment thereof, comprising (i) a heavy chain variable region (VH) that comprises therein the amino acid sequence of SEQ ID NO.:34, the amino acid sequence of SEQ ID NO.:35 or SEQ ID NO.:36, and the amino acid sequence of SEQ ID NO.:37; and (ii) a light chain variable region (VL) that comprises therein the amino acid sequence any one of SEQ ID NOs.:41, 40, 42, and 43, the amino acid sequence according to any one of SEQ ID NOs:49, 44-48, and 50-53, and the amino sequence according to SEQ ID NO.:55 or 56,

wherein, optionally, the VL comprises a R60N substitution mutation, a R60A substitution mutation, a R60K substitution mutation, a S64A substitution mutation, a I74A substitution mutation, or any combination thereof, relative to SEQ ID NO.:58 and wherein the amino acid numbering of the substitution mutation(s) is according to SEQ ID NO.:58, and still further optionally wherein the VL does not comprise any further mutation(s) relative to SEQ ID NO.:58,

and wherein the antibody or antigen-binding fragment thereof is capable of binding to the antigenic loop region of HBsAg and, optionally, neutralizing infection by a hepatitis B virus (HBV) of genotype D, A, B, C, E, F, G, H, I, or J, or any combination thereof.

In some embodiments, the antibody or antigen-binding fragment comprises: (i) in the VH, the amino acid sequences according to SEQ ID NOs.:34, 35, and 37, respectively, and in the VL, the amino acid sequences according to SEQ ID NOs.:41, 45, and 55, respectively; (ii) in the VH, the amino acid sequences according to SEQ ID NOs.:34, 35, and 37, and in the VL, the amino acid sequences according to SEQ ID NOs.: 41, 46, and 55, respectively; (iii) in the VH, the amino acid sequences according to SEQ ID NOs.:34, 35, and 37, respectively, and in the VL, the amino acid sequences according to SEQ ID NOs.: 41, 47, and 55, respectively; (iv) in the VH, the amino acid sequences according to SEQ ID NOs.:34, 35, and 37, respectively, and in the VL, the amino acid sequences according to SEQ ID NOs.:41, 48, and 55, respectively; (v) in the VH, the amino acid sequences according to SEQ ID NOs.:34, 35, and 37, respectively, and in the VL, the amino acid sequences according to SEQ ID NOs.:41, 49, and 55, respectively; (vi) in the VH, the amino acid sequences according to SEQ ID NOs.:34, 35, and 37, respectively, and in the VL, the amino acid sequences according to SEQ ID NOs.:41, 50, and 55, respectively; (vii) in the VH, the amino acid sequences according to SEQ ID NOs.:34, 35, and 37, respectively, and in the VL, the amino acid sequences according to SEQ ID NOs.:41, 51, and 55, respectively; (viii) in the VH, the amino acid sequences according to SEQ ID NOs.:34, 35, and 37, respectively, and in the VL, the amino acid sequences according to SEQ ID NOs.:41, 52, and 55, respectively; or (ix) in the VH, the amino acid sequences according to SEQ ID NOs.:34, 35, and 37, respectively, and in the VL, the amino acid sequences according to SEQ ID NOs.:41, 53, and 55, respectively.

In certain embodiments, the present disclosure provides an antibody, or an antigen-binding fragment thereof, comprising (i) a heavy chain variable region (VH) comprising a CDRH1 amino acid sequence according to SEQ ID NO.:34, a CDRH2 amino acid sequence according to SEQ ID NO.:35 or 36, and a CDRH3 amino acid sequence according to SEQ ID NO.:37; and (ii) a light chain variable region (VL) comprising a CDRL1 amino acid sequence set forth in any one of SEQ ID NOs.:40-43, a CDRL2 amino acid sequence according to any one of SEQ ID NOs:45-53, and a CDRL3 amino acid sequence according to SEQ ID NO.:55 or 56, wherein the CDRs are according to CCG, wherein the antibody or antigen-binding fragment thereof is capable of binding to the antigenic loop region of HBsAg and neutralizing infection by a hepatitis B virus (HBV) of genotype D, A, B, C, E, F, G, H, I, or J, or any combination thereof.

In certain embodiments, the CDRH1, CDRH2, CDRH3, CDRL1, CDRL2, and CDRL3 amino acid sequences are according to SEQ ID NOs.: (i) 34, 35, 37, 41, 45, and 55, respectively; (ii) 34, 35, 37, 41, 46, and 55, respectively; (iii) 34, 35, 37, 41, 47, and 55, respectively; (iv) 34, 35, 37, 41, 48, and 55, respectively; (v) 34, 35, 37, 41, 49, and 55, respectively; (vi) 34, 35, 37, 41, 50, and 55, respectively; (vii) 34, 35, 37, 41, 51, and 55, respectively; (viii) 34, 35, 37, 41, 52, and 55, respectively; or (ix) 34, 35, 37, 41, 53, and 55, respectively, wherein CDRs are according to CCG.

Table 1 provides the CDR amino acid SEQ ID NOs. of certain antibodies, wherein CDRs are defined according to CCG.

TABLE 1
CDR (CCG numbering) amino acid SEQ
ID NOs. of Certain Antibodies

| Antibody | CDRH1 | CDRH2 | CDRH3 | CDRL1 | CDRL2 | CDRL3 |
|---|---|---|---|---|---|---|
| HBC34-v35 | 34 | 35 | 37 | 41 | 44 | 55 |
| HBC34-v36 | 34 | 35 | 37 | 41 | 45 | 55 |
| HBC34-v37 | 34 | 35 | 37 | 41 | 46 | 55 |
| HBC34-v38 | 34 | 35 | 37 | 41 | 47 | 55 |
| HBC34-v39 | 34 | 35 | 37 | 41 | 48 | 55 |
| HBC34-v40 | 34 | 35 | 37 | 41 | 49 | 55 |
| HBC34-v41 | 34 | 35 | 37 | 41 | 50 | 55 |
| HBC34-v42 | 34 | 35 | 37 | 41 | 51 | 55 |
| HBC34-v43 | 34 | 35 | 37 | 41 | 52 | 55 |
| HBC34-v44 | 34 | 35 | 37 | 41 | 53 | 55 |
| HBC34-v45 | 34 | 35 | 37 | 41 | 44 | 55 |
| HBC34-v46 | 34 | 35 | 37 | 41 | 44 | 55 |
| HBC34-v47 | 34 | 35 | 37 | 41 | 51 | 55 |
| HBC34-v48 | 34 | 35 | 37 | 41 | 44 | 55 |
| HBC34-v49 | 34 | 35 | 37 | 41 | 51 | 55 |
| HBC34-v50 | 34 | 35 | 37 | 41 | 44 | 55 |

In certain embodiments, an antibody or antigen-binding fragment comprises CDRH1, CDRH2, CDRH3, CDRL1, CDRL2, and CDRL3 of: HBC34-v36; HBC34-v37; HBC34-v38; HBC34-v39; HBC34-v40; HBC34-v41; HBC34-v42; HBC34-v43; HBC34-v44; HBC34-v45; HBC34-v46; HBC34-v47; HBC34-v48; HBC34-v49; or HBC34-v50, wherein the CDRs are according to CCG, optionally wherein the VL further comprises a R60N substitution mutation, a R60A substitution mutation, a R60K substitution mutation, a S64A substitution mutation, a I74A substitution mutation, or any combination thereof, relative to SEQ ID NO.:58 and wherein the amino acid numbering of the substitution mutation(s) is according to SEQ ID NO.:58, and further optionally wherein the VL does not comprise any other mutation(s) relative to SEQ ID NO.:58.

Table 2 provides the CDR amino acid SEQ ID NOs. of certain antibodies, wherein CDRs are defined according to IMGT (short and long versions of CDRH2 and CDRL2 are disclosed).

TABLE 2
CDR (IMGT numbering) amino acid
SEQ ID NOs. of Certain Antibodies

| | | CDRH2 | | | CDRL2 | |
|---|---|---|---|---|---|---|
| | | (short/ | | | (short/ | |
| Antibody | CDRH1 | long) | CDRH3 | CDRL1 | long) | CDRL3 |
| HBC34-v35 | 150 | 151/152 | 153 | 154 | 155/163 | 173 |
| HBC34-v36 | 150 | 151/152 | 153 | 154 | 156/164 | 173 |
| HBC34-v37 | 150 | 151/152 | 153 | 154 | 157/165 | 173 |
| HBC34-v38 | 150 | 151/152 | 153 | 154 | 158/166 | 173 |
| HBC34-v39 | 150 | 151/152 | 153 | 154 | 159/167 | 173 |
| HBC34-v40 | 150 | 151/152 | 153 | 154 | 156/168 | 173 |
| HBC34-v41 | 150 | 151/152 | 153 | 154 | 158/169 | 173 |
| HBC34-v42 | 150 | 151/152 | 153 | 154 | 160/170 | 173 |
| HBC34-v43 | 150 | 151/152 | 153 | 154 | 161/171 | 173 |
| HBC34-v44 | 150 | 151/152 | 153 | 154 | 162/172 | 173 |
| HBC34-v45 | 150 | 151/152 | 153 | 154 | 155/163 | 173 |
| HBC34-v46 | 150 | 151/152 | 153 | 154 | 155/163 | 173 |
| HBC34-v47 | 150 | 151/152 | 153 | 154 | 160/170 | 173 |
| HBC34-v48 | 150 | 151/152 | 153 | 154 | 155/163 | 173 |
| HBC34-v49 | 150 | 151/152 | 153 | 154 | 160/170 | 173 |
| HBC34-v50 | 150 | 151/152 | 153 | 154 | 155/163 | 173 |

In certain embodiments, an antibody or antigen-binding fragment comprises CDRH1, CDRH2, CDRH3, CDRL1, CDRL2, and CDRL3 of: HBC34-v36; HBC34-v37; HBC34-v38; HBC34-v39; HBC34-v40; HBC34-v41; HBC34-v42; HBC34-v43; HBC34-v44; HBC34-v45; HBC34-v46; HBC34-v47; HBC34-v48; HBC34-v49; or HBC34-v50, wherein the CDRs are according to IMGT, optionally wherein the VL further comprises a R60N substitution mutation, a R60A substitution mutation, a R60K substitution mutation, a S64A substitution mutation, a I74A substitution mutation, or any combination thereof, relative to SEQ ID NO.:58 and wherein the amino acid numbering of the substitution mutation(s) is according to SEQ ID NO.:58, and further optionally wherein the VL does not comprise any other mutation(s) relative to SEQ ID NO.:58.

Table 3 provides the VH and VL amino acid SEQ ID NOs. of certain antibodies.

TABLE 3
VH and VL amino acid SEQ ID NOs. of Certain Antibodies

| Antibody | VH | VL |
|---|---|---|
| HBC34-v35 | 38 | 57 |
| HBC34-v36 | 38 | 58 |
| HBC34-v37 | 38 | 59 |
| HBC34-v38 | 38 | 60 |
| HBC34-v39 | 38 | 61 |
| HBC34-v40 | 38 | 62 |
| HBC34-v41 | 38 | 63 |
| HBC34-v42 | 38 | 64 |
| HBC34-v43 | 38 | 65 |
| HBC34-v44 | 38 | 66 |
| HBC34-v45 | 38 | 67 |
| HBC34-v46 | 38 | 68 |
| HBC34-v47 | 38 | 69 |
| HBC34-v48 | 38 | 70 |
| HBC34-v49 | 38 | 71 |
| HBC34-v50 | 38 | 72 |

In certain embodiments, an antibody or antigen-binding fragment comprises the VH and VL amino acid sequences of: HBC34-v36; HBC34-v37; HBC34-v38; HBC34-v39; HBC34-v40; HBC34-v41; HBC34-v42; HBC34-v43; HBC34-v44; HBC34-v45; HBC34-v46; HBC34-v47; HBC34-v48; HBC34-v49; or HBC34-v50.

In certain embodiments, antibody or antigen-binding fragment comprises a heavy chain variable domain (VH) and a light chain variable domain (VL), wherein: (i) the VH comprises or consists of an amino acid sequence having at least 90% (i.e., 90%, 91%, 92%, 93%, 94%, 95%, 96%, 97%, 98%, 99%, or 100%, or any non-integer value therebetween) identity to the amino acid sequence set forth in SEQ ID NO.: 38 or 39; and/or (ii) the VL comprises or consists of an amino acid sequence having at least 90% (i.e., 90%, 91%, 92%, 93%, 94%, 95%, 96%, 97%, 98%, 99%, or 100%, or any non-integer value therebetween) identity to the amino acid sequence set forth in any one of SEQ ID NOs.: 58-66, 69, 71, or 72. In particular embodiments, the VH and the VL comprise or consist of amino acid sequences having at least 90% (i.e., 90%, 91%, 92%, 93%, 94%, 95%, 96%, 97%, 98%, 99%, or any non-integer value therebetween) identity to the amino acid sequences set forth in SEQ ID NOs.: (i) 38 and 58, respectively; (ii) 38 and 59, respectively; (iii) 38 and 60, respectively; (iv) 38 and 61, respectively; (v) 38 and 62, respectively; (vi) 38 and 63, respectively; (vii) 38 and 64, respectively; (viii) 38 and 65, respectively; (ix) 38 and 66, respectively; (x) 38 and 71, respectively; or (xi) 38 and 72, respectively. As a non-limiting example, in certain embodiments, the VH comprises an amino acid sequence having at least 90% identity to SEQ ID NO.:38 and the VL comprises an amino acid sequence having at least 90% identity to SEQ ID NO.:62.

In some embodiments, the VH comprises or consists of the amino acid sequence set forth in SEQ ID NO.: 38 or 39; and/or the VL comprises or consists of the amino acid sequence set forth in any one of SEQ ID NOs.: 58-66, 69, 71, or 72. In particular embodiments, the VH and the VL comprise or consist of the amino acid sequences set forth in SEQ ID NOs.: (i) 38 and 58, respectively; (ii) 38 and 59, respectively; (iii) 38 and 60, respectively; (iv) 38 and 61, respectively; (v) 38 and 62, respectively; (vi) 38 and 63, respectively; (vii) 38 and 64, respectively; (viii) 38 and 65, respectively; (ix) 38 and 66, respectively; (x) 38 and 71, respectively; or (xi) 38 and 72, respectively.

In certain embodiments, an antibody or antigen-binding fragment comprises a VH comprising or consisting of the amino acid sequence set forth in SEQ ID NO.:38 and a VL comprising or consisting of the amino acid sequence set forth in any one of SEQ ID NOs.:58-72.

In certain embodiments, an antibody or antigen-binding fragment comprises a VH comprising or consisting of the amino acid sequence set forth in SEQ ID NO.:38 and a VL comprising or consisting of the amino acid sequence set forth in any one of SEQ ID NOs.:59-72.

In another aspect, the present disclosure provides an antibody or antigen-binding fragment, comprising: a heavy chain variable domain (VH) and a light chain variable domain (VL), wherein the VH and the VL comprise or consist of the amino acid sequences set forth in SEQ ID NOs.: (i) 38 and 67, respectively; or (ii) 38 and 68, respectively, wherein the antibody or antigen-binding fragment thereof is capable of binding to the antigenic loop region of HBsAg and neutralizing infection by a hepatitis B virus (HBV) of genotype D, A, B, C, E, F, G, H, I, or J, or any combination thereof.

Also provided is an antibody or antigen-binding fragment comprising a VH according to SEQ ID NO.:38 or 39 and a VL variant of any one of SEQ ID NOs.:57-72 that comprises any one or more of the following mutations (in framework region 3, as determined by CCG numbering): R60A, R60N, R60K, S64A, I74A. In certain further embodiments, the VL variant does not comprise any further mutations as compared to SEQ ID NO.:57-72 (respectively))

Also provided is an antibody or antigen-binding fragment comprising a VH according to SEQ ID NO.:38 and a VL variant of any one of SEQ ID NOs.:57-72 that comprises a substitution mutation (such as, for example, a conservative amino acid substitution, or a mutation to a germline-encoded amino acid) at Q78, D81, or both (CCG numbering).

Also provided is an antibody or antigen-binding fragment comprising a VH according to SEQ ID NO.:39 and a VL variant of any one of SEQ ID NOs.:57-72 that comprises a substitution mutation (such as, for example, a conservative amino acid substitution, or a mutation to a germline-encoded amino acid) at Q78, D81, or both (CCG numbering).

As discussed further herein in, presently disclosed antibodies and antigen-binding fragments have a reduced propensity to form aggregates (e.g., dimers), and/or have improved productivity (e.g., higher titer) in a host cell, and/or have similar or substantially identical or even improved: binding to HBsAg; HBV neutralization; and/or thermostability, as compared to a reference antibody disclosed herein.

It will be understood that a “reference” antibody or antigen-binding fragment refers to an antibody or antigen-binding fragment that is identical to the subject antibody or antigen-binding fragment, respectively, with the exception of the identified or enumerated features (e.g., differences in CDR and/or variable region framework sequence(s)). In some embodiments, the reference antibody comprises the CDRH1, CDRH2, CDRH3, CDRL1, CDRL2, and CDRL3 amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

As a non-limiting example, an antibody or antigen-binding fragment of the present disclosure can be an IgG1 isotype and comprise a wild-type IgG1 Fc moiety, and a reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57, and is the IgG1 isotype and comprises a wild-type IgG1 Fc moiety. It will further be understood that when comparing a presently disclosed antibody or antigen-binding fragment with a reference antibody or antigen-binding fragment under certain conditions, the conditions (e.g., amount of starting material, temperature, buffer, identity of host cell line, culture conditions, duration of a relevant time period, codon-optimization of an encoding polynucleotide, or the like) will, unless explicitly stated otherwise, be identical as between the presently disclosed molecule(s) and the reference molecule(s), or as close to identical as the conditions permit (e.g., two antibodies may differ in their amino acid sequences by one or a number of amino acids, but will be otherwise identical, and will be encoded by a comparable polynucleotide (e.g., each antibody can be encoded by a respective codon-optimized polynucleotide).

As a non-limiting example, presently disclosed antibodies and antigen-binding fragments produce fewer aggregates (e.g., in the form of antibody:antibody dimers, antibody:antigen-binding fragment dimers, or antigen-binding fragment:antigen-binding fragment dimers), and/or have a higher production titer in a host cell, as compared to a reference antibody or antigen-binding fragment, respectively.

In this context, a dimer is a complex or aggregate comprising two antibody or antigen-binding fragment molecules (e.g., an antibody:antibody dimer, a Fab:Fab dimer, or an antibody:Fab dimer). As discussed further herein, dimerization in this context is distinct from typical associations between antibody heavy chain and light chain components, or between two antibody heavy chain polypeptides, that occur in the formation of an intact tetrameric antibody, Fv, or Fab and may involve associations between two monomers. Accordingly, it will be understood that in the present context, a “dimer” or does not refer to the association of an antibody heavy chain with an antibody light chain to provide a half-antibody comprising a functional Fab, and also does not include association of two heavy chains of an antibody (e.g., hinge-hinge and Fc-Fc) or VH-VL associations (e.g. that occur via disulfide bonds), such as in a Fv or in a Fab.

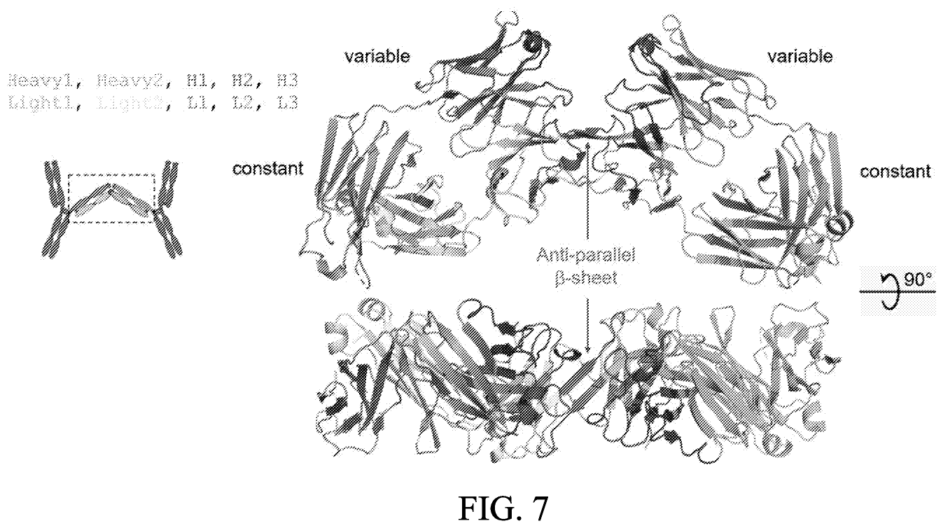

In certain embodiments, a dimer is formed by association between the VLs of two discrete antibody or antigen-binding fragment molecules. An illustration of a dimer formed by association of two VLs of discrete antibody molecules is shown in present FIG. 7. Such dimerization can, for example, reduce binding valency and/or binding affinity and/or avidity and/or neutralization potency of one or both of the antibody or antigen-binding fragment molecules comprised therein. In general, an increased presence of such dimers in a composition comprising a plurality of antibodies or antigen-binding fragments reduces the overall binding and/or neutralizing potency of the composition.

Antibody or antigen-binding fragment dimers can be identified using, known techniques, such as, for example, size-exclusion chromatography. A dimer will have a molecular weight that is higher than the molecular weight of each individual (monomer) subunit thereof, and will typically equal or approximate the sum of the molecular weights of each individual subunit thereof. For example, a homo-dimer (i.e., which comprises two antibody molecules that are identical or substantially identical in their amino acid sequences) will generally have a molecular weight that is about twice the molecular weight of each monomeric subunit thereof. For example, a typical human IgG1 immunoglobulin molecule has a molecular weight of around 150 kilodaltons (for example, with each of the two heavy chains weighing around 50 kilodaltons, and each of the two light chains weighing around 25 kilodaltons), and a dimer comprising two such immunoglobulin molecules will have a molecular weight of around 300 kilodaltons. Of course, it will be understood that one antibody may have a slightly or somewhat different molecular weight than a different antibody of the same general structure and isotype, e.g., due at least in-part to any differences in the respective amino acid sequences.

As another, non-limiting, example, an antibody molecule may have a molecular weight of between 140 kilodaltons and 160 kilodaltons, and an antibody dimer comprising two antibody molecules may have a molecular weight of between 280 kilodaltons and 320 kilodaltons. Dimers may be referred-to as “high-molecular weight species” or “HMWS”.

The presence of dimers in a composition or sample comprising a plurality of antibody (and/or antigen-binding fragment) molecules can be evaluated using, for example, absolute size exclusion chromatography (aSEC). The amount of dimer in a composition or sample can be expressed as the percentage of total antibody or antigen-binding fragment molecules in the composition or sample that are comprised in a dimer. By way of illustration, for an antibody composition comprising 12% dimers, 88% of the total antibody molecules in the sample are present as monomers.

In any of the presently disclosed embodiments, in a sample comprising a plurality of the antibody or antigen-binding fragment (i.e., a plurality of antibody or antigen-binding fragment molecules), less than 12%, 11% or less, 10% or less, 9% or less, 8% or less, 7% or less, 6% or less, 5% or less, 4% or less, 3% or less, or 2% or less of the plurality is comprised in a dimer when the sample has been incubated for about 120 to about 168 hours at about 40° C., wherein, optionally, the presence of dimer is determined by absolute size-exclusion chromatography.

In any of the presently disclosed embodiments, incubation of a plurality of presently disclosed antibody or antigen-binding fragment molecules results in reduced formation of dimers as compared to incubation of a plurality of a reference antibody or antigen-binding fragment molecules, wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57, and wherein, optionally, the presence of antibody dimer is determined by absolute size-exclusion chromatography. Such a reference antibody or antigen-binding fragment (e.g., Fv, Fab) can form dimers that, in some embodiments, collectively comprise more than 2%, 3% or more, 4% or more, 5% or more, 6% or more, 7% or more, 8% or more, 9% or more, 10% or more, 11% or more, or up to 12% of the antibody or antigen-binding fragment molecules in a sample (e.g., when incubated for about 120 to about 168 hours at about 40° C.). In other words, in some embodiments, up to 12% or more of reference antibody or antigen-binding fragment molecules are comprised in a dimer, while a lesser percentage, preferably 2% or less, of presently disclosed antibody or antigen-binding fragment molecules are comprised in a dimer.

In some embodiments, a presently disclosed antibody or antigen-binding fragment forms a lower amount of dimer, and/or forms dimers at a reduced frequency and/or as a lower percentage of total antibody or antigen-binding fragment molecules in a sample or composition, (e.g., as determined using Size Exclusion Chromatography) as compared to a reference antibody: (i) in a 5-day, a 15-day, and/or a 32-day incubation at 4° C.; (ii) in a 5-day, a 15-day, and/or a 32-day incubation at 25° C.; and/or (iii) in a 5-day, a 15-day, and/or a 32-day incubation at 40° C., wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

In some embodiments, the percentage of presently disclosed antibody or antigen-binding fragment molecules in a composition that are comprised in a dimer is less than ⅘, less than ¾, less than ½, less than ⅓, less than ¼, less than ⅕, less than ⅙, less than 1/7, less than ⅛, less than 1/9, or less than 1/10 the percentage of the reference antibody molecules in a composition that are present in a dimer, respectively. As a non-limiting example, following a 32-day (768-hour) incubation at 40° C., 22% or more of the reference antibody molecules in a composition can be comprised in a dimer, while 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, or 2% or less of presently disclosed antibody or antigen-binding fragment molecules in a composition are comprised in a dimer, respectively.

In some embodiments, a host cell (e.g., a CHO cell such as an ExpiCHO cell) transfected with a polynucleotide encoding a presently disclosed antibody or antigen-binding fragment provides 1.5× or more, 2× or more, 3× or more, or 4× or more the amount of antibody or antigen-binding fragment, respectively, (e.g., measured as a concentration in mg/mL) than a reference host cell transfected with a polynucleotide encoding a reference antibody or antigen-binding fragment, wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

In some embodiments, a presently disclosed antibody or antigen-binding fragment thereof is produced in transfected cells at a higher titer as compared to a reference antibody or antigen-binding fragment is produced in reference transfected cells, wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

In some embodiments, a presently disclosed antibody or antigen-binding fragment thereof is produced in transfected cells at titers of at least 1.5-fold, at least 2-fold, at least 3-fold, or at least 4-fold, higher than the titer at which a reference antibody or antigen-binding fragment is produced, wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

In any of the presently disclosed embodiments, the antibody or antigen-binding fragment is capable of binding to a HBsAg (e.g., of subtype adw) with an EC50 (ng/ml) of 3.5 or less, about 3.2 or less, less than 3.0, less than 2.5, less than 2.0, less than 1.5, or less than 1.0. In some embodiments, the antibody or antigen-binding fragment is capable of binding to a HBsAg (e.g., of subtype adw) with an EC50 (ng/ml) of less than 3.5, less than 3.4, less than 3.3, less than 3.2, less than 3.1, less than 3.0, less than 2.9, less than 2.8, less than 2.7, less than 2.6, less than 2.5, less than 2.4, less than 2.3, less than 2.1, less than 2.0, less than 1.9, less than 1.8, less than 1.7, less than 1.6, less than 1.5, less than 1.4, less than 1.3, less than 1.2, less than 1.1, or less than 1.0. In some embodiments, the antibody or antigen-binding fragment is capable of binding to a HBsAg (e.g., of subtype adw) with an EC50 (ng/ml) of between 0.9 and 2.0, or of between 0.9 and 1.9, or of between 0.9 and 1.8, or of between 0.9 and 1.7, or of between 0.9 and 1.6, or of between 0.9 and 1.5, or of between 0.9 and 1.4, or of between 0.9 and 1.3, or of between 0.9 and 1.2, or of between 0.9 and 1.1, or of between 0.9 and 1.0, or of between 1.0 and 2.0. In certain embodiments, the antibody or antigen-binding fragment is capable of binding to a HBsAg (e.g., of subtype adw) with an EC50 (ng/ml) of 2.0 or less. In some embodiments, a binding EC50 is determined by ELISA (e.g., direct antigen-binding ELISA assay, with binding curves determined by fitting the curves using Graphpad prism).

In any of the presently disclosed embodiments, the antibody or antigen-binding fragment thereof is capable of neutralizing hepatitis B virus infection with a neutralization of infection EC50 of less than 20 ng/ml, preferably 15 ng/ml or less, more preferably 10 ng/mL or less. In some embodiments, the antibody or antigen-binding fragment thereof is capable of neutralizing hepatitis B virus infection with a neutralization of infection EC50 of 18, 17, 16, 15, 14, 13, 12, 11, 10, 9, 8, or 7 ng/mL. In some embodiments, the antibody or antigen-binding fragment thereof is capable of neutralizing hepatitis B virus infection with a neutralization of infection EC50 that is lower than the neutralization of infection EC50 (using the same assay) of a reference antibody or antigen-binding fragment that comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57. In some embodiments, a neutralization of infection EC50 is determined following incubation of cultured cells, e.g., differentiated HepaRG cells, with a fixed amount of HBV in the presence or absence of the antibody to be tested. In such an embodiment, incubation may be carried out, for example, for 16 hours at 37° C. That incubation may be performed in a medium (e.g. supplemented with 4% PEG 8000). After incubation, cells may be washed and further cultivated. To measure virus infectivity, the levels of hepatitis B surface antigen (HBsAg) and/or hepatitis B e antigen (HBeAg) secreted into the culture supernatant, e.g. from day 7 to day 11 post-infection, may be determined by enzyme-linked immunosorbent assay (ELISA). Levels of HBsAg and/or HBeAg from treated cells can be compared to those of untreated cells to determine the presence and extent of neutralization.

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) can have the ability to recognize and bind antigen, although typically at a lower affinity than the entire binding site.

“Single-chain Fv” also abbreviated as “sFv” or “scFv”, are antibody fragments that comprise the VH and VL antibody domains connected into a single polypeptide chain. In some embodiments, the scFv polypeptide comprises a polypeptide linker disposed between and linking the VH and VL domains that enables the scFv to retain or form the desired structure for antigen binding. Such a peptide linker can be incorporated into a fusion polypeptide using standard techniques well known in the art. Additionally or alternatively, Fv can have a disulfide bond formed between and stabilizing the VH and the VL. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL-linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both.

Peptide linker sequences may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46 (1985); Murphy et al., Proc. Natl. Acad. Sci. USA 83:8258 8262 (1986); U.S. Pat. Nos. 4,935,233, and 4,751,180. Other illustrative and non-limiting examples of linkers may include, for example, those disclosed by Chaudhary et al., Proc. Natl. Acad. Sci. USA 87:1066-1070 (1990), and Bird et al., Science 242:423-426 (1988)) and a pentamer of four glycine residues linked in series, the C-terminal glycine of the series being linked to a single serine, when present in a single iteration or repeated 1 to 5 or more times, or more. Any suitable linker may be used, and in general can be about 3, 4, 5, 6, 7, 8, 9, 10, 11, 12, 13, 14, 15, 16, 17, 18, 19, 20, 21, 22, 15 23, 24, 25, 26, 27, 28, 29, 30, 40, 50, 60, 70, 80, 90, or 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human.

scFv can be constructed using any combination of the VH and VL sequences or any combination of the CDRH1, CDRH2, CDRH3, CDRL1, CDRL2, and CDRL3 sequences disclosed herein. In some embodiments, linker sequences are not required; for example, when the first and second polypeptides have non-essential N-terminal amino acid regions that can be used to separate the functional domains and prevent steric interference.

In some embodiments, an antibody or antigen-binding fragment comprises a light chain constant region (or a portion or fragment thereof), a heavy chain constant region (or a portion or fragment thereof), or both. The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). The Fc region of an antibody heavy chain is described further herein. In any of the presently disclosed embodiments, an antibody or antigen-binding fragment of the present disclosure comprises any one or more of CL, a CH1, a CH2, and a CH3. In certain embodiments, a CL comprises an amino acid sequence having at least 90%, 91%, 92%, 93%, 94%, 95%, 96%, 97%, 98%, 99%, or 100% identity to the amino acid sequence of SEQ ID NO.:79. In certain embodiments, a CH1-CH2-CH3 comprises an amino acid sequence having 90%, 91%, 92%, 93%, 94%, 95%, 96%, 97%, 98%, 99%, or 100% identity to the amino acid sequence of SEQ ID NO.:73, or a variant thereof that comprises one or more of the following amino acid substitutions (EU numbering): G236A; A330L; I332E; M428L; N434S. Fc moieties are described elsewhere herein.

It will be understood that, for example, production in a mammalian cell line can remove one or more C-terminal lysine of an antibody heavy chain (see, e.g., Liu et al. mAbs 6(5):1145-1154 (2014)). Accordingly, an antibody or antigen-binding fragment of the present disclosure can comprise a heavy chain, a CH1-CH3, a CH3, or an Fc polypeptide wherein a C-terminal residue is present or is absent; in other words, encompassed are embodiments wherein the C-terminal residue of a heavy chain, a CH1-CH3, or an Fc moiety is not a lysine, and embodiments where a lysine is the C-terminal residue. In certain embodiments, a composition comprises a plurality of an antibody and/or an antigen-binding fragment of the present disclosure, wherein one or more antibody or antigen-binding fragment does not comprise a lysine residue at the C-terminal end of the heavy chain, CH1-CH3, or Fc moiety, and wherein one or more antibody or antigen-binding fragment comprises a lysine residue at the C-terminal end of the heavy chain, CH1-CH3, or Fc moiety.

A “Fab” (fragment antigen binding) is the part of an antibody that binds to antigens and includes the variable region and CH1 of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known. Fab fragments may be joined, e.g., by a peptide linker, to form a single chain Fab, also referred to herein as “scFab.” In these embodiments, an inter-chain disulfide bond that is present in a native Fab may not be present, and the linker serves in full or in part to link or connect the Fab fragments in a single polypeptide chain. A heavy chain-derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment-linker-light chain Fab fragment) or (light chain Fab fragment-linker-heavy chain Fab fragment). Peptide linkers and exemplary linker sequences for use in scFabs are discussed in further detail herein.

In any of the presently disclosed embodiments, the antibody, or the antigen-binding fragment thereof, comprises a human antibody, a monoclonal antibody, a purified antibody, a single chain antibody, a Fab, a Fab′, a F(ab′)2, a Fv, or a scFv.

Fragments of the antibodies described herein can be obtained from the antibodies by methods that include digestion with enzymes, such as pepsin or papain, and/or by cleavage of disulfide bonds by chemical reduction. Alternatively, fragments of the antibodies can be obtained by cloning and expression of part of the sequences of the heavy or light chains. The present disclosure encompasses single-chain Fv fragments (scFv) derived from the heavy and light chains of an antibody as described herein, including, for example, an scFv comprising the CDRs (and, optionally, the variable regions) from an antibody according to the present description, heavy or light chain monomers and dimers (i.e., VH-VL dimer, HC-LC dimer, HC-HC dimer), single domain heavy chain antibodies, single domain light chain antibodies, as well as single chain antibodies, in which the heavy and light chain variable domains or regions are joined by a peptide linker.

In certain embodiments, an antibody according to the present disclosure, or an antigen binding fragment thereof, comprises a purified antibody, a monoclonal antibody, a single chain antibody, Fab, Fab′, F(ab′)2, Fv or scFv.

Antibodies and antigen binding fragments of the present disclosure may, in embodiments, be multispecific (e.g., bispecific, trispecific, tetraspecific, or the like), and may be provided in any multispecific format, as disclosed herein. In certain embodiments, an antibody or antigen-binding fragment of the present disclosure is a multispecific antibody, such as a bispecific or trispecific antibody. Formats for bispecific antibodies are disclosed in, for example, Spiess et al., Mol. Immunol. 67(2):95 (2015), and in Brinkmann and Kontermann, mAbs 9(2):182-212 (2017), which bispecific formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-IgGs, IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, and DVI-IgG (four-in-one). A bispecific or multispecific antibody may comprise a HBV- and/or HDV-specific binding domain of the instant disclosure in combination with another HBV- and/or HDV-specific binding domain of the instant disclosure, or in combination with a different binding domain that specifically binds to HBV and/or HDV (e.g., at a same or a different epitope), or with a binding domain that specifically binds to a different antigen.

Antibody fragments of the disclosure may impart monovalent or multivalent interactions and be contained in a variety of structures as described above. For instance, scFv molecules may be synthesized to create a trivalent “triabody” or a tetravalent “tetrabody”. The scFv molecules may include a domain of the Fc region resulting in bivalent minibodies. In addition, the sequences of the disclosure may be a component of multispecific molecules in which the sequences of the disclosure target the epitopes of the disclosure and other regions of the molecule bind to other targets. Exemplary molecules include, but are not limited to, bispecific Fab2, trispecific Fab3, bispecific scFv, and diabodies (Holliger and Hudson, 2005, Nature Biotechnology 9: 1126-1136).

Antibodies or antigen-binding fragments thereof such as those described herein, including but not limited to scFv, may, in certain embodiments, be comprised in a fusion protein that is capable of specifically binding to an antigen as described herein.

As used herein, “fusion protein” refers to a protein that, in a single chain, has at least two distinct domains or motifs, wherein the domains or motifs are not naturally found together, or in the given arrangement, in a protein. A polynucleotide encoding a fusion protein may be constructed using PCR, recombinantly engineered, or the like, or such fusion proteins can be synthesized.

In some embodiments, a fusion protein is capable of expression at a surface of a host cell, e.g., a T cell, NK cell, or NK-T cell. In certain embodiments, a fusion protein comprises (i) an extracellular component comprising the antibody or antigen binding fragment thereof (e.g., a scFv); (ii) a transmembrane component (e.g., a transmembrane domain from CD4, CD8, CD27, CD28, or a functional variant or portion thereof, or any combination thereof); and (iii) an intracellular component comprising a signaling domain from a costimulatory protein, or a functional variant or portion thereof (e.g., a signaling domain from CD27, CD28, 4-1BB (CD137), OX40 (CD134), CD2, CDS, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that specifically binds with CD83, or a functional variant thereof, or any combination thereof), and/or an effector domain (e.g., from CD3ε, CD3δ, CD3ζ, CD25, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof).

In certain embodiments, a fusion protein comprising an antibody or antigen binding fragment comprises a chimeric antigen receptor molecule (CAR), which may be expressed on a cell surface of a host cell such as a T cell, a NK cell, or a NK-T cell for use in a cellular immunotherapy. CAR molecules and principles of design are described in, for example: Sadelain et al., Cancer Discov., 3(4):388 (2013); Harris and Kranz, Trends Pharmacol. Sci., 37(3):220 (2016); Stone et al., Cancer Immunol. Immunother., 63(11):1163 (2014); Xu et al., 2018 Oncotarget 9:13991; Androulla et al., 2018 Curr. Pharm. Biotechnol. Volume 19 (April 2018); Wu et al., 2016 Expert Opin. Biol. Ther. 16:1469; and Ren et al., 2017 Protein Cell 8:634, which CAR molecules, CAR designs, and CAR design principles are herein incorporated by reference in their entirety.

Throughout this disclosure, antibodies, antigen binding fragments thereof, and fusion proteins may individually or collectively (e.g., in any combination) be referred to as “binding proteins”.

Binding proteins according to the present disclosure may be provided in purified form. For example, an antibody may be present in a composition that is substantially free of other polypeptides e.g., where less than 90% (by weight), usually less than 60% and more usually less than 50% of the composition is made up of other polypeptides.

Binding proteins according to the present disclosure may be immunogenic in human and/or in non-human (or heterologous) hosts; e.g., in mice. For example, an antibody may have an idiotope that is immunogenic in non-human hosts, but not in a human host. Antibodies of the disclosure for human use include those that are not typically isolated from hosts such as mice, goats, rabbits, rats, non-primate mammals, or the like, and in some instances are not obtained by humanization or from xeno-mice. Also contemplated herein are variant forms of the disclosed antibodies, which are engineered so as to reduce known or potential immunogenicity and/or other potential liabilities, or to confer a desired structure and/or functionality of the antibody in a non-human animal, such as a mouse (e.g., a “murinized” antibody wherein one or more human amino acid residue, sequence, or motif is replaced by a residue, sequence, or motif that has reduced or abrogated immunogenicity or other liability, or has a desired structure and/or function, in a mouse; e.g., for model studies using a mouse).

As used herein, a “neutralizing antibody” (or antigen binding fragment, or fusion protein) is one that can neutralize, i.e., prevent, inhibit, reduce, impede or interfere with, the ability of a pathogen to initiate and/or perpetuate an infection in a host (e.g., host organism or host cell). The terms “neutralizing antibody” and “an antibody that neutralizes” or “antibodies that neutralize” are used interchangeably herein. These antibodies can be used alone, or in combination (e.g., two or more of the presently disclosed antibodies in a combination, or an antibody of the present disclosure in combination with another agent, which may or may not be an antibody agent, including an antibody that is capable of neutralizing an HBV B and/or HBV D infection), as prophylactic or therapeutic agents upon appropriate formulation, in association with active vaccination, as a diagnostic tool, or as a production tool as described herein. Accordingly, presently disclosed antibodies or antigen-binding fragments are capable of neutralizing infection by a HBV, a HDV, or both.

As used herein, “specifically binds” or “specific for” refers to an association or union of a binding protein (e.g., an antibody or antigen binding fragment thereof) or a binding domain to a target molecule with an affinity or Ka (i.e., an equilibrium association constant of a particular binding interaction with units of 1/M) equal to or greater than 10⁵ M⁻¹ (which equals the ratio of the on-rate [Kₒₙ] to the off rate [Koff] for this association reaction), while not significantly associating or uniting with any other molecules or components in a sample. Binding proteins or binding domains may be classified as “high-affinity” binding proteins or binding domains or as “low-affinity” binding proteins or binding domains. “High-affinity” binding proteins or binding domains refer to those binding proteins or binding domains having a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” binding proteins or binding domains refer to those binding proteins or binding domains having a Ka of up to 10⁷ M⁻¹, up to 10⁶ M⁻¹, or up to 10⁵ M⁻¹. Alternatively, affinity may be defined as an equilibrium dissociation constant (Kd) of a particular binding interaction with units of M (e.g., 10⁻⁵ M to 10⁻¹³ M). The terms “binding” and “specifically binding” and similar references do not encompass non-specific sticking.

Binding of a binding protein can be determined or assessed using an appropriate assay, such as, for example, Surface Plasmon Resonance (SPR) methods, e.g., a Biacore™ system; kinetic exclusion assays such as KinExA®; and BioLayer interferometry (e.g., using the ForteBio® Octet platform); isothermal titration calorimetry (ITC), or the like, an antigen-binding ELISA (e.g., direct or indirect) with imaging by, e.g., optical density at 450 nm, or by flow cytometry, or the like.

In certain embodiments, binding proteins according to the present disclosure can bind to the antigenic loop region of HBsAg. The envelope of the hepatitis B virus generally contains three “HBV envelope proteins” (also known as “HBsAg”, “hepatitis B surface antigen”): S protein (for “small”, also referred to as S-HBsAg), M protein (for “middle”, also referred to as M-HBsAg) and L protein (for “large”, also referred to as L-HBsAg). S-HBsAg, M-HBsAg and L-HBsAg share the same C-terminal extremity (also referred to as “S domain”, 226 amino acids), which corresponds to the S protein (5-HBsAg) and which is crucial for virus assembly and infectivity. S-HBsAg, M-HBsAg and L-HBsAg are synthesized in the endoplasmic reticulum (ER), assembled, and secreted as particles through the Golgi apparatus. The S domain comprises four predicted transmembrane (TM) domains, whereby both the N-terminus as well as the C-terminus of the S domain are exposed to the lumen. The transmembrane domains TM1 and TM2 are both believed necessary for cotranslational protein integration into the ER membrane and the transmembrane domains TM3 and TM4 are located in the C-terminal third of the S domain. The “antigenic loop region” of HBsAg is located between the predicted TM3 and TM4 transmembrane domains of the S domain of HBsAg, whereby the antigenic loop region comprises amino acids 101-172 of the S domain, which contains 226 amino acids in total (Salisse J. and Sureau C., 2009, Journal of Virology 83: 9321-9328). A determinant of infectivity resides in the antigenic loop region of HBV envelope proteins. In particular, residues between 119 and 125 of the HBsAg contain a CXXC motif, which is considered to be important for the infectivity of HBV and HDV (Jaoude G A, Sureau C, Journal of Virology, 2005; 79:10460-6).

When positions in the amino acid sequence of the S domain of HbsAg are referred to herein, such positions are made with reference to the amino acid sequence as set forth in SEQ ID NO: 3 (shown below) or to natural or artificial sequence variants thereof.

MENITSGFLGPLLVLQAGFFLLTRILTIPQSLDSWWTSLNFLGGTTVCLG
QNSQSPTSNHSPTSCPPTCPGYRWMCLRRFIIFLFILLLCLIFLLVLLDY
QGMLPVCPLIPGSSTTSTGPCRTCMTTAQGTSMYPSCCCTKPSDGNCTCI
PIPSSWAFGKFLWEWASARFSWLSLLVPFVOWFVGLSPTVWLSVIWMMWY
WGPSLYSILSPFLPLLPIFFCLWVYI
(SEQ ID NO: 3; amino acids 101 - 172 are shown
underlined)

For example, the expression “amino acids 101-172 of the S domain” refers to the amino acid residues from positions 101-172 of the polypeptide according to SEQ ID NO: 3. However, a person skilled in the art understands that mutations or variations (including, but not limited to, substitution, deletion and/or addition, for example, HBsAg of a different genotype or a different HBsAg mutant as described herein) may occur naturally in the amino acid sequence of the S domain of HBsAg or be introduced artificially into the amino acid sequence of the S domain of HBsAg without affecting its biological properties. Therefore, as used herein, the term “S domain of HBsAg” encompasses all such polypeptides including, for example, the polypeptide according to SEQ ID NO: 3 and its natural or artificial mutants. In addition, when sequence fragments of the S domain of HBsAg are described herein (e.g. amino acids 101-172 or amino acids 120-130 of the S domain of HBsAg), they include not only the corresponding sequence fragments of SEQ ID NO: 3, but also the corresponding sequence fragments of its natural or artificial mutants. For example, the phrase “amino acid residues from positions 101-172 of the S domain of HBsAg” encompasses amino acid residues from positions 101-172 of SEQ ID NO: 3 and the corresponding fragments of its mutants (natural or artificial mutants). As used herein, the phrases “corresponding sequence fragments” and “corresponding fragments” refer to fragments that are located in equal positions of sequences when the sequences are subjected to optimized alignment, namely, the sequences are aligned to obtain a highest percentage of identity.

The M protein (M-HBsAg) corresponds to the S protein extended by an N-terminal domain of 55 amino acids called “pre-S2”. The L protein (L-HBsAg) corresponds to the M protein extended by an N-terminal domain of 108 amino acids called “pre-S1” (genotype D). The pre-S1 and pre-S2 domains of the L protein can be present either at the inner face of viral particles (on the cytoplasmic side of the ER), and is believed to play a crucial role in virus assembly, or on the outer face (on the luminal side of the ER), available for the interaction with target cells and important for viral infectivity. Moreover, HBV surface proteins (HBsAgs) are not only incorporated into virion envelopes but also can spontaneously bud from ER-Golgi intermediate compartment membranes to form empty “subviral particles” (SVPs) that are released from the cell by secretion.

In some embodiments, a binding protein binds to the antigenic loop region of HBsAg, and is capable of binding to all of S-HBsAg, M-HBsAg and L-HBsAg.

In some embodiments, a binding protein neutralizes infection with hepatitis B virus and hepatitis delta virus. In some embodiments, the binding protein, reduces viral infectivity of hepatitis B virus and hepatitis delta virus.

To study and quantitate virus infectivity (or “neutralization”) in the laboratory, standard “neutralization assays” may be utilized. For a neutralization assay, animal viruses are typically propagated in cells and/or cell lines. A neutralization assay wherein cultured cells are incubated with a fixed amount of HBV or HDV in the presence (or absence) of the antibody (or antigen-binding fragment or fusion protein) to be tested may be used. In such an assay, the levels of hepatitis B surface antigen (HBsAg) or hepatitis B e antigen (HBeAg) secreted into the cell culture supernatant may be used and/or HBcAg staining may be assessed to provide a readout. For HDV, for example, delta antigen immunofluorescence staining may be assessed.

In a particular embodiment of an HBV neutralization assay, cultured cells, for example HepaRG cells, such as differentiated HepaRG cells, are incubated with a fixed amount of HBV in the presence or absence of the antibody to be tested. In such and embodiment, incubation may be carried out, for example, for 16 hours at 37° C. That incubation may be performed in a medium (e.g. supplemented with 4% PEG 8000). After incubation, cells may be washed and further cultivated. To measure virus infectivity, the levels of hepatitis B surface antigen (HBsAg) and/or hepatitis B e antigen (HBeAg) secreted into the culture supernatant, e.g. from day 7 to day 11 post-infection, may be determined by enzyme-linked immunosorbent assay (ELISA). Additionally, HBcAg staining may be assessed in an immunofluorescence assay. In an embodiment of a HDV neutralization assay, essentially the same assay as for HBV may be used, with the difference that sera from HDV carriers may be used as HDV infection inoculum on differentiated HepaRg cells (instead of HBV). For detection, delta antigen immunofluorescence staining may be used as a readout.

Embodiments of the binding proteins of the disclosure have high neutralizing potency. In certain embodiments, the concentration of an antibody as described herein required for 50% neutralization of hepatitis B virus (HBV) and hepatitis delta virus (HDV), is, for example, about 10 μg/ml or less. In other embodiments, the concentration of a binding protein required for 50% neutralization of HBV and HDV is about 5 μg/ml. In other embodiments, the concentration of a binding protein as described herein required for 50% neutralization of HBV and HDV is about 1 μg/ml. In still other embodiments, the concentration of a binding protein required for 50% neutralization of HBV and HDV is about 750 ng/ml. In yet further embodiments, the concentration of a binding protein as described herein required for 50% neutralization of HBV and HDV is 500 ng/ml or less. In such embodiments, the concentration of a binding protein as described herein required for 50% neutralization of HBV and HDV may be selected from 450 ng/ml or less, 400 ng/ml or less, 350 ng/ml or less, 300 ng/ml or less, 250 ng/ml or less, 200 ng/ml or less, 175 ng/ml or less, 150 ng/ml or less, 125 ng/ml or less, 100 ng/ml or less, 90 ng/ml or less, 80 ng/ml or less, 70 ng/ml or less, 60 ng/ml or less, 50 ng/ml or less, or less than 20 ng/ml, preferably 15 ng/ml or less, more preferably 10 ng/ml or less, such as 7 ng/ml or less.

Binding proteins according to the present disclosure, which can neutralize both HBV and HDV, are useful in the prevention and treatment of hepatitis B and hepatitis D. Infection with HDV typically occurs simultaneously with or subsequent to infection by HBV (e.g., inoculation with HDV in the absence of HBV does not cause hepatitis D since HDV requires the support of HBV for its own replication) and hepatitis D is typically observed in chronic HBV carriers.

Embodiments of disclosed binding proteins promote clearance of HBsAg and HBV. In particular embodiments, binding proteins promote clearance of both HBV and subviral particles of hepatitis B virus (SVPs). Clearance of HBsAg or of subviral particles may be assessed by measuring the level of HBsAg for example in a blood sample, e.g. from a hepatitis B patient. Similarly, clearance of HBV may be assessed by measuring the level of HBV for example in a blood sample, e.g. from a hepatitis B patient.

In the sera of patients infected with HBV, in addition to infectious particles (HBV), there is typically an excess (typically 1,000- to 100,000-fold) of empty subviral particles (SVP) composed solely of HBV envelope proteins (HBsAg) in the form of relatively smaller spheres and filaments of variable length. Subviral particles have been shown to strongly enhance intracellular viral replication and gene expression of HBV (Bruns M. et al. 1998 J Virol 72(2): 1462-1468). This is also relevant in the context of infectivity of sera containing HBV, since the infectivity depends not only on the number of viruses but also on the number of SVPs (Bruns M. et al. 1998 J Virol 72(2): 1462-1468). Moreover, an excess of subviral particles can serve as a decoy by absorbing neutralizing antibodies and therefore delay the clearance of infection. Achievement of hepatitis B surface antigen (HBsAg) loss is considered in some instances to be an ideal endpoint of treatment and the closest outcome to cure chronic hepatitis B (CHB).

Embodiments of binding proteins of the present disclosure may promote clearance of HbsAg. In certain embodiments, the binding proteins may promote clearance of subviral particles of hepatitis B virus. In some embodiments, the binding proteins may be used to treat chronic hepatitis B.

In any of the presently disclosed embodiments, a binding protein of the present disclosure is capable of binding an HBsAg of a genotype selected from the HBsAg genotypes A, B, C, D, E, F, G, H, I, and J, or any combination thereof.

In certain embodiments, binding proteins of the present disclosure are capable of binding to any 1, 2, 3, 4, 5, 6, 7, 8, 9 or 10 of the HBsAg genotypes A, B, C, D, E, F, G, H, I, and J. Examples of different HBsAg genotypes of include the following: GenBank accession number J02203 (HBV-D, ayw3); GenBank accession number FJ899792.1 (HBV-D, adw2); GenBank accession number AM282986 (HBV-A); GenBank accession number D23678 (HBV-B1 Japan); GenBank accession number AB117758 (HBV-C1 Cambodia); GenBank accession number AB205192 (HBV-E Ghana); GenBank accession number X69798 (HBV-F4 Brazil); GenBank accession number AF160501 (HBV-G USA); GenBank accession number AY090454 (HBV-H Nicaragua); GenBank accession number AF241409 (HBV-I Vietnam); and GenBank accession number AB486012 (HBV-J Borneo). Exemplary amino acid sequences of the antigenic loop region of the S domain of HBsAg of different genotypes are described herein (e.g., SEQ ID NOs.: 5-15).

In some embodiments, a binding protein is capable of binding to one or more, and in some cases at least 6 of the 10 HBsAg genotypes A, B, C, D, E, F, G, H, I, and J. In certain embodiments, a binding protein is capable of binding to at least 8 of the 10 HBsAg genotypes A, B, C, D, E, F, G, H, I, and J. In some embodiments, a binding protein is capable of binding to all 10 of the 10 HBsAg genotypes A, B, C, D, E, F, G, H, I, and J. HBV is differentiated into several genotypes, according to genome sequence. To date, eight well-known genotypes (A-H) of the HBV genome have been defined. Moreover, two other genotypes, I and J, have also been identified (Sunbul M., 2014, World J Gastroenterol 20(18): 5427-5434). The genotype is known to affect the progression of the disease and differences between genotypes in response to antiviral treatment have been determined.

In some embodiments, a binding protein according to the present disclosure is capable of binding to 1, 2, 3, 4, 5, 6, 7, 8, 9, 10, 11, 12, 13, 14, 15, 16, 17 or 18 of the HBsAg mutants having mutations in the antigenic loop region, with such mutant(s) being selected from one or more of HBsAg Y100C/P120T, HBsAg P120T, HBsAg P120T/S143L, HBsAg C121S, HBsAg R122D, HBsAg R122I, HBsAg T123N, HBsAg Q129H, HBsAg Q129L, HBsAg M133H, HBsAg M133L, HBsAg M133T, HBsAg K141E, HBsAg P142S, HBsAg S143K, HBsAg D144A, HBsAg G145R and HBsAg N146A. These mutants are naturally occurring mutants based on the S domain of HBsAg Genotype D, Genbank accession no. FJ899792 (SEQ ID NO.: 4). The mutated amino acid residue(s) in each of the mutants noted herein are indicated in the name.

SEQ ID NO.: 4:
MENVTSGFLGPLLVLQAGFFLLTRILTIPQSLDSWWTSLNFLGGTTVCLG
QNSQSPTSNHSPTSCPPTCPGYRWMCLRRFIIFLFILLLCLIFLLVLLDY
QGMLPVCPLIPGSSTTGTGPCRTCTTPAQGTSMYPSCCCTKPSDGNCTCI
PIPSSWAFGKFLWEWASARFSWLSLLVPFVQWFVGLSPTVWLSVIWMMWY
WGPSLYSTLSPFLPLLPIFFCLWVYI
(the antigenic loop region, i.e. amino acids 101 -
172, is shown underlined).

Amino acid sequences of the antigenic loop region of the S domain of HBsAg of different mutants are shown in SEQ ID NOs.: 16-33.

In certain embodiments, a binding protein as disclosed herein is capable of binding to at one or more, and in some cases at least 12 infectious HBsAg mutants selected from HBsAg Y100C/P120T, HBsAg P120T, HBsAg P120T/S143L, HBsAg C121S, HBsAg R122D, HBsAg R122I, HBsAg T123N, HBsAg Q129H, HBsAg Q129L, HBsAg M133H, HBsAg M133L, HBsAg M133T, HBsAg K141E, HBsAg P142S, HBsAg S143K, HBsAg D144A, HBsAg G145R and HBsAg N146A. In some such embodiments, a binding protein is capable of binding to at least 15 infectious HBsAg mutants selected from HBsAg Y100C/P120T, HBsAg P120T, HBsAg P120T/S143L, HBsAg C121S, HBsAg R122D, HBsAg R122I, HBsAg T123N, HBsAg Q129H, HBsAg Q129L, HBsAg M133H, HBsAg M133L, HBsAg M133T, HBsAg K141E, HBsAg P142S, HBsAg S143K, HBsAg D144A, HBsAg G145R and HBsAg N146A. In some embodiments, a binding protein is capable of binding to each of the following infectious HBsAg mutants: HBsAg Y100C/P120T; HBsAg P120T; HBsAg P120T/S143L; HBsAg C121S; HBsAg R122D; HBsAg R122I; HBsAg T123N; HBsAg Q129H; HBsAg Q129L; HBsAg M133H; HBsAg M133L; HBsAg M133T; HBsAg K141E; HBsAg P142S; HBsAg S143K; HBsAg D144A; HBsAg G145R; and HBsAg N146A.

In certain embodiments, the binding protein (e.g., including an antibody or antigen binding fragment thereof) is capable of reducing a serum concentration of HBV

DNA in a mammal having an HBV infection. In certain embodiments, the binding protein is capable of reducing a serum concentration of HBsAg in a mammal having an HBV infection. In certain embodiments, the binding protein is capable of reducing a serum concentration of HBeAg in a mammal having an HBV infection. In certain embodiments, the binding protein is capable of reducing a serum concentration of HBcrAg in a mammal having an HBV infection. In some embodiments, the binding protein is capable of reducing the serum concentration of HBV DNA, HBsAg, HBeAg, and/or HBcrAg in the mammal for about 10, 11, 12, 13, 14, 15, 16, 17, 18, 19, 20, or more days following a single administration of the binding protein.

The term “epitope” or “antigenic epitope” includes any molecule, structure, amino acid sequence, or protein determinant that is recognized and specifically bound by a cognate binding molecule, such as an immunoglobulin, chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

In some embodiments, a binding protein is capable of binding to an epitope comprising at least one, at least two, at least three, or at least four amino acids of the antigenic loop region of HbsAg. In certain embodiments, a binding protein is capable of binding at least two amino acids selected from amino acids 115-133 of the S domain of HbsAg, amino acids 120-133 of the S domain of HbsAg, or amino acids 120-130 of the S domain of HbsAg. In certain embodiments, a binding protein is capable of binding at least three amino acids selected from amino acids 115-133 of the S domain of HbsAg, amino acids 120-133 of the S domain of HbsAg, or amino acids 120-130 of the S domain of HbsAg. In some embodiments, a binding protein is capable of binding at least four amino acids selected from amino acids 115-133 of the S domain of HbsAg, amino acids 120-133 of the S domain of HbsAg, or amino acids 120-130 of the S domain of HbsAg. As used herein, the position of the amino acids (e.g. 115-133, 120-133, 120-130) refers to the S domain of HBsAg as described above, which is present in all three HBV envelope proteins S-HBsAg, M-HBsAg, and L-HBsAg, whereby S-HBsAg typically corresponds to the S domain of HBsAg.

The term “formed by” as used herein in the context of an epitope, means that the epitope to which the binding protein binds to may be linear (continuous) or conformational (discontinuous). A linear or a sequential epitope is an epitope that is recognized by an antibody according to its linear sequence of amino acids, or primary structure. A conformational epitope may be recognized according to a three-dimensional shape and protein structure. Accordingly, if the epitope is a linear epitope and comprises more than one amino acid located at positions selected from amino acid positions 115-133 or from amino acid positions 120-133 of the S domain of HBsAg, the amino acids comprised by the epitope may be located in adjacent positions of the primary structure (e.g., are consecutive amino acids in the amino acid sequence). In the case of a conformational epitope (3D structure), the amino acid sequence typically forms a 3D structure as epitope and, thus, the amino acids forming the epitope may be or may be not located in adjacent positions of the primary structure (i.e. maybe or may be not consecutive amino acids in the amino acid sequence).

In certain embodiments, an epitope to which a binding protein binds to a conformational epitope. In some embodiments, a binding protein binds to an epitope comprising at least two amino acids of the antigenic loop region of HBsAg, wherein the at least two amino acids are selected from amino acids 120-133 or from amino acids 120-130, of the S domain of HbsAg, and wherein the at least two amino acids are not located in adjacent positions (of the primary structure). In certain embodiments, a binding protein binds to an epitope comprising at least three amino acids of the antigenic loop region of HBsAg, wherein the at least three amino acids are selected from amino acids 120-133 or from amino acids 120-130, of the S domain of HbsAg, and wherein at least two of the three amino acids are not located in adjacent positions (of the primary structure). In some embodiments, a binding protein binds to an epitope comprising at least four amino acids of the antigenic loop region of HBsAg, wherein the at least four amino acids are selected from amino acids 120-133 or from from amino acids 120-130, of the S domain of HbsAg, and wherein at least two of the four amino acids are not located in adjacent positions (of the primary structure).

Amino acids to which a presently disclosed antibody, antigen binding fragment, or fusion protein binds (i.e. the amino acids forming the epitope), which are not located in adjacent positions of the primary structure, are in some cases spaced apart by one or more amino acids, to which the antibody, antigen binding fragment, or fusion protein does not bind. In some embodiments, at least one, at least two, at least three, at least four, or at least five amino acids may be located between two of the amino acids not located in adjacent positions comprised by the epitope.

In certain embodiments, a binding protein binds to an epitope comprising at least amino acids P120, C121, R122 and C124 of the S domain of HBsAg. In other embodiments, a binding protein of the present disclosure binds to an epitope comprising an amino acid sequence according to SEQ ID NO.: 115:

PCRXC

wherein X is any amino acid or no amino acid; X is any amino acid; X is T, Y, R, S, or F; X is T, Y or R; or X is T or R.

In other embodiments, a binding protein of the present disclosure binds to an epitope comprising an amino acid sequence according to SEQ ID NO.: 107:

TGPCRTC

or to an amino acid sequence sharing at least 80%, at least 90%, or at least 95% sequence identity with SEQ ID NO.: 107.

In other embodiments, a binding protein of the present disclosure binds to an epitope comprising an amino acid sequence according to SEQ ID NO.: 112:

STTSTGPCRTC

or to an amino acid sequence sharing at least 80%, at least 90% or at least 95% sequence identity with SEQ ID NO.: 112.

In certain embodiments, a binding protein of the present disclosure binds to an epitope comprising an amino acid sequence comprising at least amino acids 145-151 of the S domain of HBsAg:

(SEQ ID NO.: 108)
GNCTCIP.

In still other embodiments, a binding protein of the present disclosure binds to an epitope comprising an amino acid sequence according to SEQ ID NO: 107 and an amino acid sequence according to SEQ ID NO.: 108.

In other embodiments, a binding protein of the present disclosure binds to an epitope comprising an amino acid sequence according to SEQ ID NO.: 112 and/or an amino acid sequence according to SEQ ID NO.: 114.

As described above, an epitope to which a binding protein of the present disclosure binds may be linear (continuous) or conformational (discontinuous). In some embodiments, a binding protein of the disclosure binds to a conformational epitope, and in certain such embodiments, the conformational epitope is present only under non-reducing conditions.

In certain embodiments, binding protein of the present disclosure, binds to a linear epitope. In certain such embodiments, the linear epitope is present under both, non-reducing conditions and reducing conditions.

In particular embodiments, a binding protein of the present disclosure binds to an epitope in the antigenic loop of HBsAg formed by an amino acid sequence according to SEQ ID NO.: 1:

X₁ X₂ X₃ TC X₄ X₅ X₆A X₇G

wherein X₁, X₂, X₃, X₄, X₅, X₆ and X₇ may be any amino acid (SEQ ID NO.: 1).

In some embodiments, X₁, X₂, X₃, X₄, X₅, X₆ and X₇ are amino acids, which are conservatively substituted in comparison to amino acids 120-130 of SEQ ID NO.: 3. In some embodiments, X₁, X₂, X₃, X₄, X₅, X₆ and X₇ are amino acids, which are conservatively substituted in comparison to amino acids 20-30 of any of SEQ ID NOs.: 5-33.

In specific embodiments, X₁ of SEQ ID NO.: 1 X₁ is a small amino acid. A “small” amino acid, as used herein, refers to any amino acid selected from the group consisting of alanine, aspartic acid, asparagine, cysteine, glycine, proline, serine, threonine and valine. In certain such embodiments, X₁ is proline, serine or threonine.

In certain embodiments, X₂ of SEQ ID NO.: 1 X₂ is a small amino acid. In certain embodiments, X₂ may be selected from cystein or threonine.

In some embodiments, X₃ of SEQ ID NO.: 1 is a charged amino acid or an aliphatic amino acid. A “charged” amino acid, as used herein, refers to any amino acid selected from the group consisting of arginine, lysine, aspartic acid, glutamic acid and histidine. A “aliphatic” amino acid, as used herein, refers to any amino acid selected from the group consisting of alanine, glycine, isoleucine, leucine, and valine. In certain embodiments, X₃ is selected from arginine, lysine, aspartic acid or isoleucine.

In some embodiments, X₄ of SEQ ID NO.: 1 is a small amino acid and/or a hydrophobic amino acid. A “hydrophobic” amino acid, as used herein, refers to any amino acid selected from the group consisting of alanine, isoleucine, leucine, phenylalanine, valine, tryptophan, tyrosine, methionine, proline and glycine. In certain embodiments, X₄ is selected from methionine or threonine.

In some embodiments, X₅ of SEQ ID NO.: 1 X₅ is a small amino acid and/or a hydrophobic amino acid. In certain embodiments, X₅ is selected from threonine, alanine or isoleucine.

In some embodiments, X₆ of SEQ ID NO.: 1 X₆ is a small amino acid and/or a hydrophobic amino acid. In certain embodiments, X₆ is selected from threonine, proline or leucine.

In some embodiments, X₇ of SEQ ID NO.: 1 is a polar amino acid or an aliphatic amino acid. A “polar” amino acid, as used herein, refers to any amino acid selected from the group consisting of aspartic acid, asparagine, arginine, glutamic acid, histidine, lysine, glutamine, tryptophan, tyrosine, serine, and threonine. In certain such embodiments, X₇ is glutamine, histidine or leucine.

In some embodiments, a binding protein according to the present disclosure binds to an epitope in the antigenic loop of HBsAg formed by an amino acid sequence according to SEQ ID NO.: 2:

X₁ X₂ X₃ TC X₄ X₅ X₆A X₇G

wherein

X₁ is P, T or S,

X₂ is C or S,

X₃ is R, K, D or I,

X₄ is M or T,

X₅ is T, A or I,

X₆ is T, P or L, and

X₇ is Q, H or L

(SEQ ID NO.: 2).

With regard to the epitopes formed by the amino acid sequences according to SEQ ID NO.: 1 or 2, it is noted that the term “formed by” as used herein is not intended to imply that a disclosed binding protein necessarily binds to each and every amino acid of SEQ ID NO.: 1 or 2. In particular, a binding protein may bind only to some of the amino acids of SEQ ID NO.: 1 or 2, whereby other amino acid residues may act as “spacers”.

In particular embodiments, a binding protein according to the present disclosure binds to an epitope in the antigenic loop of HBsAg formed by one or more, two or more, three or more, or four or more amino acids of an amino acid sequence selected from SEQ ID NOs.: 5-33 shown below in Table 4.

In some embodiments, binding protein according to the present disclosure binds to an antigenic loop region of HBsAg having an amino acid sequence according to any one or more of SEQ ID NOs.: 5-33 shown below in Table 4, or to a sequence variant thereof. In certain embodiments, a binding protein according to the present disclosure binds to all of the antigenic loop variants of HBsAg having an amino acid sequence according to any of SEQ ID NOs.: 5-33 shown below in Table 4.

TABLE 4
Amino acid sequences of the antigenic loop region of the S domain of HBsAg
(residues 101-172 of the S domain of HBsAg - except for SEQ ID NO: 16, which refers
to residues 100-172 of the S domain of HBsAg in order to include the relevant
mutation) of the different genotypes and mutants as used herein.

| Name | SEQ ID NO. | Amino acid sequence |
|---|---|---|
| J02203 (D, ayw3) | 5 | QGMLPVCPLIPGSSTTSTGPCRTCMTTAQGTS |
| | | MYPSCCCTKPSDGNCTCIPIPSSWAFGKFLWE |
| | | WASARFSW |
| FJ899792 (D, | 6 | QGMLPVCPLIPGSSTTGTGPCRTCTTP |
| adw2) | | AQGTSMYPSCCCTKPSDGNCTCIPIPS |
| | | SWAFGKFLWEWASARFSW |
| AM282986 | 7 | QGMLPVCPLIPGTTTTSTGPCKTCTTPAQGNS |
| (A) | | MFPSCCCTKPSDGNCTCIPIPSSWAFAKYLWE |
| | | WASVRFSW |
| D23678 (B1) | 8 | QGMLPVCPLIPGSSTTSTGPCKTCTTPAQGTS |
| | | MFPSCCCTKPTDGNCTCIPIPSSWAFAKYLWE |
| | | WASVRFSW |
| AB117758 (C1) | 9 | QGMLPVCPLLPGTSTTSTGPCKTCTIPAQGTS |
| | | MFPSCCCTKPSDGNCTCIPIPSSWAFARFLWE |
| | | WASVRFSW |
| AB205192 (E) | 10 | QGMLPVCPLIPGSSTTSTGPCRTCTTLAQGTS |
| | | MFPSCCCSKPSDGNCTCIPIPSSWAFGKFLWE |
| | | WASARFSW |
| X69798 (F4) | 11 | QGMLPVCPLLPGSTTTSTGPCKTCTTLAQGTS |
| | | MFPSCCCSKPSDGNCTCIPIPSSWALGKYLWE |
| | | WASARFSW |
| AF160501 (G) | 12 | QGMLPVCPLIPGSSTTSTGPCKTCTTPAQGNS |
| | | MYPSCCCTKPSDGNCTCIPIPSSWAFAKYLWE |
| | | WASVRFSW |
| AY090454 (H) | 13 | QGMLPVCPLLPGSTTTSTGPCKTCTTLAQGTS |
| | | MFPSCCCTKPSDGNCTCIPIPSSWAFGKYLWE |
| | | WASARFSW |
| AF241409 (I) | 14 | QGMLPVCPLIPGSSTTSTGPCKTCTTPAQGNS |
| | | MYPSCCCTKPSDGNCTCIPIPSSWAFAKYLWE |
| | | WASARFSW |
| AB486012 (J) | 15 | QGMLPVCPLLPGSTTTSTGPCRTCTITAQGTS |
| | | MFPSCCCTKPSDGNCTCIPIPSSWAFAKFLWE |
| | | WASVRFSW |
| HBsAg | 16 | CQGMLPVCPLIPGSSTTGTGTCRTCTTPAQGT |
| Y100C/P120T | | SMYPSCCCTKPSDGNCTCIPIPSSWAFGKFLW |
| | | EWASARFSW |
| HBsAg P120T | 17 | QGMLPVCPLIPGSSTTGTGTCRTCTTPAQGTS |
| | | MYPSCCCTKPSDGNCTCIPIPSSWAFGKFLWE |
| | | WASARFSW |
| HBsAg | 18 | QGMLPVCPLIPGSSTTGTGTCRTCTTPAQGTS |
| P120T/S143L | | MYPSCCCTKPLDGNCTCIPIPSSWAFGKFLWE |
| | | WASARFSW |
| HBsAg C121S | 19 | QGMLPVCPLIPGSSTTGTGPSRTCTTPAQGTS |
| | | MYPSCCCTKPSDGNCTCIPIPSSWAFGKFLWE |
| | | WASARFSW |
| HBsAg R122D | 20 | QGMLPVCPLIPGSSTTGTGPCDTCTTPAQGTS |
| | | MYPSCCCTKPSDGNCTCIPIPSSWAFGKFLWE |
| | | WASARFSW |
| HBsAg R122I | 21 | QGMLPVCPLIPGSSTTGTGPCITCTTPAQGTSM |
| | | YPSCCCTKPSDGNCTCIPIPSSWAFGKFLWEW |
| | | ASARFSW |
| HBsAg T123N | 22 | QGMLPVCPLIPGSSTTGTGPCRNCTTPAQGTS |
| | | MYPSCCCTKPSDGNCTCIPIPSSWAFGKFLWE |
| | | WASARFSW |
| HBsAg Q129H | 23 | QGMLPVCPLIPGSSTTGTGPCRTCTTPAHGTS |
| | | MYPSCCCTKPSDGNCTCIPIPSSWAFGKFLWE |
| | | WASARFSW |
| HBsAg Q129L | 24 | QGMLPVCPLIPGSSTTGTGPCRTCTTPALGTS |
| | | MYPSCCCTKPSDGNCTCIPIPSSWAFGKFLWE |
| | | WASARFSW |
| HBsAg M133H | 25 | QGMLPVCPLIPGSSTTGTGPCRTCTTPAQGTS |
| | | HYPSCCCTKPSDGNCTCIPIPSSWAFGKFLWE |
| | | WASARFSW |
| HBsAg M133L | 26 | QGMLPVCPLIPGSSTTGTGPCRTCTTPAQGTSL |
| | | YPSCCCTKPSDGNCTCIPIPSSWAFGKFLWEW |
| | | ASARFSW |
| HBsAg M133T | 27 | QGMLPVCPLIPGSSTTGTGPCRTCTTPAQGTST |
| | | YPSCCCTKPSDGNCTCIPIPSSWAFGKFLWEW |
| | | ASARFSW |
| HBsAg K141E | 28 | QGMLPVCPLIPGSSTTGTGPCRTCTTPAQGTS |
| | | MYPSCCCTEPSDGNCTCIPIPSSWAFGKFLWE |
| | | WASARFSW |
| HBsAg P142S | 29 | QGMLPVCPLIPGSSTTGTGPCRTCTTPAQGTS |
| | | MYPSCCCTKSSDGNCTCIPIPSSWAFGKFLWE |
| | | WASARFSW |
| HBsAg S143K | 30 | QGMLPVCPLIPGSSTTGTGPCRTCTTPAQGTS |
| | | MYPSCCCTKPKDGNCTCIPIPSSWAFGKFLWE |
| | | WASARFSW |
| HBsAg D144A | 31 | QGMLPVCPLIPGSSTTGTGPCRTCTTPAQGTS |
| | | MYPSCCCTKPSAGNCTCIPIPSSWAFGKFLWE |
| | | WASARFSW |
| HBsAg G145R | 32 | QGMLPVCPLIPGSSTTGTGPCRTCTTPAQGTS |
| | | MYPSCCCTKPSDRNCTCIPIPSSWAFGKFLWE |
| | | WASARFSW |
| HBsAgN146A | 33 | QGMLPVCPLIPGSSTTGTGPCRTCTTPAQGTS |
| | | MYPSCCCTKPSDGACTCIPIPSSWAFGKFLWE |
| | | WASARFSW |

### Fc Moiety

In some embodiments, a binding protein (e.g., antibody or an antigen binding fragment thereof) of the present disclosure comprises an Fc moiety (also referred to as an Fc polypeptide). In certain embodiments, the Fc moiety may be derived from human origin, e.g., from human IgG1, IgG2, IgG3, and/or IgG4, or from another Ig class or isotype. In specific embodiments, an antibody or antigen binding fragment can comprise an Fc moiety derived from human IgG1. In particular embodiments, the Fc moiety comprises, or is derived from (e.g., comprises one or more mutations relative to), IgG1m17, 1 (IgHG1*01) allotype.

As used herein, the term “Fc moiety” refers to a sequence comprising, consisting of, consisting essentially of, or derived from a portion of an immunoglobulin heavy chain beginning in the hinge region just upstream of the papain cleavage site (e.g., residue 216 by EU numbering in native IgG, taking the first residue of heavy chain constant region to be 114) and ending at the C-terminus of the immunoglobulin heavy chain. Accordingly, an Fc moiety may be a complete Fc moiety or a portion (e.g., a domain) thereof. In certain embodiments, a complete Fc moiety comprises a hinge domain, a CH2 domain, and a CH3 domain (e.g., EU amino acid positions 216-446). As noted herein, an additional lysine residue (K) is sometimes present at the extreme C-terminus of the Fc moiety, but is often cleaved from a mature antibody. Amino acid positions within an Fc moiety have been numbered according to the EU numbering system of Kabat, see e.g., Kabat et al., “Sequences of Proteins of Immunological Interest”, U.S. Dept. Health and Human Services, 1983 and 1987. Amino acid positions of an Fc moiety can also be numbered according to the IMGT numbering system (including unique numbering for the C-domain and exon numbering) and the Kabat numbering system.

In some embodiments, an Fc moiety comprises at least one of: a hinge (e.g., upper, middle, and/or lower hinge region) domain, a CH2 domain, a CH3 domain, or a variant, portion, or fragment thereof. In some embodiments, an Fc moiety comprises at least a hinge domain, a CH2 domain or a CH3 domain. In further embodiments, the Fc moiety is a complete Fc moiety. The amino acid sequence of an exemplary Fc moiety of human IgG1 isotype is provided in SEQ ID NO.:73. The Fc moiety may also comprise one or more amino acid insertions, deletions, or substitutions relative to a naturally occurring Fc moiety. For example, at least one of a hinge domain, CH2 domain, or CH3 domain, or a portion thereof, may be deleted. For example, an Fc moiety may comprise or consist of: (i) hinge domain (or a portion thereof) fused to a CH2 domain (or a portion thereof), (ii) a hinge domain (or a portion thereof) fused to a CH3 domain (or a portion thereof), (iii) a CH2 domain (or a portion thereof) fused to a CH3 domain (or a portion thereof), (iv) a hinge domain (or a portion thereof), (v) a CH2 domain (or a portion thereof), or (vi) a CH3 domain or a portion thereof.

An Fc moiety of the present disclosure may be modified such that it varies in amino acid sequence from the complete Fc moiety of a naturally occurring immunoglobulin molecule, while retaining or enhancing at least one desirable function conferred by the naturally occurring Fc moiety, and/or reducing an undesired function of a naturally occurring Fc moiety. Such functions include, for example, Fc receptor (FcR) binding, antibody half-life modulation (e.g., by binding to FcRn), ADCC function, protein A binding, protein G binding, and complement binding. Portions of naturally occurring Fc moieties which are involved with such functions have been described in the art.

For example, to activate the complement cascade, the C1q protein complex can bind to at least two molecules of IgG1 or one molecule of IgM when the immunoglobulin molecule(s) is attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcεR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologuous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils.

FcγRIIA is found on many cells involved in killing (e.g. macrophages, monocytes, neutrophils) and is believed to activate the killing process. FcγRIIB is believed to play a role in inhibitory processes and is found on B-cells, macrophages and on mast cells and eosinophils. Importantly, it has been shown that 75% of all FcγRIIB is found in the liver (Ganesan, L. P. et al., 2012: “FcγRIIb on liver sinusoidal endothelium clears small immune complexes,” Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

In some embodiments, the antibodies disclosed herein and the antigen binding fragments thereof comprise an Fc moiety for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen binding fragments thereof, comprise an engineered Fc moiety with the mutations S267E and L328F, in particular as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933.

On B cells, FcγRIIB seems to function to suppress further immunoglobulin production and isotype switching to, for example, the IgE class. On macrophages, FcγRIIB is thought to inhibit phagocytosis as mediated through FcγRIIA. On eosinophils and mast cells, the B form may help to suppress activation of these cells through IgE binding to its separate receptor.

Regarding FcγRI binding, modification in native IgG of at least one of E233-G236, P238, D265, N297, A327 and P329 can reduce binding to FcγRI. IgG2 residues at positions 233-236, substituted into corresponding positions IgG1 and IgG4, reduces binding of IgG1 and IgG4 to FcγRI by 10³-fold and eliminated the human monocyte response to antibody-sensitized red blood cells (Armour, K. L., et al. Eur. J. Immunol. 29 (1999) 2613-2624).

Regarding FcγRII binding, reduced binding for FcγRIIA is found, e.g., for IgG mutation of at least one of E233-G236, P238, D265, N297, A327, P329, D270, Q295, A327, R292 and K414.

Two allelic forms of human FcγRIIA are the “H131” variant, which binds to IgG1 Fc with high affinity, and the “R131” variant, which binds to IgG1 Fc with low affinity. See, e.g., Bruhns et al., Blood 113:3716-3725 (2009).

Regarding FcγRIII binding, reduced binding to FcγRIIIA is found, e.g., for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above-mentioned mutation sites, and methods for measuring binding to FcγRI and FcγRIIA, are described in Shields, R. L., et al., J. Biol. Chem. 276 (2001) 6591-6604.

Two allelic forms of human FcγRIIIA are the “F158” variant, which binds to IgG1 Fc with low affinity, and the “V158” variant, which binds to IgG1 Fc with high affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRT appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

Also contemplated are mutations that increase binding affinity of an Fc moiety of the present disclosure to a (i.e., one or more) Fcγ receptor (e.g., as compared to a reference Fc moiety or antibody containing the same that does not comprise the mutation(s)). See, e.g., Delillo and Ravetch, Cell 161(5):1035-1045 (2015) and Ahmed et al., J. Struc. Biol. 194(1):78 (2016), the Fc mutations and techniques of which are incorporated herein by reference.

In any of the herein disclosed embodiments, a binding protein can comprise a (e.g., IgG1 or IgG1-derived) Fc moiety comprising a mutation selected from (EU numbering) G236A; S239D; A330L; and I332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/I332E. In some embodiments, the Fc moiety does not comprise S239D. In some embodiments, the Fc moiety comprises a native Serine at position 239.

In certain embodiments, the Fc moiety may comprise or consist of at least a portion of an Fc moiety that is involved in binding to FcRn binding. In certain embodiments, the Fc moiety comprises one or more amino acid modifications that improve binding affinity for (e.g., enhance binding to) FcRn (e.g., at a pH of about 6.0) and, in some embodiments, thereby extend in vivo half-life of a molecule comprising the Fc moiety (e.g., as compared to a reference Fc moiety or antibody that is otherwise the same but does not comprise the modification(s)). In certain embodiments, the Fc moiety comprises or is derived from a IgG Fc and a half-life-extending mutation comprises any one or more of: M428L; N434S; N434H; N434A; N434S; M252Y; S254T; T256E; T250Q; P257I Q311I; D376V; T307A; E380A (EU numbering). In certain embodiments, a half-life-extending mutation comprises M428L/N434S (also referred to herein as “MLNS”). In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A.

In some embodiments, a binding protein includes a Fc moiety that comprises the substitution mutations M428L/N434S. In some embodiments, a binding protein includes a Fc moiety that comprises the substitution mutations G236A/A330L/I332E. In certain embodiments, a binding protein includes a (e.g., IgG) Fc moiety that comprises a G236A mutation, an A330L mutation, and a I332E mutation (GAALIE), and does not comprise a S239D mutation (e.g., comprises a native S at position 239). In particular embodiments, a binding protein includes an Fc moiety that comprises the substitution mutations: M428L/N434S and G236A/A330L/I332E, and optionally does not comprise S239D (e.g., can comprise a native S at position 329). In certain embodiments, a binding protein includes a Fc moiety that comprises the substitution mutations: M428L/N434S and G236A/S239D/A330L/I332E. In certain further embodiments, a binding protein comprises substitution mutations in a Fc moiety, wherein the substitution mutations consist of, or consist essentially of: M428L/N434S, G236A/S239D/A330L/I332E, or G236A/S239D/A330L/I332E/M428L/N434S.

In any of the presently disclosed embodiments, a binding protein of the present disclosure includes a Fc moiety comprising a GAALIE mutation and has enhanced binding to a human FcγRIIa and/or a human FcγRIIIa, as compared to a reference polypeptide (i.e., a polypeptide, which may be a binding protein, that includes a Fc moiety that does not comprise the GAALIE mutation).

In certain embodiments, the reference polypeptide includes a Fc moiety that is a wild-type Fc moiety (e.g., of the same isotype) or is a Fc moiety that comprises one or more substitution mutation (or insertion or deletion), provided that the substitution mutation is not or does not comprise GAALIE. In certain embodiments, the reference polypeptide does not comprise a substitution mutation that is known or believed to affect binding to a human FcγRIIa and/or to a human FcγRIIIa.

Binding between polypeptides, such as binding between a Fc moiety (or a binding protein comprising the same) and a human Fcγ Receptor, such as human FcγRIIA, human FcγRIIIA, or human Fc FcγRIIB, or a complement protein, such as C1q, can be determined or detected using methods known in the art. For example, a biolayer interferometry (BLI) assay can be performed using an Octet® RED96 (ForteBio, Fremont, Calif. USA) instrument according to manufacturer's instructions to determine real-time association and dissociation between a first polypeptide of interest (e.g., a Fc moiety comprising a GAALIE mutation) and a second polypeptide of interest (e.g., a FcγRIIA (H131), a FcγRIIA (R131), a FcγRIIIA (F158), a FcγRIIIA (V158), or a FcγRIIb) that is captured on a sensor substrate.

In certain embodiments, a binding protein includes a Fc moiety comprising a GAALIE mutation and has enhanced binding to a human FcγRIIA (H131), a human FcγRIIA (R131), a human FcγRIIIA (F158), a human FcγRIIIA (V158), or any combination thereof, as compared to a reference polypeptide that includes a Fc moiety that does not comprise the GAALIE mutation. In certain embodiments, enhanced binding is determined by an increase (e.g., one or more of: a higher peak signal; a greater rate of association; a slower rate of dissociation; a greater area under the curve) in signal shift versus the reference binding protein in a BLI assay. In certain embodiments, the BLI assay comprises use of Octet® RED96 (ForteBio, Fremont, Calif. USA) instrument. In further embodiments, the BLI assay comprises a tagged human FcγR captured onto an anti-penta-tag sensor and exposed to the binding protein. In some embodiments, the binding protein comprises a IgG Fab and the BLI assay further comprises exposing the captured human FcγR to the binding protein in the presence of an anti-IgG Fab binding fragment to cross-link the binding proteins through the Fab fragment.

In certain embodiments, a binding protein includes a Fc moiety comprising a GAALIE mutation and has enhanced binding to a human FcγRIIA (H131), a human FcγRIIA (R131), a human FcγRIIIA (F158), and/or a human FcγRIIIA (V158) as compared to a reference polypeptide, wherein the enhanced binding can comprise a signal shift (nanometers) in a BLI assay of 1.5, 2, 2.5, 3, or more times greater than the signal shift observed using the reference binding protein.

In certain embodiments, a binding protein includes a Fc moiety comprising a GAALIE mutation and has enhanced binding to a human FcγRIIA (H131), a human FcγRIIA (R131), a human FcγRIIIA (F158), and a human FcγRIIIA (V158), as compared to a reference polypeptide.

In any of the presently disclosed embodiments, a binding protein includes a Fc moiety comprising a GAALIE mutation and has reduced binding to a human FcγRIIB, as compared to a reference polypeptide. In certain embodiments, a binding protein includes a Fc moiety comprising a GAALIE mutation and does not bind to a human FcγRIIB, as determined, for example, by the absence of a statistically significant signal shift versus baseline in a BLI assay.

In any of the presently disclosed embodiments, a binding protein includes a Fc moiety comprising a GAALIE mutation and has reduced binding to a human C1q (complement protein), as compared to a reference polypeptide. In certain embodiments, a binding protein includes a Fc moiety comprising a GAALIE mutation and does not bind to a human C1q, as determined by the absence of a statistically significant signal shift versus baseline in a BLI assay.

In any of the presently disclosed embodiments, a binding protein includes a Fc moiety comprising a GAALIE mutation and activates a human FcγRIIA, a human FcγRIIIA, or both, to a greater degree than does a reference polypeptide (i.e., a polypeptide, which may be a HBsAg-specific binding protein, that includes a Fc moiety that does not comprise the GAALIE mutation). In certain embodiments, the reference polypeptide includes a Fc moiety that is a wild-type Fc moiety or that comprises one or more substitution mutation, provided that the substitution mutation is not GAALIE.

Activation of a human FcγR can be determined or detected using methods known in the art. For example, a well-validated, commercially available bioreporter assay involves incubating a HBsAg-specific binding protein with a recombinant HBsAg (Engerix B, GlaxoSmithKline) in the presence of Jurkat effector cells (Promega; Cat. no: G9798) stably expressing (i) a FcγR of interest and (ii) firefly luciferase reporter under the control of a NFAT response element. Binding of Fc to cell surface-expressed FcγR drives NFAT-mediated expression of luciferase reporter gene. Luminescence is then measured with a luminometer (e.g., Bio-Tek) using the Bio-Glo-™ Luciferase Assay Reagent (Promega) according to the manufacturer's instructions. Activation is expressed as the average of relative luminescence units (RLU) over the background by applying the following formula: (RLU at concentration [x] of binding protein (e.g., mAbs)−RLU of background).

In certain embodiments, a binding protein includes a Fc moiety comprising a GAALIE mutation activates a human FcγRIIA (H131), a human FcγRIIA (R131), a human FcγRIIIA (F158), and/or a human FcγRIIIA (V158) to a greater degree than does a reference polypeptide. In certain embodiments, a greater degree of activation refers to a higher peak luminescence and/or a greater luminescence area under the curve, as determined using a luminescence bioreporter assay as described herein. In certain embodiments, a binding protein includes a Fc moiety comprising a GAALIE mutation and activates a human FcγRIIA (H131), a human FcγRIIA (R131), and a human FcγRIIIA (F158) to a greater degree than does a reference polypeptide, wherein the greater degree of activation comprises to a peak RLU that is 1.5, 2, 2.5, 3, or more times greater than the peak RLU observed using the reference binding protein.

In any of the presently disclosed embodiments, a binding protein includes a Fc moiety comprising a GAALIE mutation does not activate a human FcγRIIB, as determined by the absence of a statistically significant and/or measurable RLU in a luminescence bioreporter assay as described above.

In any of the presently disclosed embodiments, a binding protein includes a Fc moiety comprising a GAALIE mutation and activates a human natural killer (NK) cell in the presence of HBsAg to a greater degree than does a reference polypeptide. In certain embodiments, activation of a NK cell is determined by CD107a expression (e.g., by flow cytometry). In certain embodiments, the NK cell comprises a cell that comprises V158/V158 homozygous, a F158/F158 homozygous, or a V158/F158 heterozygous FcγRIIIa genotype.

It will be appreciated that any binding protein including a Fc moiety comprising a GAALIE mutation according to the present disclosure can perform or possess any one or more of the features described herein; e.g., enhanced binding to a human FcγRIIA and/or a human FcγRIIIA as compared to a reference polypeptide; reduced binding to a human FcγRIIB as compared to a reference polypeptide (and/or no binding to a human FcγRIIB); reduced binding to a human C1q as compared to a reference polypeptide (and/or no binding to a human C1q); activates a FcγRIIA, a human FcγRIIIA, or both, to a greater degree than does a reference polypeptide; does not activate a human FcγRIIB; and/or activates a human natural killer (NK) cell in the presence of HBsAg to a greater degree than does a reference polypeptide (e.g., an antibody that is specific for HBsAg and includes a Fc moiety that does not comprise a GAALIE mutation).

In certain embodiments, a binding protein of the present disclosure includes a Fc moiety comprising a GAALIE mutation and: (i) has enhanced binding to a human FcγRIIA, a human FcγRIIIA, or both, as compared to a reference polypeptide that includes a Fc moiety that does not comprise G236A/A330L/I332E, wherein the human FcγRIIA is optionally H131 or R131, and/or the human FcγRIIIA is optionally F158 or V158; (ii) has reduced binding to a human FcγRIIB, as compared to a reference polypeptide that includes a Fc moiety that does not comprise G236A/A330L/I332E; (iii) does not bind to a human FcγRIIB; (iv) has reduced binding to a human C1q, as compared to a reference polypeptide that includes a Fc moiety that does not comprise G236A/A330L/I332E; (v) does not bind to a human C1q; (vi) activates a FcγRIIA, a human FcγRIIIA, or both, to a greater degree than does a reference polypeptide that includes a Fc moiety that does not comprise G236A/A330L/I332E, wherein the human FcγRIIA is optionally H131 or R131, and/or the human FcγRIIIA is optionally F158 or V158; (vii) does not activate a human FcγRIIB; (viii) activates a human natural killer (NK) cell in the presence of HBsAg to a greater degree than does a reference polypeptide that includes a Fc moiety that does not comprise G236A/A330L/I332E, wherein the reference polypeptide is optionally an antibody that binds to an HB Ag, optionally an HBsAg (ix) is capable of binding to an HBsAg variant comprising HBsAg-Y100C/P120T, HBsAg-P120T, HBsAg-P120S/S143L, HBsAg-C121S, HBsAg-R122D, HBsAg-R122I, HBsAg-T123N, HBsAg-Q129H, HBsAg-Q129L, HBsAg-M133H, HBsAg-M133L, HBsAg-M133T, HBsAg-K141E, HBsAg-P142S, HBsAg-S143K, HBsAg-D144A, HBsAg-G145R, HBsAg-N146A, or any combination thereof; (x) has improved binding to an HBsAg variant comprising HBsAg-Y100C/P120T, HBsAg-P120T, HBsAg-P120S/S143L, HBsAg-C121S, HBsAg-R122D, HBsAg-R122I, HBsAg-T123N, HBsAg-Q129H, HBsAg-Q129L, HBsAg-M133H, HBsAg-M133L, HBsAg-M133T, HBsAg-K141E, HBsAg-P142S, HBsAg-S143K, HBsAg-D144A, HBsAg-G145R, HBsAg-N146A, or any combination thereof, as compared to a reference antibody or antigen binding fragment that binds to HBsAg and that includes a Fc moiety that does not comprise G236A/A330L/I332E.

Alternatively or additionally, the Fc moiety of a binding protein of the disclosure can comprise at least a portion known in the art to be required for Protein A binding; and/or the Fc moiety of an antibody of the disclosure comprises at least the portion of an Fc molecule known in the art to be required for protein G binding. In some embodiments, a retained function comprises the clearance of HBsAg and HBVg. Accordingly, in certain embodiments, an Fc moiety comprises at least a portion known in the art to be required for FcγR binding. As outlined above, an Fc moiety may thus at least comprise (i) the lower hinge site of native IgG Fc, in particular amino acid residues L, L, G, G (234-237, EU numbering), and (ii) the adjacent region of the CH2 domain of native IgG Fc, in particular a loop and strands in the upper CH2 domain adjacent to the lower hinge region, e.g. in a region of P331, for example a region of at least 3, 4, 5, 6, 7, 8, 9, or 10 consecutive amino acids in the upper CH2 domain of native IgG Fc around P331, e.g. between amino acids 320 and 340 (EU numbering) of native IgG Fc.

In some embodiments, a binding protein according to the present disclosure comprises an Fc region. As used herein, the term “Fc region” refers to the portion of an immunoglobulin formed by two or more Fc moieties of antibody heavy chains. For example, an Fc region may be monomeric or “single-chain” Fc region (i.e., a scFc region). Single chain Fc regions are comprised of Fc moieties linked within a single polypeptide chain (e.g., encoded in a single contiguous nucleic acid sequence). Exemplary scFc regions are disclosed in WO 2008/143954 A2, and are incorporated by reference herein. The Fc region can be or comprise a dimeric Fc region; it will be understood that a dimeric Fc region is not the same as an undesired (e.g., antibody:antibody, antibody:antigen-binding fragment, or antigen-binding fragment:antigen-binding fragment) dimer, such as described above and illustrated, in one embodiment, in FIG. 7. In certain preferred embodiments, an antibody or antigen-binding fragment comprises a dimeric Fc region, while producing few antibody- or antigen-binding fragment-containing dimers.

A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties (e.g., one Fc monomer of the dimeric Fc region comprises at least one amino acid modification (e.g., substitution, deletion, insertion, or chemical modification) that is not present in the other Fc monomer, or one Fc monomer may be truncated as compared to the other).

Presently disclosed Fc moieties may comprise Fc sequences or regions of the same or different class and/or subclass. For example, Fc moieties may be derived from an immunoglobulin (e.g., a human immunoglobulin) of an IgG1, IgG2, IgG3 or IgG4 subclass, or from any combination thereof. In certain embodiments, the Fc moieties of Fc region are of the same class and subclass. However, the Fc region (or one or more Fc moieties of an Fc region) may also be chimeric, whereby a chimeric Fc region may comprise Fc moieties derived from different immunoglobulin classes and/or subclasses. For example, at least two of the Fc moieties of a dimeric or single-chain Fc region may be from different immunoglobulin classes and/or subclasses. In certain embodiments, a dimeric Fc region can comprise sequences from two or more different isotypes or subclasses; e.g., a SEEDbody (“strand-exchange engineered domains”) see Davis et al., Protein Eng. Des. Sel. 23(4):195 (2010).

Additionally or alternatively, chimeric Fc regions may comprise one or more chimeric Fc moieties. For example, the chimeric Fc region or moiety may comprise one or more portions derived from an immunoglobulin of a first subclass (e.g., an IgG1, IgG2, or IgG3 subclass) while the remainder of the Fc region or moiety is of a different subclass. For example, an Fc region or moiety of an Fc polypeptide may comprise a CH2 and/or CH3 domain derived from an immunoglobulin of a first subclass (e.g., an IgG1, IgG2 or IgG4 subclass) and a hinge region from an immunoglobulin of a second subclass (e.g., an IgG3 subclass). For example, the Fc region or moiety may comprise a hinge and/or CH2 domain derived from an immunoglobulin of a first subclass (e.g., an IgG4 subclass) and a CH3 domain from an immunoglobulin of a second subclass (e.g., an IgG1, IgG2, or IgG3 subclass). For example, the chimeric Fc region may comprise an Fc moiety (e.g., a complete Fc moiety) from an immunoglobulin for a first subclass (e.g., an IgG4 subclass) and an Fc moiety from an immunoglobulin of a second subclass (e.g., an IgG1, IgG2 or IgG3 subclass). For example, the Fc region or moiety may comprise a CH2 domain from an IgG4 immunoglobulin and a CH3 domain from an IgG1 immunoglobulin. For example, the Fc region or moiety may comprise a CH1 domain and a CH2 domain from an IgG4 molecule and a CH3 domain from an IgG1 molecule. For example, the Fc region or moiety may comprise a portion of a CH2 domain from a particular subclass of antibody, e.g., EU positions 292-340 of a CH2 domain. For example, an Fc region or moiety may comprise amino acids a positions 292-340 of CH2 derived from an IgG4 moiety and the remainder of CH2 derived from an IgG1 moiety (alternatively, 292-340 of CH2 may be derived from an IgG1 moiety and the remainder of CH2 derived from an IgG4 moiety).

It will also be appreciated that any antibody, antigen-binding fragment, or Fc region or moiety of the present disclosure can be of any allotype and/or haplotype. For example, human Immunoglobulin G allotypes include those disclosed in Jefferis and LeFranc, mAbs 1(4):1-7 (2009), which allotypes (including G1m (1(a); 2(x); 3(f); and 17(z)); G2m (23(n)); G3m (21(g1); 28(g5); 11(b0); 5(b2); 13(b3); 14(b4); 10(b5); 15(s); 16(t); 6(c3); 24(c5); 26(u); and 27(v)); A2m (1 and 2); and Km (1; 2; and 3) and haplotypes, and resultant amino acid sequences, and combinations thereof, are incorporated herein by reference. In certain embodiments, an antibody, antigen-binding fragment, or Fc region or moiety of the present disclosure comprises a IgG1 allotype g1m17, kl.

Moreover, an Fc region or moiety may (additionally or alternatively) for example comprise a chimeric hinge region. For example, the chimeric hinge may be derived, e.g. in part, from an IgG1, IgG2, or IgG4 molecule (e.g., an upper and lower middle hinge sequence) and, in part, from an IgG3 molecule (e.g., an middle hinge sequence). In another example, an Fc region or moiety may comprise a chimeric hinge derived, in part, from an IgG1 molecule and, in part, from an IgG4 molecule. In another example, the chimeric hinge may comprise upper and lower hinge domains from an IgG4 molecule and a middle hinge domain from an IgG1 molecule. Such a chimeric hinge may be made, for example, by introducing a proline substitution (Ser228Pro) at EU position 228 in the middle hinge domain of an IgG4 hinge region. In another embodiment, the chimeric hinge can comprise amino acids at EU positions 233-236 are from an IgG2 antibody and/or the Ser228Pro mutation, wherein the remaining amino acids of the hinge are from an IgG4 antibody (e.g., a chimeric hinge of the sequence ESKYGPPCPPCPAPPVAGP (SEQ ID NO.:74)). Further chimeric hinges which may be used in the Fc moiety of the antibody according to the present disclosure are described in US 2005/0163783 A1.

In some embodiments of the binding proteins disclosed herein, the Fc moiety, or the Fc region, comprises or consists of an amino acid sequence derived from a human immunoglobulin sequence (e.g., from an Fc region or Fc moiety from a human IgG molecule). However, polypeptides may comprise one or more amino acids from another mammalian species. For example, a primate Fc moiety or a primate binding site may be included in the subject polypeptides. Alternatively, one or more murine amino acids may be present in the Fc moiety or in the Fc region.

In some embodiments, an antibody is provided that comprises: a heavy chain (HC) comprising or consisting of the amino acid sequence set forth in SEQ ID NO.:75, optionally with the C-terminal lysine removed, and a light chain (LC), wherein the LC comprises or consists of (i) the VL amino acid sequence set forth in any one of SEQ ID NOs.:58-72 and (ii) the CL amino acid sequence set forth in SEQ ID NO.:79.

In some embodiments, an antibody is provided that comprises: a heavy chain (HC) comprising or consisting of the amino acid sequence set forth in SEQ ID NO.:76, optionally with the C-terminal lysine removed, and a light chain (LC), wherein the LC comprises or consists of (i) the VL amino acid sequence set forth in any one of SEQ ID NOs.:58-72 and (ii) the CL amino acid sequence set forth in SEQ ID NO.:79.

In some embodiments, an antibody is provided that comprises: a heavy chain (HC) comprising or consisting of the amino acid sequence set forth in SEQ ID NO.:77, optionally with the C-terminal lysine removed, and a light chain (LC), wherein the LC comprises or consists of (i) the VL amino acid sequence set forth in any one of SEQ ID NOs.:58-72 and (ii) the CL amino acid sequence set forth in SEQ ID NO.:79.

In some embodiments, an antibody is provided that comprises: a heavy chain (HC) comprising or consisting of the amino acid sequence set forth in SEQ ID NO.:78, optionally with the C-terminal lysine removed, and a light chain (LC), wherein the LC comprises or consists of (i) the VL amino acid sequence set forth in any one of SEQ ID NOs.:58-72 and (ii) the CL amino acid sequence set forth in SEQ ID NO.:79.

Nucleic Acid Molecules/Polynucleotides

In another aspect, the disclosure provides a nucleic acid molecule comprising a polynucleotide encoding an antibody, antigen binding fragment, or fusion protein according to the present disclosure. It will be understood that, for example, a first nucleic acid molecule can encode a heavy chain of an antibody, and a second nucleic acid molecule can encode a light chain of an antibody; these first and second nucleic acid molecules can still be referred-to as “a polynucleotide” or “a nucleic acid molecule” that encodes the antibody. In other words, a polynucleotide or nucleic acid molecule includes embodiments, wherein portions (e.g., chains) of an antibody or antigen-binding fragment are encoded by separate nucleic acid molecules and/or by separate portions of nucleci acid molecules. Exemplary polynucleotide sequences are provided in SEQ ID NOs.:80-99. In some embodiments, a polynucleotide encoding an antibody heavy chain comprises or consists of the polynucleotide sequence set forth in SEQ ID NO.:81, and a polynucleotide encoding an antibody VL or LC comprises the polynucleotide sequence set forth in any one of SEQ ID NOs.:85-99. In other embodiments, a polynucleotide encoding an antibody heavy chain comprises or consists of the polynucleotide sequence set forth in SEQ ID NO.:83, and a polynucleotide encoding an antibody VL or LC comprises the polynucleotide sequence set forth in any one of SEQ ID NOs.:85-99. In still other embodiments, a polynucleotide encoding an antibody heavy chain comprises or consists of the polynucleotide sequence set forth in SEQ ID NO.:84, and a polynucleotide encoding an antibody VL or LC comprises the polynucleotide sequence set forth in any one of SEQ ID NOs.:85-99.

Due to the redundancy of the genetic code, the present disclosure also comprises sequence variants of these nucleic acid sequences and in particular such sequence variants, which encode the same amino acid sequences.

In certain embodiments, a polynucleotide or nucleic acid molecule comprises a nucleotide sequence sharing at least 50% (i.e., 50%, 55%, 60%, 65%, 70%, 75%, 80%, 85%, 90%, 91%, 92%, 93%, 94%, 95%, 96%, 97%, 98%, 99%, or 100%) identity to the nucleotide sequence according to any one of SEQ ID NOs.:80-99, wherein the nucleotide sequence is codon optimized for expression by a host cell.

In particular embodiments, a nucleic acid molecule according to the present disclosure comprises or consists of a nucleic acid sequence according to any one of SEQ ID NOs: SEQ ID NOs.:80-99.

In certain embodiments, a polynucleotide comprises a VH-encoding nucleotide sequence having at least 50% identity to the amino acid sequence set forth in SEQ ID NO.:81 and a VL-encoding nucleotide sequence having at least 50% identity to the amino acid sequence set forth in any one of SEQ ID NOs.:85-97.

In any of the presently disclosed embodiments, a polynucleotide can comprise a VH-CH1-hinge-CH2-CH3-encoding nucleotide sequence according to SEQ ID NO:84. In some embodiments, a polynucleotide comprises a CL-encoding nucleotide sequence having at least 50% identity to the amino acid sequence set forth in SEQ ID NO:98 or 99.

Vectors

Further included within the scope of the disclosure are vectors, for example, expression vectors, that comprise a nucleic acid molecule according to the present disclosure.

The term “vector” refers to a construct comprising a nucleic acid molecule. A vector in the context of the present disclosure is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a desired antibody or antibody fragment thereof according to the present description.

As used herein, “expression vector” refers to a DNA construct containing a nucleic acid molecule that is operably linked to a suitable control sequence capable of effecting the expression of the nucleic acid molecule in a suitable host. Such control sequences include a promoter (e.g., a heterologous promoter) to effect transcription, an optional operator sequence to control such transcription, a sequence encoding suitable mRNA ribosome binding sites, and sequences which control termination of transcription and translation. Any of the elements of an expression vector that contribute to transcription of a nucleic acid molecule of interest may be heterologous to the vector. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

A cloning vector is typically a vector that contains a cloning site, which may be used to incorporate nucleic acid sequences into the vector. A cloning vector may be, e.g., a plasmid vector or a bacteriophage vector.

A transfer vector may be a vector which is suitable for transferring nucleic acid molecules into cells or organisms, for example, viral vectors. A vector in the context of the present disclosure may be, e.g., an RNA vector or a DNA vector. A vector may be a DNA molecule. For example, a vector in the sense of the present application comprises a cloning site, a selection marker, such as an antibiotic resistance factor, and a sequence suitable for multiplication of the vector, such as an origin of replication.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

Cells

In a further aspect, the present disclosure also provides a cell (also referred to as a “host cell”) expressing an antibody, antigen-binding fragment, or fusion protein according to the present disclosure; or comprising a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells. In certain such embodiments, the cells are a mammalian cell line such as CHO cells (e.g., DHFR-CHO cells (Urlaub et al., PNAS 77:4216 (1980), CHO-KSV, ExpiCHO), human embryonic kidney cells (e.g., HEK293T cells), PER.C6 cells, YO cells, Sp2/0 cells. NSO cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TM cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

Insect cells useful expressing a binding protein of the present disclosure are known in the art and include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J. Biotechnol. 153(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing a binding protein of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In some embodiments, a fusion protein is expressed at a cell surface by an immune cell, e.g., a T cell, NK cell, or NK-T cell, or any subtype thereof.

Any protein expression system compatible with the disclosure may be used to produce the disclosed binding proteins. Suitable expression systems include transgenic animals described in Gene Expression Systems, Academic Press, eds. Fernandez et al., 1999.

In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine etc. In certain embodiments, the introduction is non-viral.

Moreover, cells of the present disclosure may be transfected stably or transiently with the vector according to the present description, e.g. for expressing an antibody, or an antigen binding fragment thereof, according to the present description. In such embodiments, the cells are stably transfected with the vector as described herein encoding a binding protein. Alternatively, cells may be transiently transfected with a vector according to the present disclosure encoding a binding protein according to the present description. In any of the presently disclosed embodiments, a polynucleotide may be heterologous to the host cell.

In a related aspect, the present disclosure provides methods for producing an antibody, antigen-binding fragment, or fusion protein, wherein the methods comprise culturing a host cell of the present disclosure under conditions and for a time sufficient to produce the antibody, antigen-binding fragment, or fusion protein.

Accordingly, the present disclosure also provides recombinant host cells that heterologously express an antibody, antigen-binding fragment, or fusion protein of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody or antigen binding fragment in nature. Moreover, the host cell may impart a post-translational modification (PTM; e.g., glysocylation or fucosylation) on the binding protein that is not present in a native state of the binding protein (or in a native state of a parent binding protein from which the subject binding protein was engineered or derived). Such a PTM may result in a functional difference (e.g., reduced immunogenicity). Accordingly, a binding protein of the present disclosure that is produced by a host cell as disclosed herein may include one or more post-translational modification that is distinct from the binding protein or parent binding protein in its native state (e.g., a human antibody produced by a CHO cell can comprise a post-translational modification that is distinct from the antibody when isolated from the human and/or produced by the native human B cell or plasma cell).

### Optional Further Features of Antibodies, Antigen-Binding Fragments, and Fusion Proteins

Antibodies, antigen-binding fragments, and fusion proteins of the disclosure may be coupled, for example, to a drug for delivery to a treatment site or coupled to a detectable label to facilitate imaging of a site comprising cells of interest. Methods for coupling antibodies to drugs and detectable labels are well known in the art, as are methods for imaging using detectable labels. Labeled antibodies may be employed in a wide variety of assays, employing a wide variety of labels. Detection of the formation of an antibody-antigen complex between an antibody (or antigen binding fragment or fusion protein) of the disclosure and an epitope of interest on HBsAg, in particular on the antigenic loop region of HBsAg, can be facilitated by attaching a detectable substance to the antibody. Suitable detection means include the use of labels such as radionuclides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, β-galactosidase, or acetylcholinesterase; examples of suitable prosthetic group complexes include streptavidin/biotin and avidin/biotin;

examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include 125I, 131I, 35S, or 3H. Such labeled reagents may be used in a variety of well-known assays, such as radioimmunoassays, enzyme immunoassays, e.g., ELISA, fluorescent immunoassays, and the like. Labeled antibodies, antigen binding fragments, and fusion proteins according to the present disclosure may be thus be used in such assays for example as described in U.S. Pat. Nos. 3,766,162; 3,791,932; 3,817,837; and 4,233,402.

An antibody, antigen-binding fragment, or fusion protein according to the present disclosure may be conjugated to a therapeutic moiety such as a cytotoxin, a therapeutic agent, or a radioactive metal ion or radioisotope. Examples of radioisotopes include, but are not limited to, I-131, I-123, I-125, Y-90, Re-188, Re-186, At-211, Cu-67, Bi-212, Bi-213, Pd-109, Tc-99, In-111, and the like. Such conjugates can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin.

Techniques for conjugating such therapeutic moiety to antibodies are well known. See, for example, Arnon et al. (1985) “Monoclonal Antibodies for Immunotargeting of Drugs in Cancer Therapy”, in Monoclonal Antibodies and Cancer Therapy, ed. Reisfeld et al. (Alan R. Liss, Inc.), pp. 243-256; ed. Hellstrom et al. (1987) “Antibodies for Drug Delivery,” in Controlled Drug Delivery, ed. Robinson et al. (2d ed; Marcel Dekker, Inc.), pp. 623-653; Thorpe (1985) “Antibody Carriers of Cytotoxic Agents in Cancer Therapy: A Review,” in Monoclonal Antibodies '84: Biological and Clinical Applications, ed. Pinchera et al. pp. 475-506 (Editrice Kurtis, Milano, Italy, 1985); “Analysis, Results, and Future Prospective of the Therapeutic Use of Radiolabeled Antibody in Cancer Therapy,” in Monoclonal Antibodies for Cancer Detection and Therapy, ed. Baldwin et al. (Academic Press, New York, 1985), pp. 303-316; and Thorpe et al. (1982) Immunol. Rev. 62:119-158.

Alternatively, an antibody, antibody fragment, or fusion protein, can be conjugated to a second antibody, or antibody fragment thereof, (or second fusion protein) to form a heteroconjugate, e.g., as described in U.S. Pat. No. 4,676,980. In addition, linkers may be used between the labels and the antibodies of the description, e.g., as described in U.S. Pat. No. 4,831,175. Antibodies, antigen-binding fragments, and fusion proteins may be directly labeled with radioactive iodine, indium, yttrium, or other radioactive particle known in the art, e.g., as described in U.S. Pat. No. 5,595,721. Treatment may consist of a combination of treatment with conjugated and non-conjugated antibodies, antigen binding fragments, and/or fusion proteins, administered simultaneously or subsequently e.g., as described in WO00/52031; WO00/52473.

Antibodies, antigen-binding fragments, and fusion proteins as described herein may also be attached to a solid support. Additionally, the antibodies of the present disclosure, functional antibody fragments thereof, or fusion proteins, can be chemically modified by covalent conjugation to a polymer to, for example, increase their circulating half-life. Examples of polymers, and methods to attach them to peptides, are shown in U.S. Pat. Nos. 4,766,106; 4,179,337; 4,495,285 and 4,609,546. In some embodiments, the polymers may be selected from polyoxyethylated polyols and polyethylene glycol (PEG). PEG is soluble in water at room temperature and has the general formula: R(O—CH₂—CH₂)ₙO—R, wherein R can be hydrogen, or a protective group such as an alkyl or alkanol group. In certain embodiments, the protective group may have between 1 and 8 carbons. For example, the protective group may be methyl. The symbol n is a positive integer. In one embodiment, n is between 1 and 1,000. In another embodiment n is between 2 and 500. In some embodiments, the PEG has an average molecular weight selected from between 1,000 and 40,000, between 2,000 and 20,000, and between 3,000 and 12,000. Furthermore, PEG may have at least one hydroxy group, for example the PEG may have a terminal hydroxy group. For example, it is the terminal hydroxy group which is activated to react with a free amino group on the inhibitor. However, it will be understood that the type and amount of the reactive groups may be varied to achieve a covalently conjugated PEG/antibody of the present description.

Water-soluble polyoxyethylated polyols may also be utilized in the context of the antibodies and antigen binding fragments described herein. They include polyoxyethylated sorbitol, polyoxyethylated glucose, polyoxyethylated glycerol (POG), and the like. In one embodiment, POG is used. Without being bound by any theory, because the glycerol backbone of polyoxyethylated glycerol is the same backbone occurring naturally in, for example, animals and humans in mono-, di-, triglycerides, this branching would not necessarily be seen as a foreign agent in the body. POG may have a molecular weight in the same range as PEG. Another drug delivery system that can be used for increasing circulatory half-life is the liposome. Methods of preparing liposome delivery systems are known to one of skill in the art. Other drug delivery systems are known in the art and are described in, for example, referenced in Poznansky et al. (1980) and Poznansky (1984).

Antibodies, antigen-binding fragments, and fusion proteins of the disclosure may be provided in purified form. Typically, the antibody, antigen-binding fragment, or fusion protein will be present in a composition that is substantially free of other polypeptides e.g., where less than 90% (by weight), usually less than 60% and more usually less than 50% of the composition is made up of other polypeptides.

Antibodies, fusion proteins, or antigen-binding fragments of the disclosure may be immunogenic in non-human (or heterologous) hosts e.g., in mice. In particular, the antibodies, antigen-binding fragments, or fusion proteins may have an idiotope that is immunogenic in non-human hosts, but not in a human host. In particular, such molecules of the disclosure for human use include those that cannot be easily isolated from hosts such as mice, goats, rabbits, rats, non-primate mammals, etc. and cannot generally be obtained by humanization or from xeno-mice.

### Production of Antibodies, Antigen Binding Fragments, and Fusion Proteins

Antibodies, antigen-binding fragments, and fusion proteins according to the disclosure can be made by any method known in the art. For example, the general methodology for making monoclonal antibodies using hybridoma technology is well known (Kohler, G. and Milstein, C., 1975; Kozbar et al. 1983). In one embodiment, the EBV immortalization method described in WO2004/076677 is used.

In one embodiment, antibodies are produced using a method described in WO 2004/076677. In such methods, B cells producing the antibody are transformed with EBV and a polyclonal B cell activator. Additional stimulants of cellular growth and differentiation may optionally be added during the transformation step to further enhance the efficiency. These stimulants may be cytokines such as IL-2 and IL-15. In one aspect, IL-2 is added during the immortalization step to further improve the efficiency of immortalization, but its use is not essential. The immortalized B cells produced using these methods can then be cultured using methods known in the art and antibodies isolated therefrom.

Another method for producing antibodies is described in WO 2010/046775. In such a method, plasma cells are cultured in limited numbers, or as single plasma cells in microwell culture plates. Antibodies can be isolated from the plasma cell cultures. Further, from the plasma cell cultures, RNA can be extracted and PCR can be performed using methods known in the art. The VH and VL regions of the antibodies can be amplified by RT-PCR (reverse transcriptase PCR), sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

Standard techniques of molecular biology may be used to prepare DNA sequences encoding the antibodies, antigen-binding fragments, or fusion proteins of the present description. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody or fusion protein molecules of the present disclosure or fragments thereof. Bacterial, for example E. coli, and other microbial systems may be used, in part, for expression of antibody fragments such as Fab and F(ab′)2 fragments, and especially Fv fragments and single chain antibody fragments, for example, single chain Fvs. Eukaryotic, e.g., mammalian, host cell expression systems may be used for production of larger antibody molecules, including complete antibody molecules. Suitable mammalian host cells include, but are not limited to, those exemplary host cells and cell lines disclosed herein.

The present disclosure also provides a process for the production of an antibody, antigen-binding fragment, or fusion protein molecule according to the present disclosure comprising culturing a host cell comprising a vector encoding a nucleic acid of the present disclosure under conditions suitable for expression of protein from DNA encoding the antibody molecule of the present description, and isolating the antibody molecule.

An antibody molecule or antibody fragment may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Alternatively, antibodies, antigen-binding fragments, and fusion proteins according to the disclosure may be produced by (i) expressing a nucleic acid sequence according to the disclosure in a host cell, e.g. by use of a vector according to the present description, and (ii) isolating the expressed desired product. Additionally, the method may include (iii) purifying the isolated antibody, antigen-binding fragment, or fusion protein. Transformed B cells and cultured plasma cells may be screened for those producing antibodies, antigen-binding fragments, or fusion proteins of the desired specificity or function.

Screening may be carried out by any immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, or the like.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

The immortalized B cell clones or the transfected host-cells of described herein can be used in various ways e.g., as a source of monoclonal antibodies, as a source of nucleic acid (DNA or mRNA) encoding a monoclonal antibody of interest, for research, etc.

Inhibitors of HBV Protein Expression and Delivery Systems

The present disclosure also provides inhibitors of HBV protein expression for use in combination therapy methods and compositions a for treating HBV, wherein the combination therapy comprises a binding protein as provided herein. In certain embodiments, the inhibitor of HBV gene expression is an RNAi agent. As used herein, the term “RNA interference agent” or “RNAi agent” refers to an agent that contains RNA as that term is defined herein, and which mediates the targeted cleavage of an RNA transcript via an RNA-induced silencing complex (RISC) pathway. In some embodiments, an RNAi agent as described herein effects inhibition of expression of an HBV gene.

In one aspect, an RNA interference agent includes a single-stranded RNA that interacts with a target RNA sequence to direct the cleavage of the target RNA. Without wishing to be bound to a particular theory, long double-stranded RNA (dsRNA) introduced into plants and invertebrate cells is broken down into siRNA by a Type III endonuclease known as Dicer (Sharp, et al., Genes Dev. 15:485 (2001)). Dicer, a ribonuclease-III-like enzyme, processes the dsRNA into 19-23 base pair short interfering RNAs (siRNAs) with characteristic two base 3′ overhangs (Bernstein, et al., Nature 409:363 (2001)). The siRNAs are then incorporated into an RNA-induced silencing complex (RISC) where one or more helicases unwind the siRNA duplex, enabling the complementary antisense strand to guide target recognition (Nykanen, et al., Cell 107:309 (2001)). Upon binding to the appropriate target mRNA, one or more endonucleases within the RISC cleaves the target to induce silencing (Elbashir, et al, Genes Dev. 15:188 (2001)). Thus, in one aspect the technology described herein relates to a single stranded RNA that promotes the formation of a RISC complex to effect silencing of the target gene.

The terms “silence,” “inhibit the expression of,” “down-regulate the expression of,” “suppress the expression of,” and the like, in so far as they refer to an HBV gene, herein refer to the at least partial reduction of the expression of an HBV gene, as manifested by a reduction of the amount of HBV mRNA which can be isolated from or detected in a first cell or group of cells in which an HBV gene is transcribed and which has or have been treated with an inhibitor of HBV gene expression, such that the expression of the HBV gene is inhibited, as compared to a second cell or group of cells substantially identical to the first cell or group of cells but which has or have not been so treated (control cells). The degree of inhibition can be measured, by example, as the difference between the degree of mRNA expression in a control cell minus the degree of mRNA expression in a treated cell. Alternatively, the degree of inhibition can be given in terms of a reduction of a parameter that is functionally linked to HBV gene expression, e.g., the amount of protein encoded by an HBV gene, or the number of cells displaying a certain phenotype, e.g., an HBV infection phenotype such as HBV infection, HBV protein expression (such as hepatitis B surface antigen, HBsAg), or changes in cellular gene expression reflecting HBV gene expression (e.g., Smc5/6 expression and localization). The degree of inhibition may also be measured using a cell engineered to express a reporter gene reflecting HBV RNA expression. In principle, HBV gene silencing can be determined in any cell expressing the HBV gene, e.g., an HBV-infected cell or a cell engineered to express the HBV gene, and by any appropriate assay.

The level of HBV RNA that is expressed by a cell or group of cells, or the level of circulating HBV RNA, may be determined using any method known in the art for assessing mRNA expression, such as the rtPCR method provided in Example 2 of International Application Publication No. WO 2016/077321A1 and U.S. Patent Application No. US2017/0349900A1, which methods are incorporated herein by reference. In some embodiments, the level of expression of an HBV gene (e.g., total HBV RNA, an HBV transcript, e.g., HBV 3.5 kb transcript) in a sample is determined by detecting a transcribed polynucleotide, or portion thereof, e.g., RNA of the HBV gene. RNA may be extracted from cells using RNA extraction techniques including, for example, using acid phenol/guanidine isothiocyanate extraction (RNAzol B; Biogenesis), RNeasy RNA preparation kits (Qiagen®), or PAXgene (PreAnalytix, Switzerland). Typical assay formats utilizing ribonucleic acid hybridization include nuclear run-on assays, RT-PCR, RNase protection assays (Melton et al., Nuc. Acids Res. 12:7035), northern blotting, in situ hybridization, and microarray analysis. Circulating HBV mRNA may be detected using methods the described in International Application Publication No. WO 2012/177906A1 and U.S. Patent Application No. US2014/0275211A1, which methods are incorporated herein by reference.

As used herein, “target sequence” refers to a contiguous portion of the nucleotide sequence of an mRNA molecule formed during the transcription of an HBV gene, including mRNA that is a product of RNA processing of a primary transcription product. The target portion of the sequence will be at least long enough to serve as a substrate for RNAi-directed cleavage at or near that portion. For example, the target sequence will generally be from 9-36 nucleotides in length, e.g., 15-30 nucleotides in length, including all sub-ranges there between. As non-limiting examples, the target sequence can be from 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 18-30 nucleotides, 18-26 nucleotides, 18-23 nucleotides, 18-22 nucleotides, 18-21 nucleotides, 18-20 nucleotides, 19-30 nucleotides, 19-26 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides, 20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an RNAi agent, e.g., within an siRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, an siRNA comprising one oligonucleotide 21 nucleotides in length, and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from non-Watson-Crick base pairs and/or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary,” and “substantially complementary” herein can be used with respect to the base matching between the sense strand and the antisense strand of an siRNA, or between the antisense strand of an RNAi agent and a target sequence, as will be understood from the context of their use. As used herein, a polynucleotide that is “substantially complementary” to at least part of a messenger RNA (mRNA) refers to a polynucleotide that is substantially complementary to a contiguous portion of the mRNA of interest (e.g., an mRNA encoding an HBV protein). For example, a polynucleotide is complementary to at least a part of an HBV mRNA if the sequence is substantially complementary to a non-interrupted portion of the HBV mRNA.

a. siRNAs

In some embodiments, the RNAi agent comprises an siRNA. The term “siRNA,” as used herein, refers to an RNAi that includes an RNA molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range there between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, and 21-22 base pairs. siRNAs generated in the cell by processing with Dicer and similar enzymes are generally in the range of 19-22 base pairs in length. The term “double-stranded RNA” or “dsRNA,” is also used herein synonymously to refer to an siRNA as described above.

One strand of the duplex region of an siRNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of an siRNA are comprised by separate RNA molecules, those molecules need not, but can be covalently connected. Where the two strands are connected covalently by means other than a hairpin loop, the connecting structure is referred to as a “linker.”

The term “antisense strand” or “guide strand” refers to the strand of an RNAi agent, e.g., an siRNA, which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches can be in the internal or terminal regions of the molecule.

Generally, the most tolerated mismatches are in the terminal regions, e.g., within 5, 4, 3, or 2 nucleotides of the 5′ and/or 3′ terminus.

The term “sense strand” or “passenger strand” as used herein, refers to the strand of an RNAi that includes a region that is substantially complementary to a region of the antisense strand as that term is defined herein.

In another aspect, the agent is a single-stranded antisense RNA molecule. The antisense RNA molecule can have 15-30 nucleotides complementary to the target. For example, the antisense RNA molecule may have a sequence of at least 15, 16, 17, 18, 19, 20, 21, or more contiguous nucleotides from one of the antisense sequences disclosed herein.

The skilled artisan will recognize that the term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The RNA can be modified in the nucleobase structure or in the ribose-phosphate backbone structure, e.g., as described in greater detail below. However, siRNA molecules comprising ribonucleoside analogs or derivatives retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate, or a non-natural base comprising nucleoside, or any combination thereof. Alternatively, an RNA molecule can comprise at least two modified ribonucleosides, at least 3, at least 4, at least 5, at least 6, at least 7, at least 8, at least 9, at least 10, at least 15, at least 20, or more, up to the entire length of the siRNA molecule. The modifications need not be the same for each of such a plurality of modified ribonucleosides in an RNA molecule. In some embodiments, modified RNAs contemplated for use in methods and compositions described herein are peptide nucleic acids (PNAs) that have the ability to form the required duplex structure and that permit or mediate the specific degradation of a target RNA via a RISC pathway.

In some embodiments, a modified ribonucleoside includes a deoxyribonucleoside. For example, an RNAi agent can comprise one or more deoxynucleosides, including, for example, a deoxynucleoside overhang(s), or one or more deoxynucleosides within the double-stranded portion of an siRNA. However, the term “RNAi agent” as used herein does not include a fully DNA molecule.

As used herein, the term “nucleotide overhang” refers to at least one unpaired nucleotide that protrudes from the duplex structure of an RNAi agent, e.g., an siRNA. For example, when a 3′-end of one strand of an siRNA extends beyond the 5′-end of the other strand, or vice versa, there is a nucleotide overhang. An siRNA can comprise an overhang of at least one nucleotide; alternatively the overhang can comprise at least two nucleotides, at least three nucleotides, at least four nucleotides, at least five nucleotides, or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′ end, 3′ end, or both ends of either an antisense or sense strand of an siRNA.

In some embodiments, the antisense strand of an siRNA has a 1-10 nucleotide overhang at the 3′ end and/or the 5′ end. In some embodiments, the sense strand of an siRNA has a 1-10 nucleotide overhang at the 3′ end and/or the 5′ end. In some other embodiments, one or more of the nucleotides in the overhang is replaced with a nucleoside thiophosphate.

In some embodiments, at least one end of an siRNA has a single-stranded nucleotide overhang of 1 to 4, generally 1 or 2 nucleotides. siRNAs having at least one nucleotide overhang can have unexpectedly superior inhibitory properties relative to their blunt-ended counterparts.

The terms “blunt” or “blunt ended” as used herein in reference to an siRNA mean that there are no unpaired nucleotides or nucleotide analogs at a given terminal end of an siRNA, i.e., no nucleotide overhang. One or both ends of an siRNA can be blunt. Where both ends of an siRNA are blunt, the siRNA is said to be “blunt ended.” A “blunt ended” siRNA is an siRNA that is blunt at both ends, i.e., has no nucleotide overhang at either end of the molecule. Most often such a molecule will be double-stranded over its entire length.

In certain embodiments, the combination therapy described herein includes one or more RNAi agents that inhibit the expression of the HBV gene. In some embodiments, the RNAi agent includes short interfering ribonucleic acid (siRNA) molecules for inhibiting the expression of an HBV gene in a mammal, e.g., in an HBV-infected human, where the siRNA includes an antisense strand having a region of complementarity which is complementary to at least a part of an mRNA formed in the expression of an HBV gene, and where the region of complementarity is 30 nucleotides or less in length, generally 19-24 nucleotides in length, and where the siRNA, upon contact with a cell expressing the HBV gene, inhibits the expression of the HBV gene by at least 10% as assayed by, for example, a PCR or branched DNA (bDNA)-based method, or by a protein-based method, such as by Western blot. Expression of an HBV gene in cell culture or expression of a cellular gene as a surrogate for HBV gene expression (e.g., Smc5/6), such as in COS cells, HeLa cells, primary hepatocytes, HepG2 cells, primary cultured cells or in a biological sample from a subject, can be assayed by measuring HBV mRNA levels, such as by bDNA or TaqMan assay, or by measuring protein levels, such as by immunofluorescence analysis, using, for example, Western Blotting or flow cytometric techniques.

An siRNA includes two RNA strands that are complementary and hybridize to form a duplex structure under conditions in which the siRNA will be used. One strand of an siRNA (the antisense strand) includes a region of complementarity that is substantially complementary, and generally fully complementary, to a target sequence. The target sequence can be derived from the sequence of an mRNA formed during the expression of an HBV gene. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the siRNA is between 15 and 20 nucleotides in length, inclusive, and in other embodiments, the siRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). siRNAs having duplexes as short as 9 base pairs can, under some circumstances, mediate RNAi-directed RNA cleavage. Most often a target will be at least 15 nucleotides in length. In certain embodiments, the target is 15-30 nucleotides in length.

One of skill in the art will also recognize that the duplex region is a primary functional portion of an siRNA, e.g., a duplex region of 9 to 36, e.g., 15-30 base pairs. Thus, in some embodiments, to the extent that it becomes processed to a functional duplex of e.g., 15-30 base pairs that targets a desired RNA for cleavage, an RNA molecule or complex of RNA molecules having a duplex region greater than 30 base pairs is an siRNA. Thus, an ordinarily skilled artisan will recognize that in some embodiments, then, a miRNA is an siRNA. In some other embodiments, an siRNA is not a naturally occurring miRNA. In some embodiments, an RNAi agent useful to target expression of an HBV gene is not generated in the target cell by cleavage of a larger double-stranded RNA.

An siRNA as described herein can be synthesized by standard methods known in the art, e.g., by use of an automated DNA synthesizer, such as are commercially available from, for example, Biosearch, Applied Biosystems, Inc.

In some embodiments, the RNAi agent comprises an siRNA that targets and inhibits expression of an HBV mRNA. In some embodiments, the RNAi agent comprises an siRNA that targets and inhibits expression of an mRNA encoded by an HBV genome according to NCBI Reference Sequence NC_003977.2 (GenBank Accession No. GI:21326584) (SEQ ID NO:116). Transcription of the HBV genome results in polycistronic, overlapping RNAs, and therefore, in some embodiments, an siRNA of the combination therapy targeting a single HBV gene may result in significant inhibition of expression of most or all HBV transcripts. In some embodiments the mRNA target of the siRNA may be an mRNA encoded by: P gene, nucleotides 2309-3182 and 1-1625 of NC_003977.1; S gene (encoding L, M, and S proteins), nucleotides 2850-3182 and 1-837 of NC_003977; X protein, nucleotides 1376-1840 of NC_003977; and/or C gene, nucleotides 1816-2454 of NC_003977.

In some embodiments, the siRNA targets and inhibits expression of an mRNA encoded by the X gene of HBV. In some embodiments, the RNAi agent or siRNA targets an mRNA encoded by a portion of the HBV genome comprising the sequence GTGTGCACTTCGCTTCAC (SEQ ID NO:117), which corresponds to nucleotides 1579-1597 of NC_003977.2 (GenBank Accession No. GI:21326584) (SEQ ID NO:116).

In still further embodiments, the siRNA has a sense strand comprising 5′-GUGUGCACUUCGCUUCACA-3′ (SEQ ID NO:118) and an antisense strand comprising 5′-UGUGAAGCGAAGUGCACACUU-3′ (SEQ ID NO:119).

In certain embodiments, the inhibitor of HBV gene expression comprises an siRNA comprising a sense strand and an antisense strand, wherein the sense strand comprises SEQ ID NO:118, or a sequence that differs by not more than 4, not more than 3, not more than 2, or not more than 1 nucleotides from SEQ ID NO:118; and wherein the antisense strand comprises SEQ ID NO:119, or a sequence that differs by not more than 4, not more than 3, not more than 2, or not more than 1 nucleotides from SEQ ID NO:119.

In one aspect, an siRNA will include at least two nucleotide sequences, a sense and an antisense sequence, whereby: the sense sequence comprises SEQ ID NO:118, and the corresponding antisense sequence comprises SEQ ID NO:119. In this aspect, one of the two sequences is complementary to the other of the two sequences, with one of the sequences being substantially complementary to a sequence of an mRNA generated in the expression of an HBV gene. As such, in this aspect, an siRNA will include two oligonucleotides, where one oligonucleotide is described as the sense strand, and the second oligonucleotide is described as the corresponding antisense strand of the sense strand. As described elsewhere herein and as known in the art, the complementary sequences of an siRNA can also be contained as self-complementary regions of a single nucleic acid molecule, as opposed to being on separate oligonucleotides.

In still further embodiments, the siRNA has a sense strand comprising 5′-GGUGGACUUCUCUCAAUUUUA-3′ (SEQ ID NO:120) and an antisense strand comprising 5′-UAAAAUUGAGAGAAGUCCACCAC-3′ (SEQ ID NO:121).

In certain embodiments, the inhibitor of HBV gene expression comprises an siRNA comprising a sense strand and an antisense strand, wherein the sense strand comprises SEQ ID NO:120, or a sequence that differs by not more than 4, not more than 3, not more than 2, or not more than 1 nucleotides from SEQ ID NO:120; and wherein the antisense strand comprises SEQ ID NO:121, or a sequence that differs by not more than 4, not more than 3, not more than 2, or not more than 1 nucleotides from SEQ ID NO:121.

In one aspect, an siRNA will include at least two nucleotide sequences, a sense and an antisense sequence, whereby: the sense sequence comprises SEQ ID NO:120, and the corresponding antisense sequence comprises SEQ ID NO:121. In this aspect, one of the two sequences is complementary to the other of the two sequences, with one of the sequences being substantially complementary to a sequence of an mRNA generated in the expression of an HBV gene. As such, in this aspect, an siRNA will include two oligonucleotides, where one oligonucleotide is described as the sense strand, and the second oligonucleotide is described as the corresponding antisense strand of the sense strand. As described elsewhere herein and as known in the art, the complementary sequences of an siRNA can also be contained as self-complementary regions of a single nucleic acid molecule, as opposed to being on separate oligonucleotides.

The skilled person is well aware that siRNAs having a duplex structure of between 20 and 23, but specifically 21, base pairs have been hailed as particularly effective in inducing RNA interference (Elbashir, et al., EMBO 20:6877-88 (2001)). However, others have found that shorter or longer RNA duplex structures can be effective as well. In the embodiments described above, siRNAs described herein can include at least one strand of a length of minimally 21 nucleotides. In some embodiments, shorter duplexes having one of the sequences of SEQ ID NO:118, SEQ ID NO:119, SEQ ID NO:120, or SEQ ID NO:121 minus only a few nucleotides on one or both ends are similarly effective as compared to the siRNAs described above. Hence, siRNAs having a partial sequence of at least 15, 16, 17, 18, 19, 20, or more contiguous nucleotides from one or both of SEQ ID NO:118 and SEQ ID NO:119, and differing in their ability to inhibit the expression of an HBV gene by not more than 5, 10, 15, 20, 25, or 30% inhibition from an siRNA comprising the full sequence, are contemplated according to the technology described herein. Also within the present disclosure are siRNAs having a partial sequence of at least 15, 16, 17, 18, 19, 20, or more contiguous nucleotides from one or both of SEQ ID NO:120 and SEQ ID NO:121, and differing in their ability to inhibit the expression of an HBV gene by not more than 5, 10, 15, 20, 25, or 30% inhibition from an siRNA comprising the full sequence, are contemplated according to the technology described herein.

In addition, the siRNAs provided in herein identify a site in an HBV gene transcript that is susceptible to RISC-mediated cleavage. As such, the technology described herein further features RNAi agents that target within one of such sequences. As used herein, an RNAi agent is said to target within a particular site of an RNA transcript if the RNAi promotes cleavage of the transcript anywhere within that particular site. In some embodiments, the RNAi agent includes at least 15 contiguous nucleotides from one or both of the sequences of SEQ ID NO:118 and SEQ ID NO:119, coupled to additional nucleotide sequences taken from the region contiguous to the selected sequence in the HBV gene. In some embodiments, the RNAi agent includes at least 15 contiguous nucleotides from one or both of the sequences of SEQ ID NO:120 and SEQ ID NO:121, coupled to additional nucleotide sequences taken from the region contiguous to the selected sequence in the HBV gene.

While a target sequence is generally 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an RNAi agent, mediate the best inhibition of target gene expression. It is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified, e.g., SEQ ID NO:118, SEQ ID NO:119, SEQ ID NO:120, or SEQ ID NO:121, further optimization could be achieved by systematically either adding or removing nucleotides to generate longer or shorter sequences and testing those and sequences generated by walking a window of the longer or shorter size up or down the target RNA from that point. Again, coupling this approach to generating new candidate targets with testing for effectiveness of RNAi agents based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

An RNAi agent as described herein can contain one or more mismatches to the target sequence. In some embodiments, an RNAi agent as described herein contains no more than 3 mismatches. In some embodiments, if the antisense strand of the RNAi agent contains mismatches to a target sequence, the area of mismatch is not located in the center of the region of complementarity. In particular embodiments, if the antisense strand of the RNAi agent contains mismatches to the target sequence, the mismatch is restricted to within the last 5 nucleotides from either the 5′ or 3′ end of the region of complementarity. For example, for a 23 nucleotide RNAi agent RNA strand which is complementary to a region of an HBV gene, the RNA strand may not contain any mismatch within the central 13 nucleotides. The methods described herein or methods known in the art can be used to determine whether an RNAi agent containing a mismatch to a target sequence is effective in inhibiting the expression of an HBV gene. Consideration of the efficacy of RNAi agents with mismatches in inhibiting expression of an HBV gene is important, especially if the particular region of complementarity in the HBV gene is known to have polymorphic sequence variation.

b. Chemically Modified RNAi Agents

In some embodiments, the RNA of an RNAi agent, e.g., an siRNA, is chemically modified to enhance stability or other beneficial characteristics. The nucleic acids featured in the technology described herein can be synthesized and/or modified by methods well established in the art, such as those described in “Current protocols in nucleic acid chemistry,” Beaucage, S. L., et al. (Edrs.), John Wiley & Sons, Inc., New York, N.Y., USA, which methods are incorporated herein by reference.

Modifications include, for example, (a) end modifications, e.g., 5′ end modifications (phosphorylation, conjugation, inverted linkages, etc.), 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

It is not necessary for all positions in a given compound to be uniformly modified, and in fact more than one of the aforementioned modifications can be incorporated in a single compound or even at a single nucleoside within an RNAi agent. The technology described herein also includes RNAi agent compounds that are chimeric compounds. “Chimeric” RNAi agent compounds or “chimeras,” in the context of this disclosure, are RNAi agent compounds, such as siRNAs, which contain two or more chemically distinct regions, each made up of at least one monomer unit, i.e., a nucleotide in the case of an siRNA compound. These RNAi agents typically contain at least one region wherein the RNA is modified so as to confer upon the RNAi agent increased resistance to nuclease degradation, increased cellular uptake, and/or increased binding affinity for the target nucleic acid. An additional region of the RNAi agent can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of RNAi agent inhibition of gene expression. Consequently, comparable results can often be obtained with shorter RNAi agents when chimeric siRNAs are used, compared to phosphorothioate deoxy siRNAs hybridizing to the same target region. Cleavage of the RNA target can be routinely detected by gel electrophoresis and, if necessary, associated nucleic acid hybridization techniques known in the art.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts, and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464; each of which is herein incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH2 component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439; each of which is herein incorporated by reference for teachings relevant to such methods of preparation.

In other embodiments, suitable RNA mimetics suitable are contemplated for use in RNAi agents, in which both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262; each of which is incorporated herein by reference for teachings relevant to such methods of preparation. Further teaching of PNA compounds can be found, for example, in Nielsen, et al. (Science, 254:1497-1500 (1991)).

Some embodiments featured in the technology described herein include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —N(CH₃)—CH₂—CH₂— [wherein the native phosphodiester backbone is represented as —O—P—O—CH₂—] of U.S. Pat. No. 5,489,677, and the amide backbones of U.S. Pat. No. 5,602,240. In some embodiments, the RNAs featured herein have morpholino backbone structures of U.S. Pat. No. 5,034,506.

Modified RNAs can also contain one or more substituted sugar moieties. The RNAi agents, e.g., siRNAs, featured herein can include one of the following at the 2′ position: OH; F; O-, S-, or N-alkyl; O-, S-, or N-alkenyl; O-, S- or N-alkynyl; or O-alkyl-O-alkyl; wherein the alkyl, alkenyl, and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)·ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, siRNAs include one of the following at the 2′ position: C₁ to C₁₀ lower alkyl, substituted lower alkyl, alkaryl, aralkyl, O-alkaryl or O-aralkyl, SH, SCH₃, OCN, CI, Br, CN, CF₃, OCF₃, SOCH₃, SO₂CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an RNAi agent, or a group for improving the pharmacodynamic properties of an RNAi agent, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin, et al., Helv. Chim. Acta 78:486-504 (1995)), i.e., an alkoxy-alkoxy group. Another exemplary modification is 2′-dimethylaminooxyethoxy, i.e., a O(CH₂)₂ON(CH₃)₂ group, also known as 2′-DMAOE, and 2′-dimethylaminoethoxyethoxy (also known in the art as 2*-O-dimethylaminoethoxyethyl or 2*-DMAEOE), i.e., 2*-O—CH₂—O—CH₂—N(CH₂)₂.

Other exemplary modifications include 2′-methoxy (2′-OCH₃), 2′-aminopropoxy (2-OCH₂CH₂CH₂NH₂), and 2′-fluoro (2′-F). Similar modifications can also be made at other positions on the RNA of an RNAi agent, particularly the 3′ position of the sugar on the 3′ terminal nucleotide or in 2′-5′ linked siRNAs and the 5′ position of the 5′ terminal nucleotide. RNAi agents can also have sugar mimetics such as cyclobutyl moieties in place of the pentofuranosyl sugar.

Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920; each of which is incorporated herein by reference for teachings relevant to such methods of preparation.

An RNAi agent can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl, and other 5-substituted uracils and cytosines, 7-methylguanine and 7-methyladenine, 8-azaguanine and 8-azaadenine, 7-deazaguanine and 7-daazaadenine, and 3-deazaguanine and 3-deazaadenine. Further nucleobases include those disclosed in U.S. Pat. No. 3,687,808, those disclosed in Modified Nucleosides in Biochemistry, Biotechnology and Medicine (Herdewijn, P. ed. Wiley-VCH, (2008)); those disclosed in The Concise Encyclopedia Of Polymer Science And Engineering (pages 858-859, Kroschwitz, J. L, ed. John Wiley & Sons (1990)), those disclosed by Englisch et al. (Angewandte Chemie, International Edition, 30, 613 (1991)), and those disclosed by Sanghvi, Y S. (Chapter 15, dsRNA Research and Applications, pages 289-302, Crooke, S. T. and Lebleu, B., Ed., CRC Press (1993)). Certain of these nucleobases are particularly useful for increasing the binding affinity of the oligomeric compounds featured in the technology described herein. These include 5-substituted pyrimidines, 6-azapyrimidines and N-2, N-6, and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, pp. 276-278 (1993)) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, U.S. Pat. Nos. 3,687,808; 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088; each of which is incorporated herein by reference for teachings relevant to such methods of preparation.

The RNA of an RNAi agent can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J., et al., Nucleic Acids Research 33(0:439-47 (2005); Mook, O. R., et al., Mol Cane Ther 6(3):833-43 (2007); Grunweller, A., et al, Nucleic Acids Research 31(12):3185-93 (2003)).

Representative U.S. patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845; each of which is incorporated herein by reference for teachings relevant to such methods of preparation.

In certain embodiments, the combination therapy includes an siRNA that is modified to include one or more adenosine-glycol nucleic acid (“GNA”). A description of adenosine-GNA can be found, for example, in Zhang, et al. (JACS 127(12):4174-75 (2005)).

In some embodiments, the present disclosure provides methods and related compositions, wherein the RNAi is an siRNA comprising an oligonucleotide sequence having one or more modified nucleotides. Abbreviations for nucleotide monomers in modified nucleic acid sequences as used herein are provided in Table 5.

TABLE 5
Abbreviations of nucleotide monomers used in modified nucleic
acid sequence representation. It will be understood that, unless
otherwise indicated, these monomers, when present in an oligonucleotide,
are mutually linked by 5′-3′-phosphodiester bonds.

| Abbreviation | Nucleotide(s) |
|---|---|
| A | adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | uridine-3′-phosphate |
| Uf | 2′-fluorouridine-3′-phosphate |
| Ufs | 2′-fluorouridine-3′-phosphorothioate |
| Us | uridine-3′-phosphorothioate |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| | (also referred to as “Hyp-(GalNAc-alkyl)3”) |
| (Agn) | adenosine-glycol nucleic acid (GNA) |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |

In some embodiments, the inhibitor of HBV gene expression comprises an siRNA, wherein the siRNA has a sense strand comprising 5′-gsusguGfcAfCfUfucgcuucacaL96-3′ (SEQ ID NO:122) and an antisense strand comprising 5′-usGfsugaAfgCfGfaaguGfcAfcacsusu-3′ (SEQ ID NO:123).

In still further embodiments, the siRNA has a sense strand comprising 5′-gsusguGfcAfCfUfucgcuucacaL96-3′ (SEQ ID NO:124) and an antisense strand comprising 5′-usGfsuga(Agn)gCfGfaaguGfcAfcacsusu-3′ (SEQ ID NO:125).

In certain embodiments, the inhibitor of HBV gene expression comprises an siRNA comprising a sense strand and an antisense strand, wherein the sense strand comprises SEQ ID NO:122 or SEQ ID NO:124, or a sequence that differs by not more than 4, not more than 3, not more than 2, or not more than 1 nucleotide from SEQ ID NO:122 or SEQ ID NO:124, respectively.

In certain embodiments, the inhibitor of HBV gene expression comprises an siRNA comprising a sense strand and an antisense strand, wherein the antisense strand comprises SEQ ID NO:123 or SEQ ID NO:125, or a sequence that differs by not more than 4, not more than 3, not more than 2, or not more than 1 nucleotide from SEQ ID NO:123 or SEQ ID NO:125, respectively.

In some embodiments, the inhibitor of HBV gene expression comprises an siRNA, wherein the siRNA has a sense strand comprising 5′-gsgsuggaCfuUfCfUfcucaAfUfuuuaL96-3′ (SEQ ID NO:126) and an antisense strand comprising 5′-usAfsaaaUfuGfAfgagaAfgUfccaccsasc-3′ (SEQ ID NO:127).

In certain embodiments, the inhibitor of HBV gene expression comprises an siRNA comprising a sense strand and an antisense strand, wherein the sense strand comprises SEQ ID NO:126, or a sequence that differs by not more than 4, not more than 3, not more than 2, or not more than 1 nucleotide from SEQ ID NO:126.

c. Ligand-Conjugated RNAi Agents

In some embodiments, the RNAi agent includes modifications involving chemically linking to the RNA one or more ligands, moieties, or conjugates that enhance the activity, cellular distribution, or cellular uptake of the RNAi agent. Such moieties include but are not limited to lipid moieties such as a cholesterol moiety (Letsinger, et al., Proc. Natl. Acid. Sci. USA 86:6553-56 (1989)), cholic acid (Manoharan, et al., Biorg. Med. Chem. Let. 4:1053-60 (1994)), a thioether, e.g., beryl-S-tritylthiol (Manoharan, et al., Ann. N.Y. Acad. Sci. 660:306-9 (1992); Manoharan, et al., Biorg. Med. Chem. Let. 3:2765-70 (1993)), a thiocholesterol (Oberhauser, et al., Nucl. Acids Res. 20:533-38 (1992)), an aliphatic chain, e.g., dodecandiol or undecyl residues (Saison-Behmoaras, et al., EMBO J 10:1111-18 (1991); Kabanov, et al., FEBS Lett. 259:327-30 (1990); Svinarchuk, et al., Biochimie 75:49-54 (1993)), a phospholipid, e.g., di-hexadecyl-rac-glycerol or triethyl-ammonium 1,2-di-O-hexadecyl-rac-glycero-3-phosphonate (Manoharan, et al., Tetrahedron Lett. 36:3651-54 (1995); Shea, et al., Nucl. Acids Res. 18:3777-83 (1990)), a polyamine or a polyethylene glycol chain (Manoharan, et al., Nucleosides & Nucleotides 14:969-73 (1995)), or adamantane acetic acid (Manoharan, et al., Tetrahedron Lett. 36:3651-54 (1995)), a palmityl moiety (Mishra, et al., Biochim. Biophys. Acta 1264:229-37 (1995)), or an octadecylamine or hexylamino-carbonyloxycholesterol moiety (Crooke, et al., J. Pharmacol. Exp. Ther. 277:923-37 (1996)).

In some embodiments, a ligand alters the distribution, targeting, or lifetime of an RNAi agent into which it is incorporated. In some embodiments, a ligand provides an enhanced affinity for a selected target, e.g., molecule, cell, or cell type, compartment, e.g., a cellular or organ compartment, tissue, organ, or region of the body, as, e.g., compared to a species absent such a ligand. In such embodiments, the ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a liver cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic. Other examples of ligands include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic molecules (e.g., cholesterol, cholic acid, adamantane acetic acid, 1-pyrene butyric acid, dihydrotestosterone, 1,3-Bis-0(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine), peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, and AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, and multivalent fucose. The ligand can be, for example, a lipopolysaccharide, an activator of p38 MAP kinase, or an activator of NF-KB.

The ligand can be a substance, e.g., a drug, which can increase the uptake of the RNAi agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In another aspect, the ligand is a moiety, e.g., a vitamin, which is taken up by a target cell, e.g., a liver cell. Exemplary vitamins include vitamin A, E, and K. Other exemplary vitamins include are B vitamin, e.g., folic acid, B12, riboflavin, biotin, pyridoxal, or other vitamins or nutrients taken up by target cells such as liver cells. Also included are HSA and low density lipoprotein (LDL).

In some embodiments, a ligand attached to an RNAi agent as described herein acts as a pharmacokinetic (PK) modulator. As used herein, a “PK modulator” refers to a pharmacokinetic modulator. PK modulators include lipophiles, bile acids, steroids, phospholipid analogues, peptides, protein binding agents, PEG, vitamins, etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin, etc. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable to the technology described herein as ligands (e.g., as PK modulating ligands). In addition, aptamers that bind serum components (e.g., serum proteins) are also suitable for use as PK modulating ligands in the embodiments described herein.

(i) Lipid conjugates. In some embodiments, the ligand or conjugate is a lipid or lipid-based molecule. A lipid or lipid-based ligand can (a) increase resistance to degradation of the conjugate, (b) increase targeting or transport into a target cell or cell membrane, and/or (c) can be used to adjust binding to a serum protein, e.g., HSA. Such a lipid or lipid-based molecule may bind a serum protein, e.g., human serum albumin (HSA). An HSA-binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In some embodiments, the lipid based ligand binds HSA. The lipid based ligand may bind to HSA with a sufficient affinity such that the conjugate will be distributed to a non-kidney tissue. In certain particular embodiments, the HSA-ligand binding is reversible.

In some other embodiments, the lipid based ligand binds HSA weakly or not at all, such that the conjugate will be distributed to the kidney. Other moieties that target to kidney cells can also be used in place of or in addition to the lipid based ligand.

(ii) Cell Permeation Peptide and Agents. In another aspect, the ligand is a cell-permeation agent, such as a helical cell-permeation agent. In some embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. In some embodiments, the helical agent is an alpha-helical agent. In certain particular embodiments, the helical agent has a lipophilic and a lipophobic phase.

A “cell permeation peptide” is capable of permeating a cell, e.g., a microbial cell, such as a bacterial or fungal cell, or a mammalian cell, such as a human cell. A microbial cell-permeating peptide can be, for example, an alpha-helical linear peptide (e.g., LL-37 or Ceropin PI), a disulfide bond-containing peptide (e.g., α-defensin, β-defensin, or bactenecin), or a peptide containing only one or two dominating amino acids (e.g., PR-39 or indolicidin).

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to RNAi agents can affect pharmacokinetic distribution of the RNAi, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO:128). An RFGF analogue (e.g., amino acid sequence AALLPVLLAAP (SEQ ID NO:129) containing a hydrophobic MTS can also be a targeting moiety. The peptide moiety can be a “delivery” peptide, which can carry large polar molecules including peptides, oligonucleotides, and proteins across cell membranes. For example, sequences from the HIV Tat protein (GRKKRRQRRRPPQ (SEQ ID NO:130) and the Drosophila Antennapedia protein (RQIKIWFQNRRMKWK (SEQ ID NO:131) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam, et al., Nature 354:82-84 (1991)).

A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni, et al., Nucl. Acids Res. 31:2717-24 (2003)).

(iii) Carbohydrate Conjugates. In some embodiments, the RNAi agent oligonucleotides described herein further comprise carbohydrate conjugates. The carbohydrate conjugates may be advantageous for the in vivo delivery of nucleic acids, as well as compositions suitable for in vivo therapeutic use. As used herein, “carbohydrate” refers to a compound which is either a carbohydrate per se made up of one or more monosaccharide units having at least 6 carbon atoms (which can be linear, branched, or cyclic) with an oxygen, nitrogen, or sulfur atom bonded to each carbon atom; or a compound having as a part thereof a carbohydrate moiety made up of one or more monosaccharide units each having at least six carbon atoms (which can be linear, branched, or cyclic), with an oxygen, nitrogen, or sulfur atom bonded to each carbon atom. Representative carbohydrates include the sugars (mono-, di-, tri-, and oligosaccharides containing from about 4-9 monosaccharide units), and polysaccharides such as starches, glycogen, cellulose, and polysaccharide gums. Specific monosaccharides include C5 and above (in some embodiments, C5-C8) sugars; and di- and trisaccharides include sugars having two or three monosaccharide units (in some embodiments, C5-C8).

In some embodiments, the carbohydrate conjugate is selected from the group consisting of:

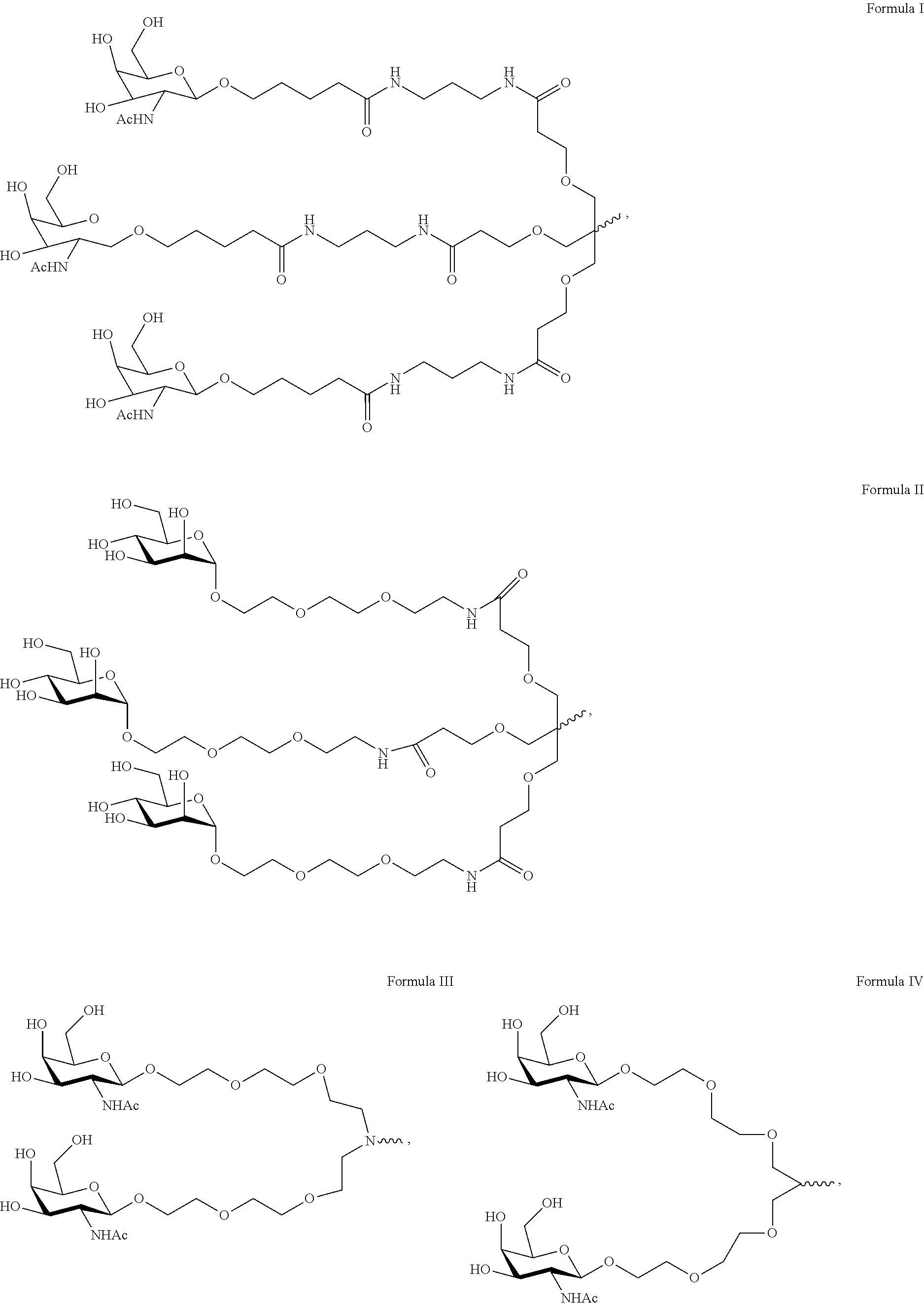

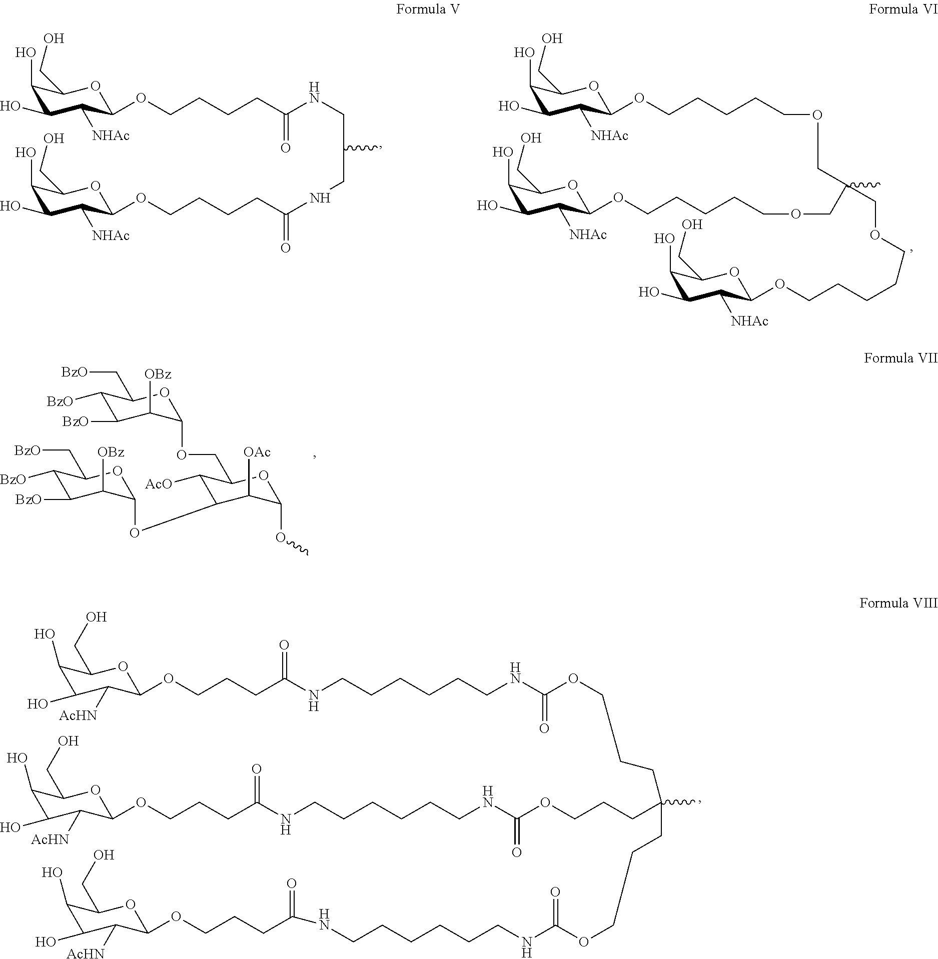

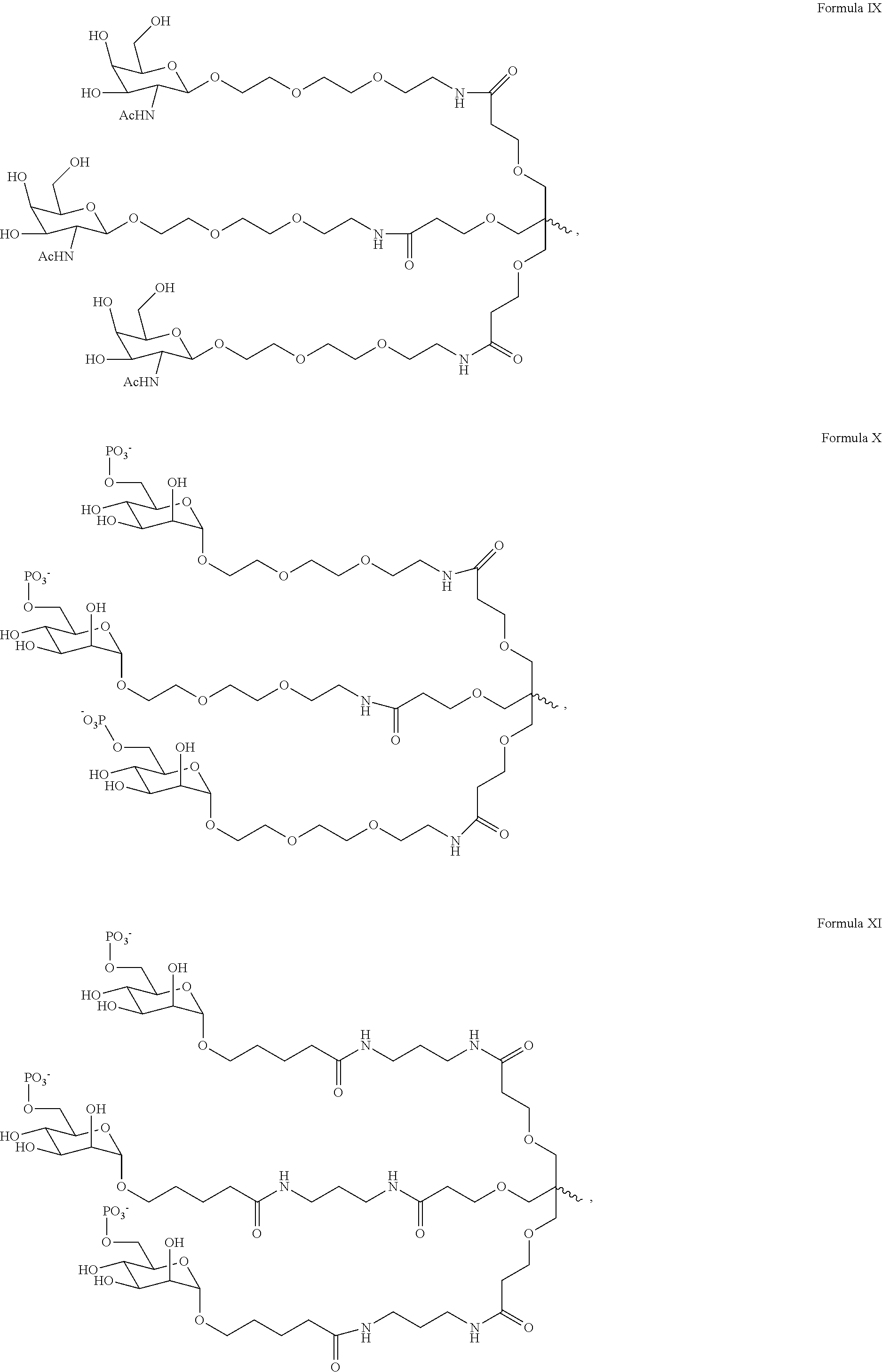

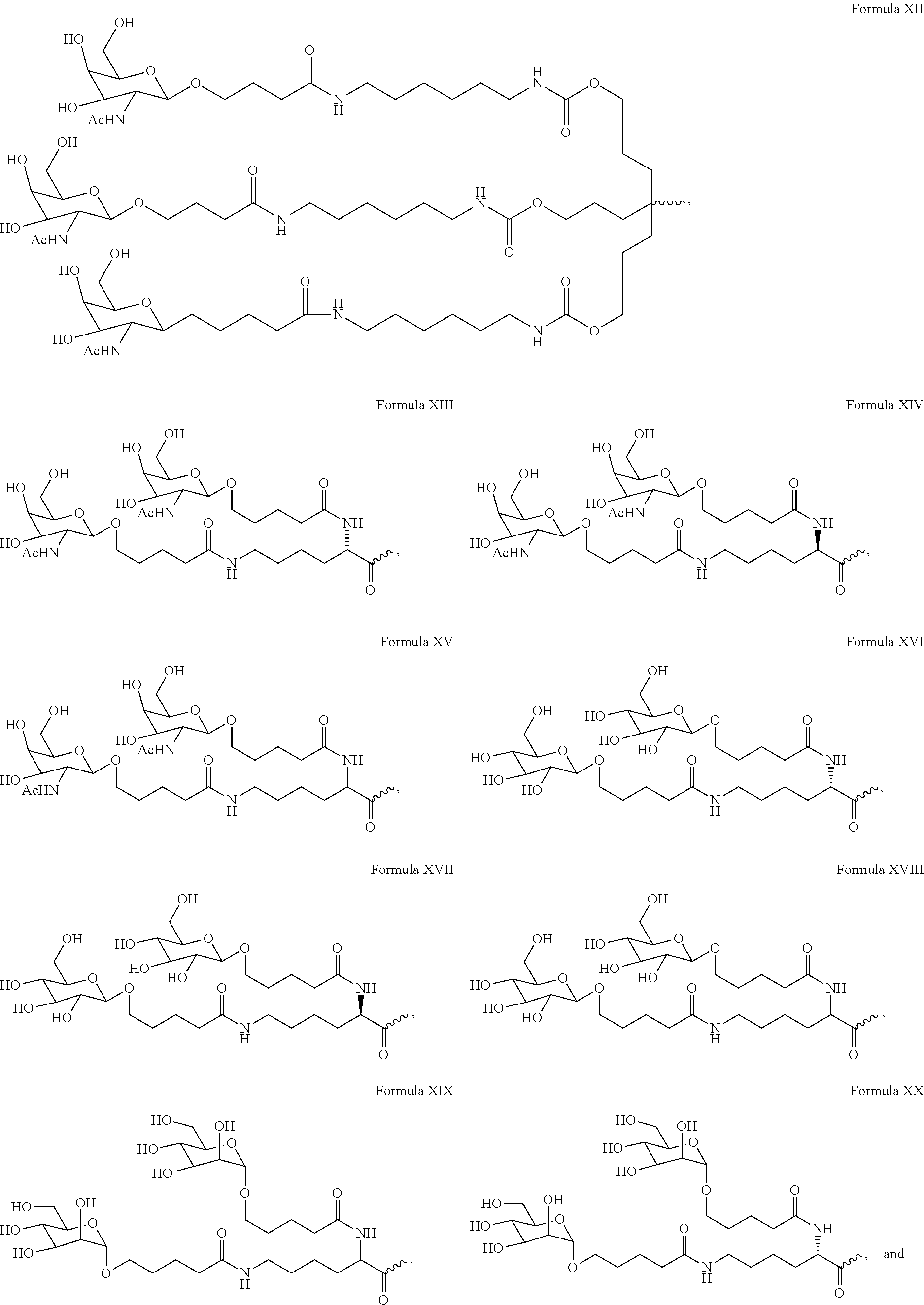

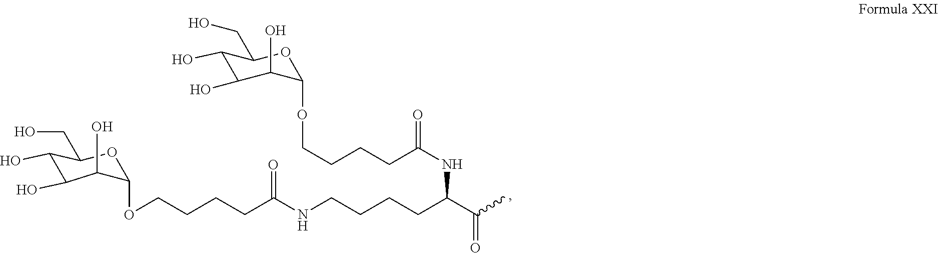

Another representative carbohydrate conjugate for use in the embodiments described herein includes, but is not limited to,

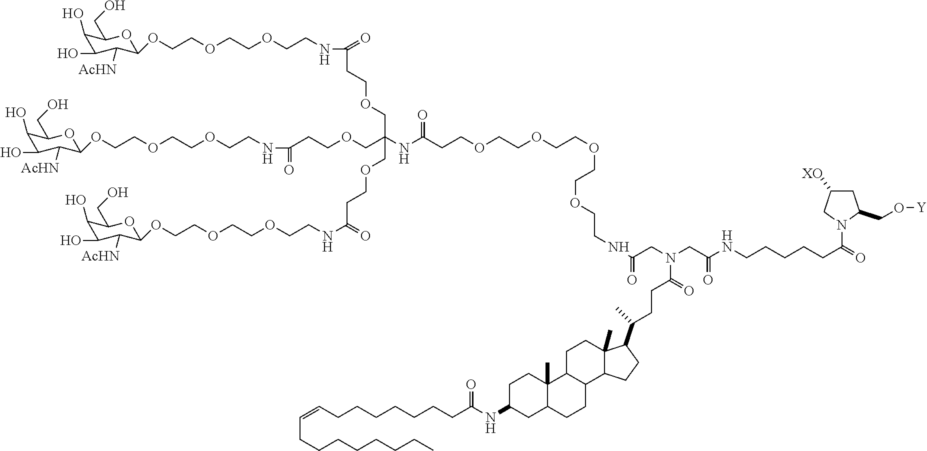

(Formula XXII), wherein when one of X or Y is an oligonucleotide, the other is a hydrogen.

In some embodiments, the carbohydrate conjugate further comprises another ligand such as, but not limited to, a PK modulator, an endosomolytic ligand, or a cell permeation peptide.

(iv) Linkers. In some embodiments, the conjugates described herein can be attached to the RNAi agent oligonucleotide with various linkers that can be cleavable or non-cleavable.

The term “linker” or “linking group” means an organic moiety that connects two parts of a compound. Linkers typically comprise a direct bond or an atom such as oxygen or sulfur, a unit such as NR8, C(O), C(O)NH, SO, SO2, SO2NH, or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, and alkynylhereroaryl, which one or more methylenes can be interrupted or terminated by O, S, S(O), SO2, N(R8), C(O), substituted or unsubstituted aryl, substituted or unsubstituted heteroaryl, or substituted or unsubstituted heterocyclic; where R8 is hydrogen, acyl, aliphatic, or substituted aliphatic. In certain embodiments, the linker is between 1-24 atoms, between 4-24 atoms, between 6-18 atoms, between 8-18 atoms, or between 8-16 atoms.

A cleavable linking group is one which is sufficiently stable outside the cell, but which upon entry into a target cell is cleaved to release the two parts the linker is holding together. In certain embodiments, the cleavable linking group is cleaved at least times, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases. A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a particular pH, thereby releasing the cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, liver-targeting ligands can be linked to the cationic lipids through a linker that includes an ester group. Liver cells are rich in esterases, and therefore the linker will be cleaved more efficiently in liver cells than in cell types that are not esterase-rich. Other cell types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It can be desirable to also test the candidate cleavable linking group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell-free systems, in cells, in cell culture, in organ or tissue culture, or in whole animals. It can be useful to make initial evaluations in cell-free or culture conditions and to confirm by further evaluations in whole animals. In certain embodiments, useful candidate compounds are cleaved at least 2, at least 4, at least 10 or at least 100 times faster in the cell (or under in vitro conditions selected to mimic intracellular conditions) as compared to blood or serum (or under in vitro conditions selected to mimic extracellular conditions).

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular RNAi moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In some embodiments, candidate compounds are cleaved by at most 10% in the blood. In certain embodiments, useful candidate compounds are degraded at least 2, at least 4, at least 10, or at least 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-based cleavable linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. In certain embodiments, the phosphate-based linking groups are selected from: —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—. In particular embodiments, the phosphate-linking group is —O—P(O)(OH)—O—. These candidates can be evaluated using methods analogous to those described above.

Acid cleavable linking groups are linking groups that are cleaved under acidic conditions. In some embodiments, acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes, can provide a cleaving environment for acid cleavable linking groups. Examples of acid cleavable linking groups include but are not limited to hydrazones, esters, and esters of amino acids. Acid cleavable groups can have the general formula —C═N—, C(O)O, or —OC(O). In some embodiments, the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-based cleavable linking groups are cleaved by enzymes such as esterases and amidases in cells. Examples of ester-based cleavable linking groups include but are not limited to esters of alkylene, alkenylene, and alkynylene groups. Ester cleavable linking groups have the general formula —C(O)O—, or —OC(O)—. These candidates can be evaluated using methods analogous to those described above.

Peptide-based cleavable linking groups are cleaved by enzymes such as peptidases and proteases in cells. Peptide-based cleavable linking groups are peptide bonds formed between amino acids to yield oligopeptides (e.g., dipeptides, tripeptides, etc.) and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

Representative carbohydrate conjugates with linkers include, but are not limited to,

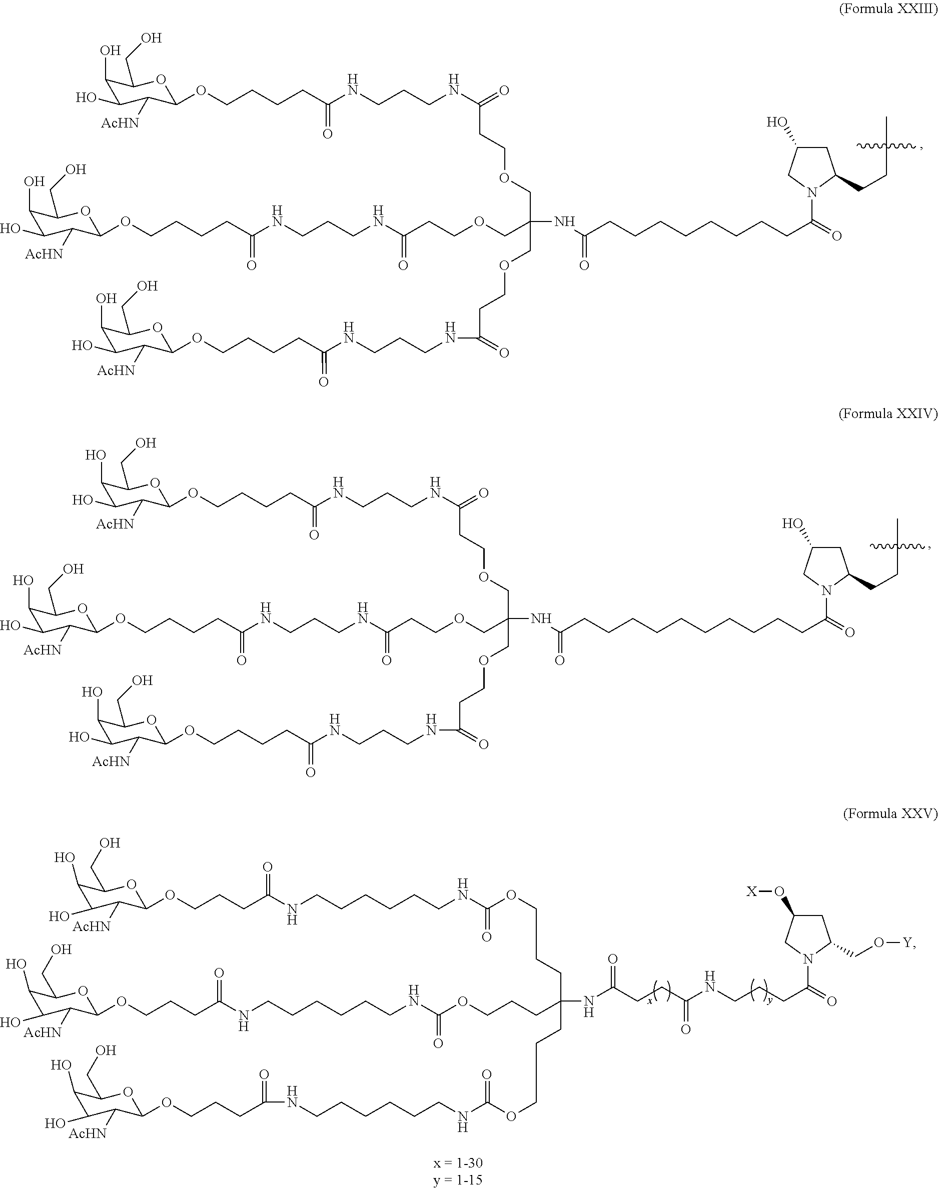

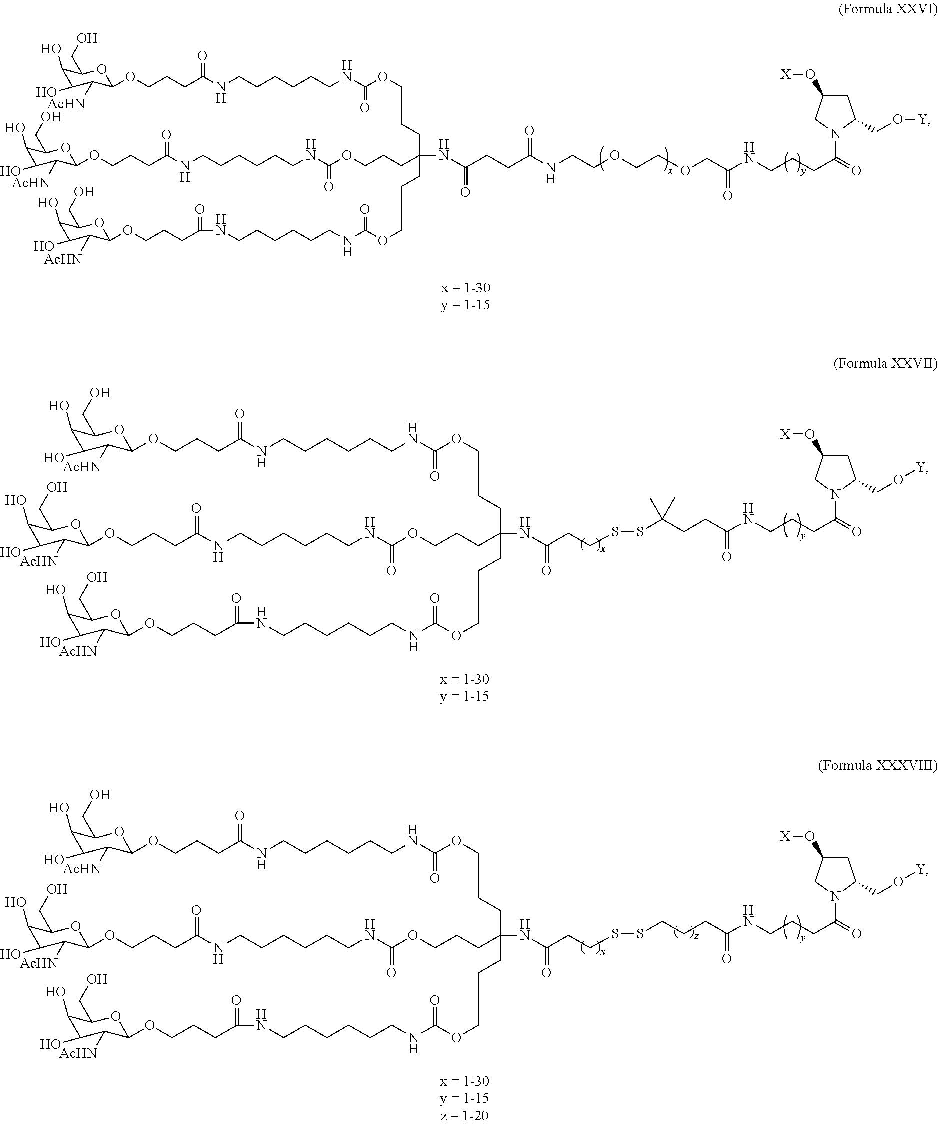

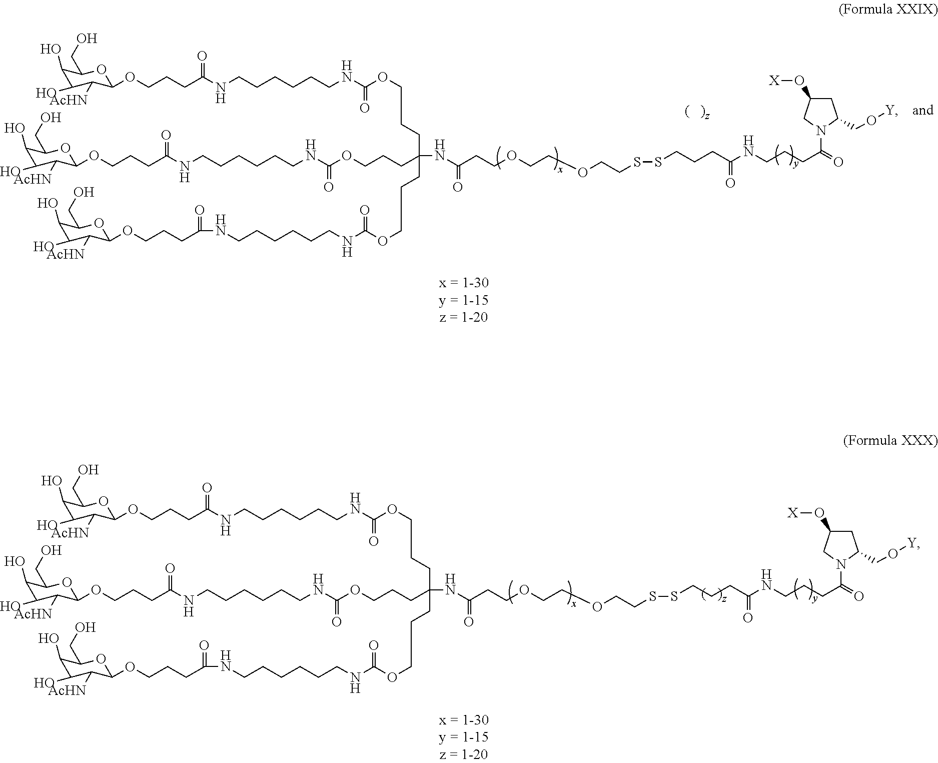

wherein when one of X or Y is an oligonucleotide, the other is a hydrogen.

In certain embodiments of the compositions and methods, a ligand is one or more “GalNAc” (N-acetylgalactosamine) derivatives attached through a bivalent or trivalent branched linker. For example, in some embodiments the siRNA is conjugated to a GalNAc ligand as shown in the following structure:

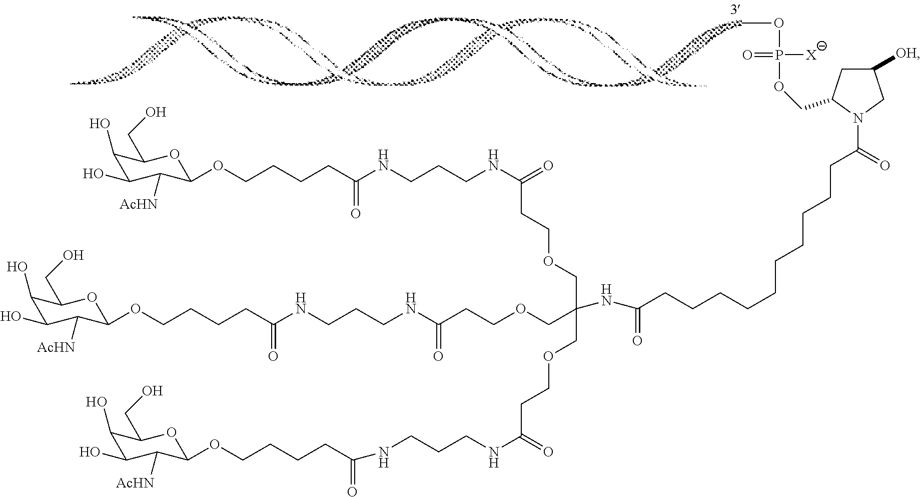

wherein X is O or S.

In some embodiments, the sense strand of the siRNA is conjugated to a ligand attached at the 3′ terminus of the sense strand through a linker as shown in the following structure:

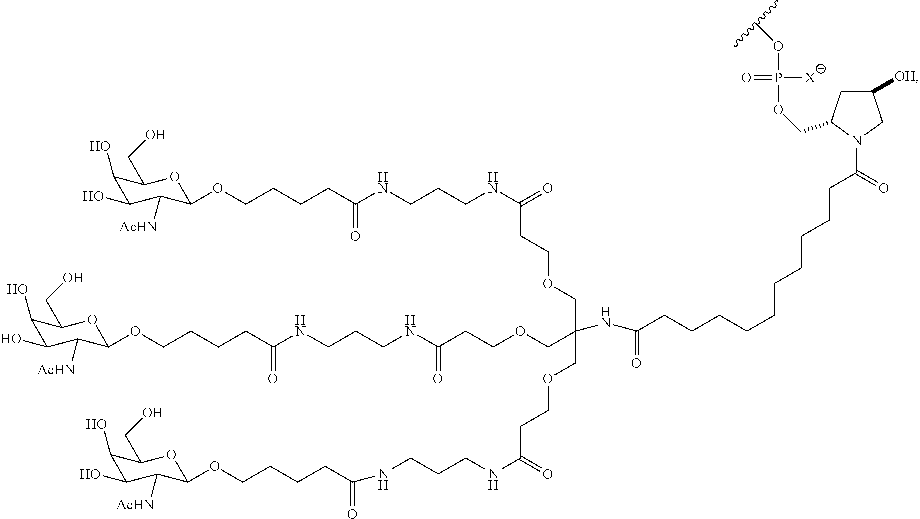

wherein X is O or S.

In some embodiments, the combination therapy includes an siRNA that is conjugated to a bivalent or trivalent branched linker selected from the group of structures shown in any of formula (XXXI)-(XXXIV):

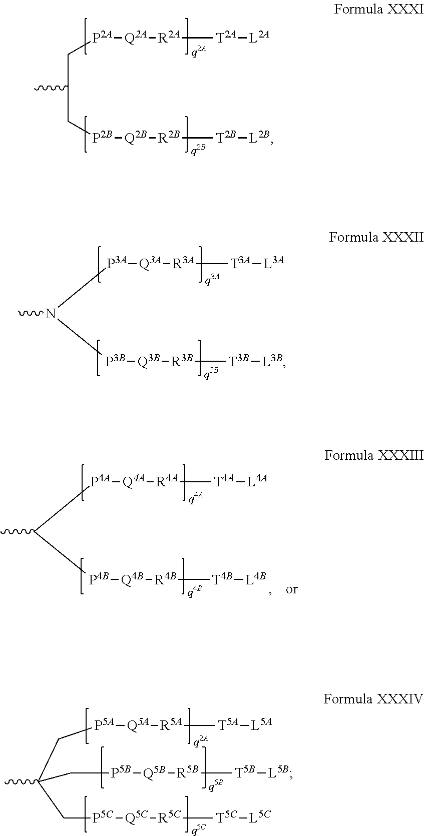

wherein:

q2A, q2B, q3A, q3B, q4A, q4B, q5A, q5B, and q5C represent independently for each occurrence 0-20 and wherein the repeating unit can be the same or different;

P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, and T^{5C} are each independently for each occurrence absent, CO, NH, O, S, OC(O), NHC(O), CH₂, CH₂NH, or CH₂O;

Q^{2A}, Q^{2B}, Q^{3A}, Q^{3B}, Q^{4A}, Q^{4B}, Q^{5A}, Q^{5B}, and Q^{5C} are independently for each occurrence absent, alkylene, or substituted alkylene wherein one or more methylenes can be interrupted or terminated by one or more of O, S, S(O), SO₂, N(R^{N}), C(R′)═C(R″), C≡C or C(O);

R^{2A}, R^{2B}, R^{3A}, R^{3B}, R^{4A}, R^{4B}, R^{5A}, R^{5B}, and R^{5C} are each independently for each occurrence absent, NH, O, S, CH₂, C(O)O, C(O)NH, NHCH(R^{a})C(O), —C(O)—CH(R^{a})—NH—, CO, CH═N—O,

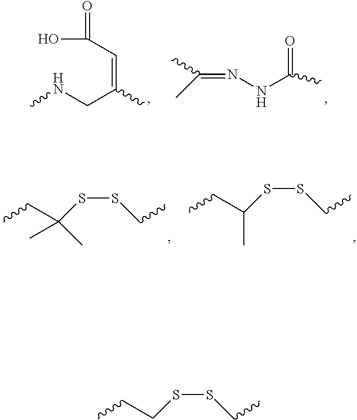

or heterocyclyl;

L^{2A}, L^{2B}, L^{3A}, L^{3B}, L^{4A}, L^{4B}, L^{5A}, L^{5B}, and L^{5C} represent the ligand; i.e., each independently for each occurrence a monosaccharide (such as GalNAc), disaccharide, trisaccharide, tetrasaccharide, oligosaccharide, or polysaccharide; and IV is H or amino acid side chain. Trivalent conjugating GalNAc derivatives are particularly useful for use with RNAi agents for inhibiting the expression of a target gene, such as those of formula (XXXIV):

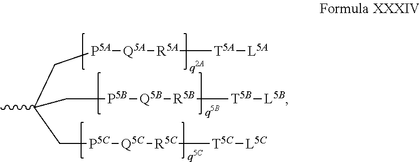

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas I, VI, X, IX, and XII.

Representative U.S. patents that teach the preparation of RNA conjugates include U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; and 7,037,646; each of which is incorporated herein by reference for teachings relevant to such methods of preparation.

In certain instances, the RNA of an RNAi agent can be modified by a non-ligand group. A number of non-ligand molecules have been conjugated to RNAi agents in order to enhance the activity, cellular distribution or cellular uptake of the RNAi agents, and procedures for performing such conjugations are available in the scientific literature. Such non-ligand moieties have included lipid moieties, such as cholesterol (Kubo, T., et al., Biochem. Biophys. Res. Comm. 365(1):54-61 (2007); Letsinger, et al., Proc. Natl. Acad. Sci. USA 86:6553 (1989)), cholic acid (Manoharan, et al., Bioorg. Med. Chem. Lett. 4:1053 (1994)), a thioether, e.g., hexyl-S-tritylthiol (Manoharan, et al., Ann. N.Y. Acad. Sci. 660:306 (1992); Manoharan, et al., Bioorg. Med. Chem. Let. 3:2765 (1993)), a thiocholesterol (Oberhauser, et al., Nucl. Acids Res. 20:533 (1992)), an aliphatic chain, e.g., dodecandiol or undecyl residues (Saison-Behmoaras, et al., EMBO J. 10:111 (1991); Kabanov, et al., FEBS Lett. 259:327 (1990); Svinarchuk, et al., Biochimie 75:49 (1993)), a phospholipid, e.g., di-hexadecyl-rac-glycerol or triethylammonium 1,2-di-O-hexadecyl-rac-glycero-3-H-phosphonate (Manoharan, et al., Tetrahedron Lett. 36:3651 (1995); Shea, et al., Nucl. Acids Res. 18:3777 (1990)), a polyamine or a polyethylene glycol chain (Manoharan, et al., Nucleosides & Nucleotides 14:969 (1995)), or adamantane acetic acid (Manoharan, et al., Tetrahedron Lett. 36:3651 (1195)), a palmityl moiety (Mishra, et al., Biochim. Biophys. Acta 1264:229 (1995)), or an octadecylamine or hexylamino-carbonyl-oxycholesterol moiety (Crooke, et al., J. Pharmacol. Exp. Ther. 277:923 (1996)).

Typical conjugation protocols involve the synthesis of an RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

d. RNAi Agent Delivery

“Introducing into a cell,” when referring to an RNAi agent, means facilitating or effecting uptake or absorption into the cell, as is understood by those skilled in the art.

Absorption or uptake of an RNAi agent can occur through unaided diffusive or active cellular processes, or by auxiliary agents or devices. The meaning of this term is not limited to cells in vitro; an RNAi agent can also be “introduced into a cell,” wherein the cell is part of a living organism. In such an instance, introduction into the cell will include the delivery to the organism. For example, for in vivo delivery, an RNAi agent can be injected into a tissue site or administered systemically. In vivo delivery can also be by a beta-glucan delivery system, such as those described in U.S. Pat. Nos. 5,032,401 and 5,607,677, and U.S. Publication No. 2005/0281781, which are incorporated herein by reference for teachings relevant to such delivery systems. In vitro introduction into a cell includes methods known in the art such as electroporation and lipofection. Further approaches are described herein below or are known in the art.

The delivery of an RNAi agent to a subject in need thereof can be achieved in a number of different ways. In vivo delivery can be performed directly by administering a composition comprising an RNAi agent, e.g., an siRNA, to a subject. Alternatively, delivery can be performed indirectly by administering one or more vectors that encode and direct the expression of the RNAi agent. These alternatives are discussed further below.

In general, any method of delivering a nucleic acid molecule can be adapted for use with an RNAi agent (see, e.g., Akhtar S. and Julian R L., Trends Cell. Biol. 2(5):139-44 (1992) and WO94/02595, which are incorporated herein by reference for teachings relevant to such methods of delivery). Three factors that are particularly important in successfully delivering an RNAi agent in vivo: (a) biological stability of the delivered molecule, (2) preventing nonspecific effects, and (3) accumulation of the delivered molecule in the target tissue. The nonspecific effects of an RNAi agent can be minimized by local administration, for example, by direct injection or implantation into a tissue (as a non-limiting example, a tumor) or topically administering the preparation. Local administration to a treatment site maximizes local concentration of the agent, limits the exposure of the agent to systemic tissues that can otherwise be harmed by the agent or that can degrade the agent, and permits a lower total dose of the RNAi agent to be administered. Several studies have shown successful knockdown of gene products when an RNAi agent is administered locally. For example, intraocular delivery of a VEGF siRNA by intravitreal injection in cynomolgus monkeys (Tolentino, M. J., et al., Retina 24:132-38 (2004)) and subretinal injections in mice (Reich, S. J., et al., Mol. Vis. 9:210-16 (2003)) were both shown to prevent neovascularization in an experimental model of age-related macular degeneration. In addition, direct intratumoral injection of an siRNA in mice reduces tumor volume (Pille, J., et al., Mol. Ther. 11:267-74 (2005)) and can prolong survival of tumor-bearing mice (Kim, W. J., et al., Mol. Ther. 14:343-50 (2006); Li, S., et al., Mol. Ther. 15:515-23 (2007)). RNA interference has also shown success with local delivery to the CNS by direct injection (Dorn, G., et al., Nucleic Acids 32:e49 (2004); Tan, P. H., et al., Gene Ther. 12:59-66 (2005); Makimura, H., et al., BMC Neurosci. 3:18 (2002); Shishkina, G. T., et al., Neuroscience 129:521-28 (2004); Thakker, E. R., et al. Proc. Natl. Acad. Sci. U.S.A. 101:17270-75 (2004); Akaneya, Y., et al., J. Neurophysiol. 93:594-602 (2005)) and to the lungs by intranasal administration (Howard, K. A., et al., Mol. Ther. 14:476-84 (2006); Zhang, X., et al., J. Biol. Chem. 279:10677-84 (2004); Bitko, V., et al., Nat. Med. 11:50-55 (2005)). For administering an RNAi agent systemically for the treatment of a disease, the RNA can be modified or alternatively delivered using a drug delivery system; both methods act to prevent the rapid degradation of the siRNA by endo- and exo-nucleases in vivo. Modification of the RNA or the pharmaceutical carrier can also permit targeting of the RNAi agent composition to the target tissue and avoid undesirable off-target effects. RNAi agents can be modified by chemical conjugation to lipophilic groups such as cholesterol to enhance cellular uptake and prevent degradation. For example, an RNAi agent directed against ApoB conjugated to a lipophilic cholesterol moiety was injected systemically into mice and resulted in knockdown of apoB mRNA in both the liver and jejunum (Soutschek, J., et al., Nature 432:173-78 (2004)). In some other embodiments, the RNAi agent can be delivered using drug delivery systems such as a nanoparticle, a dendrimer, a polymer, liposomes, or a cationic delivery system. Positively charged cationic delivery systems typically facilitate binding of an RNAi agent (negatively charged) and enhance interactions at the negatively charged cell membrane to permit efficient uptake of an RNAi agent by the cell. Cationic lipids, dendrimers, or polymers can either be bound to an RNAi, or induced to form a vesicle or micelle (see, e.g., Kim, S, H., et al., Journal of Controlled Release 129(2):107-16 (2008)) that encases an RNAi agent. The formation of vesicles or micelles further prevents degradation of the RNAi agent when administered systemically. Methods for making and administering cationic-RNAi agent complexes are well within the abilities of one skilled in the art (see, e.g., Sorensen, D. R., et al., J. Mol. Biol 327:761-66 (2003); Verma, U. N., et al., Clin. Cancer Res. 9:1291-1300 (2003); Arnold, A. S. et al., J. Hypertens. 25:197-205 (2007); which methods are incorporated herein by reference). Some non-limiting examples of drug delivery systems useful for systemic delivery of RNAi agents include DOTAP (Sorensen, D. R., et al. (2003), supra; Verma, U. N., et al., (2003), supra), Oligofectamine, “solid nucleic acid lipid particles” (Zimmermann, T. S., et al., Nature 441:111-14 (2006)), cardiolipin (Chien, P. Y., et al., Cancer Gene Ther. 12:321-28 (2005); Pal, A., et al., Int J. Oncol. 26: 1087-91 (2005)), polyethylenimine (Bonnet, M. E., et al., Pharm. Res. 25(12):2972-82; Aigner, A., J. Biomed. Biotechnol. 2006(4):71659 (2006)), Arg-Gly-Asp (RGD) peptides (Liu, S., Mol. Pharm. 3:472-487 (2006)), and polyamidoamines (Tomalia, D. A., et al., Biochem. Soc. Trans. 35:61-7 (2007); Yoo, H., et al., Pharm. Res. 16:1799-1804 (1999)).

As used herein, the term “SNALP” refers to a stable nucleic acid-lipid particle. A SNALP represents a vesicle of lipids coating a reduced aqueous interior comprising a nucleic acid such as an RNAi agent or a plasmid from which an RNAi agent is transcribed. SNALPs are described, e.g., in U.S. Patent Application Publication Nos. US2006/0240093 and US2007/0135372, and in International Application Publication No. WO 2009/082817. These applications are incorporated herein by reference for teachings relevant to SNALPs.

In some embodiments, an RNAi forms a complex with cyclodextrin for systemic administration. Methods for administration and pharmaceutical compositions of RNAis and cyclodextrins can be found in U.S. Pat. No. 7,427,605, which is incorporated herein by reference for teachings relevant to such compositions and methods. In some embodiments, a gene encoding an RNAi is encoded and expressed from an expression vector. Examples of vectors and their use in deliverying RNAis are described in U.S. Patent Application No. US2017/0349900A1, which examples are incorporated herein by reference.

e. Pharmaceutical Compositions and Formulation of RNAi Agents

In some embodiments, provided herein are pharmaceutical compositions containing an RNAi agent, as described herein, and a pharmaceutically acceptable carrier or excipient. The pharmaceutical composition containing the RNAi agent is useful in a combination therapy to treat HBV infection or reduce HBV viral load in a subject. Such pharmaceutical compositions are formulated based on the mode of delivery. For example, compositions may be formulated for systemic administration via parenteral delivery, e.g., by intravenous (IV) delivery, or for direct delivery into the brain parenchyma, e.g., by infusion into the brain, such as by continuous pump infusion.

In some contexts, a “pharmaceutically acceptable carrier” or “excipient” is a pharmaceutically acceptable solvent, suspending agent, or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutically acceptable carriers or excipients include, but are not limited to, binding agents (e.g., pregelatinized maize starch, polyvinylpyrrolidone, hydroxypropyl methylcellulose); fillers (e.g., lactose and other sugars, microcrystalline cellulose, pectin, gelatin, calcium sulfate, ethyl cellulose, polyacrylates, calcium hydrogen phosphate); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate); disintegrants (e.g., starch, sodium starch glycolate); and wetting agents (e.g., sodium lauryl sulphate).

In some embodiments, pharmaceutically acceptable organic or inorganic excipients suitable for non-parenteral administration which do not deleteriously react with nucleic acids can also be used to formulate the compositions of the present disclosure. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone, and the like.

In certain contexts, formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents, and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone, and the like.

In some embodiments, the pharmaceutical compositions containing an RNAi agent described herein are administered in dosages sufficient to inhibit expression of an HBV gene. In general, a suitable dose of an RNAi agent will be in the range of 0.001 to 200.0 milligrams per kilogram body weight of the recipient per day, and more typically in the range of 1 to 50 mg per kilogram body weight per day. For example, an siRNA can be administered at 0.01 mg/kg, 0.05 mg/kg, 0.5 mg/kg, 1 mg/kg, 1.5 mg/kg, 2 mg/kg, 3 mg/kg, 10 mg/kg, 20 mg/kg, 30 mg/kg, 40 mg/kg, or 50 mg/kg per single dose. The pharmaceutical composition can be administered once daily, or the RNAi agent can be administered as two, three, or more sub-doses at appropriate intervals throughout the day or even using continuous infusion or delivery through a controlled release formulation. In that case, the RNAi agent contained in each sub-dose must be correspondingly smaller in order to achieve the total daily dosage. The dosage unit can also be compounded for delivery over several days, e.g., using a conventional sustained release formulation which provides sustained release of the RNAi over a several day period. Sustained release formulations are well known in the art and are particularly useful for delivery of agents at a particular site, such as could be used with the agents of the technology described herein. In this embodiment, the dosage unit contains a corresponding multiple of the daily dose.

The effect of a single dose on the level of expression of an HBV gene can be long-lasting, such that subsequent doses are administered at not more than 3, 4, or 5 day intervals, or at not more than 1, 2, 3, or 4 week intervals.

The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual RNAi agents encompassed by the technology described herein can be made using conventional methodologies or on the basis of in vivo testing using an appropriate animal model, as described elsewhere herein.

Mouse models are available for the study of HBV infection, and such models can be used for in vivo testing of RNAi, as well as for determining a dose that is effective at reducing HBV gene expression.

In some embodiments, administration of pharmaceutical compositions and formulations described herein can be topical (e.g., by a transdermal patch), pulmonary (e.g., by inhalation or insufflation of powders or aerosols, including by nebulizer); intratracheal; intranasal; epidermal and transdermal; oral; or parenteral. Parenteral administration includes intravenous, intraarterial, subcutaneous, intraperitoneal, and intramuscular injection or infusion; subdermal administration (e.g., via an implanted device); or intracranial administration (e.g., by intraparenchymal, intrathecal, or intraventricular, administration).

In certain embodiments, an RNAi agent used in a combination therapy for treating HBV as disclosed herein is delivered subcutaneously.

In some embodiments, RNAi agents can be delivered in a manner to target a particular tissue, such as the liver (e.g., the hepatocytes of the liver).

Pharmaceutical compositions and formulations for topical administration can include transdermal patches, ointments, lotions, creams, gels, drops, suppositories, sprays, liquids, and powders. Conventional pharmaceutical carriers, aqueous, powder, or oily bases, thickeners, and the like can be necessary or desirable. Coated condoms, gloves, and the like can also be useful. Suitable topical formulations include those in which the RNAis featured in the technology described herein are in admixture with a topical delivery agent such as lipids, liposomes, fatty acids, fatty acid esters, steroids, chelating agents, and surfactants. Suitable lipids and liposomes include neutral (e.g., dioleoylphosphatidyl DOPE ethanolamine, dimyristoylphosphatidyl choline DMPC, distearolyphosphatidyl choline), negative (e.g., dimyristoylphosphatidyl glycerol DMPG), and cationic (e.g., dioleoyltetramethylaminopropyl DOTAP and dioleoylphosphatidyl ethanolamine DOTMA). RNAi agents can be encapsulated within liposomes or can form complexes thereto, in particular to cationic liposomes. Alternatively, RNAi agents can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-20 alkyl ester (e.g., isopropylmyristate IPM), monoglyceride, diglyceride, or pharmaceutically acceptable salt thereof. Examples of topical formulations are described in detail in U.S. Pat. No. 6,747,014, which is incorporated herein by reference for teachings relevant to such topical formulations.

Vesicles, such as liposomes, may be used in formulations for delivering RNAi agents disclosed herein; such formulation may have desirable properties such as specificity and the duration of action. As used herein, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes can possess the advantage of being able to fuse to the cell wall. Non-cationic liposomes, although not able to fuse as efficiently with the cell wall, may be taken up by macrophages in vivo. Important considerations in the preparation of liposome formulations are lipid surface charge, vesicle size, and the aqueous volume of the liposomes.

In some embodiments, liposomal delivery may have the following advantageous properties: being highly deformable and able to pass through fine pores in the skin; biocompatibility and biodegradabilty; ability to incorporate a wide range of water- and lipid-soluble drugs; ability to protect encapsulated drugs in their internal compartments from metabolism and degradation (Rosoff, in Pharmaceutical Dosage Forms, Lieberman, Rieger and Banker (Eds.), Marcel Dekker, Inc., New York, N.Y., volume 1, p. 245 (1998)); for topical delivery, reduced side-effects related to high systemic absorption of the administered drug, increased accumulation of the administered drug at the desired target, and the ability to administer a wide variety of drugs, both hydrophilic and hydrophobic, into the skin; and ability to deliver agents including high-molecular weight nucleic acids, analgesics, antibodies, and hormones to the skin.

Liposomes fall into two broad classes. Cationic liposomes are positively charged liposomes which interact with the negatively charged nucleic acid molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang, et al., Biochem. Biophys. Res. Commun. 147, 980-985 (1987)).

Liposomes that are pH-sensitive or negatively-charged, entrap nucleic acids rather than complex with them. Since both the DNA and the lipid are similarly charged, repulsion rather than complex formation occurs. Nevertheless, some DNA is entrapped within the aqueous interior of these liposomes. pH-sensitive liposomes have been used to deliver nucleic acids to cell monolayers in culture (e.g., Zhou, et al., Journal of Controlled Release 19, 269-74 (1992)).

In some embodiments, a liposomal composition is formed from phosphatidylcholine (PC), such as, for example, soybean PC and egg PC. In some embodiments, liposomal compositions include phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions can be formed from dimyristoyl phosphatidylglycerol, while anionic fusogenic liposomes can be formed from dioleoyl phosphatidylethanolamine (DOPE). In still other embodiments, a liposomal composition is formed from mixtures of phospholipid and/or phosphatidylcholine and/or cholesterol.

In some embodiments, liposomal drug formulations are delivered topically to the skin.

In some embodiments, an RNAi agent used in a combination therapy described herein is fully encapsulated in a lipid formulation, e.g., to form a SPLP, pSPLP, SNALP, or other nucleic acid-lipid particle. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SNALPs and SPLPs may be used for systemic applications, as they exhibit extended circulation lifetimes following intravenous (i.v.) injection and accumulate at distal sites (e.g., sites physically separated from the administration site). SPLPs include “pSPLP,” which include an encapsulated condensing agent-nucleic acid complex as set forth in International Application Publication No. WO 00/03683. The particles of the technology described herein typically have a mean diameter of about 50 nm to about 150 nm, more typically about 60 nm to about 130 nm, more typically about 70 nm to about 110 nm, and most typically about 70 nm to about 90 nm, and are substantially nontoxic. In addition, in some embodiments, nucleic acids when present in the nucleic acid-lipid particles are resistant in aqueous solution to degradation with a nuclease. Nucleic acid-lipid particles and related methods of preparation are disclosed in, e.g., U.S. Pat. Nos. 5,976,567; 5,981,501; 6,534,484; 6,586,410; 6,815,432; and International Application Publication No. WO 96/40964.

In some embodiments, the RNAi agent is delivered via a liposome or other lipid formulation, wherein the lipid to drug ratio (mass/mass ratio) (e.g., lipid to siRNA ratio) is in the range of from about 1:1 to about 50:1, from about 1:1 to about 25:1, from about 3:1 to about 15:1, from about 4:1 to about 10:1, from about 5:1 to about 9:1, or about 6:1 to about 9:1.

Pharmaceutical Compositions Comprising Antibodies, Antigen-Binding Fragments, Fusion Proteins, Polynucleotides, Vectors, and/or Host Cells

The present disclosure also provides a pharmaceutical composition comprising an antibody, antigen-binding fragment, or fusion protein, according to the present disclosure, a nucleic acid according to the present disclosure, a vector according to the present disclosure and/or a cell according to the present disclosure. In certain embodiments, a pharmaceutical composition further comprises an inhibitor of HBV protein expression and delivery system (e.g., an RNAi agent).

Pharmaceutical compositions may also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In general, pharmaceutically acceptable carriers in a pharmaceutical composition according to the present disclosure may be active components or inactive components.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions of the disclosure may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin™, for reconstitution with sterile water containing a preservative). The composition may be prepared for topical administration e.g., as an ointment, cream or powder. The composition may be prepared for oral administration e.g., as a tablet or capsule, as a spray, or as a syrup (optionally flavored). The composition may be prepared for pulmonary administration e.g., as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a subject. For example, a lyophilized antibody may be provided in kit form with sterile water or a sterile buffer.

In particular embodiments, the active ingredient in a composition according to the present disclosure is an antibody molecule, an antibody fragment or variant or derivative thereof, in particular the active ingredient in the composition is an antibody, an antibody fragment, a fusion protein, or variants and derivatives thereof, as described herein. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions of the disclosure may have a pH between 5.5 and 8.5, and in some embodiments this may be between 6 and 8. In other embodiments, the pH of a pharmaceutical composition as described herein may be about 7. The pH may be maintained by the use of a buffer. The composition may be sterile and/or pyrogen free. The composition may be isotonic with respect to humans. In certain embodiments, pharmaceutical compositions of the disclosure are supplied in hermetically sealed containers.

Within the scope of the disclosure are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody molecule may be in dry form, for reconstitution before use with an appropriate sterile liquid. A vehicle is typically understood to be a material that is suitable for storing, transporting, and/or administering a compound, such as a pharmaceutically active compound, in particular the antibodies according to the present description. For example, the vehicle may be a physiologically acceptable liquid, which is suitable for storing, transporting, and/or administering a pharmaceutically active compound, in particular the antibodies according to the present description. Once formulated, the compositions of the present disclosure can be administered directly to the subject. In one embodiment the compositions are adapted for administration to mammalian, e.g., human subjects.

The pharmaceutical compositions described herein may be administered by any number of routes including, but not limited to, oral, intravenous, intramuscular, intraarterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions of the description. In specific embodiments, the pharmaceutical composition may be prepared for oral administration, e.g. as tablets, capsules and the like, for topical administration, or as injectable, e.g. as liquid solutions or suspensions. Solid forms suitable for solution in, or suspension in, liquid vehicles prior to injection may also be utilized, e.g. that the pharmaceutical composition is in lyophilized form.

For injection, e.g. intravenous, cutaneous or subcutaneous injection, or injection at the site of affliction, the active ingredient can be provided be in the form of a parenterally acceptable aqueous solution which is pyrogen-free and has suitable pH, isotonicity and stability. Preservatives, stabilizers, buffers, antioxidants and/or other additives may be included, as required.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody or antigen-binding fragment as herein disclosed such that a suitable dosage will be obtained. Typically, this amount is at least 0.01% of the antibody or antigen-binding fragment in the composition. When intended for oral administration, this amount may be varied to be between 0.1 and about 70% of the weight of the composition. Certain oral pharmaceutical compositions contain between about 4% and about 75% of the antibody or antigen-binding fragment. In certain embodiments, pharmaceutical compositions and preparations according to the present invention are prepared so that a parenteral dosage unit contains between 0.01 to 10% by weight of antibody or antigen-binding fragment prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

In certain embodiments, a composition comprises a first vector comprising a first plasmid, and a second vector comprising a second plasmid, wherein the first plasmid comprises a polynucleotide encoding a heavy chain, VH, or VH+CH, and a second plasmid comprises a polynucleotide encoding the cognate light chain, VL, or VL+CL of the antibody or antigen-binding fragment thereof. In certain embodiments, a composition comprises a polynucleotide (e.g., mRNA) coupled to a suitable delivery vehicle or carrier. Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. Nanomed Nanobiotechnol. 11(2):e1530 (2019)). Principles, reagents, and techniques for designing appropriate mRNA and formulating mRNA-LNP and delivering the same are described in, for example, Pardi et al. (J Control Release 2/7345-351 (2015)); Thess et al. (Mol Ther 23: 1456-1464 (2015)); Thran et al. (EMBO Mol Med 9(10):1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol:polyethylene glycol lipid), and subcutaneous, intramuscular, intradermal, intravenous, intraperitoneal, and intratracheal administration of the same, are incorporated herein by reference.

The pharmaceutical compositions may be prepared by methodology well known in the pharmaceutical art. For example, a composition intended to be administered by injection can be prepared by combining a composition that comprises an antibody, antigen-binding fragment thereof, or other composition as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody or antigen-binding fragment thereof in the aqueous delivery system.

Whether it is a polypeptide, peptide, or nucleic acid molecule, cell, or other pharmaceutically useful compound according to the present disclosure that is to be given to an individual, administration is generally in a “prophylactically effective amount” or a “therapeutically effective amount” or an “effective amount” (as the case may be), this being sufficient to show a benefit to the individual (e.g., improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner). When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously.

Compositions are administered in an effective amount (e.g., to treat a SARS-CoV-2 infection), which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

Generally, a therapeutically effective daily dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). Other doses for antibodies or antigen-binding fragments are provided herein.

For polynucleotides, vectors, host cells, and related compositions of the present disclosure, a therapeutically effective dose may be different than for an antibody or antigen-binding fragment.

The actual amount administered, and rate and time-course of administration, will depend on the nature and severity of what is being treated. For injection, the pharmaceutical composition according to the present disclosure may be provided for example in a pre-filled syringe.

Pharmaceutical compositions as disclosed herein may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the inventive transporter cargo conjugate molecule as defined above, is combined with emulsifying and suspending agents. If desired, certain sweetening, flavoring or coloring agents may also be added.

The pharmaceutical compositions according to the present description may also be administered topically, especially when the target of treatment includes areas or organs readily accessible by topical application, e.g., including diseases of the skin or of any other accessible epithelial tissue. Suitable topical formulations are readily prepared for each of these areas or organs. For topical applications, the pharmaceutical composition may be formulated in a suitable ointment, containing the inventive pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the pharmaceutical composition can be formulated in a suitable lotion or cream. In the context of the present description, suitable carriers include, but are not limited to, mineral oil, sorbitan monostearate, polysorbate 60, cetyl esters wax, cetearyl alcohol, 2-octyldodecanol, benzyl alcohol and water.

Doses may be expressed in relation to the bodyweight. Thus, a dose which is expressed as [g, mg, or other unit]/kg (or g, mg etc.) usually refers to [g, mg, or other unit] “per kg (or g, mg etc.) bodyweight”, even if the term “bodyweight” or “body weight” is not explicitly mentioned.

In specific embodiments, in a single dose, e.g. a daily, weekly or monthly dose, the amount of the antibody, or the antigen binding fragment thereof, in the pharmaceutical composition does not exceed 1 g. In certain such embodiments, the single dose does not exceed a dose selected from 500 mg, 250 mg, 100 mg, and 50 mg. Further embodiments of doses are provided herein.

In certain embodiments, a method comprises administering the antibody, antigen-binding fragment, polynucleotide, vector, host cell, or composition to the subject at 2, 3, 4, 5, 6, 7, 8, 9, 10 times, or more.

In certain embodiments, a method comprises administering the antibody, antigen-binding fragment, or composition to the subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the antibody, antigen-binding fragment, polynucleotide, vector, host cell, or composition at least one time prior to the subject

Compositions comprising an antibody, antigen-binding fragment, polynucleotide, vector, host cell, or composition of the present disclosure may also be administered simultaneously with, prior to, or after administration of one or more other therapeutic agents. Such combination therapy may include administration of a single pharmaceutical dosage formulation which contains a compound of the invention and one or more additional active agents, as well as administration of compositions comprising an antibody or antigen-binding fragment of the disclosure and each active agent in its own separate dosage formulation. For example, an antibody or antigen-binding fragment thereof as described herein and the other active agent can be administered to the patient together in a single oral dosage composition such as a tablet or capsule, or each agent administered in separate oral dosage formulations. Similarly, an antibody or antigen-binding fragment as described herein and the other active agent can be administered to the subject together in a single parenteral dosage composition such as in a saline solution or other physiologically acceptable solution, or each agent administered in separate parenteral dosage formulations. Where separate dosage formulations are used, the compositions comprising an antibody or antigen-binding fragment and one or more additional active agents can be administered at essentially the same time, i.e., concurrently, or at separately staggered times, i.e., sequentially and in any order; combination therapy is understood to include all these regimens.

In some embodiments, a composition or kit as described herein further comprises (i) a polymerase inhibitor, wherein the polymerase inhibitor optionally comprises Lamivudine, Adefovir, Entecavir, Telbivudine, Tenofovir, or any combination thereof; (ii) an interferon, wherein the interferon optionally comprises IFNbeta and/or IFNalpha; (iii) a checkpoint inhibitor, wherein the checkpoint inhibitor optionally comprises an anti-PD-1 antibody or antigen binding fragment thereof, an anti-PD-L1 antibody or antigen binding fragment thereof, and/or an anti-CTLA4 antibody or antigen binding fragment thereof; (iv) an agonist of a stimulatory immune checkpoint molecule; or (v) any combination of (i)-(iv). In some embodiments, a kit comprises a composition or combination as described herein, and further comprises instructions for using the component to prevent, treat, attenuate, and/or diagnose a hepatitis B infection and/or a hepatitis D infection.

In certain embodiments, a composition of the present disclosure (e.g., antibody, antigen-binding fragment, host cell, nucleic acid, vector, or pharmaceutical composition) is used in combination with a PD-1 inhibitor, for example a PD-1-specific antibody or binding fragment thereof, such as pidilizumab, nivolumab, pembrolizumab, MEDI0680 (formerly AMP-514), AMP-224, BMS-936558 or any combination thereof. In certain embodiments, a composition of the present disclosure is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof. In certain embodiments, a composition of the present disclosure is used in combination with a LAG3 inhibitor, such as LAG525, IMP321, IMP701, 9H12, BMS-986016, or any combination thereof. In certain embodiments, a composition of the present disclosure is used in combination with an inhibitor of CTLA4. In particular embodiments, an a composition of the present disclosure is used in combination with a CTLA4 specific antibody or binding fragment thereof, such as ipilimumab, tremelimumab, CTLA4-Ig fusion proteins (e.g., abatacept, belatacept), or any combination thereof. In certain embodiments, a composition of the present disclosure is used in combination with a B7-H3 specific antibody or binding fragment thereof, such as enoblituzumab (MGA271), 376.96, or both. An anti-B7-H3 antibody binding fragment may be a scFv or fusion protein comprising the same, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1. In certain embodiments, a composition of the present disclosure is used in combination with an inhibitor of CD244. In certain embodiments, a composition of the present disclosure is used in combination with an inhibitor of BLTA, HVEM, CD160, or any combination thereof. Anti CD-160 antibodies are described in, for example, PCT Publication No. WO 2010/084158. In certain embodiments, a composition of the present disclosure is used in combination with an inhibitor of TIM3. In certain embodiments, a composition of the present disclosure is used in combination with an inhibitor of Ga19. In certain embodiments, a composition of the present disclosure is used in combination with an inhibitor of adenosine signaling, such as a decoy adenosine receptor. In certain embodiments, a composition of the present disclosure is used in combination with an inhibitor of A2aR. In certain embodiments, a composition of the present disclosure is used in combination with an inhibitor of KIR, such as lirilumab (BMS-986015). In certain embodiments, a composition of the present disclosure is used in combination with an inhibitor of an inhibitory cytokine (typically, a cytokine other than TGFβ) or Treg development or activity. In certain embodiments, a composition of the present disclosure is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof. In certain embodiments, a composition of the present disclosure is used in combination with an arginase inhibitor, such as N(omega)-Nitro-L-arginine methyl ester (L-NAME), N-omega-hydroxy-nor-1-arginine (nor-NOHA), L-NOHA, 2(S)-amino-6-boronohexanoic acid (ABH), S-(2-boronoethyl)-L-cysteine (BEC), or any combination thereof. In certain embodiments, a composition of the present disclosure is used in combination with an inhibitor of VISTA, such as CA-170 (Curis, Lexington, Mass.). In certain embodiments, a composition of the present disclosure is used in combination with an inhibitor of TIGIT such as, for example, COM902 (Compugen, Toronto, Ontario Canada), an inhibitor of CD155, such as, for example, COM701 (Compugen), or both. In certain embodiments, a composition of the present disclosure is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526. In certain embodiments, a composition of the present disclosure is used in combination with a LAIR1 inhibitor. In certain embodiments a composition of the present disclosure is used in combination with an inhibitor of CEACAM-1, CEACAM-3, CEACAM-5, or any combination thereof.

In certain embodiments, a composition of the present disclosure is used in combination with an agent that increases the activity (i.e., is an agonist) of a stimulatory immune checkpoint molecule. For example, a composition of the present disclosure can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof).

In any of the embodiments disclosed herein, a method may comprise administering a composition of the present disclosure with one or more agonist of a stimulatory immune checkpoint molecule, including any of the foregoing, singly or in any combination.

An antibody, antigen binding fragment, or fusion protein according to the present disclosure can be present either in the same pharmaceutical composition as the additional active component or, the antibody, antigen binding fragment, or fusion protein according to the present disclosure may be included in a first pharmaceutical composition and the additional active component may be included in a second pharmaceutical composition different from the first pharmaceutical composition.

### Uses

In a further aspect, the present disclosure provides methods for the use of an antibody, an antigen binding fragment, a fusion protein, a nucleic acid, a vector, a cell, a pharmaceutical composition, a combination (e.g., of a presently disclosed antibody or antigen-binding fragment with a presently disclosed inhibitor of HBV protein expression and delivery system (e.g., an RNAi agent), or a kit according to the present disclosure in the (i) prophylaxis, treatment or attenuation of hepatitis B and/or hepatitis D; or in (ii) diagnosis of hepatitis B and/or hepatitis D (e.g., in a human subject).

Methods of diagnosis (e.g., in vitro, ex vivo) may include contacting an antibody, antibody fragment (e.g., antigen binding fragment), or fusion protein with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood. The methods of diagnosis may also include the detection of an antigen/antibody or antigen/fusion protein complex, in particular following the contacting of an antibody, antibody fragment, or fusion protein with a sample. Such a detection step is typically performed at the bench, i.e. without any contact to the human or animal body. Examples of detection methods are well-known to the person skilled in the art and include, e.g., ELISA (enzyme-linked immunosorbent assay).

The disclosure also provides the use of (i) an antibody, an antibody fragment, fusion protein, or variants and derivatives thereof according to the disclosure, (ii) host cell (which can be an immortalized B cell) according to the disclosure, (iii) a nucleic acid or a vector according to the present disclosure (iv) a pharmaceutical composition of the present disclosure or (v) a combination (e.g., of a presently disclosed antibody or antigen-binding fragment with a presently disclosed inhibitor of HBV protein expression and delivery system (e.g., an RNAi agent)) in (a) the manufacture of a medicament for the prevention, treatment or attenuation of hepatitis B and/or hepatitis D or for (b) diagnosis of hepatitis B and/or hepatitis D.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the affected human or animal to have a reduced duration or quality of life.

As used herein, reference to “treatment” of a subject or patient is intended to include prevention, prophylaxis, attenuation, amelioration and therapy, and refers to medical management of a disease, disorder, or condition of a subject. Benefits of treatment can include improved clinical outcome; lessening or alleviation of symptoms associated with a disease; decreased occurrence of symptoms; improved quality of life; longer disease-free status; diminishment of extent of disease; stabilization of disease state; delay of disease progression; remission; survival; prolonged survival; or any combination thereof. The terms “subject” or “patient” are used interchangeably herein to mean all mammals, including humans. Examples of subjects include humans, cows, dogs, cats, horses, goats, sheep, pigs, and rabbits. In certain embodiments, the patient is a human. The subjects can be male or female and can be any suitable age, including infant, juvenile, adolescent, adult, and geriatric subjects.

The disclosure also provides an antibody, antigen binding fragment, or fusion protein according to the present disclosure, a nucleic acid according to the present disclosure, a vector according to the present disclosure, a cell according to the present disclosure a pharmaceutical composition according, and/or a combination (e.g., of a presently disclosed antibody or antigen-binding fragment with a presently disclosed inhibitor of HBV protein expression and delivery system (e.g., an RNAi agent) of the present disclosure for use as a medicament for the prevention or treatment of hepatitis B and/or hepatitis D. It also provides the use of an antibody, antigen binding fragment, or fusion protein of the disclosure in the manufacture of a medicament for treatment of a subject and/or diagnosis in a subject. It also provides a method for treating a subject (e.g., a human subject), comprising administering to the subject an effective amount of a composition or combination as described herein. In some embodiments, the subject may be a human. One way of checking efficacy of therapeutic treatment involves monitoring disease symptoms after administration of the composition. Treatment can be a single dose schedule or a multiple dose schedule.

In one embodiment, an antibody, antigen-binding fragment, fusion protein, host cell (e.g., immortalized B cell clone, or T cell, NK-T cell, or NK cell that expresses a fusion protein), pharmaceutical composition, or combination according to the disclosure is administered to a subject in need of such treatment. Such a subject includes, but is not limited to, one who is particularly at risk of or susceptible to hepatitis B and/or hepatitis D.

Antibodies, antigen binding fragments, fusion proteins, polynucleotides, vectors, host cells, pharmaceutical compositions, and combinations of the same, according to the present disclosure may also be used in a kit for the prevention, treatment, attenuation, and/or diagnosis of hepatitis B and/or hepatitis D. In some embodiments, a kit further comprises instructions for using the component to prevent, treat, attenuate, and/or diagnose a hepatitis B infection and/or a hepatitis D infection. Further, the epitope in the antigenic loop region of HBsAg, which is capable of binding an antibody, antigen binding fragment, or fusion protein of the disclosure as described herein may be used in a kit for monitoring the efficacy of application procedures by detecting the presence or determining the titer of protective anti-HBV antibodies.

In certain embodiments, a composition or a kit of this disclosure further comprises: a polymerase inhibitor, wherein the polymerase inhibitor optionally comprises Lamivudine, Adefovir, Entecavir, Telbivudine, Tenofovir, or any combination thereof; (ii) an interferon, wherein the interferon optionally comprises IFNbeta and/or IFNalpha; (iii) a checkpoint inhibitor, wherein the checkpoint inhibitor optionally comprises an anti-PD-1 antibody or antigen binding fragment thereof, an anti-PD-L1 antibody or antigen binding fragment thereof, and/or an anti-CTLA4 antibody or antigen binding fragment thereof; (iv) an agonist of a stimulatory immune checkpoint molecule; or (v) any combination of (viii)-(xii).

In some embodiments, an antibody, an antigen binding fragment, or fusion protein according to the present disclosure, a nucleic acid according to the present disclosure, the vector according to the present disclosure, a cell according to the present disclosure, a pharmaceutical composition according to the present disclosure, and/or a combination (e.g., of a presently disclosed antibody or antigen-binding fragment with a presently disclosed inhibitor of HBV protein expression and delivery system (e.g., an RNAi agent) of the present disclosure is used in treatment or attenuation of chronic hepatitis B infection.

In particular embodiments, an antibody, antigen binding fragment, or fusion protein according to the present disclosure (i) neutralizes HBV infection, (ii) binds to L-HBsAg (the large HBV envelope protein, which is present in infectious HBV particles), thereby preventing spreading of HBV, (iii) binds to S-HBsAg, thereby promoting clearance of subviral particles (SVP) and/or (iv) can induce seroconversion, i.e. an active immune response to the virus.

In particular embodiments, an antibody, antigen binding fragment, or fusion protein according to the present disclosure, a nucleic acid according to the present disclosure, a vector according to the present disclosure, a cell according to the present disclosure, or a pharmaceutical composition according to the present disclosure, may be used in prevention of hepatitis B (re-)infection after liver transplantation in particular for hepatitis B induced liver failure.

In further embodiments an antibody, antigen binding fragment thereof, or fusion protein according to the present disclosure, a nucleic acid according to the present disclosure, a vector according to the description provided herein, a cell according to the present disclosure, a pharmaceutical composition, and/or a combination (e.g., of a presently disclosed antibody or antigen-binding fragment with a presently disclosed inhibitor of HBV protein expression and delivery system (e.g., an RNAi agent) according to the present disclosure, may be used in prevention/prophylaxis of hepatitis B in non-immunized subjects. This is for example in case of (an assumed) accidental exposure to HBV (post-exposure prophylaxis). The term “non-immunized subjects” includes subjects, who never received a vaccination and are, thus, not immunized, and subjects, who did not show an immune response (e.g., no measurable anti-hepatitis B antibodies) after vaccination.

In some embodiments, an antibody, antigen binding fragment, or fusion protein according to the present disclosure, the nucleic acid according to the present disclosure, a vector according to the present disclosure, a cell according to the present disclosure, a pharmaceutical composition according to the present disclosure, or a combination (e.g., of a presently disclosed antibody or antigen-binding fragment with a presently disclosed inhibitor of HBV protein expression and delivery system (e.g., an RNAi agent) of the present disclosure, is used in prophylaxis of hepatitis B in haemodialysed patients.

In some embodiments, an antibody, an antigen binding fragment, or fusion protein according to the present disclosure, a nucleic acid according to the present disclosure, a vector according to the present disclosure, a cell according to the present disclosure, a pharmaceutical composition according to the present disclosure, or a combination (e.g., of a presently disclosed antibody or antigen-binding fragment with a presently disclosed inhibitor of HBV protein expression and delivery system (e.g., an RNAi agent) of the present disclosure, is used in prevention of hepatitis B in a newborn. In such embodiments, an antibody, or an antigen binding fragment thereof, according to the present disclosure, a nucleic acid according to the present disclosure, a vector according to the present disclosure, a cell according to the present disclosure, a pharmaceutical composition according to the present disclosure, or a combination (e.g., of a presently disclosed antibody or antigen-binding fragment with a presently disclosed inhibitor of HBV protein expression and delivery system (e.g., an RNAi agent) of the present disclosure, may be administered at birth or as soon as possible after birth. The administration may be repeated until seroconversion following vaccination.

Moreover, the present disclosure also provides the use of an antibody, antigen binding fragment, or fusion protein according to the present disclosure, a nucleic acid according to the present disclosure, a vector according to the present disclosure, a cell according to the present disclosure or a pharmaceutical composition according to the present disclosure in the diagnosis (e.g. in vitro, ex vivo, or in vivo) of hepatitis B and/or hepatitis D.

In addition, the use of an antibody, antigen binding fragment, or fusion protein according to the present disclosure, a nucleic acid according to the present disclosure, a vector according to the present disclosure, a cell according to the present disclosure or a pharmaceutical composition according to the present disclosure in determining whether an isolated blood sample is infected with hepatitis B virus and/or hepatitis delta virus is provided.

As described above, methods of diagnosis may include contacting an antibody, antibody fragment, or fusion protein with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood. The methods of diagnosis may also include the detection of an antigen/antibody complex, in particular following the contacting of an antibody or an antibody fragment with a sample. Such a detection step is typically performed at the bench, i.e. without any contact to the human or animal body. Examples of detection methods are well-known to the person skilled in the art and include, e.g., ELISA (enzyme-linked immunosorbent assay).

The present disclosure also provides a method of treating, preventing and/or attenuating hepatitis B and/or hepatitis D in a subject, wherein the method comprises administering to the subject an antibody, antigen binding fragment, or fusion protein according to the present disclosure, a nucleic acid according to the present disclosure, a vector according to the present disclosure, a cell according to the present disclosure, a pharmaceutical composition according to the present disclosure, and/or a combination (e.g., of a presently disclosed antibody or antigen-binding fragment with a presently disclosed inhibitor of HBV protein expression and delivery system (e.g., an RNAi agent) of the present disclosure. In certain embodiments, a method further comprises administering to the subject one or more of: (vii) a polymerase inhibitor, wherein the polymerase inhibitor optionally comprises Lamivudine, Adefovir, Entecavir, Telbivudine, Tenofovir, or any combination thereof; (viii) an interferon, wherein the interferon optionally comprises IFNbeta and/or IFNalpha; (ix) a checkpoint inhibitor, wherein the checkpoint inhibitor optionally comprises an anti-PD-1 antibody or antigen binding fragment thereof, an anti-PD-L1 antibody or antigen binding fragment thereof, and/or an anti-CTLA4 antibody or antigen binding fragment thereof; (x) an agonist of a stimulatory immune checkpoint molecule; or (xi) any combination of (vii)-(x).

In some embodiments, the hepatitis B infection is a chronic hepatitis B infection. In some embodiments, the subject has received a liver transplant. In some embodiments, the subject is non-immunized against hepatitis B. In certain embodiments, the subject is a newborn. In some embodiments, the subject is undergoing or has undergone hemodialysis.

The present disclosure also provides a method of treating a subject who has received a liver transplant comprising administering to the subject who has received the liver transplant an effective amount of an antibody, an antigen binding fragment, or fusion protein according to the present disclosure, a nucleic acid according to the present disclosure, a vector according to the present disclosure, a cell according to the present disclosure, a pharmaceutical composition according to the present disclosure, or a combination (e.g., of a presently disclosed antibody or antigen-binding fragment with a presently disclosed inhibitor of HBV protein expression and delivery system (e.g., an RNAi agent) of the present disclosure.

Also provided herein are methods for detecting the presence or absence of an epitope in a correct conformation in an anti-hepatitis-B and/or an anti-hepatitis-D vaccine, wherein the methods comprise: (i) contacting the vaccine with an antibody, antigen-binding fragment, or fusion protein of any one of the present disclosure; and (ii) determining whether a complex comprising an antigen and the antibody, or comprising an antigen and the antigen binding fragment, or comprising an antigen and the fusion protein, has been formed.

The term “vaccine” as used herein is typically understood to be a prophylactic or therapeutic material providing at least one antigen, such as an immunogen. The antigen or immunogen may be derived from any material that is suitable for vaccination. For example, the antigen or immunogen may be derived from a pathogen, such as from bacteria particles, virus particles, a tumor (including a solid or liquid tumor), or other cancerous tissue. The antigen or immunogen stimulates the body's adaptive immune system to provide an adaptive immune response. In certain embodiments, an “antigen” or an “immunogen” refers to a substance which may be recognized by the immune system, e.g. by the adaptive immune system, and which is capable of triggering an antigen-specific immune response, e.g. by formation of antibodies and/or antigen-specific T cells as part of an adaptive immune response. In some embodiments, an antigen may be or may comprise a peptide or protein which may be presented by an MEW complex (e.g., MHC class I; MHC class II) to T cells. In certain embodiments, the antigen comprises a HBV and/or HBD antigen; e.g., an HBsAg antigen.

Some embodiments of the present disclosure provide methods of treating chronic HBV infection or an HBV-associated disease in a subject in need thereof, comprising: (i) administering to the subject an agent that reduces HBV antigenic load; and (ii) administering to the subject an anti-HBV antibody or antigen-binding fragment thereof. In certain embodiments, the agent that reduces HBV antigenic load is administered before the anti-HBV antibody or antigen-binding fragment thereof. In certain embodiments, administering the agent that reduces HBV antigenic load before the anti-HBV antibody or antigen-binding fragment thereof causes the viral load to be reduced when the anti-HBV antibody or antigen-binding fragment thereof is administered. In certain embodiments, the therapeutically effective amount of the anti-HBV antibody or antigen-binding fragment thereof of the combination therapy is less than a therapeutically effective amount of the anti-HBV antibody or antigen-binding fragment thereof delivered when the agent that reduces HBV antigenic load has not been administered to the subject (e.g., when the anti-HBV antibody or antigen-binding fragment thereof is administered alone as a monotherapy). In some embodiments, the agent that reduces HBV antigenic load is an RNAi agent (e.g., an siRNA) that inhibits expression of an HBV transcript.

In certain embodiments, the present disclosure provides a method of treating a chronic HBV infection or HBV-associated disease in a subject in need thereof, comprising: administering to the subject an agent that reduces HBV antigenic load; and administering to the subject an anti-HBV antibody or antigen-binding fragment thereof; and further comprising measuring the amount of HBsAg present in a blood sample from the subject before and after administering the agent that reduces HBV antigenic load, wherein a decrease in HBsAg indicates reduced expression of the at least one HBV gene.

In certain embodiments, the present disclosure provides an agent that reduces HBV antigenic load for use in the treatment of a chronic HBV infection or an HBV-associated disease in a subject, wherein the subject is subsequently administered an anti-HBV antibody or antigen-binding fragment thereof. In certain other embodiments, the present disclosure provides an anti-HBV antibody or antigen-binding fragment thereof for use in the treatment of a chronic HBV infection or an HBV-associated disease in a subject, and the subject has been previously administered an agent that reduces HBV antigenic load. In further embodiments, expression of at least one HBV gene is reduced after administration of the agent that reduces HBV antigenic load, and the anti-HBV antibody or antigen-binding fragment thereof is administered to the subject when expression of the at least one HBV gene is reduced.

In certain embodiments, the present disclosure provides the use of an agent that reduces HBV antigenic load and/or an anti-HBV antibody or antigen-binding fragment thereof in the manufacture of a medicament for the treatment of a chronic HBV infection or an HBV-associated disease.

Some embodiments of the present disclosure provide methods of treating chronic HBV infection or an HBV-associated disease in a subject in need thereof, comprising: (i) administering to the subject an inhibitor of HBV gene expression; and (ii) administering to the subject an anti-HBV antibody or antigen-binding fragment thereof. In certain embodiments, the inhibitor of HBV gene expression is administered before the anti-HBV antibody. In certain embodiments, administering the inhibitor of HBV gene expression before the anti-HBV antibody or antigen-binding fragment thereof causes the viral load to be reduced when the anti-HBV antibody is administered. In certain embodiments, the therapeutically effective amount of the anti-HBV antibody of the combination therapy is less than a therapeutically effective amount of the anti-HBV antibody or antigen-binding fragment thereof delivered when the inhibitor of HBV gene expression has not been administered to the subject (e.g., when the anti-HBV antibody or antigen-binding fragment thereof is administered alone as a monotherapy).

In certain embodiments, expression of at least one HBV gene is reduced after administering the inhibitor of HBV gene expression, and the anti-HBV antibody or antigen-binding fragment thereof is administered to the subject when expression of the at least one HBV gene is reduced. In particular embodiments, the at least one HBV gene is HBV X gene and/or HBsAg.

In certain embodiments, the present disclosure provides a method of treating a chronic HBV infection or HBV-associated disease in a subject in need thereof, comprising: administering to the subject an inhibitor of HBV gene expression; and administering to the subject an anti-HBV antibody or antigen-binding fragment thereof; and further comprising measuring the amount of HBsAg present in a blood sample from the subject before and after administering the inhibitor of HBV expression, wherein a decrease in HBsAg indicates reduced expression of the at least one HBV gene.

In certain embodiments, the present disclosure provides an inhibitor of HBV gene expression for use in the treatment of a chronic HBV infection or an HBV-associated disease in a subject, wherein the subject is subsequently administered an anti-HBV antibody or antigen-binding fragment thereof. In certain other embodiments, the present disclosure provides an anti-HBV antibody or antigen-binding fragment thereof for use in the treatment of a chronic HBV infection or an HBV-associated disease in a subject, and the subject has been previously administered an inhibitor of gene expression. In further embodiments, expression of at least one HBV gene is reduced after administration of the inhibitor of HBV gene expression, and the anti-HBV antibody or antigen-binding fragment thereof is administered to the subject when expression of the at least one HBV gene is reduced.

In certain embodiments, the present disclosure provides the use of an inhibitor of HBV gene expression and/or an anti-HBV antibody or antigen-binding fragment thereof in the manufacture of a medicament for the treatment of a chronic HBV infection or an HBV-associated disease.

In any of the above methods, compositions for use, or uses in manufacture, the methods and compositions may be used for treating a chronic HBV infection.

In certain embodiments, the inhibitor of HBV gene expression is administered in a single dose, two doses, three doses, four doses, or five doses. In certain particular embodiments, at least the first dose of the inhibitor of HBV gene expression is administered prior to administering the anti-HBV antibody or antigen-binding fragment thereof.

In certain embodiments, the inhibitor of HBV gene expression is administered in a single dose, two doses, three doses, four doses, or five doses, six doses, seven doses, or eight doses. The dose or doses may be administered, for example, twice daily, once daily, every two days, every three days, twice per week, once per week, every other week, every four weeks, or once per month.

In certain embodiments, administering the anti-HBV antibody or antigen-binding fragment thereof comprises administering the anti-HBV or antigen-binding fragment thereof antibody twice per week, once per week, every other week, every two weeks, or once a month.

In certain embodiments, administering the anti-HBV antibody or antigen-binding fragment thereof comprises administering at least two doses of a therapeutically effective amount of the anti-HBV antibody or antigen-binding fragment thereof. In certain further embodiments, the at least two doses are administered twice per week, once per week, every other week, every two weeks, or once a month.

In certain embodiments, administering the anti-HBV antibody or antigen-binding fragment thereof begins at least 1 week after administering the inhibitor of HBV gene expression. In certain embodiments, administering the anti-HBV antibody begins 2 weeks after administering the inhibitor of HBV gene expression. In certain embodiments, administering the anti-HBV antibody or antigen-binding fragment thereof begins 8 weeks after administering the inhibitor of HBV gene expression.

In certain embodiments, the anti-HBV antibody or antigen-binding fragment thereof and the inhibitor of HBV gene expression are each administered subcutaneously.

In particular embodiments of the above methods, compositions for use, or uses in manufacture, the anti-HBV antibody or antigen-binding fragment thereof may recognize HBV genotypes A, B, C, D, E, F, G, H, I, and J.

In particular embodiments of the above methods, compositions for use, or uses in manufacture, the anti-HBV antibody or antigen-binding fragment thereof may be a human antibody or antigen-binding fragment thereof; a monoclonal antibody or antigen-binding fragment thereof; or a bispecific antibody or antigen-binding fragment thereof, with a first specificity for HBsAg and a second specificity that stimulates an immune effector (e.g., a second specificity that stimulates cytotoxicity or a vaccinal effect). In certain other embodiments of the above methods, compositions for use, or uses in manufacture disclosed herein, the anti-HBV antibody is a monoclonal antibody.

In particular embodiments of the above methods, compositions for use, or uses in manufacture, the anti-HBV antibody or antigen-binding fragment thereof comprises a non-natural variant of HBC34 as disclosed herein. For example, in certain embodiments, the anti-HBV antibody (i) a heavy chain variable region (VH) comprising a CDRH1 amino acid sequence according to SEQ ID NO.:34, a CDRH2 amino acid sequence according to SEQ ID NO.:35 or 36, and a CDRH3 amino acid sequence according to SEQ ID NO.:37; and (ii) a light chain variable region (VL) comprising a CDRL1 amino acid sequence set forth in any one of SEQ ID NOs.:40-43, a CDRL2 amino acid sequence according to any one of SEQ ID NOs:45-53, and a CDRL3 amino acid sequence according to SEQ ID NO.:55 or 56, wherein the CDRs are determined according to the CCG numbering system, and wherein the antibody or antigen-binding fragment thereof is capable of binding to the antigenic loop region of HBsAg and neutralizing infection by a hepatitis B virus (HBV) of genotype D, A, B, C, E, F, G, H, I, or J, or any combination thereof.

In certain embodiments, the CDRH1, CDRH2, CDRH3, CDRL1, CDRL2, and CDRL3 amino acid sequences are according to SEQ ID NOs.: (i) 34, 35, 37, 41, 45, and 55, respectively; (ii) 34, 35, 37, 41, 46, and 55, respectively; (iii) 34, 35, 37, 41, 47, and 55, respectively; (iv) 34, 35, 37, 41, 48, and 55, respectively; (v) 34, 35, 37, 41, 49, and 55, respectively; (vi) 34, 35, 37, 41, 50, and 55, respectively; (vii) 34, 35, 37, 41, 51, and 55, respectively; (viii) 34, 35, 37, 41, 52, and 55, respectively; or (ix) 34, 35, 37, 41, 53, and 55, respectively.

In certain further embodiments, antibody or antigen-binding fragment comprises a heavy chain variable domain (VH) and a light chain variable domain (VL), wherein: (i) the VH comprises or consists of an amino acid sequence having at least 90% (i.e., 90%, 91%, 92%, 93%, 94%, 95%, 96%, 97%, 98%, 99%, or 100%, or any non-integer value therebetween) identity to the amino acid sequence set forth in SEQ ID NO.: 38 or 39; and/or (ii) the VL comprises or consists of an amino acid sequence having at least 90% (i.e., 90%, 91%, 92%, 93%, 94%, 95%, 96%, 97%, 98%, 99%, or 100%, or any non-integer value therebetween) identity to the amino acid sequence set forth in any one of SEQ ID NOs.: 58-66, 69, 71, or 72.

In some embodiments, the VH comprises or consists of the amino acid sequence set forth in SEQ ID NO.: 38 or 39; and/or the VL comprises or consists of the amino acid sequence set forth in any one of SEQ ID NOs.: 58-66, 69, 71, or 72.

In particular embodiments, the VH and the VL comprise or consist of the amino acid sequences set forth in SEQ ID NOs.: (i) 38 and 58, respectively; (ii) 38 and 59, respectively; (iii) 38 and 60, respectively; (iv) 38 and 61, respectively; (v) 38 and 62, respectively; (vi) 38 and 63, respectively; (vii) 38 and 64, respectively; (viii) 38 and 65, respectively; (ix) 38 and 66, respectively; (x) 38 and 71, respectively; or (xi) 38 and 72, respectively.

In another aspect, the present disclosure provides an antibody or antigen-binding fragment thereof, comprising: a heavy chain variable domain (VH) and a light chain variable domain (VL), wherein the VH and the VL comprise or consist of the amino acid sequences set forth in SEQ ID NOs.: (i) 38 and 67, respectively; or (ii) 38 and 68, respectively, wherein the antibody or antigen-binding fragment thereof is capable of binding to the antigenic loop region of HBsAg and neutralizing infection by a hepatitis B virus (HBV) of genotype D, A, B, C, E, F, G, H, I, or J, or any combination thereof.

Also provided is an antibody or antigen-binding fragment comprising a VH according to SEQ ID NO.:38 or 39 and a VL variant of any one of SEQ ID NOs.:57-72 that comprises one or more of the following mutations in framework region 3 relative to SEQ ID NO.:57-72, respectively, as determined by CCG numbering: R60A, R60N, R60K, S64A, I74A. In some embodiments, no further mutation relative to SEQ ID NO.:57-72, respectively, is comprised in the variant.

Also provided is an antibody or antigen-binding fragment comprising a VH according to SEQ ID NO.:38 or 39 and a VL variant of any one of SEQ ID NOs.:57-72 that comprises a substitution mutation (such as, for example, a conservative amino acid substitution, or a mutation to a germline-encoded amino acid) at Q78, D81, or both. In some embodiments, no further mutation relative to SEQ ID NO.:57-72, respectively, is comprised in the variant.

In any of the presently disclosed embodiments, in a sample comprising a plurality of the antibody or antigen-binding fragment, less than 12%, 11% or less, 10% or less, 9% or less, 8% or less, 7% or less, 6% or less, 5% or less, 4% or less, 3% or less, or 2% or less of the plurality is comprised in an antibody dimer when the sample has been incubated for about 120 to about 168 hours at about 40° C., wherein, optionally, the presence of antibody dimer is determined by absolute size-exclusion chromatography. As used herein, an antibody dimer or multimer is a complex comprising two or more of an antibody or antigen-binding fragment of the present disclosure (e.g., an antibody:antibody dimer, a Fab:Fab dimer, or an antibody:Fab dimer).

In certain embodiments, a therapeutically effective amount of the anti-HBV antibody or antigen-binding fragment is less than a therapeutically effective amount of the anti-HBV antibody or antigen-binding fragment delivered when the inhibitor of HBV gene expression has not been administered to the subject. For example, the combination therapy may lower the effective dose of the anti-HBV antibody or antigen-binding fragment, as compared to administration of the anti-HBV antibody or antigen-binding fragment alone.

In certain embodiments, the anti-HBV antibody or antigen-binding fragment is administered in at least two separate doses. In particular embodiments, the at least two doses are administered twice per week, once per week, every other week, every two weeks, or once a month.

In certain embodiments, the subject is a human and a therapeutically effective amount of the anti-HBV antibody is administered; wherein the therapeutically effective amount is from about 3 mg/kg to about 30 mg/kg.

In particular embodiments of the above methods, compositions for use, or uses in manufacture, the inhibitor is an RNAi agent that inhibits expression of an HBV transcript. In some embodiments, inhibition of expression of an HBV transcript is measured by rtPCR. In some embodiments, inhibition of expression of an HBV transcript is measured by a reduction in protein levels as measured by ELISA.

In certain embodiments, the RNAi agent comprises a sense strand and an antisense strand forming a double-stranded region, wherein the sense strand comprises at least 15 contiguous nucleotides differing by no more than 3 nucleotides from nucleotides 1579-1597 of SEQ ID NO:116. In certain embodiments, the RNAi agent comprises a sense strand and an antisense strand, wherein the sense strand comprises nucleotides 1579-1597 of SEQ ID NO:116.

In particular embodiments of the above methods, compositions for use, or uses in manufacture, at least one strand of the RNAi agent may comprise a 3′ overhang of at least 1 nucleotide or at least 2 nucleotides.

In particular embodiments of the above methods, compositions for use, or uses in manufacture, the double-stranded region of the RNAi agent may be 15-30 nucleotide pairs in length; 17-23 nucleotide pairs in length; 17-25 nucleotide pairs in length; 23-27 nucleotide pairs in length; 19-21 nucleotide pairs in length; or 21-23 nucleotide pairs in length.

In particular embodiments of the above methods, compositions for use, or uses in manufacture, each strand of the RNAi agent may be 15-30 nucleotides or 19-30 nucleotides.

In particular embodiments of the above methods, compositions for use, or uses in manufacture, the RNAi agent is an siRNA. In particular embodiments, the siRNA inhibits expression of an HBV transcript that encodes an HBsAg protein, an HBcAg protein, and HBx protein, or an HBV DNA polymerase protein. In certain embodiments, the siRNA binds to at least 15 contiguous nucleotides of a target encoded by: P gene, nucleotides 2309-3182 and 1-1625 of NC_003977.2; S gene (encoding L, M, and S proteins), nucleotides 2850-3182 and 1-837 of NC_003977.2; HBx, nucleotides 1376-1840 of NC_003977.2; or C gene, nucleotides 1816-2454 of NC_003977.2.

In particular embodiments of the above methods, compositions for use, or uses in manufacture, the RNAi agent is an siRNA, and the antisense strand of the siRNA comprises at least 15 contiguous nucleotides or 19 contiguous nucleotides of the nucleotide sequence of 5′-UGUGAAGCGAAGUGCACACUU-3′ (SEQ ID NO:119). In some embodiments, the antisense strand of the siRNA comprises the nucleotide sequence of 5′-UGUGAAGCGAAGUGCACACUU-3′ (SEQ ID NO:119). In some embodiments, the antisense strand consists of the nucleotide sequence of 5′-UGUGAAGCGAAGUGCACACUU-3′ (SEQ ID NO:119). In some embodiments, the sense strand of the siRNA comprises the nucleotide sequence of 5′-GUGUGCACUUCGCUUCACA-3′ (SEQ ID NO:118). In some embodiment, the sense strand of the siRNA consists of the nucleotide sequence of 5′-GUGUGCACUUCGCUUCACA-3′ (SEQ ID NO:118).

In particular embodiments of the above methods, compositions for use, or uses in manufacture, the RNAi agent is an siRNA, and the antisense strand of the siRNA comprises at least 15 contiguous nucleotides or 19 contiguous nucleotides of the nucleotide sequence of 5′-UAAAAUUGAGAGAAGUCCACCAC-3′ (SEQ ID NO:121). In some embodiments, the antisense strand of the siRNA comprises the nucleotide sequence of 5′-UAAAAUUGAGAGAAGUCCACCAC-3′ (SEQ ID NO:121). In some embodiments, the antisense strand consists of the nucleotide sequence of 5′-UAAAAUUGAGAGAAGUCCACCAC-3′ (SEQ ID NO:121). In some embodiments, the sense strand of the siRNA comprises the nucleotide sequence of 5′-GGUGGACUUCUCUCAAUUUUA-3′ (SEQ ID NO:120). In some embodiment, the sense strand of the siRNA consists of the nucleotide sequence of 5′-GGUGGACUUCUCUCAAUUUUA-3′ (SEQ ID NO:120).

In particular embodiments of the above methods, compositions for use, or uses in manufacture, the RNAi agent is an siRNA, wherein substantially all of the nucleotides of said sense strand and substantially all of the nucleotides of said antisense strand are modified nucleotides, and wherein said sense strand is conjugated to a ligand attached at the 3′-terminus. In particular embodiments, the ligand is one or more GalNAc derivatives attached through a monovalent linker, bivalent branched linker, or trivalent branched linker. In certain embodiments, the GalNAc derivative attached through a linker is or comprises:

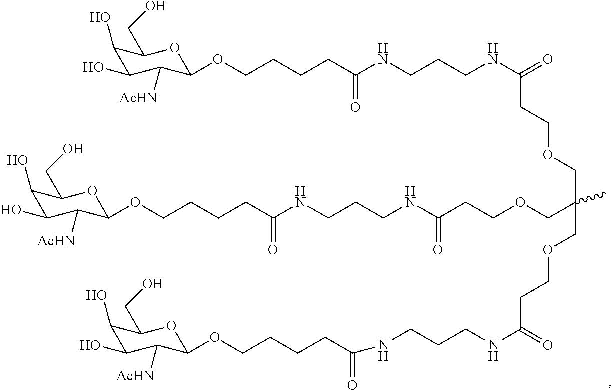 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In particular embodiments, the siRNA is conjugated to the ligand as shown in the following schematic (i.e., the GalNAc derivative attached through a linker is):

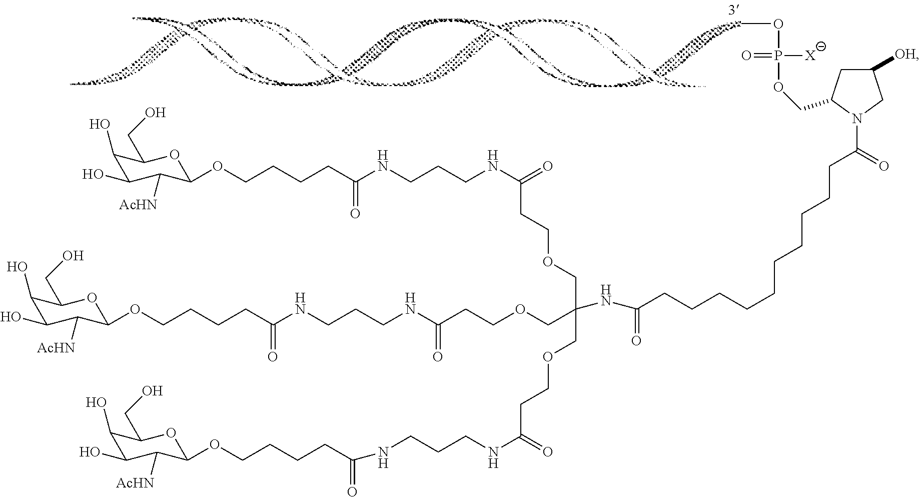

wherein X is O or S.

In particular embodiments of the above methods, compositions for use, or uses in manufacture, the RNAi agent is an siRNA, wherein at least one nucleotide of the siRNA is a modified nucleotide comprising a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, an adenosine-glycol nucleic acid, or a nucleotide comprising a 5′-phosphate mimic. In certain embodiments, the siRNA comprises a phosphate backbone modification, a 2′ ribose modification, 5′ triphosphate modification, or a GalNAc conjugation modification. In certain embodiments, the phosphate backbone modification comprises a phosphorothioate bond. In certain embodiments, the 2′ ribose modification comprises a fluoro or —O-methyl substitution.

In particular embodiments of the above methods, compositions for use, or uses in manufacture, the RNAi agent is an siRNA having a sense strand comprising 5′-gsusguGfcAfCfUfucgcuucacaL96-3′ (SEQ ID NO:122) and an antisense strand comprising 5′-usGfsugaAfgCfGfaaguGfcAfcacsusu-3′ (SEQ ID NO:123),

wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;

Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;

s is a phosphorothioate linkage; and

L96 is N-[tris(GalNAc-alkyl)-amidodecanoyl)]-4-hydroxyprolinol.

In particular embodiments of the above methods, compositions for use, or uses in manufacture, the RNAi agent is an siRNA having a sense strand comprising 5′-gsusguGfcAfCfUfucgcuucacaL96-3′ (SEQ ID NO:124) and an antisense strand comprising 5′-usGfsuga(Agn)gCfGfaaguGfcAfcacsusu-3′ (SEQ ID NO:125) wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;

(Agn) is adenosine-glycol nucleic acid (GNA);

s is a phosphorothioate linkage; and

L96 is N-[tris(GalNAc-alkyl)-amidodecanoyl)]-4-hydroxyprolinol.

In particular embodiments of the above methods, compositions for use, or uses in manufacture, the RNAi agent is an siRNA having a sense strand comprising 5′-gsgsuggaCfuUfCfUfcucaAfUfuuuaL96-3′ (SEQ ID NO:126) and an antisense strand comprising 5′-usAfsaaaUfuGfAfgagaAfgUfccaccsasc-3′ (SEQ ID NO:127),

wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;

Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;

s is a phosphorothioate linkage; and

L96 is N-[tris(GalNAc-alkyl)-amidodecanoyl)]-4-hydroxyprolinol.

In particular embodiments of the above methods, compositions for use, or uses in manufacture, the subject is a human and a therapeutically effective amount of RNAi or siRNA is administered to the subject; and wherein the effective amount of the RNAi or siRNA is from about 1 mg/kg to about 8 mg/kg.

In some embodiments of the methods, compositions for use, or uses disclosed herein, the siRNA is administered to the subject twice daily, once daily, every two days, every three days, twice per week, once per week, every other week, every four weeks, or once per month. In some embodiments, wherein the siRNA is administered to the subject every four weeks.

In certain embodiments, the methods include administering two inhibitors of HBV gene expression with an anti-HBV antibody. The two inhibitors of HBV gene expression may be two siRNAs, such as two siRNAs that target different HBV genes. The two different HBV genes may, for example, be HBsAg, and HBV X. The two inhibitors of HBV gene expression may be administered simultaneously. In certain embodiments, two siRNAs each directed to an HBV gene are administered, and the first siRNA has an antisense strand comprising SEQ ID NO:119, SEQ ID NO:123, or SEQ ID NO:125; and the second siRNA comprises an siRNA having a sense strand that comprises at least 15 contiguous nucleotides of nucleotides 2850-3182 of SEQ ID NO:116. In certain embodiments, two siRNAs each directed to an HBV gene are administered, and the first siRNA has an antisense strand comprising SEQ ID NO:121 or SEQ ID NO:127; and the second siRNA comprises an siRNA having a sense strand that comprises at least 15 contiguous nucleotides of nucleotides 2850-3182 of SEQ ID NO:116. In certain embodiments, two siRNAs each directed to an HBV gene are administered, and the first siRNA has an antisense strand comprising SEQ ID NO:119, SEQ ID NO:123 or SEQ ID NO:125; and the second siRNA has an antisense strand comprising SEQ ID NO:121 or SEQ ID NO:127. In certain embodiments, the first siRNA has a sense strand comprising SEQ ID NO:118, SEQ ID NO:122, or SEQ ID NO:124; and the second siRNA has a sense strand comprising SEQ ID NO:120 or SEQ ID NO:126.

In certain embodiments, the anti-HBV antibody and the inhibitor of HBV gene expression exhibit a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

In some embodiments, an RNAi agent targeting an HBV mRNA is administered to a subject having an HBV infection, and/or an HBV-associated disease, such that the expression of one or more HBV genes, HBV ccc DNA levels, HBV antigen levels, HBV viral load levels, ALT, and/or AST, e.g., in a cell, tissue, blood, or fluid of the subject are reduced by at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 62%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more.

In some embodiments, an RNAi agent targeting an HBV mRNA is administered to a subject having an HBV infection, and/or an HBV-associated disease, and inhibits HBV gene expression by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or about 100%, i.e., to below the level of detection of the assay.

In some embodiments, the combination therapy according to the present disclosure comprises administering a nucleot(s)ide analog as a third component. As used herein, the term “nucelot(s)ide analog” (or “polymerase inhibitor” or “reverse transcriptase inhibitor”) is an inhibitor of DNA replication that is structurally similar to a nucleotide or nucleoside and specifically inhibits replication of the HBV cccDNA and does not significantly inhibit the replication of the host (e.g., human) DNA. Such inhibitors include tenofovir disoproxil fumarate (TDF), tenofovir alafenamide (TAF), lamivudine, adefovir dipivoxil, entecavir (ETV), telbivudine, AGX-1009, emtricitabine (FTC), clevudine, ritonavir, dipivoxil, lobucavir, famvir, N-Acetyl-Cysteine (NAC), PC1323, theradigm-HBV, thymosin-alpha, ganciclovir, besifovir (ANA-380/LB-80380), and tenofvir-exaliades (TLX/CMX157). In certain embodiments, the nucleot(s)ide analog is entecavir (ETV). Nucleot(s)ide analogs are commercially available from a number of sources and are used in the methods provided herein according to their label indication (e.g., typically orally administered at a specific dose) or as determined by a skilled practitioner in the treatment of HBV.

The anti-HBV antibody or the inhibitor of HBV gene expression can be present either in the same pharmaceutical composition as the third active component or, the anti-HBV antibody, the inhibitor of HBV gene expression, and the third active component are present in three different pharmaceutical compositions. Such different pharmaceutical compositions may be administered either combined/simultaneously or at separate times or at separate locations (e.g., separate parts of the body).

The present disclosure also provides the following Embodiments:

Embodiment 1. An antibody, or an antigen-binding fragment thereof, comprising: (i) a heavy chain variable region (VH) that comprises therein the amino acid sequence of SEQ ID NO.:34, the amino acid sequence of SEQ ID NO.:35 or SEQ ID NO.:36, and the amino acid sequence of SEQ ID NO.:37; and (ii) a light chain variable region (VL) that comprises therein the amino acid sequence any one of SEQ ID NOs.:41, 40, 42, and 43, the amino acid sequence according to any one of SEQ ID NOs:49, 44-48, and 50-53, and the amino sequence according to SEQ ID NO.:55 or 56, wherein, optionally, the VL comprises a R60N substitution mutation, a R60A substitution mutation, a R60K substitution mutation, a S64A substitution mutation, a I74A substitution mutation, or any combination thereof, relative to SEQ ID NO.:58 and wherein the amino acid numbering of the substitution mutation(s) is according to SEQ ID NO.:58, and still further optionally wherein the VL does not comprise any further mutation(s) relative to SEQ ID NO.:58, and wherein the antibody or antigen-binding fragment thereof is capable of binding to the antigenic loop region of HBsAg and, optionally, neutralizing infection by a hepatitis B virus (HBV) of genotype D, A, B, C, E, F, G, H, I, or J, or any combination thereof.

Embodiment 2. The antibody or antigen-binding fragment of Embodiment 1, comprising: (i) in the VH, the amino acid sequences according to SEQ ID NOs.:34, 35, and 37, respectively, and in the VL, the amino acid sequences according to SEQ ID NOs.:41, 49, and 55, respectively; (ii) in the VH, the amino acid sequences according to SEQ ID NOs.:34, 35, and 37, and in the VL, the amino acid sequences according to SEQ ID NOs.: 41, 46, and 55, respectively; (iii) in the VH, the amino acid sequences according to SEQ ID NOs.:34, 35, and 37, respectively, and in the VL, the amino acid sequences according to SEQ ID NOs.: 41, 47, and 55, respectively; (iv) in the VH, the amino acid sequences according to SEQ ID NOs.:34, 35, and 37, respectively, and in the VL, the amino acid sequences according to SEQ ID NOs.:41, 48, and 55, respectively; (v) in the VH, the amino acid sequences according to SEQ ID NOs.:34, 35, and 37, respectively, and in the VL, the amino acid sequences according to SEQ ID NOs.:41, 45, and 55, respectively; (vi) in the VH, the amino acid sequences according to SEQ ID NOs.:34, 35, and 37, respectively, and in the VL, the amino acid sequences according to SEQ ID NOs.:41, 50, and 55, respectively; (vii) in the VH, the amino acid sequences according to SEQ ID NOs.:34, 35, and 37, respectively, and in the VL, the amino acid sequences according to SEQ ID NOs.:41, 51, and 55, respectively; (viii) in the VH, the amino acid sequences according to SEQ ID NOs.:34, 35, and 37, respectively, and in the VL, the amino acid sequences according to SEQ ID NOs.:41, 52, and 55, respectively; or (ix) in the VH, the amino acid sequences according to SEQ ID NOs.:34, 35, and 37, respectively, and in the VL, the amino acid sequences according to SEQ ID NOs.:41, 53, and 55, respectively.

Embodiment 3. An antibody, or an antigen-binding fragment thereof, comprising: (i) a heavy chain variable region (VH) comprising a CDRH1 amino acid sequence according to SEQ ID NO.:34, a CDRH2 amino acid sequence according to SEQ ID NO.:35 or 36, and a CDRH3 amino acid sequence according to SEQ ID NO.:37; and (ii) a light chain variable region (VL) comprising a CDRL1 amino acid sequence according to any one of SEQ ID NOs.:40-43, a CDRL2 amino acid sequence according to any one of SEQ ID NOs:49, 44-48, and 50-53, and a CDRL3 amino acid sequence according to SEQ ID NO.:55 or 56, wherein CDRs are defined according to the CCG numbering system, and wherein the antibody or antigen-binding fragment thereof is capable of binding to the antigenic loop region of HBsAg and, optionally, neutralizing infection by a hepatitis B virus (HBV) of genotype D, A, B, C, E, F, G, H, I, or J, or any combination thereof, provided that the antibody or antigen-binding fragment does not comprise CDRH1, CDRH2, CDRH3, CDRL1, CDRL2, and CDRL3 amino acid sequences according to SEQ ID NOs.:34, 35, 37, 41, 44, and 45, respectively.

Embodiment 4. The antibody or antigen-binding fragment of Embodiment 3, wherein the CDRH1, CDRH2, CDRH3, CDRL1, CDRL2, and CDRL3 amino acid sequences are according to SEQ ID NOs.: (i) 34, 35, 37, 41, 49, and 55, respectively; (ii) 34, 35, 37, 41, 46, and 55, respectively; (iii) 34, 35, 37, 41, 47, and 55, respectively; (iv) 34, 35, 37, 41, 48, and 55, respectively; (v) 34, 35, 37, 41, 45, and 55, respectively; (vi) 34, 35, 37, 41, 50, and 55, respectively; (vii) 34, 35, 37, 41, 51, and 55, respectively; (viii) 34, 35, 37, 41, 52, and 55, respectively; (ix) 34, 35, 37, 41, 53, and 55, respectively; or (x) 34, 35, 37, 41, 44, and 55, respectively.

Embodiment 5. An antibody, or an antigen-binding fragment thereof, comprising a heavy chain variable region (VH) and a light chain variable region (VL), wherein the VH and the VL comprise CDRH1, CDRH2, CDRH3 and CDRL1, CDRL2, CDRL3, respectively, according to: HBC34-v40; HBC34-v36; HBC34-v37; HBC34-v38; HBC34-v39; HBC34-v41; HBC34-v42; HBC34-v43; HBC34-v44; HBC34-v45; HBC34-v46; HBC34-v47; HBC34-v48; HBC34-v49; or HBC34-v50, wherein the CDRs are defined according to IMGT numbering, optionally wherein the VL further comprises a R60N substitution mutation, a R60A substitution mutation, a R60K substitution mutation, a S64A substitution mutation, a I74A substitution mutation, or any combination thereof, relative to SEQ ID NO.:58 and wherein the amino acid numbering of the substitution mutation(s) is according to SEQ ID NO.:58, and further optionally wherein the VL does not comprise any further mutation(s) relative to SEQ ID NO.:58.

Embodiment 6. An antibody, or an antigen-binding fragment thereof, comprising a heavy chain variable region (VH) and a light chain variable region (VL), wherein the VH and the VL comprise CDRH1, CDRH2, CDRH3 and CDRL1, CDRL2, CDRL3, respectively, according to: HBC34-v40; HBC34-v36; HBC34-v37; HBC34-v38; HBC34-v39; HBC34-v41; HBC34-v42; HBC34-v43; HBC34-v44; HBC34-v45; HBC34-v46; HBC34-v47; HBC34-v48; HBC34-v49; or HBC34-v50,

wherein the CDRs are defined according to CCG numbering, optionally wherein the VL further comprises a R60N substitution mutation, a R60A substitution mutation, a R60K substitution mutation, a S64A substitution mutation, a I74A substitution mutation, or any combination thereof, relative to SEQ ID NO.:58 and wherein the amino acid numbering of the substitution mutation(s) is according to SEQ ID NO.:58, and further optionally wherein the VL does not comprise any other mutation(s) relative to SEQ ID NO.:58.

Embodiment 7. The antibody or antigen-binding fragment of any one of Embodiments 1-6, wherein: (i) the VH comprises or consists of an amino acid sequence having at least 90% identity to the amino acid sequence set forth in SEQ ID NO.: 38 or 39; and/or (ii) the VL comprises or consists of an amino acid sequence having at least 90% identity to the amino acid sequence set forth in any one of SEQ ID NOs.: 62, 58-61, 63-66, 69, 71, and 72.

Embodiment 8. The antibody or antigen-binding fragment of any one of Embodiments 1-7, wherein: (i) the VH comprises or consists of an amino acid sequence having at least 90% (i.e., 90%, 91%, 92%, 93%, 94%, 95%, 96%, 97%, 98%, 99%, or 100%, or any non-integer value therebetween) identity to the amino acid sequence set forth in SEQ ID NO.: 38 or 39; and/or (ii) the VL comprises or consists of an amino acid sequence having at least 90% (i.e., 90%, 91%, 92%, 93%, 94%, 95%, 96%, 97%, 98%, 99%, or 100%, or any non-integer value therebetween) identity to the amino acid sequence set forth in any one of SEQ ID NOs.: 62, 58-61, 63-66, 69, 71, and 72.

Embodiment 9. The antibody or antigen-binding fragment of any one of Embodiments 1-8, wherein the VH and the VL comprise or consist of amino acid sequences having at least 90% (i.e., 90%, 91%, 92%, 93%, 94%, 95%, 96%, 97%, 98%, 99%, or any non-integer value therebetween) identity to the amino acid sequences set forth in SEQ ID NOs.: (i) 38 and 62, respectively; (ii) 38 and 59, respectively; (iii) 38 and 60, respectively; (iv) 38 and 61, respectively; (v) 38 and 58, respectively; (vi) 38 and 63, respectively; (vii) 38 and 64, respectively; (viii) 38 and 65, respectively; (ix) 38 and 66, respectively; (x) 38 and 71, respectively; or (xi) 38 and 72, respectively.

Embodiment 10. An antibody, or an antigen-binding fragment thereof, comprising a heavy chain variable region (VH) that comprises or consists of the amino acid sequence of SEQ ID NO.:38 or 39, and a light chain variable region (VL) that comprises a variant of any one of SEQ ID NOs.:62, 57-61, and 63-72, wherein the variant comprises any one or more of the following mutations: R60A; R60N; R60K; S64A; and I74A, and wherein, optionally, the VL variant does not comprise any further mutations as compared to SEQ ID NO.:62, 57-61, and 63-72, respectively.

Embodiment 11. An antibody, or an antigen-binding fragment thereof, comprising a heavy chain variable region (VH) that comprises or consists of the amino acid sequence of SEQ ID NO.:38 or 39, and a light chain variable region (VL) that comprises a variant of any one of SEQ ID NOs.:62, 57-61, and 63-72, wherein the variant comprises a substitution mutation (such as, for example, a conservative amino acid substitution, or a mutation to a germline-encoded amino acid) at Q78, D81, or both, and wherein, optionally, the VL variant does not comprise any further mutations as compared to SEQ ID NO.:62, 57-61, and 63-72, respectively.

Embodiment 12. The antibody or antigen-binding fragment of any one of Embodiments 1-9, wherein: the VH comprises or consists of the amino acid sequence set forth in SEQ ID NO.: 38 or 39; and/or the VL comprises or consists of the amino acid sequence set forth in any one of SEQ ID NOs.: 62, 58-61, 63-66, 69, 71, or 72.

Embodiment 13. The antibody or antigen-binding fragment of any one of Embodiments 1-9 and 12, wherein the VH and the VL comprise or consist of the amino acid sequences set forth in SEQ ID NOs.: (i) 38 and 62, respectively; (ii) 38 and 59, respectively; (iii) 38 and 60, respectively; (iv) 38 and 61, respectively; (v) 38 and 58, respectively; (vi) 38 and 63, respectively; (vii) 38 and 64, respectively; (viii) 38 and 65, respectively; (ix) 38 and 66, respectively; (x) 38 and 71, respectively; or (xi) 38 and 72, respectively.

Embodiment 14. An antibody or antigen-binding fragment, comprising a heavy chain variable region (VH) and a light chain variable region (VL), wherein the VH and the VL comprise or consist of the amino acid sequences set forth in SEQ ID NOs.: (i) 38 and 62, respectively; (ii) 38 and 66, respectively; (iii) 38 and 67, respectively; (iv) 38 and 68, respectively; or (v) 38 and 72, respectively, wherein the antibody or antigen-binding fragment thereof is capable of binding to the antigenic loop region of HBsAg and neutralizing infection by a hepatitis B virus (HBV) of genotype D, A, B, C, E, F, G, H, I, or J, or any combination thereof.

Embodiment 15. The antibody or antigen-binding fragment of any one of Embodiments 1-14, which is capable of neutralizing infection by a hepatitis D virus (HDV).

Embodiment 16. The antibody or antigen-binding fragment of any one of Embodiments 1-15, wherein, in a sample comprising a plurality of the antibody or antigen-binding fragment, less than 12%, 11% or less, 10% or less, 9% or less, 8% or less, 7% or less, 6% or less, 5% or less, 4% or less, 3% or less, or 2% or less of the plurality is comprised in a dimer when the sample has been incubated for about 120 to about 168 hours at about 40° C., wherein, optionally, the presence of dimer is determined by absolute size-exclusion chromatography.

Embodiment 17. The antibody or antigen-binding fragment of any one of Embodiments 1-16, wherein incubation of a plurality of the antibody or antigen-binding fragment results in reduced formation of a dimer as compared to incubation of a plurality of a reference antibody or antigen-binding fragment,

wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57, and wherein, optionally, the presence of antibody dimer is determined by absolute size-exclusion chromatography.

Embodiment 18. The antibody or antigen-binding fragment of any one of Embodiments 1-17, which forms a lower amount of dimer, and/or forms dimers at a reduced frequency and/or as a lower percentage of total antibody or antigen-binding fragment molecules in a sample or composition as compared to a reference antibody:

(i) in a 5-day, a 15-day, and/or a 32-day incubation at 4° C.;

(ii) in a 5-day, a 15-day, and/or a 32-day incubation at 25° C.; and/or

(iii) in a 5-day, a 15-day, and/or a 32-day incubation at 40° C.,

wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

Embodiment 19. The antibody or antigen-binding fragment of any one of Embodiments 1-18, wherein a percentage of antibody or antigen-binding fragment molecules in a composition that are comprised in a dimer is less than ⅘, less than ¾, less than ½, less than ⅓, less than ¼, less than ⅕, less than ⅙, less than 1/7, less than ⅛, less than 1/9, or less than 1/10 the percentage of reference antibody molecules in a composition that are present in a dimer, respectively.

Embodiment 20. The antibody or antigen-binding fragment of any one of Embodiments 1-19, wherein a host cell transfected with a polynucleotide encoding the antibody or antigen-binding fragment provides 1.5× or more, 2× or more, 3× or more, or 4× or more the amount of antibody or antigen-binding fragment, respectively, than a reference host cell transfected with a polynucleotide encoding a reference antibody or antigen-binding fragment, wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

Embodiment 21. The antibody or antigen-binding fragment of any one of Embodiments 1-20, wherein the antibody or antigen-binding fragment thereof is produced in transfected cells at a higher titer as compared to a reference antibody or antigen-binding fragment is produced in reference transfected cells, wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

Embodiment 22. The antibody or antigen-binding fragment of any one of Embodiments 1-21, wherein the antibody or antigen-binding fragment thereof is produced in transfected cells at titers of at least 1.5-fold, at least 2-fold, at least 3-fold, or at least 4-fold, higher than the titer at which a reference antibody or antigen-binding fragment is produced, wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

Embodiment 23. The antibody or antigen-binding fragment of any one of Embodiments 1-22, wherein the antibody or antigen-binding fragment is capable of binding to a HBsAg (adw) with an EC50 (ng/ml) of about 3.2 or less, less than 3.0, less than 2.5, less than 2.0, less than 1.5, or less than 1.0.

Embodiment 24. The antibody or antigen-binding fragment of any one of Embodiments 1-23, wherein the antibody or antigen-binding fragment is capable of binding to a HBsAg (e.g., of subtype adw) with an EC50 (ng/ml) of less than 3.5, less than 3.4, less than 3.3, less than 3.2, less than 3.1, less than 3.0, less than 2.9, less than 2.8, less than 2.7, less than 2.6, less than 2.5, less than 2.4, less than 2.3, less than 2.1, less than 2.0, less than 1.9, less than 1.8, less than 1.7, less than 1.6, less than 1.5, less than 1.4, less than 1.3, less than 1.2, less than 1.1, or less than 1.0.

Embodiment 25. The antibody or antigen-binding fragment of any one of Embodiments 1-24, wherein the antibody or antigen-binding fragment is capable of binding to a HBsAg (e.g., of subtype adw) with an EC50 (ng/ml) of between 0.9 and 2.0, or of between 0.9 and 1.9, or of between 0.9 and 1.8, or of between 0.9 and 1.7, or of between 0.9 and 1.6, or of between 0.9 and 1.5, or of between 0.9 and 1.4, or of between 0.9 and 1.3, or of between 0.9 and 1.2, or of between 0.9 and 1.1, or of between 0.9 and 1.0, or of between 1.0 and 2.0.

Embodiment 26. The antibody or antigen-binding fragment of any one of Embodiments 1-25, wherein the antibody or antigen-binding fragment is capable of binding to a HBsAg (adw) with an EC50 (ng/ml) of 2.0 or less.

Embodiment 27. The antibody or antigen-binding fragment of any one of Embodiments 1-26, which has a hepatitis B virus neutralization of infection EC50 of less than 20 ng/ml, preferably 15 ng/ml or less, more preferably 10 ng/mL or less.

Embodiment 28. The antibody or antigen-binding fragment of any one of Embodiments 1-27, wherein the antibody or antigen-binding fragment thereof is capable of neutralizing hepatitis B virus infection with a neutralization of infection EC50 of 18, 17, 16, 15, 14, 13, 12, 11, 10, 9, 8, or 7 ng/mL.

Embodiment 29. The antibody or antigen-binding fragment of any one of Embodiments 1-28, wherein the antibody or antigen-binding fragment thereof is capable of neutralizing hepatitis B virus infection with a neutralization of infection EC50 that is lower than the neutralization of infection EC50 of a reference antibody or antigen-binding fragment that comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

Embodiment 30. The antibody or antigen-binding fragment of any one of Embodiments 1-29, wherein the antibody, or the antigen-binding fragment thereof, comprises a human antibody, a monoclonal antibody, a purified antibody, a single chain antibody, a Fab, a Fab′, a F(ab′)2, a Fv, or a scFv.

Embodiment 31. The antibody or antigen-binding fragment of any one of Embodiments 1-30, wherein the antibody or antigen-binding fragment is a multi-specific antibody or antigen-binding fragment.

Embodiment 32. The antibody or antigen-binding fragment of any one of Embodiments 1-31, wherein the antibody or antigen-binding fragment is a bispecific antibody or antigen-binding fragment.

Embodiment 33. The antibody of any one of Embodiments 1-32, or an antigen-binding fragment thereof, wherein the antibody or the antigen-binding fragment comprises a Fc moiety.

Embodiment 34. The antibody or antigen-binding fragment of Embodiment 33, wherein the Fc moiety comprises a mutation that enhances binding to FcRn as compared to a reference Fc moiety that does not comprise the mutation.

Embodiment 35. The antibody or antigen-binding fragment of Embodiment 33 or 34, wherein the Fc moiety comprises a mutation that enhances binding to a FcγR, preferably a FcγRIIA and/or a FcγRIIIA, as compared to a reference Fc moiety that does not comprise the mutation.

Embodiment 36. The antibody or antigen-binding fragment of any one of Embodiments 33-35, wherein the Fc moiety is an IgG isotype, such as IgG1, or is derived from an IgG isotype, such as IgG1.

Embodiment 37. The antibody or antigen-binding fragment of any one of Embodiments 33-36, which comprises or is derived from Ig G1m17, 1 (IgHG1*01).

Embodiment 38. The antibody or antigen-binding fragment of any one of Embodiments 34-37, wherein the mutation that enhances binding to FcRn comprises: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; or (viii) any combination of (i)-(vii), wherein amino acid numbering of the Fc moiety is according to the EU numbering system.

Embodiment 39. The antibody or antigen-binding fragment of Embodiment 38, wherein the mutation that enhances binding to FcRn comprises M428L/N434S.

Embodiment 40. The antibody or antigen-binding fragment of any one of Embodiments 35-39, wherein the mutation that enhances binding to a FcγR comprises S239D; I332E; A330L; G236A; or any combination thereof, wherein amino acid numbering of the Fc moiety is according to the EU numbering system.

Embodiment 41. The antibody or antigen-binding fragment of Embodiment 40, wherein the mutation that enhances binding to a FcγR comprises: (i) S239D/I332E; (ii) S239D/A330L/I332E; (iii) G236A/S239D/I332E; or (iv) G236A/A330L/I332E.

Embodiment 42. The antibody or antigen-binding fragment of Embodiment or 41, wherein the mutation that enhances binding to a FcγR comprises or consists of G236A/A330L/I332E, and optionally wherein the antibody or antigen-binding fragment does not comprise S239D, and wherein the antibody or antigen-binding fragment further optionally comprises a native S at position 239.

Embodiment 43. The antibody or antigen-binding fragment of any one of Embodiments 33-42, wherein the Fc moiety comprises the amino acid substitution mutations: M428L; N434S; G236A; A330L; and I332E, and optionally does not comprise S239D.

Embodiment 44. The antibody or antigen-binding fragment of any one of Embodiments 1-43, comprising a light chain constant region (CL) that comprises or consists of an amino acid sequence having at least 90%, 91%, 92%, 93%, 94%, 95%, 96%, 97%, 98%, 99%, or 100% identity to the amino acid sequence of SEQ ID NO.:79.

Embodiment 45. The antibody or antigen-binding fragment of any one of Embodiments 1-44, comprising a CH1-CH2-CH3 that comprises or consists of an amino acid sequence having 90%, 91%, 92%, 93%, 94%, 95%, 96%, 97%, 98%, 99%, or 100% identity to the amino acid sequence of SEQ ID NO.:73, or a variant thereof that comprises one or more of the following amino acid substitutions (EU numbering): G236A; A330L; I332E; M428L; N434S.

Embodiment 46. The antibody or antigen-binding fragment of Embodiment 45, wherein the CH1-CH2-CH3 has a C-terminal lysine removed.

Embodiment 47. An antibody comprising: a heavy chain (HC) comprising or consisting of the amino acid sequence set forth in SEQ ID NO.:75, optionally with the C-terminal lysine removed; and a light chain (LC), wherein the LC comprises or consists of (i) the VL amino acid sequence set forth in any one of SEQ ID NOs.:62, 58-61, and 63-72 and (ii) the CL amino acid sequence set forth in SEQ ID NO.:79.

Embodiment 48. The antibody of Embodiment 47, wherein the LC comprises the VL amino acid sequence set forth in any one of SEQ ID NOs.:62, 66, 67, and 72.

Embodiment 49. An antibody comprising: a heavy chain (HC) comprising or consisting of the amino acid sequence set forth in SEQ ID NO.:76, optionally with the C-terminal lysine removed, and a light chain (LC), wherein the LC comprises or consists of (i) the VL amino acid sequence set forth in any one of SEQ ID NOs.:62, 58-61, and 63-72 and (ii) the CL amino acid sequence set forth in SEQ ID NO.:79.

Embodiment 50. The antibody of Embodiment 49, wherein the LC comprises the VL amino acid sequence set forth in any one of SEQ ID NOs.:62, 66, 67, and 72.

Embodiment 51. An antibody comprising: a heavy chain (HC) comprising or consisting of the amino acid sequence set forth in SEQ ID NO.:77, optionally with the C-terminal lysine removed, and a light chain (LC), wherein the LC comprises or consists of (i) the VL amino acid sequence set forth in any one of SEQ ID NOs.:62, 58-61, and 63-72 and (ii) the CL amino acid sequence set forth in SEQ ID NO.:79.

Embodiment 52. The antibody of Embodiment 51, wherein the LC comprises the VL amino acid sequence set forth in any one of SEQ ID NOs.:62, 66, 67, and 72.

Embodiment 53. An antibody comprising: a heavy chain (HC) comprising or consisting of the amino acid sequence set forth in SEQ ID NO.:78, optionally with the C-terminal lysine removed, and a light chain (LC), wherein the LC comprises or consists of (i) the VL amino acid sequence set forth in any one of SEQ ID NOs.:62, 58-61, and 63-72 and (ii) the CL amino acid sequence set forth in SEQ ID NO.:79.

Embodiment 54. The antibody of Embodiment 53, wherein the LC comprises the VL amino acid sequence set forth in any one of SEQ ID NOs.:62, 66, 67, and 72.

Embodiment 55. The antibody or antigen-binding fragment of any one of Embodiments 1-54, wherein the antibody or the antigen-binding fragment is capable of binding an HBsAg of a genotype selected from the HBsAg genotypes A, B, C, D, E, F, G, H, I, and J, or any combination thereof.

Embodiment 56. The antibody or antigen-binding fragment of any one of Embodiments 1-55, wherein the antibody or antigen-binding fragment is capable of reducing a serum concentration of HBV DNA in a mammal having an HBV infection.

Embodiment 57. The antibody or antigen-binding fragment of any one of Embodiments 1-56, wherein the antibody or antigen-binding fragment is capable of reducing a serum concentration of HBsAg in a mammal having an HBV infection.

Embodiment 58. The antibody or antigen binding fragment of any one of Embodiments 1-57, wherein the antibody or antigen-binding fragment is capable of reducing a serum concentration of HBeAg in a mammal having an HBV infection.

Embodiment 59. The antibody or antigen binding fragment of any one of Embodiments 1-58, wherein the antibody or antigen binding fragment is capable of reducing a serum concentration of HBcrAg in a mammal having an HBV infection.

Embodiment 60. A polynucleotide comprising a nucleotide sequence that encodes the antibody, or the antigen-binding fragment, of any one of Embodiments 1-59.

Embodiment 61. A polynucleotide encoding a light chain variable region (VL) and, optionally, a light chain constant region (CL) of the antibody, or the antigen-binding fragment, of any one of Embodiments 1-59.

Embodiment 62. The polynucleotide of Embodiment 61, wherein the nucleotide sequence that encodes the antibody or the antigen-binding fragment is codon optimized for expression in a host cell.

Embodiment 63. The polynucleotide of Embodiment 62, comprising a nucleotide sequence having at least 50% identity to the nucleotide sequence according to any one of SEQ ID NOs:89, 85-88, and 90-99.

Embodiment 64. The polynucleotide of any one of Embodiments 60-63, comprising (i) the polynucleotide sequence set forth in SEQ ID NO.:81 or SEQ ID NO.:82, and (ii) the polynucleotide sequence set forth in any one or more of SEQ ID NOs.:89, 85-88, and 90-99.

Embodiment 65. The polynucleotide of any one of Embodiments 60-63, comprising (i) the polynucleotide sequence set forth in SEQ ID NO.:83, and (ii) the polynucleotide sequence set forth in any one or more of SEQ ID NOs.:89, 85-88, and 90-99.

Embodiment 66. The polynucleotide of any one of Embodiments 60-63, comprising (i) the polynucleotide sequence set forth in SEQ ID NO.:84, and (ii) the polynucleotide sequence set forth in any one or more of SEQ ID NOs.:89, 85-88, and 90-99.

Embodiment 67. A vector comprising the polynucleotide of any one of Embodiments 60-66.

Embodiment 68. The vector of Embodiment 67, wherein the vector comprises a lentiviral vector or a retroviral vector.

Embodiment 69. A host cell comprising the polynucleotide of any one of Embodiments 60-66 and/or the vector of Embodiment 67 or 68.

Embodiment 70. A pharmaceutical composition comprising: (i) the antibody or antigen binding fragment of any one of Embodiments 1-59; (ii) the polynucleotide according to any one of Embodiments 60-66; (iii) the vector according to Embodiment 67 or 68; (iv) the host cell of Embodiment 69; or (v) any combination of (i)-(iv), and a pharmaceutically acceptable excipient, diluent or carrier.

Embodiment 71. A kit comprising:

(a) a component selected from: (i) the antibody or antigen-binding fragment of any one of Embodiments 1-59; (ii) the polynucleotide according to any one of Embodiments 60-66; (iii) the vector according to Embodiment 67 or 68;

(iv) the host cell of Embodiment 69; (v) the pharmaceutical composition of Embodiment 70; or (vi) any combination of (i)-(vi); and

(b) (1) instructions for using the component to prevent, treat, attenuate, and/or diagnose a hepatitis B infection and/or a hepatitis D infection and/or (2) a means for administering the component to the subject, such as a syringe.

Embodiment 72. The composition of Embodiment 70 or the kit of Embodiment 71, further comprising: (i) a polymerase inhibitor, wherein the polymerase inhibitor optionally comprises Lamivudine, Adefovir, Entecavir, Telbivudine, Tenofovir, or any combination thereof; (ii) an interferon, wherein the interferon optionally comprises IFNbeta and/or IFNalpha; (iii) a checkpoint inhibitor, wherein the checkpoint inhibitor optionally comprises an anti-PD-1 antibody or antigen binding fragment thereof, an anti-PD-L1 antibody or antigen binding fragment thereof, and/or an anti-CTLA4 antibody or antigen binding fragment thereof; (iv) an agonist of a stimulatory immune checkpoint molecule; or (v) any combination of (i)-(iv).

Embodiment 73. The composition or kit of Embodiment 72, wherein the polymerase inhibitor comprises lamivudine.

Embodiment 74. A method of producing the antibody or antigen binding fragment of any one of Embodiments 1-59, comprising culturing the host cell of Embodiment 69 under conditions and for a time sufficient to produce the antibody or antigen-binding fragment.

Embodiment 75. Use of: (i) the antibody or antigen-binding fragment of any one of Embodiments 1-59; (ii) the polynucleotide of any one of Embodiments 60-66; (iii) the vector of Embodiment 67 or 68; (iv) the host cell of Embodiment 69; and/or (v) the pharmaceutical composition of Embodiment 70, 72, or 73, in the manufacture of a medicament to prevent, treat, attenuate, and/or diagnose a hepatitis B infection and/or a hepatitis D infection in a subject.

Embodiment 76. A method of treating, preventing, and/or attenuating a hepatitis B and/or hepatitis D infection in a subject, comprising administering to the subject an effective amount of: (i) the antibody or antigen-binding fragment of any one of Embodiments 1-59; (ii) the polynucleotide of any one of Embodiments 60-66; (iii) the vector of Embodiment 67 or 68; (iv) the host cell of Embodiment 69; and/or (v) the pharmaceutical composition of Embodiment 70, 72, or 73.

Embodiment 77. The method of Embodiment 76, further comprising administering to the subject one or more of: (vi) a polymerase inhibitor, wherein the polymerase inhibitor optionally comprises Lamivudine, Adefovir, Entecavir, Telbivudine, Tenofovir, or any combination thereof; (vii) an interferon, wherein the interferon optionally comprises IFNbeta and/or IFNalpha; (viii) a checkpoint inhibitor, wherein the checkpoint inhibitor optionally comprises an anti-PD-1 antibody or antigen binding fragment thereof, an anti-PD-L1 antibody or antigen binding fragment thereof, and/or an anti-CTLA4 antibody or antigen binding fragment thereof; (ix) an agonist of a stimulatory immune checkpoint molecule; or (x) any combination of (vi)-(ix).

Embodiment 78. The method of Embodiment 76 or 77, wherein the hepatitis B infection is a chronic hepatitis B infection.

Embodiment 79. The method of any one of Embodiments 76-78, wherein the subject has received a liver transplant.

Embodiment 80. The method of any one of Embodiments 76-79, wherein the subject is non-immunized against hepatitis B.

Embodiment 81. The method of any one of Embodiments 76-80, wherein the subject is a newborn.

Embodiment 82. The method of any one of Embodiments 76-81, wherein the subject is undergoing or has undergone hemodialysis.

Embodiment 83. The method of any one of Embodiments 76-82, wherein the method comprises administering to the subject a single dose of a pharmaceutical composition comprising the antibody or antigen-binding fragment.

Embodiment 84. The method of Embodiment 83, wherein the single dose of the pharmaceutical composition comprises the antibody in a range from 2 to 18 mg/kg (subject body weight).

Embodiment 85. The method of Embodiment 83 or 84, wherein the single dose of the pharmaceutical composition comprises up to 6 mg, up to 10 mg, up to 15 mg, up to 18 mg, up to 25 mg, up to 30 mg, up to 35 mg, up to 40 mg, up to 45 mg, up to 50 mg, up to 55 mg, up to 60 mg, up to 75 mg, up to 90 mg, up to 300 mg, up to 900 mg, or up to 3000 mg of the antibody,

or wherein the single dose of the pharmaceutical composition comprises the antibody in an amount that is in a range from 1 mg to 3000 mg, or in a range from 5 mg to 3000 mg, or in a range from 10 mg to 3000 mg, or in a range from 25 mg to 3000 mg, or in a range from 30 mg to 3000 mg, or in a range from 50 mg to 3000 mg, or in a range from 60 mg to 3000 mg, or in a range from 75 mg to 3000 mg, or in a range from 90 mg to 3000 mg, or in a range from 100 mg to 3000 mg, or in a range from 150 mg to 3000 mg, or in a range from 200 mg to 3000 mg, or in a range from 300 mg to 3000 mg, or in a range from 500 mg to 3000 mg, or in a range from 750 mg to 3000 mg, or in a range from 900 mg to 3000 mg, or in a range from 1500 mg to 3000 mg, or in a range from 2000 mg to 3000 mg,

or wherein the single dose of the pharmaceutical composition comprises the antibody in an amount that is in a range from 1 mg to 900 mg, or in a range from 5 mg to 900 mg, or in a range from 10 mg to 900 mg, or in a range from 25 mg to 900 mg, or in a range from 30 mg to 900 mg, or in a range from 50 mg to 900 mg, or in a range from 60 mg to 900 mg, or in a range from 75 mg to 900 mg, or in a range from 90 mg to 900 mg, or in a range from 100 mg to 900 mg, or in a range from 150 mg to 900 mg, or in a range from 200 mg to 900 mg, or in a range from 300 mg to 900 mg, or in a range from 500 mg to 900 mg, or in a range from 750 mg to 900 mg,

or wherein the single dose of the pharmaceutical composition comprises the antibody in an amount that is wherein the single dose of the pharmaceutical composition comprises the antibody in an amount that is in a range from 1 mg to 500 mg, or in a range from 5 mg to 500 mg, or in a range from 10 mg to 500 mg, or in a range from 25 mg to 500 mg, or in a range from 30 mg to 500 mg, or in a range from 50 mg to 500 mg, or in a range from 60 mg to 500 mg, or in a range from 75 mg to 500 mg, or in a range from 90 mg to 500 mg, or in a range from 100 mg to 500 mg, or in a range from 150 mg to 500 mg, or in a range from 200 mg to 500 mg, or in a range from 300 mg to 500 mg, or in a range from 400 mg to 500 mg,

or wherein the single dose of the pharmaceutical composition comprises the antibody in an amount that is in a range from 1 mg to 300 mg, or in a range from 5 mg to 300 mg, or in a range from 10 mg to 300 mg, or in a range from 25 mg to 300 mg, or in a range from 30 mg to 300 mg, or in a range from 50 mg to 300 mg, or in a range from 60 mg to 300 mg, or in a range from 75 mg to 300 mg, or in a range from 90 mg to 300 mg, or in a range from 100 mg to 300 mg, or in a range from 150 mg to 300 mg, or in a range from 200 mg to 300 mg,

or wherein the single dose of the pharmaceutical composition comprises the antibody in an amount that is in a range from 1 mg to 200 mg, or in a range from 5 mg to 200 mg, or in a range from 10 mg to 200 mg, or in a range from 25 mg to 200 mg, or in a range from 30 mg to 200 mg, or in a range from 50 mg to 200 mg, or in a range from 60 mg to 200 mg, or in a range from 75 mg to 200 mg, or in a range from 90 mg to 200 mg, or in a range from 100 mg to 200 mg, or in a range from 150 mg to 200 mg,

or wherein the single dose of the pharmaceutical composition comprises the antibody in an amount that is in a range from 1 mg to 100 mg, or in a range from 5 mg to 100 mg, or in a range from 10 mg to 100 mg, or in a range from 25 mg to 100 mg, or in a range from 30 mg to 100 mg, or in a range from 50 mg to 100 mg, or in a range from 60 mg to 100 mg, or in a range from 75 mg to 100 mg, or in a range from 75 mg to 100 mg, or in a range from 90 mg to 100 mg,

or wherein the single dose of the pharmaceutical composition comprises the antibody in an amount that is in a range from 1 mg to 25 mg, or in a range from 5 mg to 25 mg, or in a range from 10 mg to 25 mg, or in a range from 15 mg to 25 mg, or in a range from 20 mg to 25 mg,

or wherein the single dose of the pharmaceutical composition comprises the antibody in an amount that is in a range from 1 mg to 50 mg, or in a range from 1 mg to 25 mg, or in a range from 5 mg to 50 mg, or in a range from 5 mg to 25 mg, or in a range from 10 to 50 mg, or in a range from 10 to 25 mg, or in a range from 1 to 15 mg, or in a range from 5 mg to 15 mg, or in a range from 10 mg to 15 mg, or wherein the single dose of the pharmaceutical composition comprises 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 mg, or more, of the antibody,

or wherein the single dose of the pharmaceutical composition comprises the antibody in an amount that is less than 3000 mg, less than 2500 mg, less than 2000 mg, less than 1500 mg, less than 1000 mg, less than 900 mg, less than 500 mg, less than 300 mg, less than 200 mg, less than 100 mg, less than 90 mg, less than 75 mg, less than 50 mg, less than 25 mg, or less than 10 mg, but is more than 1 mg, more than 2 mg, more than 3 mg, more than 4 mg, or more than 5 mg.

Embodiment 86. The method of any one of Embodiments 83-85, wherein the single dose of the pharmaceutical composition comprises the antibody at a concentration in a range from 100 mg/mL to 200 mg/mL, such as 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, preferably 150 mg/mL.

Embodiment 87. The method of any one of Embodiments 83-86, wherein the single dose of the pharmaceutical composition comprises about 75 mg of the antibody.

Embodiment 88. The method of any one of Embodiments 83-87, wherein the single dose of the pharmaceutical composition comprises about 90 mg of the antibody.

Embodiment 89. The method of any one of Embodiments 83-88, wherein the single dose of the pharmaceutical composition comprises up to 300 mg of the antibody.

Embodiment 90. The method of any one of Embodiments 83-89, wherein the single dose of the pharmaceutical composition comprises up to 900 mg of the antibody.

Embodiment 91. The method of any one of Embodiments 83-90, wherein the single dose of the pharmaceutical composition comprises up to 3,000 mg of the antibody.

Embodiment 92. The method of any one of Embodiments 83-91, wherein the method comprises administering the single dose by subcutaneous injection, optionally wherein the single dose comprises or consists of 6 mg of the antibody or 18 mg of the antibody.

Embodiment 93. The method of any one of Embodiments 83-92, wherein the method comprises administering the single dose by intravenous injection.

Embodiment 94. The method of any one of Embodiments 83-93, wherein the pharmaceutical composition further comprises water, optionally USP water.

Embodiment 95. The method of any one of Embodiments 83-94, wherein the pharmaceutical composition further comprises histidine, optionally at a concentration in a range from 10 mM to 40 mM, such as 20 mM, in the pharmaceutical composition.

Embodiment 96. The method of any one of Embodiments 83-95, wherein the pharmaceutical composition further comprises a disaccharide, such as sucrose, optionally at 5%, 6%, 7%, 8%, or 9%, preferably about 7% (w/v).

Embodiment 97. The method of any one of Embodiments 83-96, wherein the pharmaceutical composition further comprises a surfactant or a triblock copolymer, optionally a polysorbate or poloxamer-188, preferably polysorbate 80 (PS80), wherein, optionally, the polysorbate or poloxamer-188 is present in a range from 0.01% to 0.05% (w/v), preferably 0.02% (w/v).

Embodiment 98. The method of any one of Embodiments 83-97, wherein the pharmaceutical composition has a pH in a range from 5.8 to 6.2, in a range from 5.9 to 6.1, or of 5.8, of 5.9, of 6.0, of 6.1, or of 6.2.

Embodiment 99. The method of Embodiment 98, wherein the pharmaceutical composition comprises:

(i) the antibody at 150 mg/mL;

(ii) USP water;

(iii) 20 mM histidine;

(iv) 7% sucrose; and

(v) 0.02% PS80,

wherein the pharmaceutical composition comprises a pH of 6.

Embodiment 100. The method of any one of Embodiments 83-99, wherein the subject is an adult.

Embodiment 101. The method of Embodiment 100, wherein the subject is in a range from 18 years of age to 65 years of age.

Embodiment 102. The method of any one of Embodiments 83-101, wherein the subject weighs from 40 kg to 125 kg and/or the subject has a body mass index (BMI) from 18 to 35 kg/m².

Embodiment 103. The method of any one of Embodiments 83-102, wherein the subject has a chronic HBV infection; e.g., defined by positive serum HBsAg, HBV DNA, and/or HBeAg on 2 occasions, wherein the 2 occasions are at least 6 months apart.

Embodiment 104. The method of any one of Embodiments 83-103, wherein the subject does not have cirrhosis.

Embodiment 105. The method of Embodiment 104, wherein absence of cirrhosis is determined by:

Fibroscan evaluation (e.g., within 6 months prior to administering the single dose of the pharmaceutical composition); or

liver biopsy (e.g., within 12 months prior to administering the single dose of the pharmaceutical composition),

wherein, preferably the absence of cirrhosis is determined by the absence of Metavir F3 fibrosis or the absence of F4 cirrhosis.

Embodiment 106. The method of any one of Embodiments 83-105, wherein the subject has received a nucleos(t)ide reverse transcriptase inhibitor (NRTI), optionally within 120 days, further optionally within 60 days, prior to the single dose being administered.

Embodiment 107. The method of Embodiment 106, wherein the NRTI comprises one or more of: tenofovir; tenofovir disoproxil (e.g., tenofovir disproxil fumarate); tenofovir alafenamide; Entecavir; Lamivudine; Adefovir; and adefovir dipivoxil.

Embodiment 108. The method of any one of Embodiments 83-107, wherein the subject has a serum HBV DNA concentration of less than 100 IU/mL no more than 28 days prior to the single dose being administered.

Embodiment 109. The method of any one of Embodiments 83-108, wherein the subject has a serum HBsAg concentration of less than 3,000 IU/mL prior to the single dose being administered, and optionally less than 1,000 IU/mL prior to the single dose being administered.

Embodiment 110. The method of any one of Embodiments 83-109, wherein the subject has a serum HBsAg concentration of greater than or equal to 3,000 IU/mL no more than 28 days prior to the single dose being administered, and optionally greater than or equal to 1,000 IU/mL no more than 28 days prior to the single dose being administered.

Embodiment 111. The method of any one of Embodiments 83-110, wherein the subject was HB e-antigen (HBeAg)-negative no more than 28 days prior to the single dose being administered.

Embodiment 112. The method of any one of Embodiments 83-111, wherein the subject was negative for anti-HB antibodies no more than 28 days prior to the single dose being administered.

Embodiment 113. The method of any one of Embodiments 83-112, wherein the subject, prior to administration of the single dose:

(i) does not have fibrosis and/or does not have cirrhosis; and/or

(ii) has alanine aminotransferase (ALT)<2×Upper Limit of Normal (ULN).

Embodiment 114. The method of any one of Embodiments 83-113, wherein at 56 days following administration of the single dose, the subject has a >2-fold reduction in serum HBsAg (e.g., concentration of HBsAg in serum, e.g., as determined using an Abbott ARCHITECT assay) as compared to the subject's serum HBsAg at from 0 days to 28 days prior to administration of the single dose.

Embodiment 115. The method of any one of Embodiments 83-114, wherein following administration of the single dose (e.g., at 56 days following administration of the single dose), the subject has:

(i) has reduced or less severe intrahepatic spread of HBV as compared to a reference subject; and/or

(ii) comprises an adaptive immune response against HBV.

Embodiment 116. The method of any one of Embodiments 83-115, wherein the subject is male.

Embodiment 117. The method of any one of Embodiments 83-115, wherein the subject is female.

Embodiment 118. A pharmaceutical composition comprising the antibody or antigen-binding fragment of any one of Embodiments 1-59 at a concentration ranging from 100 mg/mL to 200 mg/mL, such as 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, preferably 150 mg/mL,

and a pharmaceutically acceptable carrier, excipient, or diluent.

Embodiment 119. The pharmaceutical composition of Embodiment 118, wherein the pharmaceutical composition comprises up to 6 mg, up to 18 mg, up to 75 mg, up to 90 mg, up to 300 mg, up to 900 mg, or up to 3000 mg of the antibody.

Embodiment 120. The pharmaceutical composition of Embodiment 118 or 119, wherein the pharmaceutical composition comprises about 75 mg of the antibody.

Embodiment 121. The pharmaceutical composition of Embodiment 118 or 119, wherein the pharmaceutical composition comprises about 90 mg of the antibody.

Embodiment 122. The pharmaceutical composition of Embodiment 118 or 119, wherein the pharmaceutical composition comprises about 300 mg of the antibody.

Embodiment 123. The pharmaceutical composition of Embodiment 118 or 119, wherein the pharmaceutical composition comprises about 900 mg of the antibody.

Embodiment 124. The pharmaceutical composition of Embodiment 118 or 119, wherein the pharmaceutical composition comprises about 3,000 mg of the antibody.

Embodiment 125. The pharmaceutical composition of any one of Embodiments 118-124, wherein the pharmaceutical composition comprises water, optionally USP water.

Embodiment 126. The pharmaceutical composition of any one of Embodiments 118-125, wherein the pharmaceutical composition comprises histidine, optionally at a concentration from 10 mM to 40 mM, such as 20 mM, in the pharmaceutical composition.

Embodiment 127. The pharmaceutical composition of any one of Embodiments 118-126, wherein the pharmaceutical composition comprises a disaccharide, such as sucrose, optionally at 5%, 6%, 7%, 8%, or 9%, preferably about 7% (w/v).

Embodiment 128. The pharmaceutical composition of any one of Embodiments 118-127, wherein the pharmaceutical composition comprises a surfactant, optionally a polysorbate, preferably polysorbate 80 (PS80), wherein, optionally, the polysorbate is present in a range from 0.01% to 0.05% (w/v), preferably 0.02% (w/v).

Embodiment 129. The pharmaceutical composition of any one of Embodiments 118-128, wherein the pharmaceutical composition has a pH ranging from 5.8 to 6.2, ranging from 5.9 to 6.1, or of 5.8, of 5.9, of 6.0, of 6.1, or of 6.2.

Embodiment 130. The pharmaceutical composition of any one of Embodiments 118-129, wherein the pharmaceutical composition comprises:

(i) the antibody at 150 mg/mL;

(ii) USP water;

(iii) 20 mM histidine;

(iv) 7% sucrose; and

(v) 0.02% PS80,

wherein the pharmaceutical composition comprises a pH of 6.

Embodiment 131. The method of any one of Embodiments 83-117, wherein following administration of the single dose, serum HBsAg of the subject is reduced as compared to baseline by 1.0 log¹⁰ IU/mL, 1.5 log¹⁰ IU/mL, or more, wherein, optionally, the reduction persists for 1, 2, 3, 4, 5, 6, 7, 8, or more days following administration of the single dose.

Embodiment 132. The method of any one of Embodiments 83-117 and 131, wherein following administration of the single dose, serum HBsAg of the subject is reduced as compared to baseline for at least 8, at least 15, at least 22, or at least 29 days.

Embodiment 133. A method for in vitro diagnosis of a hepatitis B and/or a hepatitis D infection, the method comprising:

(i) contacting a sample from a subject with an antibody or antigen-binding fragment of any one of Embodiments 1-59; and

(ii) detecting a complex comprising an antigen and the antibody, or comprising an antigen and the antigen-binding fragment.

Embodiment 134. The method of Embodiment 133, wherein the sample comprises blood isolated from the subject.

Embodiment 135. A method for detecting the presence or absence of an epitope in a correct conformation in an anti-hepatitis-B and/or an anti-hepatitis-D vaccine, the method comprising:

(i) contacting the vaccine with an antibody or antigen-binding fragment of any one of Embodiments 1-59; and

(ii) determining whether a complex comprising an antigen and the antibody, or comprising an antigen and the antigen binding fragment, has been formed.

Embodiment 136. The antibody or antigen-binding fragment of any one of Embodiments 1-59, wherein the antibody or antigen-binding fragment:

(i) has enhanced binding to a human FcγRIIA, a human FcγRIIIA, or both, as compared to a reference polypeptide that includes a Fc moiety that does not comprise G236A/A330L/I332E, wherein the human FcγRIIA is optionally H131 or R131, and/or the human FcγRIIIA is optionally F158 or V158;

(ii) has reduced binding to a human FcγRIM, as compared to a reference polypeptide that includes a Fc moiety that does not comprise G236A/A330L/I332E;

(iii) does not bind to a human FcγRIM;

(iv) has reduced binding to a human C1q, as compared to a reference polypeptide that includes a Fc moiety that does not comprise G236A/A330L/I332E;

(v) does not bind to a human C1q; (vi) activates a FcγRIIA, a human FcγRIIIA, or both, to a greater degree than does a reference polypeptide that includes a Fc moiety that does not comprise G236A/A330L/I332E, wherein the human FcγRIIA is optionally H131 or R131, and/or the human FcγRIIIA is optionally F158 or V158;

(vii) does not activate a human FcγRIM;

(viii) activates a human natural killer (NK) cell in the presence of HBsAg to a greater degree than does a reference polypeptide that includes a Fc moiety that does not comprise G236A/A330L/I332E, wherein the reference polypeptide is optionally an antibody that binds to an HB Ag, optionally an HBsAg;

(ix) is capable of binding to an HBsAg variant comprising HBsAg-Y100C/P120T, HBsAg-P120T, HBsAg-P120S/S143L, HBsAg-C121S, HBsAg-R122D, HBsAg-R122I, HBsAg-T123N, HBsAg-Q129H, HBsAg-Q129L, HBsAg-M133H, HBsAg-M133L, HBsAg-M133T, HBsAg-K141E, HBsAg-P142S, HBsAg-S143K, HBsAg-D144A, HBsAg-G145R, HBsAg-N146A, or any combination thereof; and/or (x) has improved binding to an HBsAg variant comprising HBsAg-Y100C/P120T, HBsAg-P120T, HBsAg-P120S/S143L, HBsAg-C121S, HBsAg-R122D, HBsAg-R122I, HBsAg-T123N, HBsAg-Q129H, HBsAg-Q129L, HBsAg-M133H, HBsAg-M133L, HBsAg-M133T, HBsAg-K141E, HBsAg-P142S, HBsAg-S143K, HBsAg-D144A, HBsAg-G145R, HBsAg-N146A, or any combination thereof, as compared to a reference antibody or antigen binding fragment that binds to HBsAg and that includes a Fc moiety that does not comprise G236A/A330L/I332E.

Embodiment 137. A method of treating chronic HBV infection in a subject in need thereof, comprising:

administering to the subject an agent that reduces HBV antigenic load; and

administering to the subject an anti-HBV antibody from any one of Embodiments 1-59.

Embodiment 138. A method of treating chronic HBV infection in a subject in need thereof, comprising:

administering to the subject an inhibitor of HBV gene expression; and

administering to the subject an anti-HBV antibody from any one of Embodiments 1-59.

Embodiment 139. The method according to Embodiments 137 or 138, wherein the RNAi agent comprises a sense strand and an antisense strand forming a double-stranded region, wherein the sense strand comprises at least 15 contiguous nucleotides differing by no more than 3 nucleotides from nucleotides 1579-1597 of SEQ ID NO:116.

Embodiment 140. The method according to any one of Embodiments 137-139, wherein the RNAi agent comprises a sense strand and an antisense strand, wherein the sense strand comprises nucleotides 1579-1597 of SEQ ID NO:116.

Embodiment 141. The method according to any one of Embodiments 137-140, wherein at least one strand of the RNAi agent comprises a 3′ overhang of at least 1 nucleotide.

Embodiment 142. The method according to any one of Embodiments 137-140, wherein at least one strand of the RNAi agent comprises a 3′ overhang of at least 2 nucleotides.

Embodiment 143. The method according to any one of Embodiments 137-142, wherein the double-stranded region of the RNAi agent is 15-30 nucleotide pairs in length.

Embodiment 144. The method according to any one of Embodiments 137-142, wherein the double-stranded region of the RNAi agent is 17-23 nucleotide pairs in length.

Embodiment 145. The method according to any one of Embodiments 137-142, wherein the double-stranded region of the RNAi agent is 17-25 nucleotide pairs in length.

Embodiment 146. The method according to any one of Embodiments 137-142, wherein the double-stranded region of the RNAi agent is 23-27 nucleotide pairs in length.

Embodiment 147. The method according to any one of Embodiments 137-142, wherein the double-stranded region of the RNAi agent is 19-21 nucleotide pairs in length.

Embodiment 148. The method according to any one of Embodiments 137-142, wherein the double-stranded region of the RNAi agent is 21-23 nucleotide pairs in length.

Embodiment 149. The method according to any one of Embodiments 137-142, wherein each strand of the RNAi agent has 15-30 nucleotides.

Embodiment 150. The method according to any one of Embodiments 137-142, wherein each strand of the RNAi agent has 19-30 nucleotides.

Embodiment 151. The method according to any one of the Embodiments 137-150, wherein the RNAi agent is an siRNA.

Embodiment 152. The method according to Embodiment 151, wherein the siRNA inhibits expression of an HBV transcript that encodes an HBsAg protein, an HBcAg protein, and HBx protein, or an HBV DNA polymerase protein.

Embodiment 153. The method according to Embodiment 151 or Embodiment 152, wherein the siRNA binds to at least 15 contiguous nucleotides of a target encoded by: P gene, nucleotides 2309-3182 and 1-1625 of NC_003977.2; S gene (encoding L, M, and S proteins), nucleotides 2850-3182 and 1-837 of NC_003977.2; HBx, nucleotides 1376-1840 of NC_003977.2; or C gene, nucleotides 1816-2454 of NC_003977.2.

Embodiment 154. The method according to Embodiment 151 or Embodiment 152, wherein the antisense strand of the siRNA comprises at least 15 contiguous nucleotides of the nucleotide sequence of 5′-UGUGAAGCGAAGUGCACACUU-3′ (SEQ ID NO:119).

Embodiment 155. The method according to Embodiment 151 or 152, wherein the antisense strand of the siRNA comprises at least 19 contiguous nucleotides of the nucleotide sequence of 5′-UGUGAAGCGAAGUGCACACUU-3′ (SEQ ID NO:119).

Embodiment 156. The method according to Embodiment 151 or 152, wherein the antisense strand of the siRNA comprises the nucleotide sequence of 5′-UGUGAAGCGAAGUGCACACUU-3′ (SEQ ID NO:119).

Embodiment 157. The method according to Embodiment 151 or 152, wherein the antisense strand of the siRNA consists of the nucleotide sequence of 5′-UGUGAAGCGAAGUGCACACUU-3′ (SEQ ID NO:119).

Embodiment 158. The method according to any one of Embodiments 154-157, wherein the sense strand of the siRNA comprises the nucleotide sequence of 5′-GUGUGCACUUCGCUUCACA-3′ (SEQ ID NO:118).

Embodiment 159. The method according to any one of Embodiments 154-157, wherein the sense strand of the siRNA consists of the nucleotide sequence of 5′-GUGUGCACUUCGCUUCACA-3′ (SEQ ID NO:118).

Embodiment 160. The method according to Embodiment 151 or 152, wherein the antisense strand of the siRNA comprises at least 15 contiguous nucleotides of the nucleotide sequence of 5′-UAAAAUUGAGAGAAGUCCACCAC-3′ (SEQ ID NO:121).

Embodiment 161. The method according to Embodiment 151 or 152, wherein the antisense strand of the siRNA comprises at least 19 contiguous nucleotides of the nucleotide sequence of 5′-UAAAAUUGAGAGAAGUCCACCAC-3′ (SEQ ID NO:121).

Embodiment 162. The method according to Embodiment 151 or 152, wherein the antisense strand of the siRNA comprises the nucleotide sequence of 5′-UAAAAUUGAGAGAAGUCCACCAC-3′ (SEQ ID NO:121).

Embodiment 163. The method according to Embodiment 151 or 152, wherein the antisense strand of the siRNA consists of the nucleotide sequence of 5′-UAAAAUUGAGAGAAGUCCACCAC-3′ (SEQ ID NO:121).

Embodiment 164. The method according to any one of Embodiments 154-157, wherein the sense strand of the siRNA comprises the nucleotide sequence of 5′-GGUGGACUUCUCUCAAUUUUA-3′ (SEQ ID NO:120).

Embodiment 165. The method, composition for use, or use according to any one of Embodiments 154-157, wherein the sense strand of the siRNA consists of the nucleotide sequence of 5′-GGUGGACUUCUCUCAAUUUUA-3′ (SEQ ID NO:120).

Embodiment 166. The method according to any one of Embodiments 151-165, wherein substantially all of the nucleotides of said sense strand and substantially all of the nucleotides of said antisense strand are modified nucleotides, and

wherein said sense strand is conjugated to a ligand attached at the 3′-terminus.

Embodiment 167. The method according to Embodiment 166, wherein the ligand is one or more GalNAc derivatives attached through a monovalent linker, bivalent branched linker, or trivalent branched linker.

Embodiment 168. The method according to Embodiment 166 or 167, wherein the ligand is

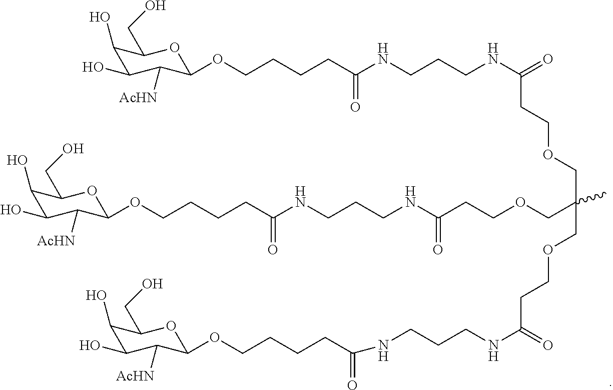 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

Embodiment 169. The method according to Embodiment 168, wherein the siRNA is conjugated to the ligand as shown in the following structure:

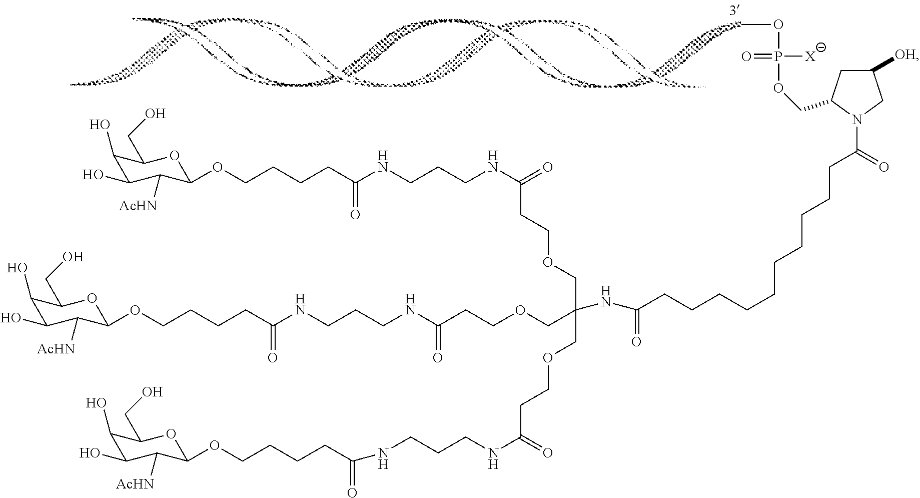

wherein X is O or S.

Embodiment 170. The method according to any one of Embodiments 151-169, wherein at least one nucleotide of the siRNA is a modified nucleotide comprising a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-0-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, an adenosine-glycol nucleic acid, or a nucleotide comprising a 5′-phosphate mimic.

Embodiment 171. The method according to any one of Embodiments 151-169, wherein the siRNA comprises a phosphate backbone modification, a 2′ ribose modification, 5′ triphosphate modification, or a GalNAc conjugation modification.

Embodiment 172. The method according to Embodiment 171, wherein the phosphate backbone modification comprises a phosphorothioate bond.

Embodiment 173. The method according to Embodiment 171 or Embodiment 172, wherein the 2′ ribose modification comprises a fluoro or —O-methyl substitution.

Embodiment 174. The method according to any one of Embodiments 151-159 and 166-173, wherein the siRNA has a sense strand comprising 5′-gsusguGfcAfCfUfucgcuucacaL96-3′ (SEQ ID NO:122) and an antisense strand comprising 5′-usGfsugaAfgCfGfaaguGfcAfcacsusu-3′ (SEQ ID NO:123), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;

Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;

s is a phosphorothioate linkage; and

L96 is N-[tris(GalNAc-alkyl)-amidodecanoyl)]-4-hydroxyprolinol.

Embodiment 175. The method according to any one of Embodiments 151-159 and 166-173, wherein the siRNA has a sense strand comprising 5′-gsusguGfcAfCfUfucgcuucacaL96-3′ (SEQ ID NO:124) and an antisense strand comprising 5′-usGfsuga(Agn)gCfGfaaguGfcAfcacsusu-3′ (SEQ ID NO:125) wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;

Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;

(Agn) is adenosine-glycol nucleic acid (GNA);

s is a phosphorothioate linkage; and

L96 is N-[tris(GalNAc-alkyl)-amidodecanoyl)]-4-hydroxyprolinol.

Embodiment 176. The method, compositions for use, or use according to any one of Embodiments 151-153 and 160-173, wherein the siRNA has a sense strand comprising 5′-gsgsuggaCfuUfCfUfcucaAfUfuuuaL96-3′ (SEQ ID NO:126) and an antisense strand comprising 5′-usAfsaaaUfuGfAfgagaAfgUfccaccsasc-3′ (SEQ ID NO:127), wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;

Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;

s is a phosphorothioate linkage; and

L96 is N-[tris(GalNAc-alkyl)-amidodecanoyl)]-4-hydroxyprolinol.

Embodiment 177. The method according to any one of Embodiments 137-176, wherein the subject is a human and a therapeutically effective amount of RNAi agent or siRNA is administered to the subject; and wherein the effective amount of the RNAi agent or siRNA is from about 1 mg/kg to about 8 mg/kg.

Embodiment 178. The method according to any one of Embodiments 137-177, wherein the RNAi agent or siRNA is administered to the subject twice daily, once daily, every two days, every three days, twice per week, once per week, every other week, every four weeks, or once per month.

Embodiment 179. The method according to any one of Embodiments 137-177, wherein the RNAi agent or siRNA is administered to the subject every four weeks.

Embodiment 180. The method according to any one of Embodiments 151-179, wherein two siRNAs each directed to an HBV gene are administered, and the first siRNA has an antisense strand comprising SEQ ID NO:119, SEQ ID NO:120, or SEQ ID NO:126; and the second siRNA comprises an siRNA having a sense strand that comprises at least 15 contiguous nucleotides of nucleotides 2850-3182 of SEQ ID NO:116.

Embodiment 181. The method according to any one of Embodiments 151-179, wherein two siRNAs directed to an HBV gene are administered, wherein the two siRNAs comprise: an siRNA directed to an HBV X gene and an siRNA directed to an HBV S gene.

Embodiment 182. The method according to any one of Embodiments 151-179, wherein two siRNAs each directed to an HBV gene are administered, and the first siRNA has an antisense strand comprising SEQ ID NO:119, SEQ ID NO:123, or SEQ ID NO:125; and the second siRNA has an antisense strand that comprises SEQ ID NO:121 or SEQ ID NO:127.

Embodiment 183. The method according to Embodiment 181, wherein the first siRNA has a sense strand comprising SEQ ID NO:118, SEQ ID NO:122, or SEQ ID NO:124; and the second siRNA has a sense strand comprising SEQ ID NO:120 or SEQ ID NO:126.

Embodiment 184. The method according to any one of Embodiments 179-183, wherein the two siRNAs are administered simultaneously.

Embodiment 185. The method according to any one of the Embodiments 137-184, further comprising administering a nucleot(s)ide analog to the subject, or wherein the subject is also administered a nucleot(s)ide analog.

Embodiment 186. The method, composition for use, or use according to Embodiment 185, wherein the nucleot(s)ide analog is tenofovir disoproxil fumarate (TDF), tenofovir alafenamide (TAF), lamivudine, adefovir dipivoxil, entecavir (ETV), telbivudine, AGX-1009, emtricitabine (FTC), clevudine, ritonavir, dipivoxil, lobucavir, famvir, N-Acetyl-Cysteine (NAC), PC1323, theradigm-HBV, thymosin-alpha, and ganciclovir, besifovir (ANA-380/LB-80380), or tenofvir-exaliades (TLX/CMX157).

In some instances, elements of the antibodies, antigen-binding fragments, fusion proteins, nucleic acids, cells, compositions, combinations, uses, and methods provided herein are described or listed with reference to embodiments or examples. However, it should be understood that the examples and embodiments described herein may be combined in various ways to create additional embodiments.

### EXAMPLES

In the following, particular examples illustrating various embodiments and aspects of the disclosure are presented. However, the present disclosure shall not to be limited in scope by the specific embodiments described herein.

### Example 1: Dimer Formation by an Anti-HBV Antibody

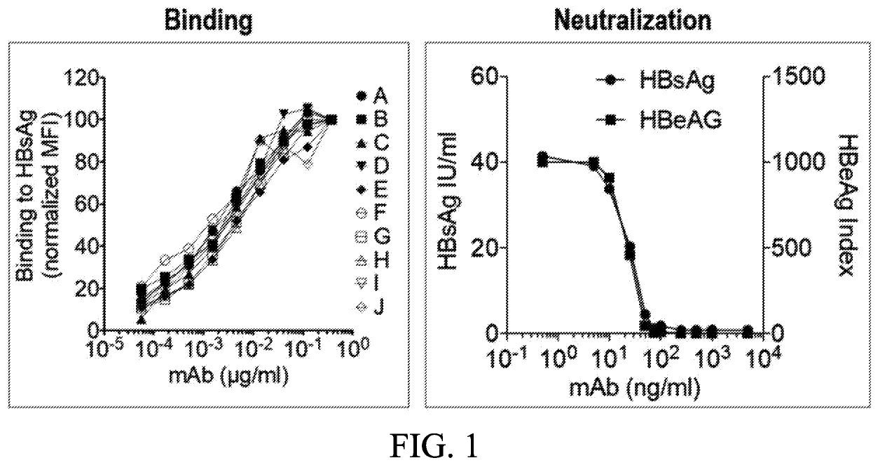

Anti-HBV antibodies are disclosed in PCT Publication No. WO 2017/060504. Engineering anti-HBV antibody “HBC34-v7” produced, inter alia, antibody “HBC34-v35” (PCT Publication No. WO 2020/132091), having VH and VL amino acid sequences according to SEQ ID NOs.:38 and 57, respectively. HBC34-v35 binds to HBsAg with picomolar affinity and potently neutralizes ten (10) HBV genotypes and Hepatitis D virus, binding to a conserved conformational epitope. Representative binding and neutralization data for HBC34-v35 (expressed as IgG1 and including the Fc mutations G236A, A330L, I332E, M428L, and N434S (EU numbering; collectively referred-to as “GAALIE-MLNS” or “GAALIE+MLNS” or “MLNS-GAALIE” or “MLNS+GAALIE)) are shown in FIG. 1.

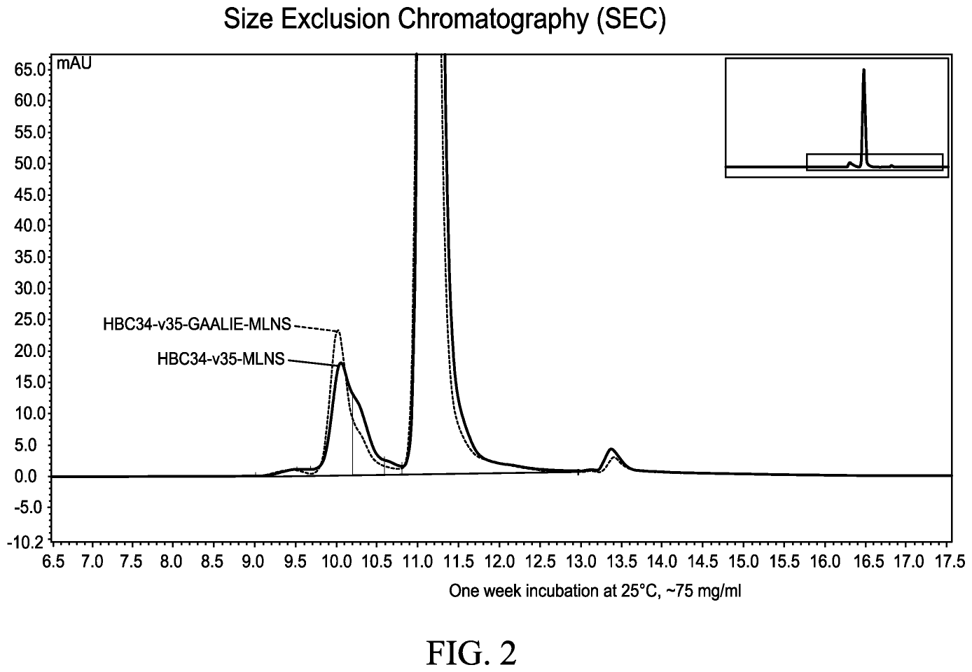

HBC34-v35 was expressed as recombinant IgG (allotype G1m17, 1) in a host cell line, purified from supernatant, and formulated for administration. Size-exclusion chromatography analysis of the formulation following a 1-week incubation revealed a peak corresponding to antibody monomer (i.e., single antibody molecules comprising two heavy chains and two light chains) and a high molecular weight species corresponding to an antibody dimer (i.e., aggregate formed by two single antibody molecules) (FIG. 2).

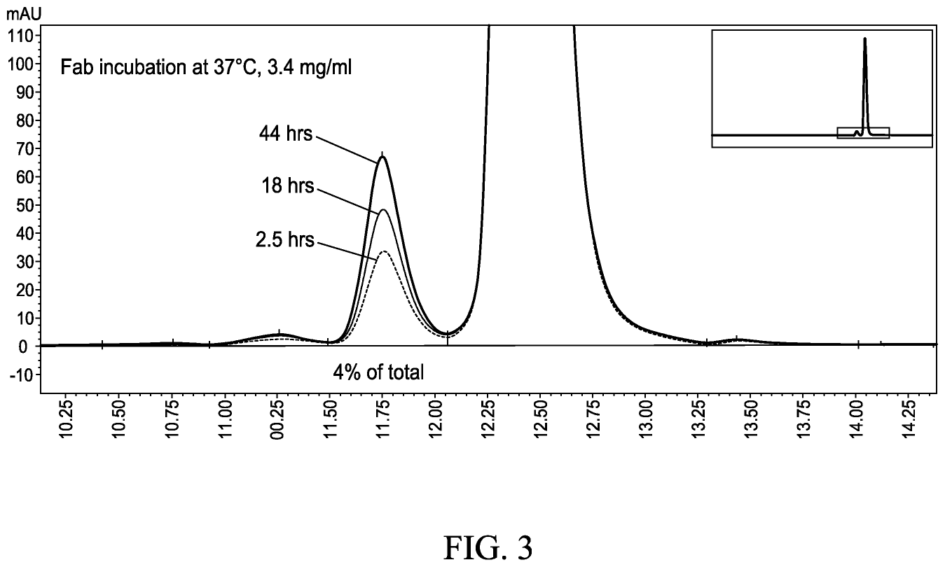

It was hypothesized that the dimer formation was mediated via Fab-Fab interactions, and that recombinant Fab should also dimerize. Size exclusion chromatography was used to purify enriched IgG dimer and Fab dimer. FIG. 3 shows that Fab dimer fraction slowly increases over time; dimer formation kinetics also increased with temperature (data not shown).

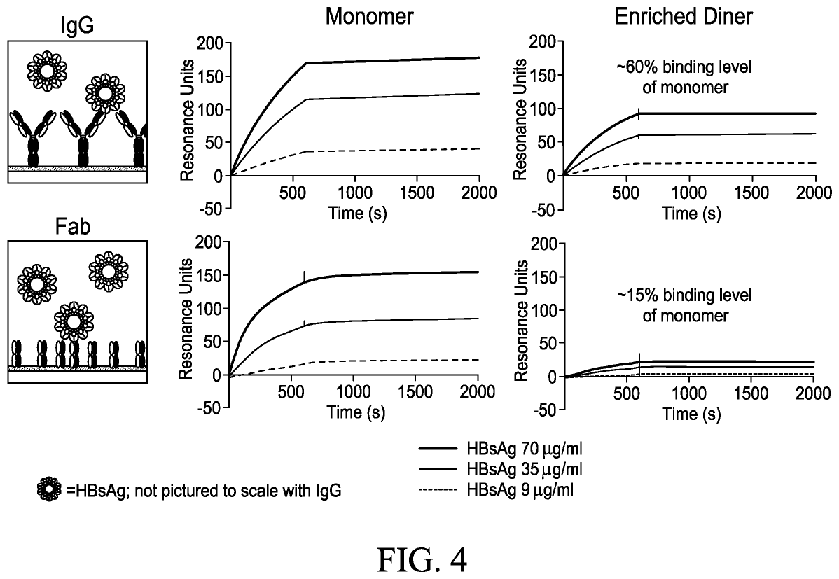

Different modes of Fab dimerization have been described (see, e.g., Plath et al. MAbs 8(5):928-940 (2016)). Depending on the mode of Fab dimerization, the Fab will either retain or lose the ability to bind antigen. For example, an IgG dimer could be expected to lose a maximum 50% of binding capacity, with two of the four Fabs being unaffected. As shown in FIG. 4, the HBC34-v35 dimer (full-length IgG or Fab) has reduced binding for HBsAg as determined by surface plasmon resonance (SPR; with similar amounts (by mass) of the monomer and dimer antibody captured on the surface), consistent with dimerization involving CDRs.

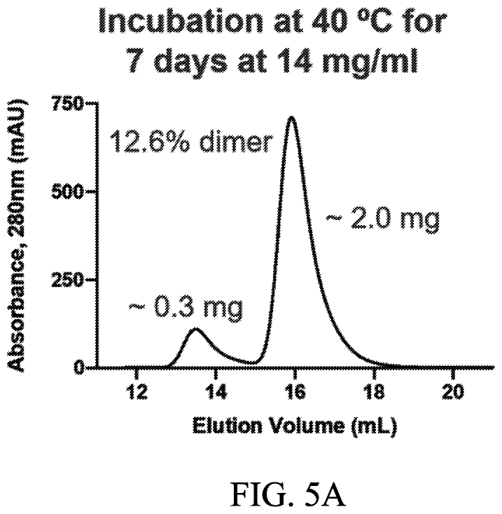

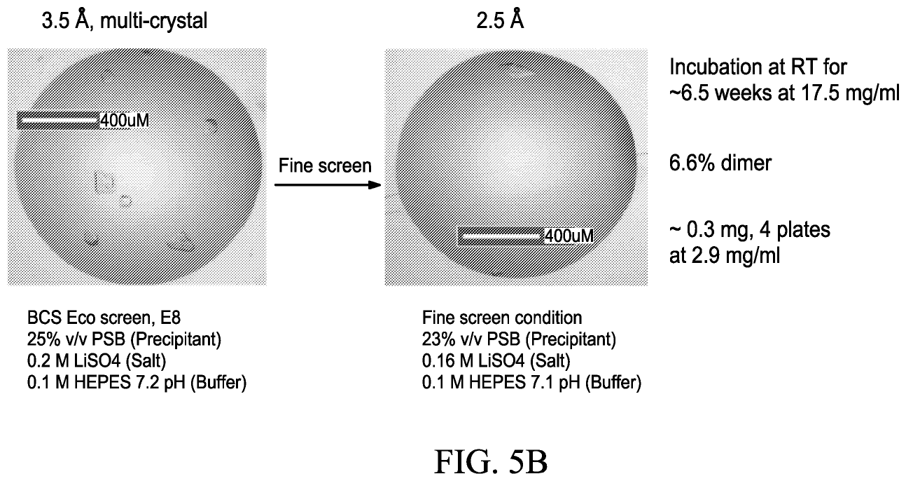

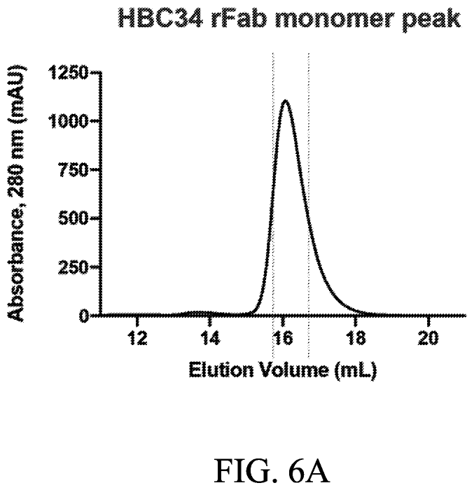

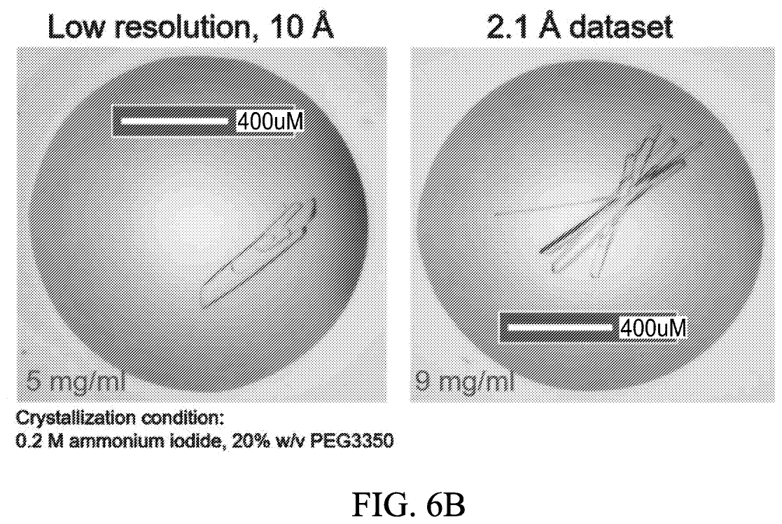

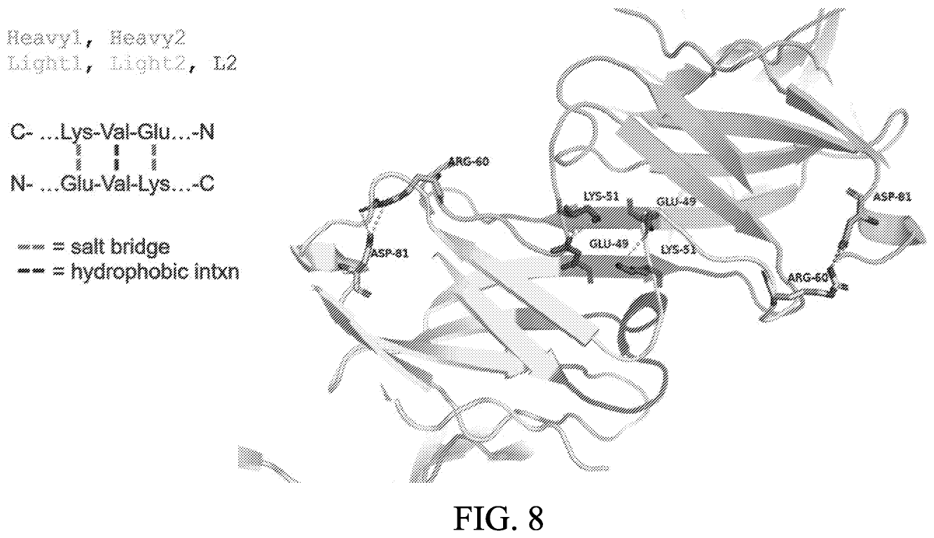

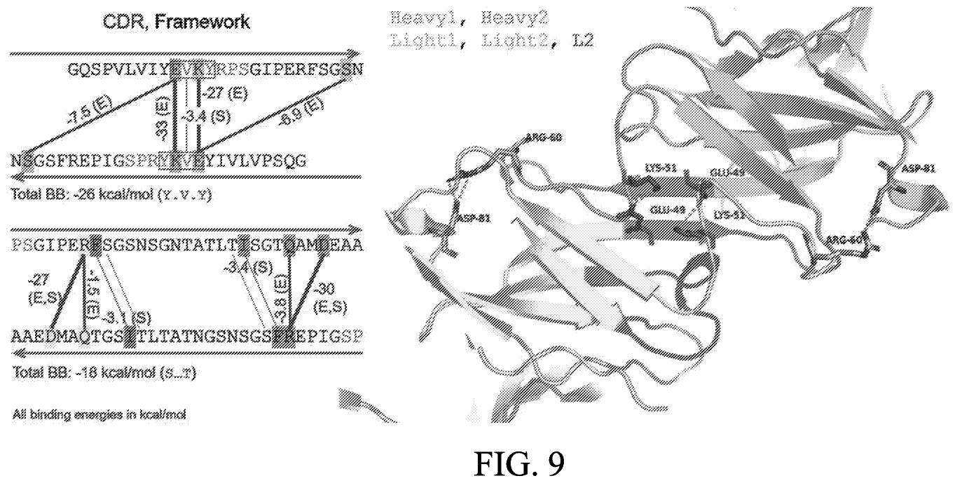

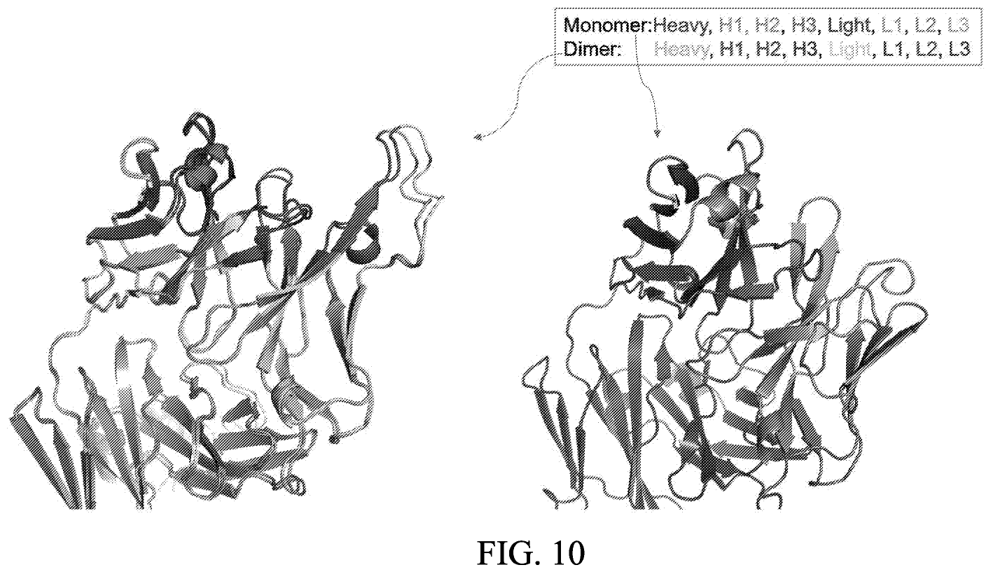

Next, crystallization of HBC34-v35 rFab dimer or monomer was performed. The rFab dimer was isolated by preparative size-exclusion chromatography (FIG. 5A). 3×96 conditions were set up at RT (concentration: 5.5 mg/ml). Crystals were obtained in three different conditions but diffracted poorly and were multi-crystal. A new round of incubation and crystallization optimization led to high quality diffraction (FIG. 5B). For rFab monomer, preparative size-exclusion chromatography was used for purification (FIG. 6A). Material obtained after the incubation at 40° C. did not yield crystals (3 trays at RT, 5 mg/mL or 9 mg/mL). A second batch of monomer was prepared without an incubation step; crystals formed at 4° C. but not RT (4 trays each, 2 concentrations) (FIG. 6B). Analysis of the Fab dimer crystal structure indicated that dimerization involves L-CDR2 (FIGS. 7-9), that the Fabs present in dimer form have a similar conformation, and that L-CDR2 undergoes a conformation change between monomer and dimer (FIG. 10). Potential interactions between L-CDR2 and framework residues were identified.

### Example 2: An Engineered Antibody with Reduced Dimer Formation

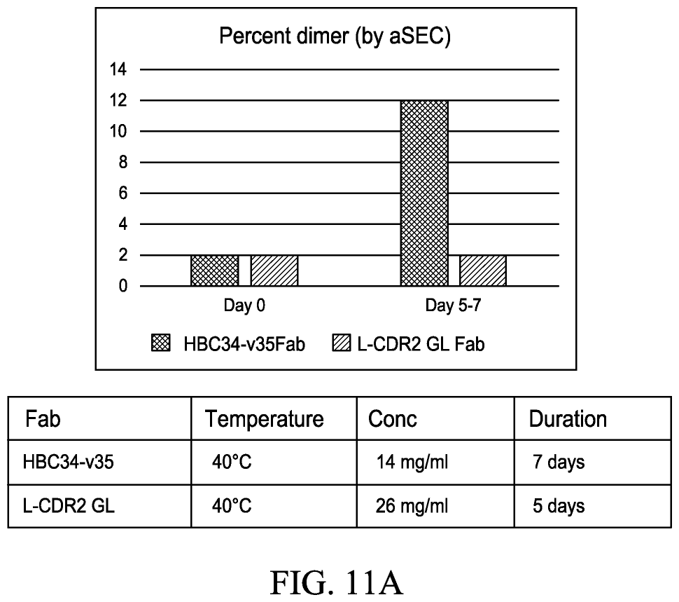

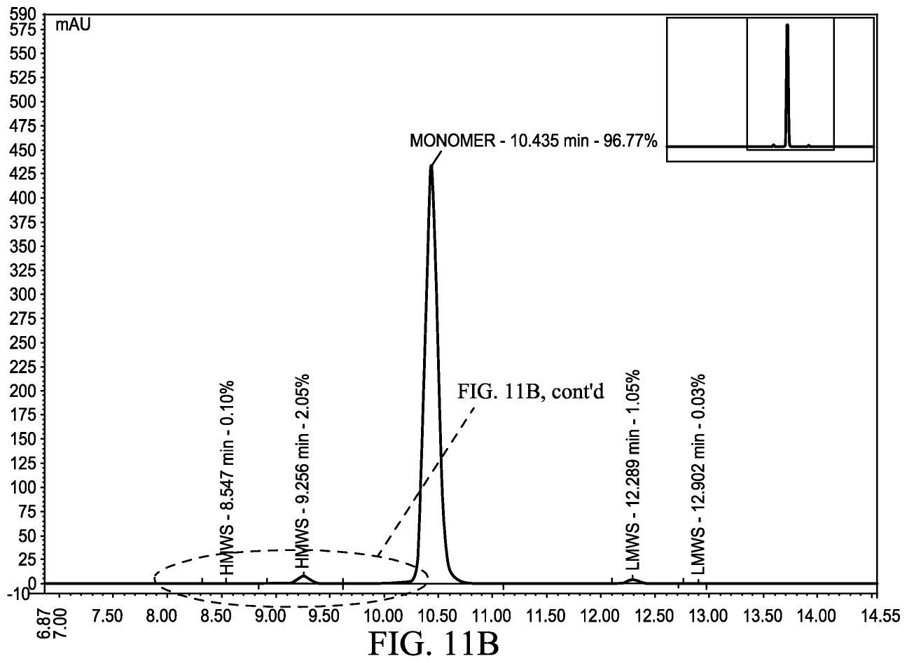

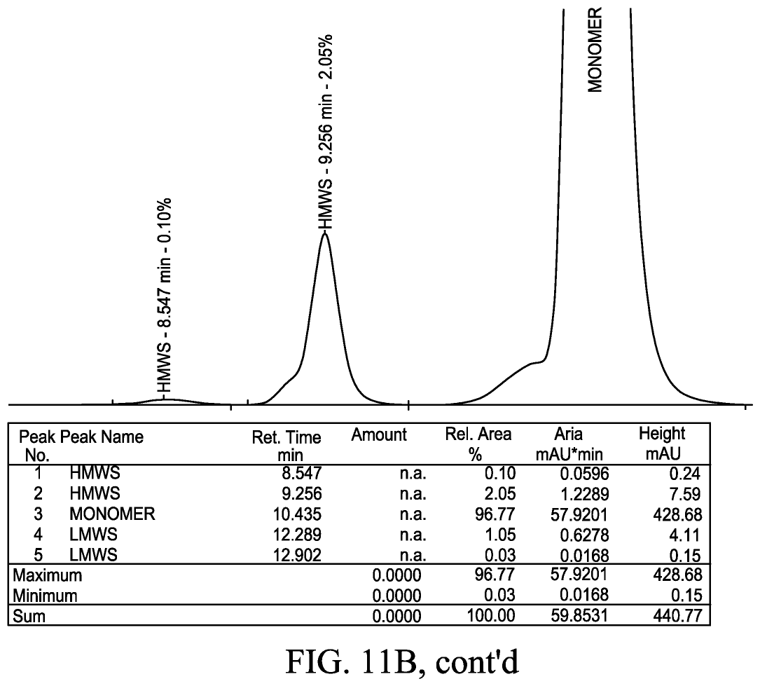

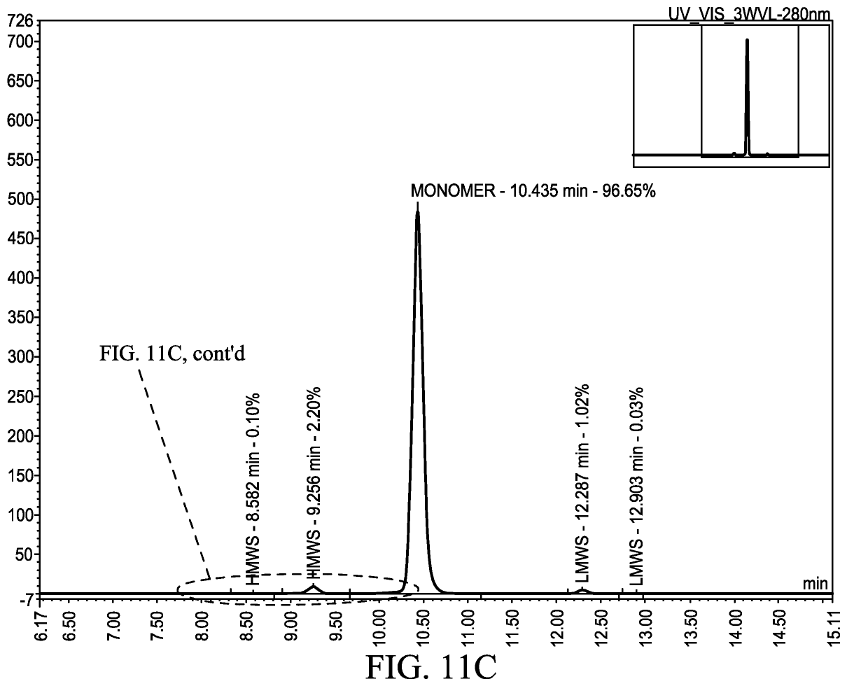

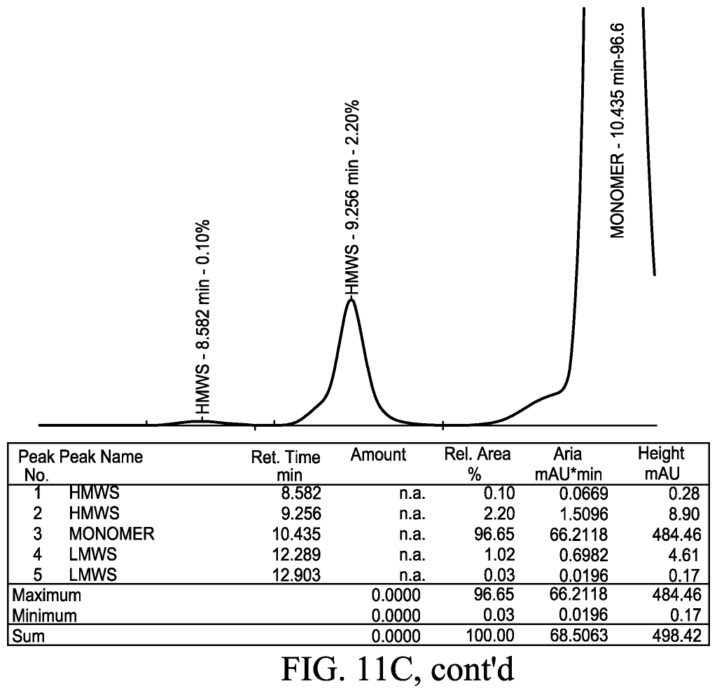

HBC34-v35 includes several mutations in the light chain compared to the germline sequence, including in L-CDR2. Three adjacent amino acids present in the L-CDR2 and believed to be involved in the Fab-Fab interactions were reverted to germline to generate the further variant antibody HBC34-v36. In separate experiments, HBC34-v35 and HBC34-v36 Fabs (>10 mg/mL) were incubated at 40° C. for 5-7 days and percent dimer was evaluated by absolute size-exclusion chromatography (aSEC). As shown in FIG. 11A, reversion to germline sequence dramatically reduced dimerization. HBC34-v36 full-length IgG at 3 mg/mL did not dimerize after 2 weeks at 40° C. (data not shown).

### Example 3: Binding and In Vitro Neutralization by Antibodies

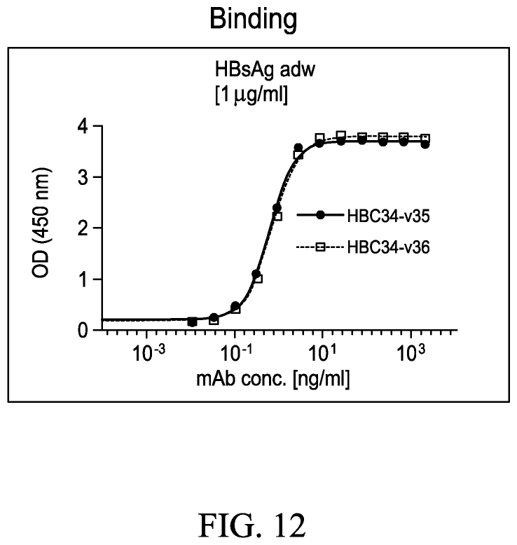

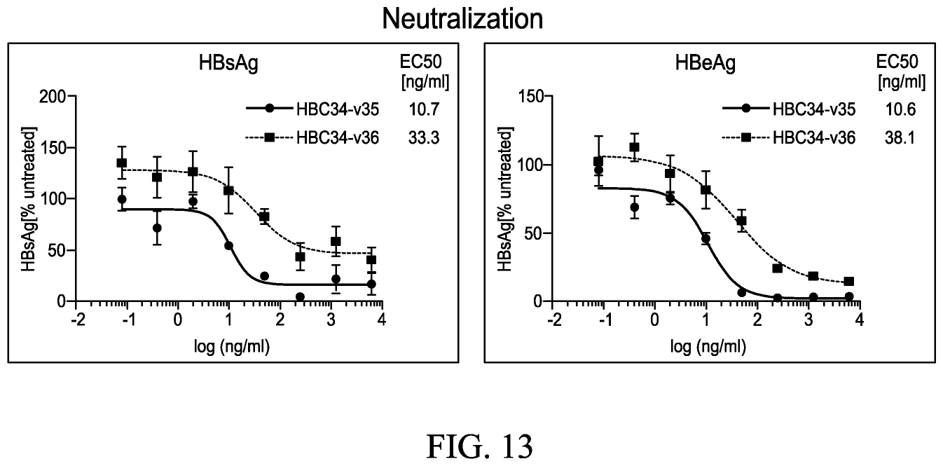

Ability of HBC34-v35 and HBC34-v36 to bind HBsAg and neutralize HBV infection was compared. Binding was assessed by ELISA and showed that HBC34-v36 has similar binding activity to HBC34-v35 (EC50=0.7 ng/mL vs. 0.6 ng/mL, respectively, FIG. 12). Neutralization was assessed by measuring the levels of HBeAg (genotype D) in the cell culture supernatant of HBV-infected HepG2 cells expressing NTCP. Data are shown in FIG. 13, and show that neutralization of HBV genotype D is approximately 3-fold weaker for HBC34-v36 than HBC34-v35. For these experiments, the antibodies included wild-type IgG1 Fc.

### Example 4: Design and Testing of Additional Engineered Antibodies

Additional engineered variant antibodies with mutations in L-CDR2 and/or in framework sequence relative to HBC34-v35 were generated, using HBC34-v36 as the starting point. These variants were termed HBC34-v37-HBC34-v50. Light chain variable region sequences from the various antibodies, and a summary of the mutations versus HBC34-v35, are provided in Table 6. CDR sequences and numbering of amino acid residues as shown in Table 6 are per the system developed by the Chemical Computing Group (chemcomp.com).

TABLE 6
VL amino acid sequences of HBC34 antibodies

| | VL amino acid sequence | Mutation(s) vs. |
|---|---|---|
| HBC34- | (L-CDR2 (CCG) underlined) | HBC34-v35 |
| v35 | SYELTQPPSVSVSPGQTVSIPCSGDKLGNKNVAWFQ | — |
| | HKPGQSPVLVIYEVKYRPSGIPERFSGSNSGNTATLT | |
| | ISGTQAMDEAAYFCQTFDSTTVVFGGGTRLTVL | |
| | (SEQ ID NO.: 57) | |
| v36 | SYELTQPPSVSVSPGQTVSIPCSGDKLGNKNVAWFQ | E49Q, V50D, |
| | HKPGQSPVLVIYQDSKRPSGIPERFSGSNSGNTATLT | K51S, Y52K |
| | ISGTQAMDEAAYFCQTFDSTTVVFGGGTRLTVL | |
| | (SEQ ID NO.: 58) | |
| v37 | SYELTQPPSVSVSPGQTVSIPCSGDKLGNKNVAWFQ | V50D, K51S, |
| | HKPGQSPVLVIYEDSKRPSGIPERFSGSNSGNTATLT | Y52K |
| | ISGTQAMDEAAYFCQTFDSTTVVFGGGTRLTVL | |
| | (SEQ ID NO : 59) | |
| v38 | SYELTQPPSVSVSPGQTVSIPCSGDKLGNKNVAWFQ | E49Q, K51S, |
| | HKPGQSPVLVIYQVSKRPSGIPERFSGSNSGNTATLT | Y52K |
| | ISGTQAMDEAAYFCQTFDSTTVVFGGGTRLTVL | |
| | (SEQ ID NO.: 60) | |
| v39 | SYELTQPPSVSVSPGQTVSIPCSGDKLGNKNVAWFQ | E49Q, V50D, |
| | HKPGQSPVLVIYQDKKRPSGIPERFSGSNSGNTATL | Y52K |
| | TISGTQAMDEAAYFCQTFDSTTVVFGGGTRLTVL | |
| | (SEQ ID NO.: 61) | |
| v40 | SYELTQPPSVSVSPGQTVSIPCSGDKLGNKNVAWFQ | E49Q, V50D, |
| | HKPGQSPVLVIYQDSYRPSGIPERFSGSNSGNTATLT | K51S |
| | ISGTQAMDEAAYFCQTFDSTTVVFGGGTRLTVL | |
| | (SEQ ID NO.: 62) | |
| v41 | SYELTQPPSVSVSPGQTVSIPCSGDKLGNKNVAWFQ | E49Q, K51S |
| | HKPGQSPVLVIYQVSYRPSGIPERFSGSNSGNTATLT | |
| | ISGTQAMDEAAYFCQTFDSTTVVFGGGTRLTVL | |
| | (SEQ ID NO.: 63) | |
| v42 | SYELTQPPSVSVSPGQTVSIPCSGDKLGNKNVAWFQ | K51S, S64A |
| | HKPGQSPVLVIYEVSYRPSGIPERFSGANSGNTATLT | |
| | ISGTQAMDEAAYFCQTFDSTTVVFGGGTRLTVL | |
| | (SEQ ID NO.: 64) | |
| v43 | SYELTQPPSVSVSPGQTVSIPCSGDKLGNKNVAWFQ | E49Q |
| | HKPGQSPVLVIYQVKYRPSGIPERFSGSNSGNTATL | |
| | TISGTQAMDEAAYFCQTFDSTTVVFGGGTRLTVL | |
| | (SEQ ID NO.: 65) | |
| v44 | SYELTQPPSVSVSPGQTVSIPCSGDKLGNKNVAWFQ | E49A |
| | HKPGQSPVLVIYAVKYRPSGIPERFSGSNSGNTATL | |
| | TISGTQAMDEAAYFCQTFDSTTVVFGGGTRLTVL | |
| | (SEQ ID NO.: 66) | |
| v45 | SYELTQPPSVSVSPGQTVSIPCSGDKLGNKNVAWFQ | R60N |
| | HKPGQSPVLVIYEVKYRPSGIPENFSGSNSGNTATLT | |
| | ISGTQAMDEAAYFCQTFDSTTVVFGGGTRLTVL | |
| | (SEQ ID NO.: 67) | |
| v46 | SYELTQPPSVSVSPGQTVSIPCSGDKLGNKNVAWFQ | R60A |
| | HKPGQSPVLVIYEVKYRPSGIPEAFSGSNSGNTATLT | |
| | ISGTQAMDEAAYFCQTFDSTTVVFGGGTRLTVL | |
| | (SEQ ID NO.: 68) | |
| v47 | SYELTQPPSVSVSPGQTVSIPCSGDKLGNKNVAWFQ | K51S, S64A, |
| | HKPGQSPVLVIYEVSYRPSGIPENFSGANSGNTATLT | R60N, I74A |
| | ASGTQAMDEAAYFCQTFDSTTVVFGGGTRLTVL | |
| | (SEQ ID NO.: 69) | |
| v48 | SYELTQPPSVSVSPGQTVSIPCSGDKLGNKNVAWFQ | R60N, S64A, |
| | HKPGQSPVLVIYEVKYRPSGIPENFSGANSGNTATL | I74A |
| | TASGTQAMDEAAYFCQTFDSTTVVFGGGTRLTVL | |
| | (SEQ ID NO.: 70) | |
| v49 | SYELTQPPSVSVSPGQTVSIPCSGDKLGNKNVAWFQ | K51S |
| | HKPGQSPVLVIYEVSYRPSGIPERFSGSNSGNTATLT | |
| | ISGTQAMDEAAYFCQTFDSTTVVFGGGTRLTVL | |
| | (SEQ ID NO.: 71) | |
| v50 | SYELTQPPSVSVSPGQTVSIPCSGDKLGNKNVAWFQ | R60K |
| | HKPGQSPVLVIYEVKYRPSGIPEKFSGSNSGNTATL | |
| | TISGTQAMDEAAYFCQTFDSTTVVFGGGTRLTVL | |
| | (SEQ ID NO.: 72) | |

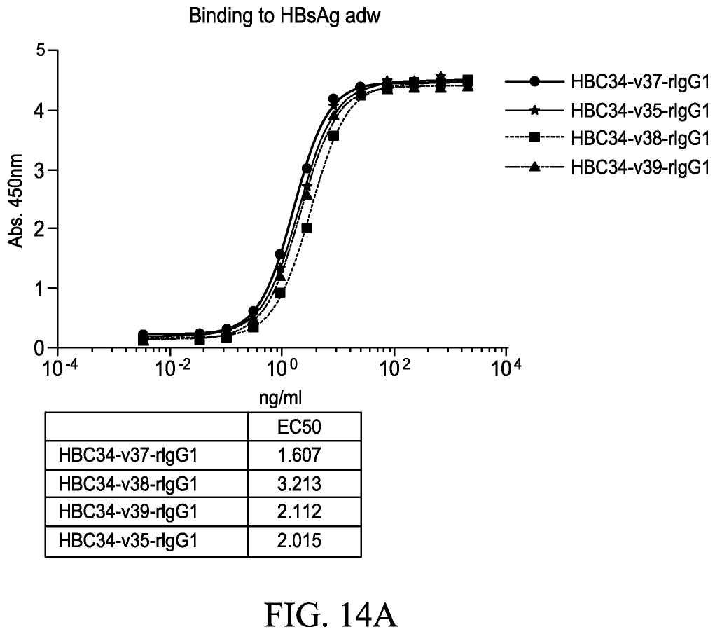

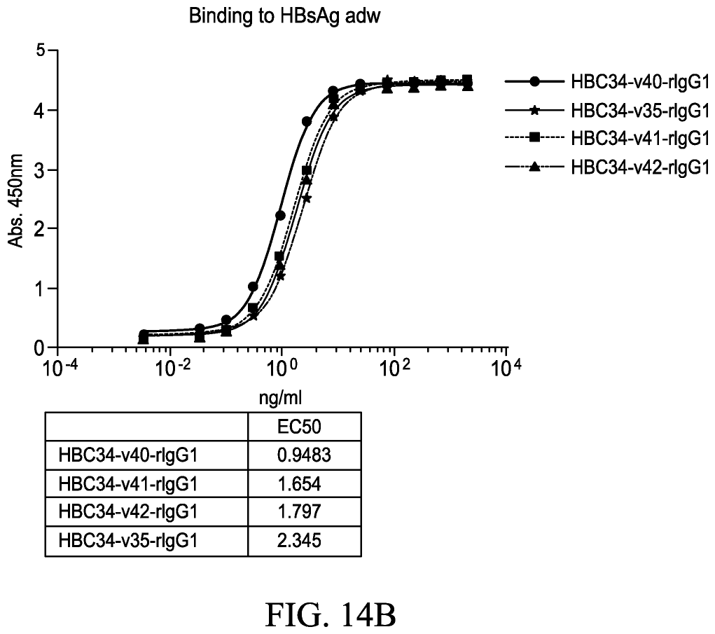

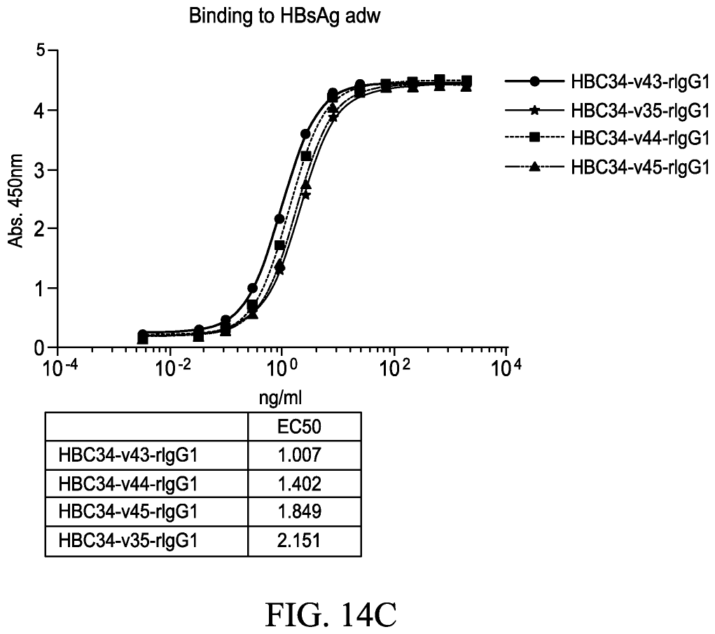

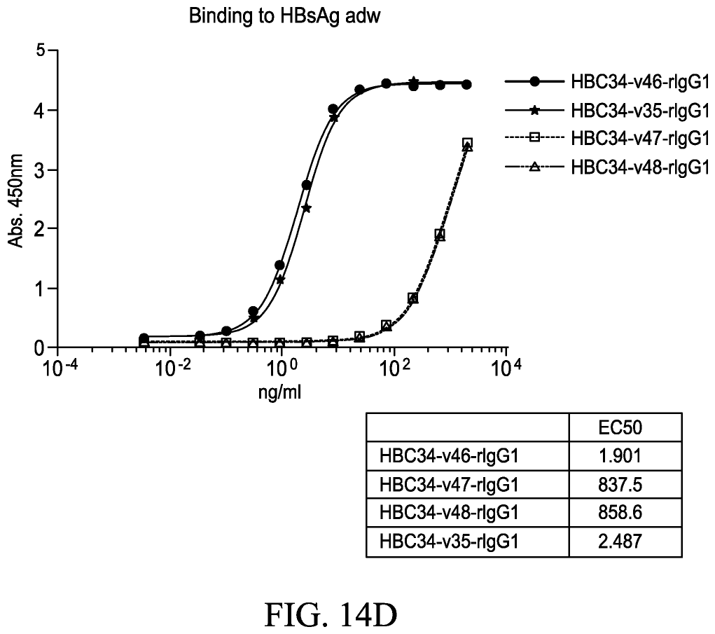

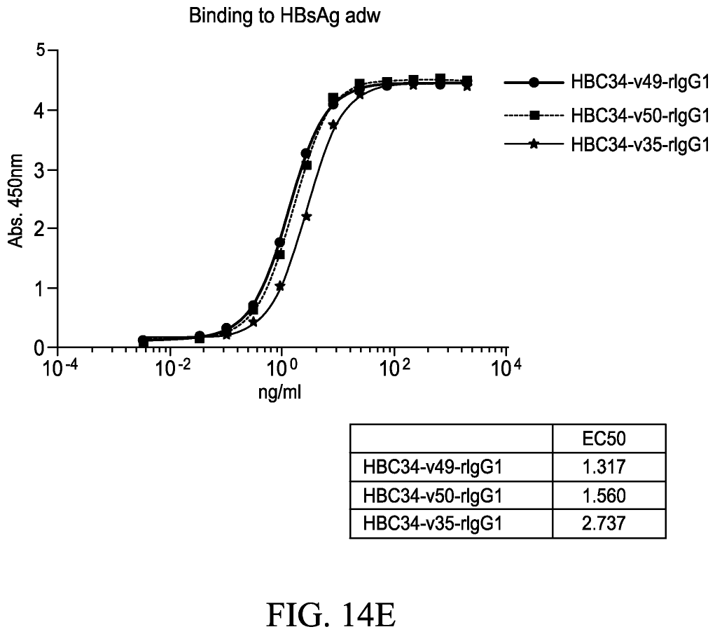

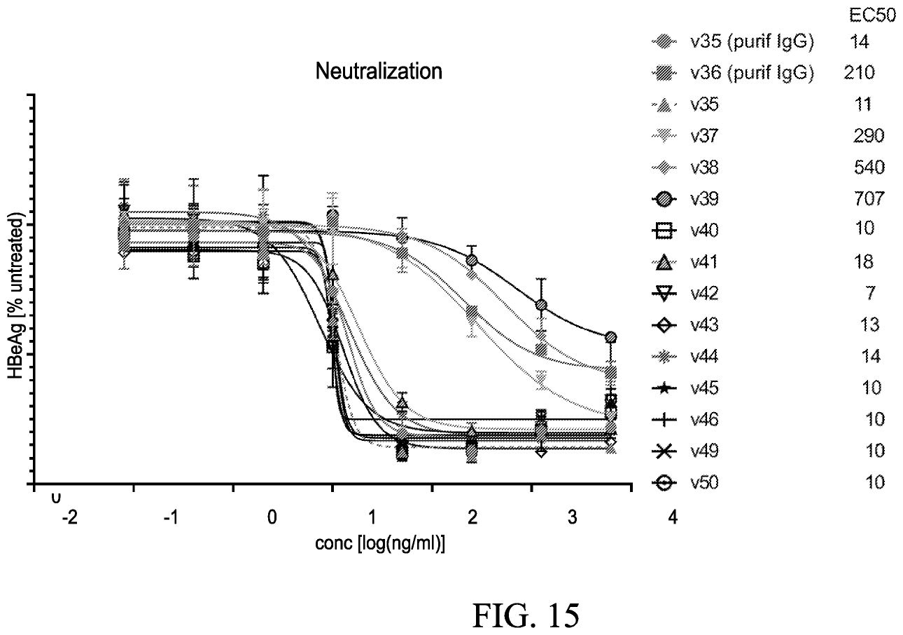

HBsAg binding and HBV neutralizing activity of HBC34-v37-HBC34-v50 was tested using assays as described herein. Results from the binding assays are provided in FIGS. 14A-14E, and show that all of the tested variant antibodies except for HBC34-v47 and HBC34-v48 had similar or even stronger binding as compared to HBC34-v35. HBC34-v47 and HBC34-v48 also had low production yields, and were not selected for further testing. Results from the neutralization assay are provided in FIG. 15, and show that several antibodies (HBC34-v40-HBC34-v46, HBC34-v49, and HBC34-v50) had similar or even improved neutralizing activity (EC50) as compared to HBC34-v35. HBC34-v36-HBC34-v39 had less potent neutralizing activity.

### Example 5: Purification of Certain Engineered Antibodies

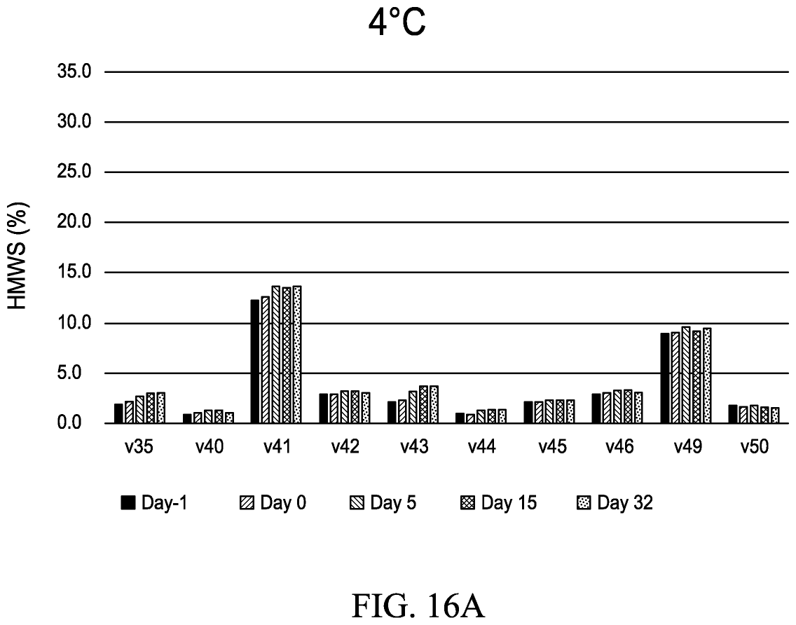

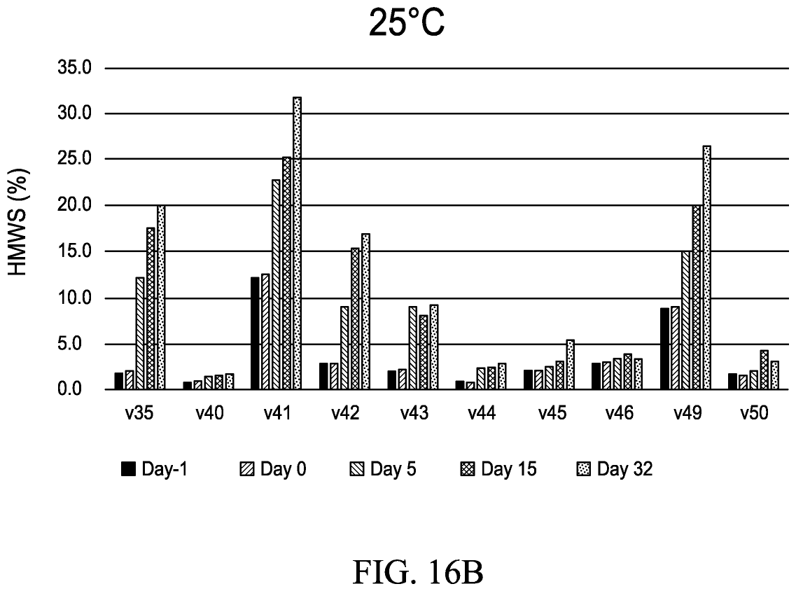

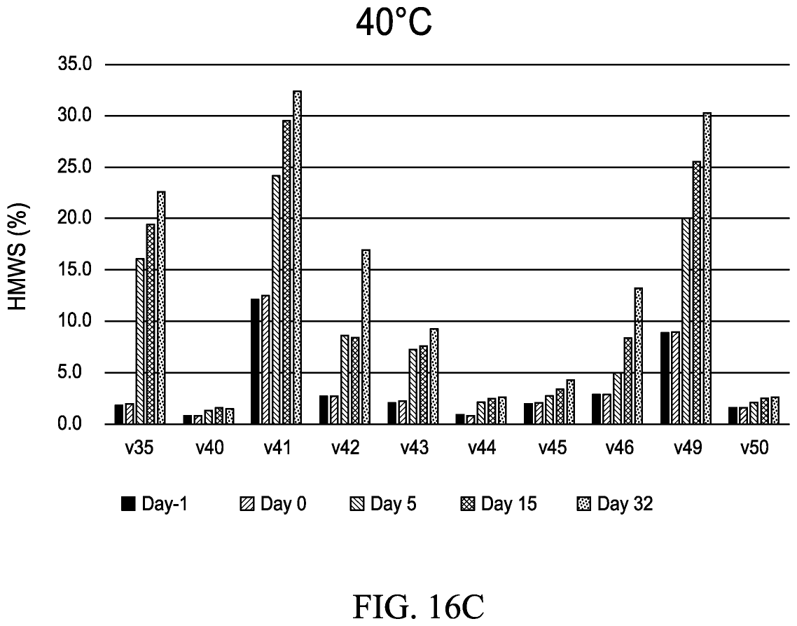

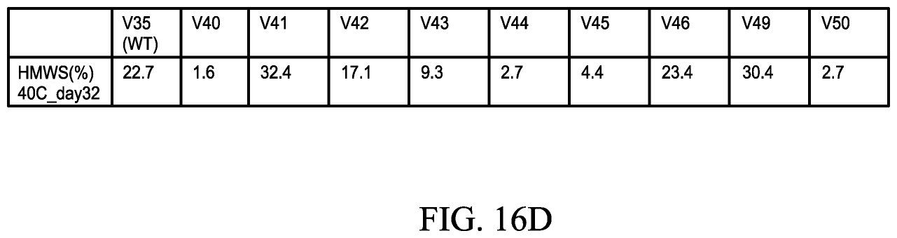

Engineered variant antibodies were evaluated for formation of aggregates following incubation at different temperatures over the course of 32 days. Nine HBC34-v35 antibody variants and parental HBC34-v35 were expressed as recombinant IgG (allotype G1m17, 1) in a host cell line and purified from supernatant. Antibodies were received later than one week after production and concentrated to 25 mg/ml. Size-exclusion chromatography (SEC) analysis was used to monitor high molecular weight species (HMWS) corresponding to an antibody dimer at day −1, day 0, day 5, day 15, and day 32. Day −1 samples were evaluated prior to concentration. Antibody compositions were incubated at 4° C. (FIG. 16A), 25° C. (FIG. 16B), or 40° C. (FIG. 16C) over the course of the 32-day analysis. A summary of the frequency of HMWS following a 32-day incubation at 40° C. is shown in FIG. 16D. Four variant antibodies (-v40, -v44, -v45, -v50) exhibited low generation of HMWS (FIG. 16D) and were selected for further studies.

### Example 6: Binding and In Vitro Neutralization by Certain Engineered Antibodies

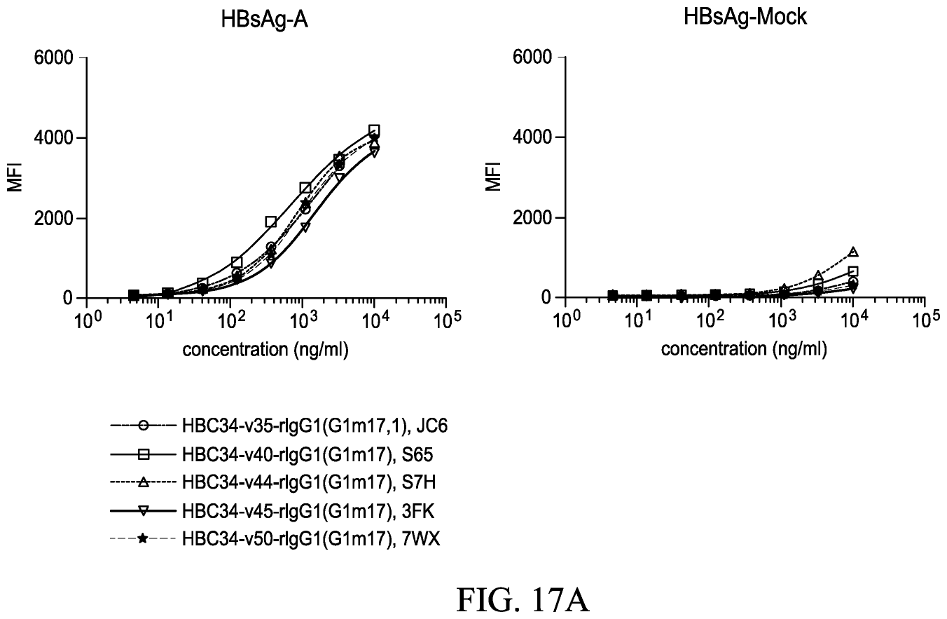

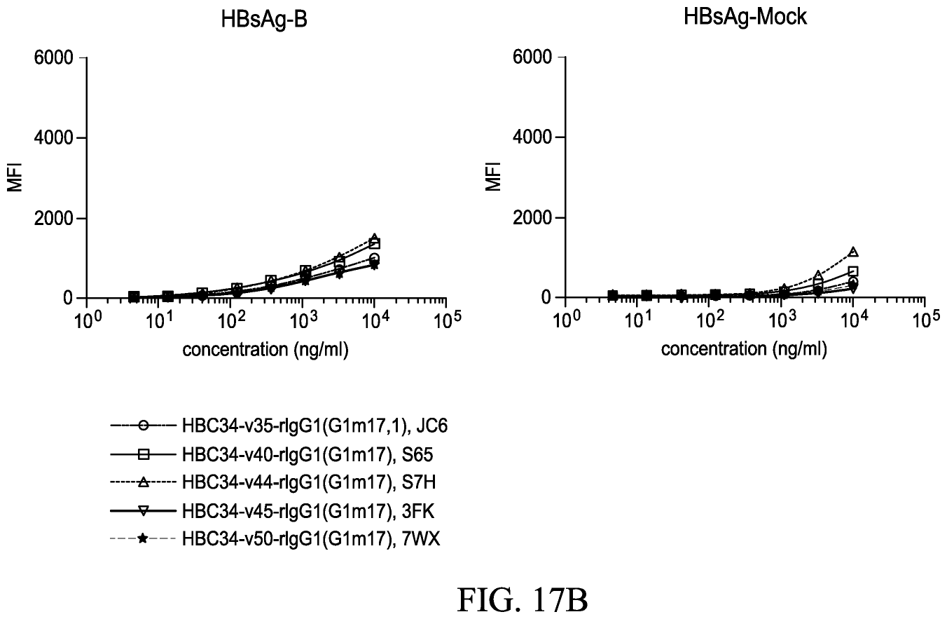

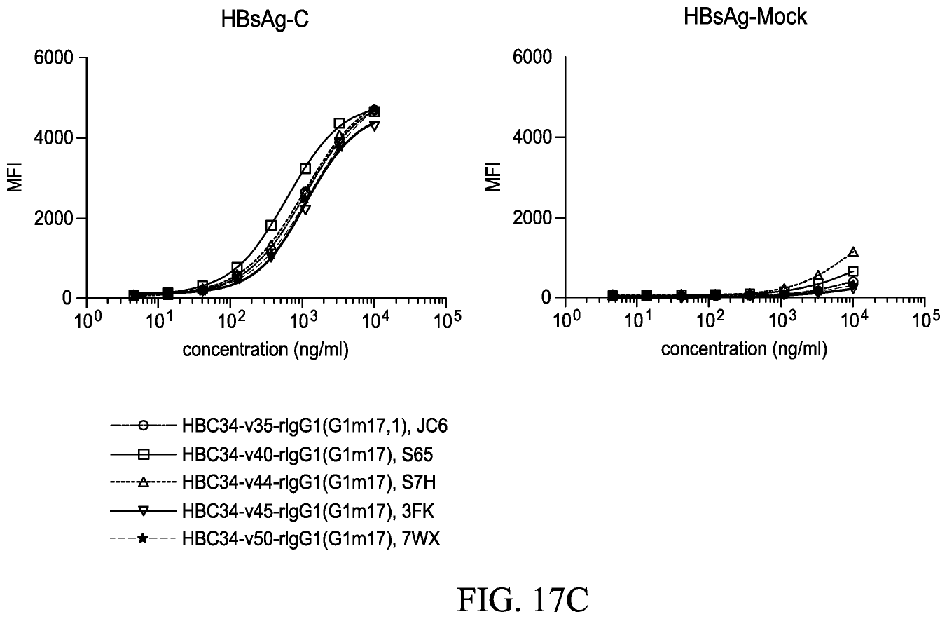

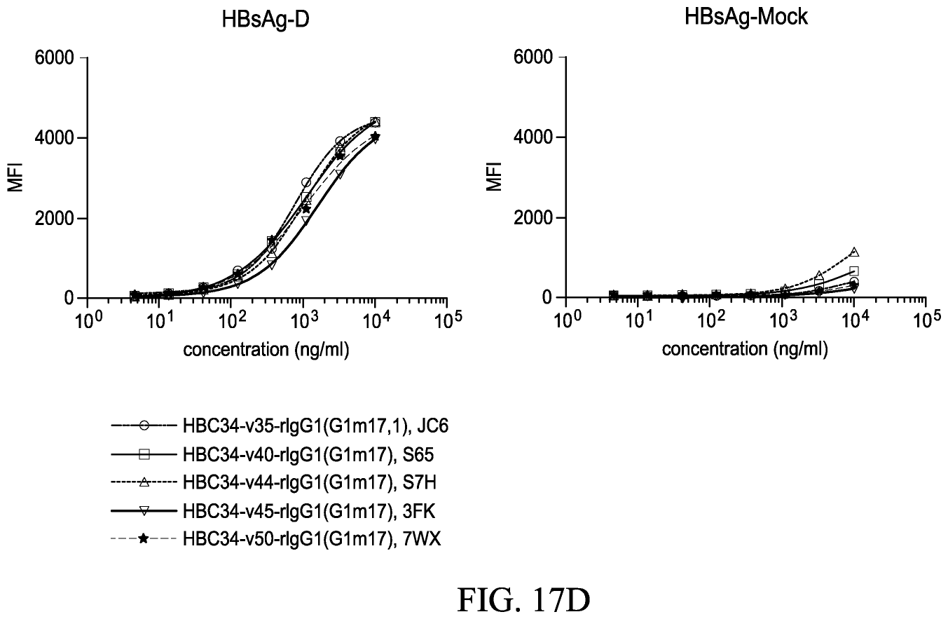

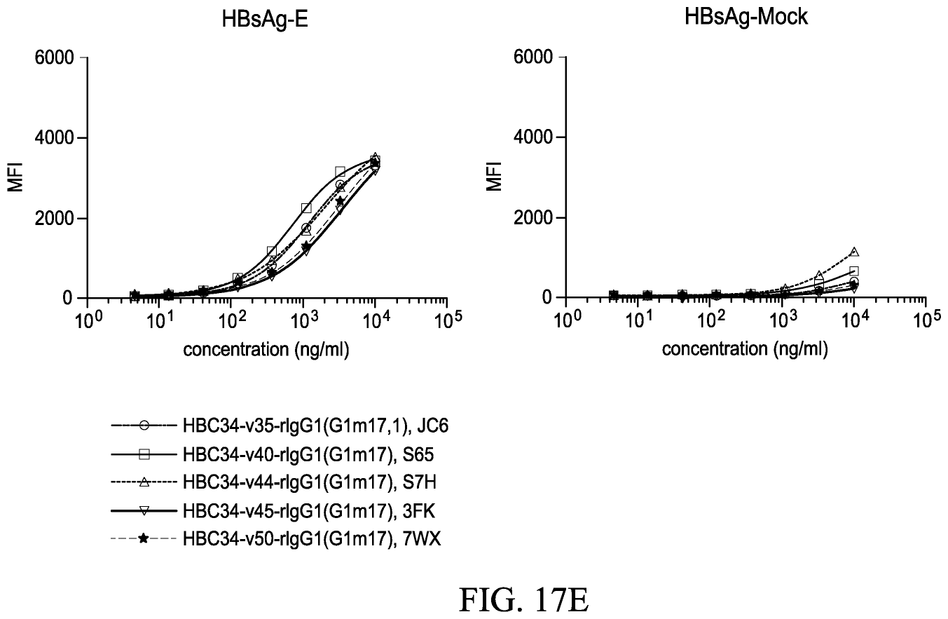

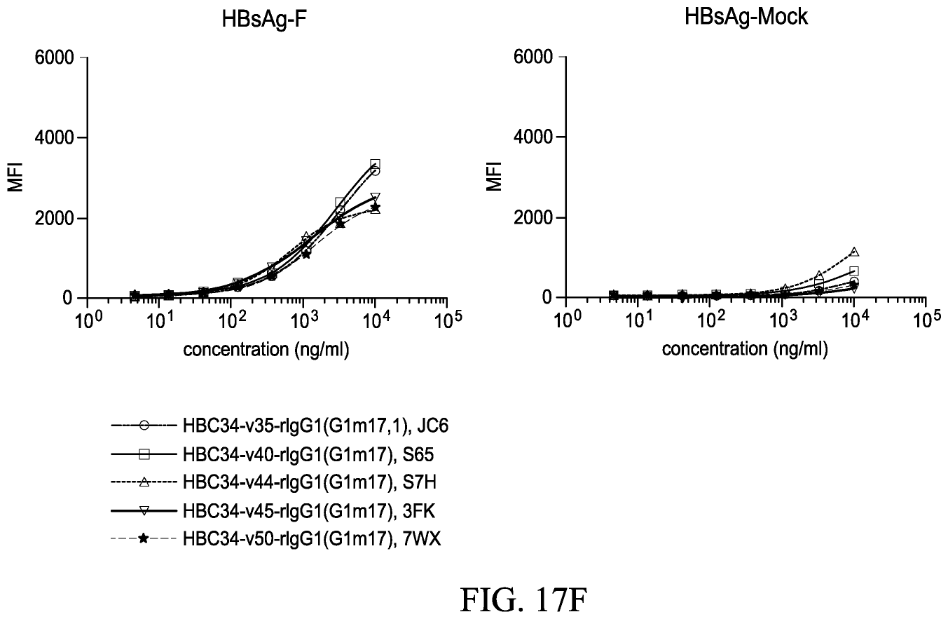

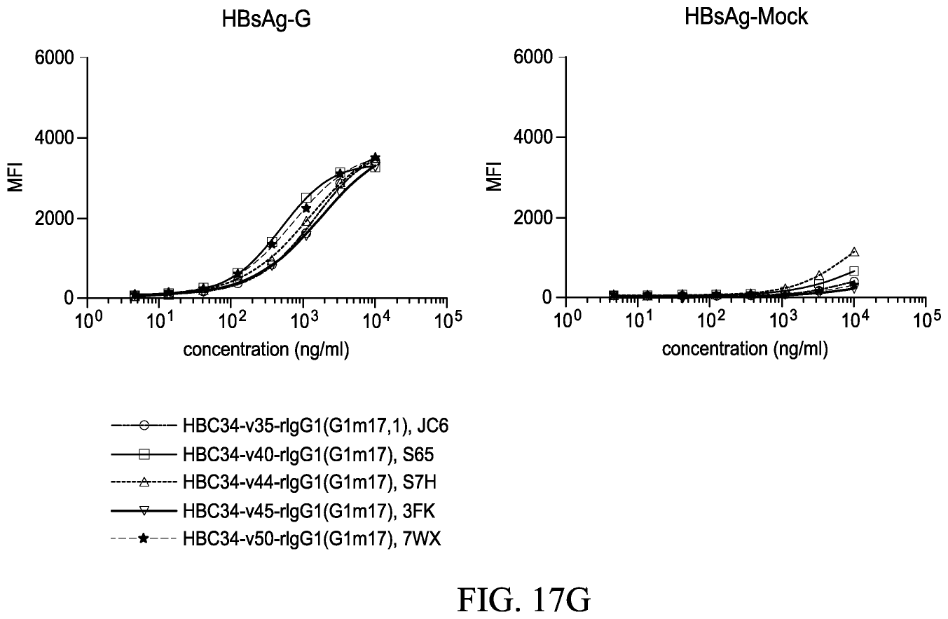

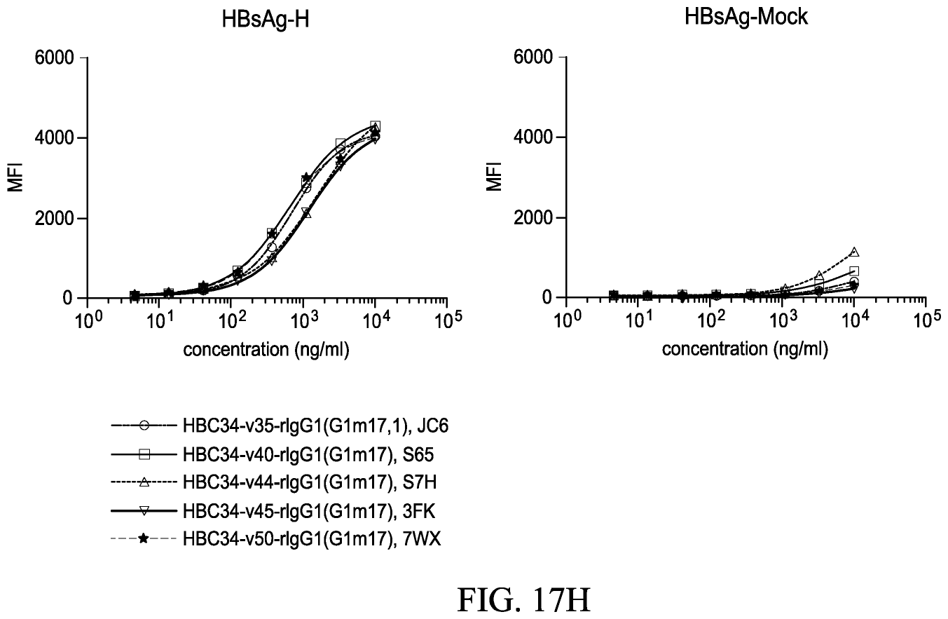

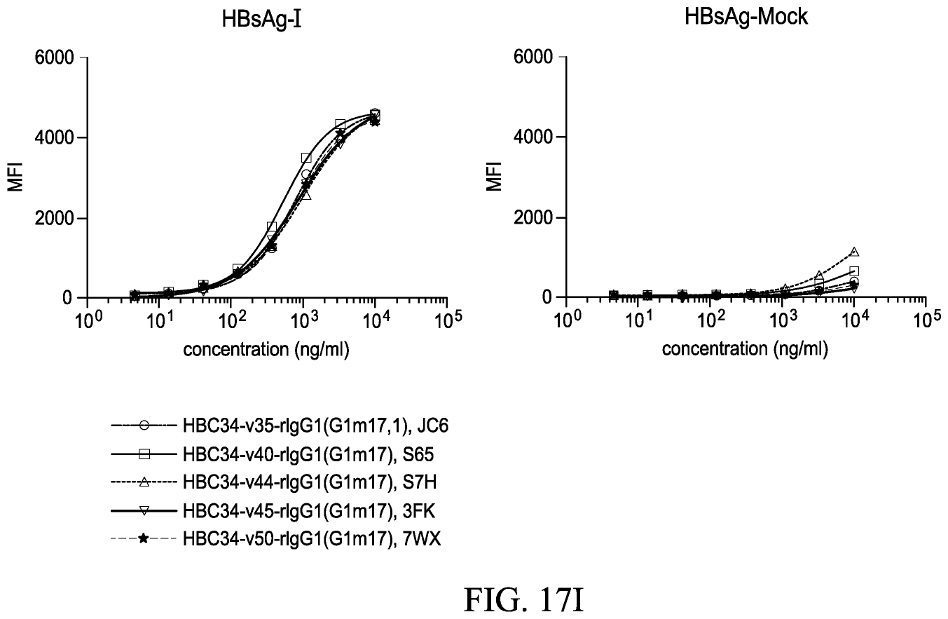

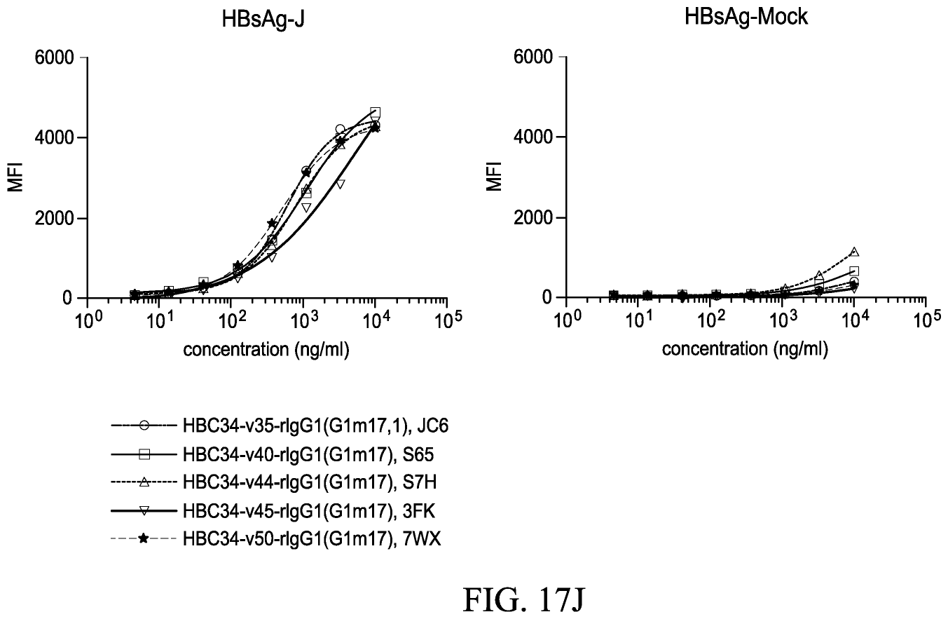

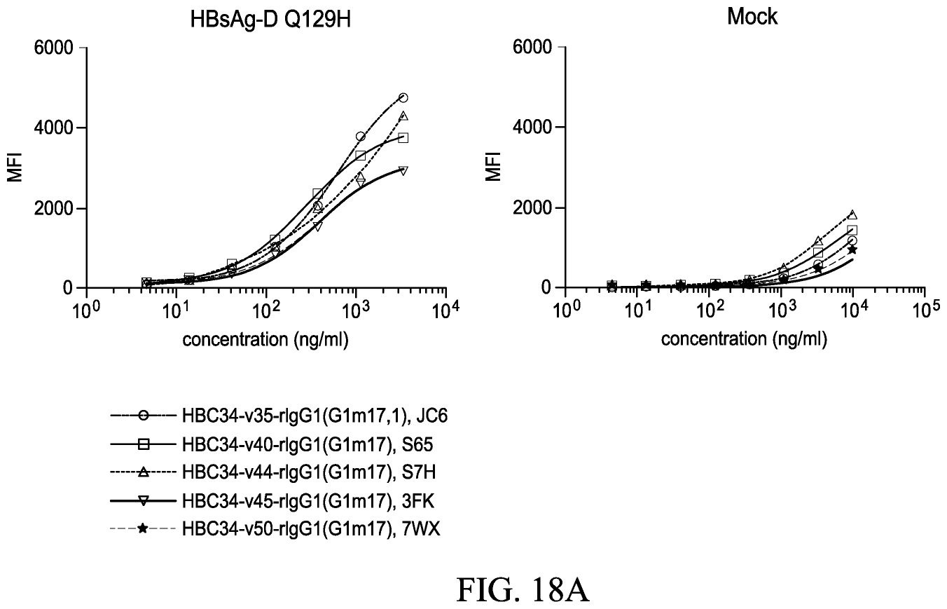

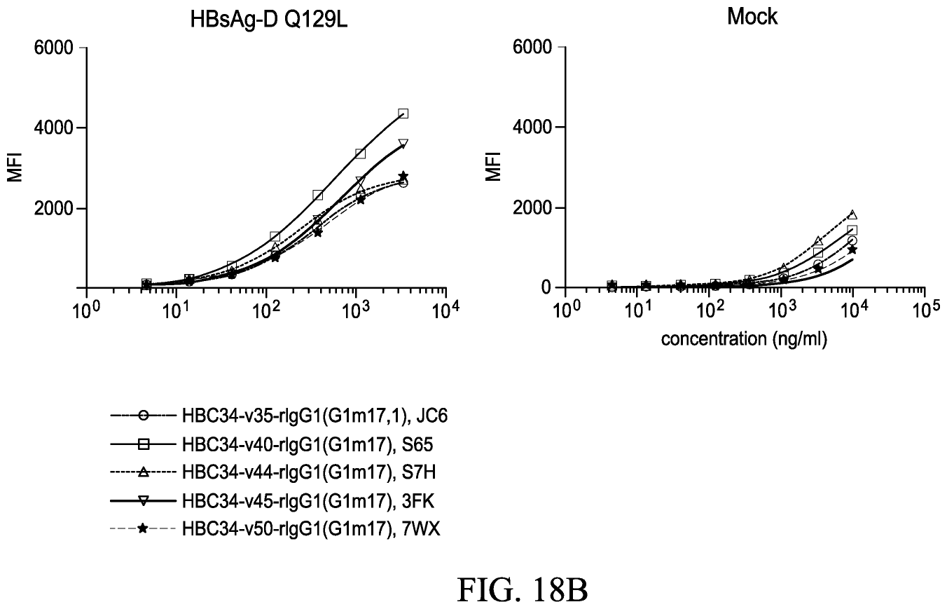

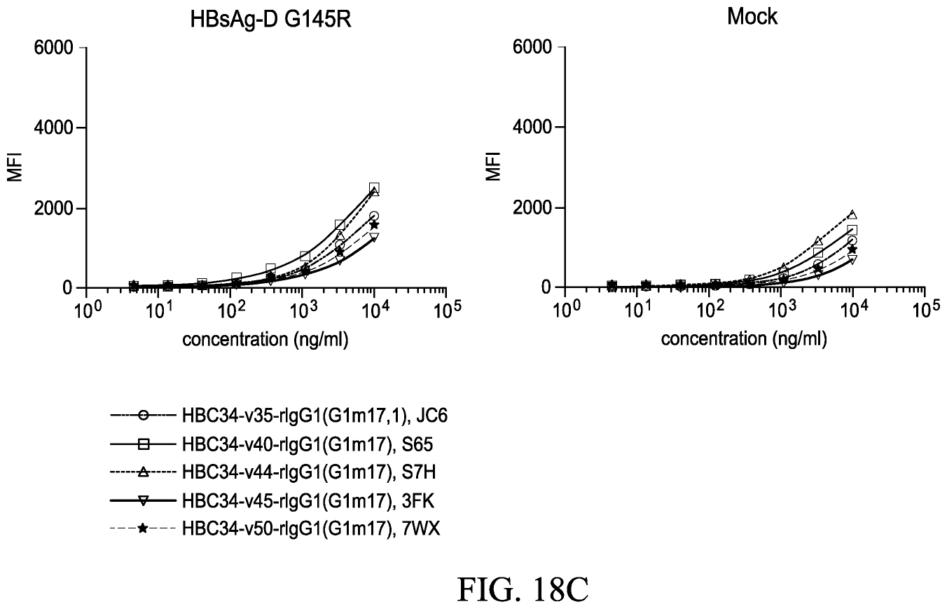

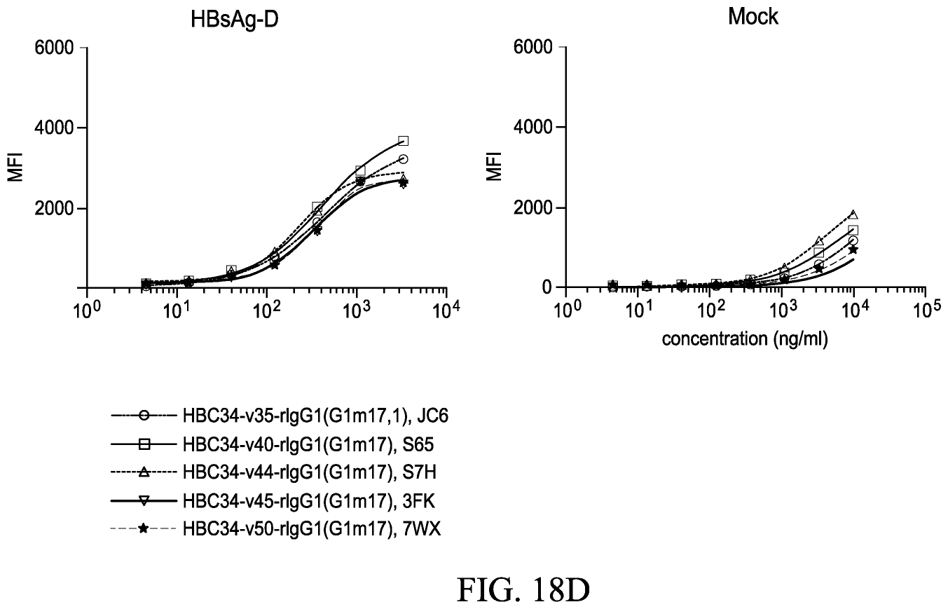

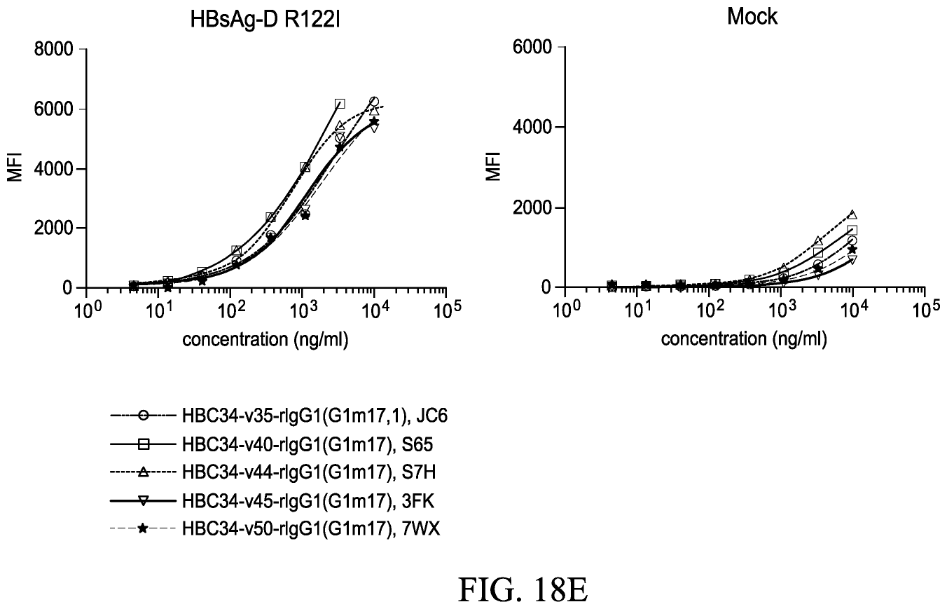

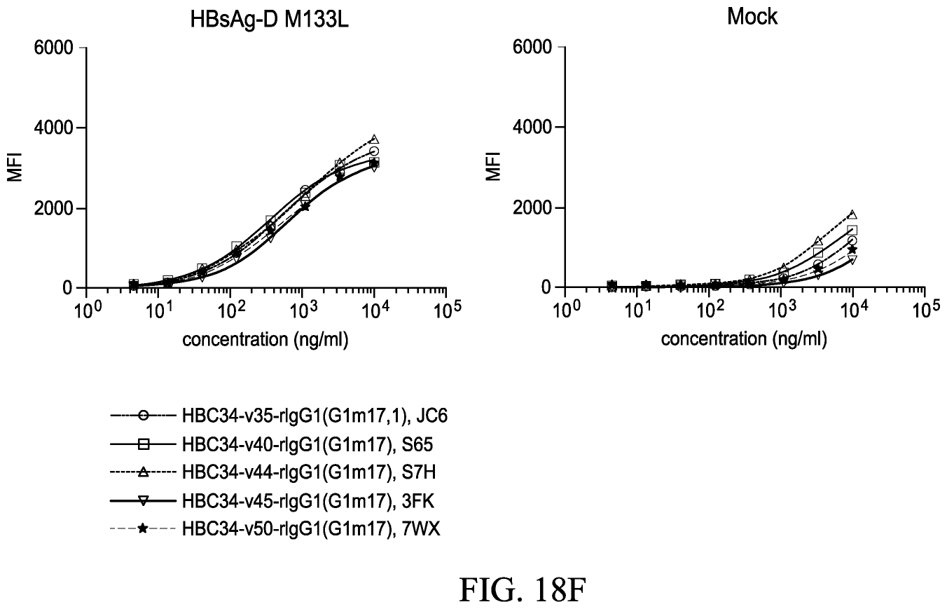

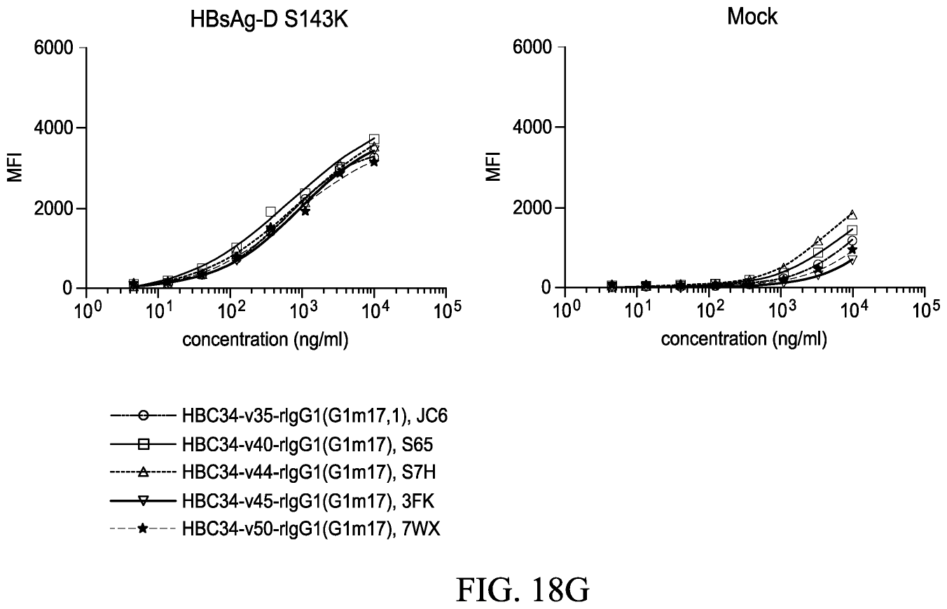

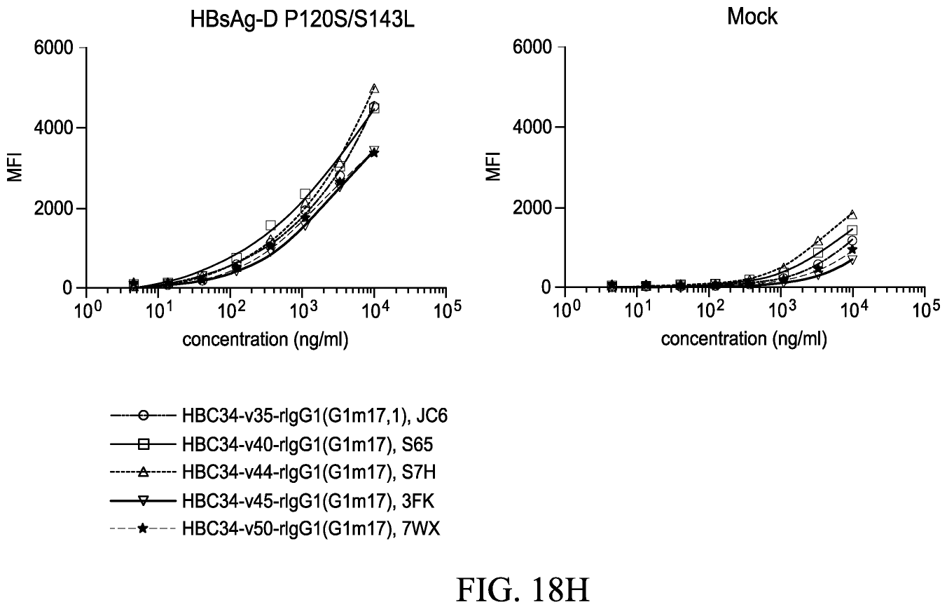

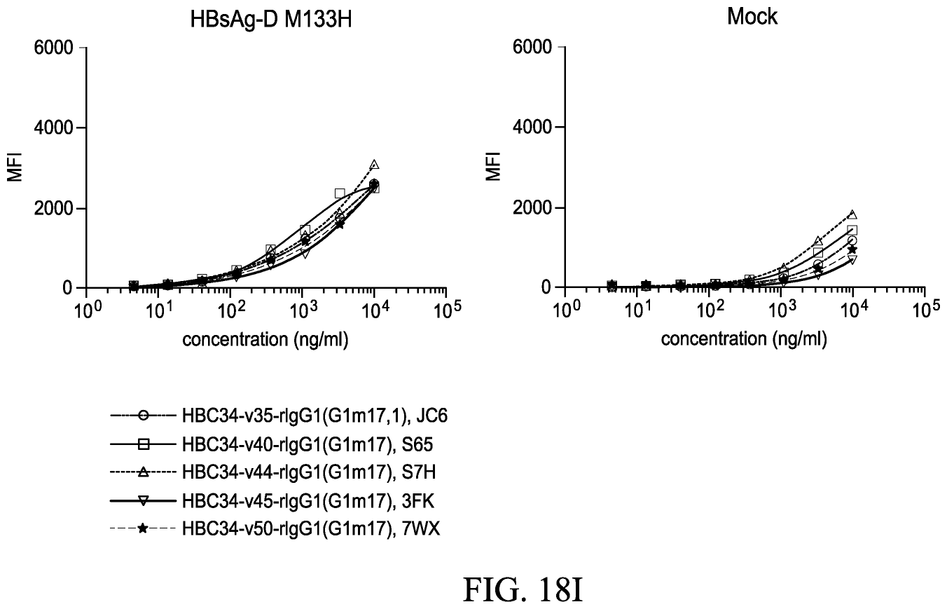

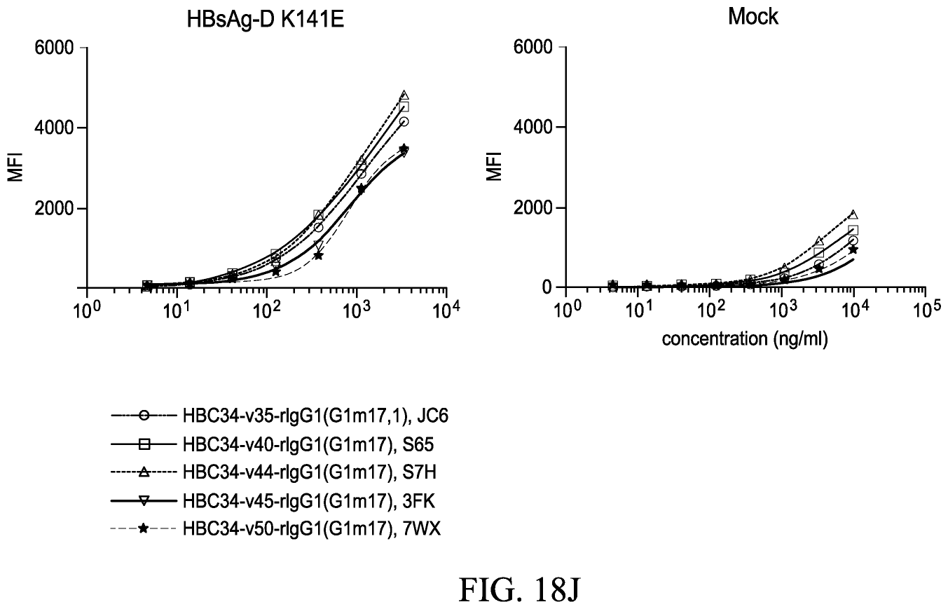

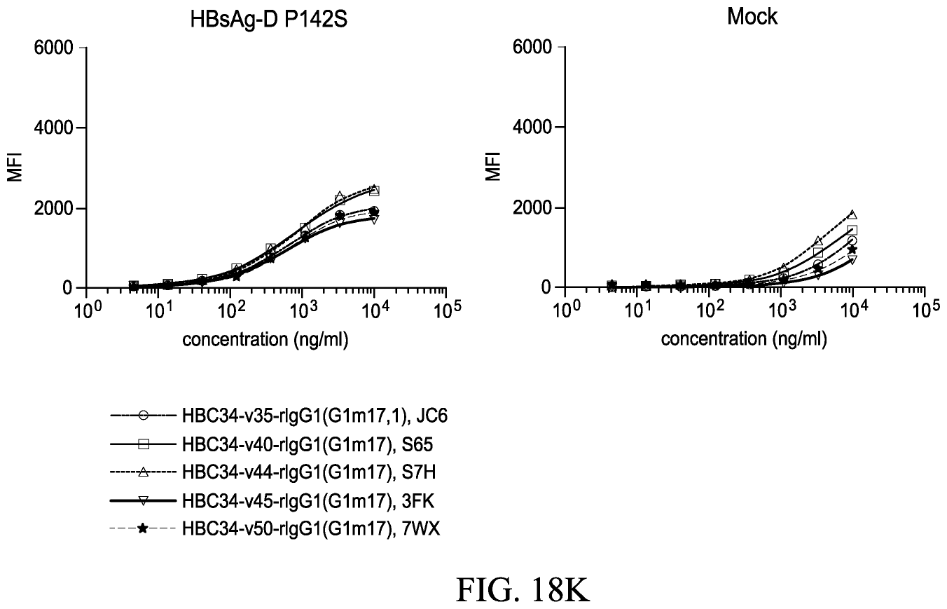

Binding of HBC34-v40, HBC34-v44, HBC34-v45, and HBC34-v50, to HBsAg from ten ((A)-(J)) genotypes was tested by FACS. HBC34-v35 was included as reference. All of the tested variants bound to HBsAg, with HBC34-v40 showing the most potent binding (FIGS. 17A-17J). Binding of HBC34-v40, HBC34-v44, HBC34-v45, and HBC34-v50 to ten HBsAg-genotype D mutants was tested by FACS. HBC34-v35 was included as reference. All of the engineered variants bound to HBsAg (FIGS. 18A-18K).

### Example 7: Production of Certain Engineered Antibodies

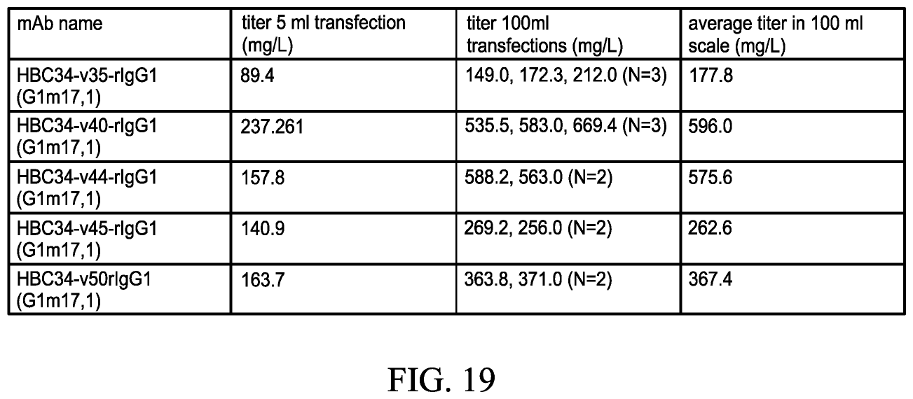

Antibody titers for HBC34-v35, HBC34-v40, HBC34-v44, HBC34-v45, and HBC34-v50 were measured to evaluate productivity in host cells. Antibodies were expressed as recombinant IgG (allotype G1m17, 1) in a host cell line and purified from supernatant. Both 5 ml- and 100 ml-scale transfection systems were evaluated, with the 100 ml system tested in duplicate or triplicate. Antibody titers from individual 5 ml-and 100 ml-scale tests as well as average titer from 100 ml-scale tests are shown in FIG. 19.

### Example 8: Thermostability of Certain Engineered Antibodies

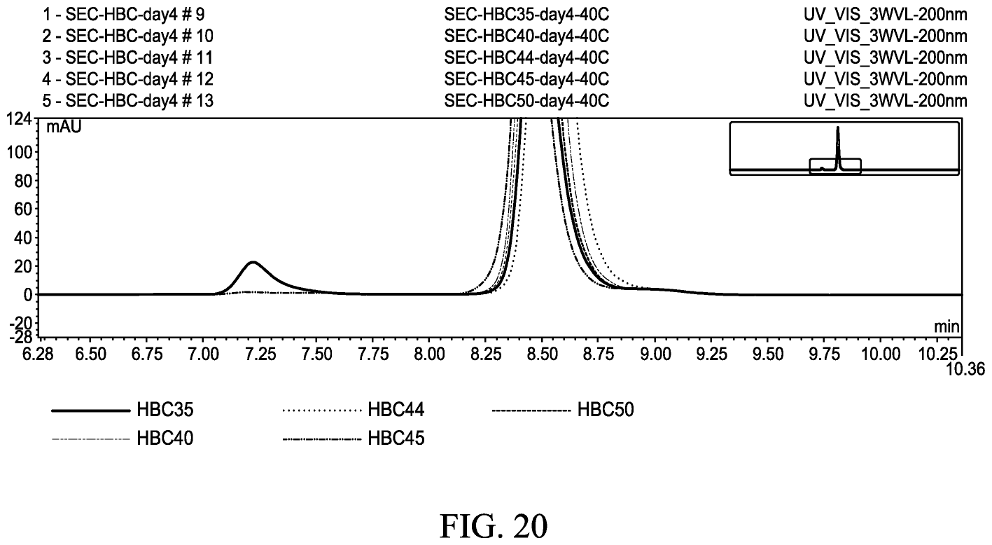

HBC34-v35, HBC34-v40, HBC34-v44, HBC34-v45, and HBC34-v50 were expressed as recombinant IgG (allotype G1m17, 1) in a host cell line and purified from supernatant. Antibodies were concentrated to 25 mg/ml and incubated at 40° C. for four days. Size-exclusion chromatography analysis was used to quantify high molecular weight species (HMWS) corresponding to an antibody dimer at day 4, as shown in FIG. 20. Only HBC34-v35 showed significant HMWS after 4 days.

### Example 9: Analysis of Light Chain Amino Acids Involved in Forming Antibody Dimers

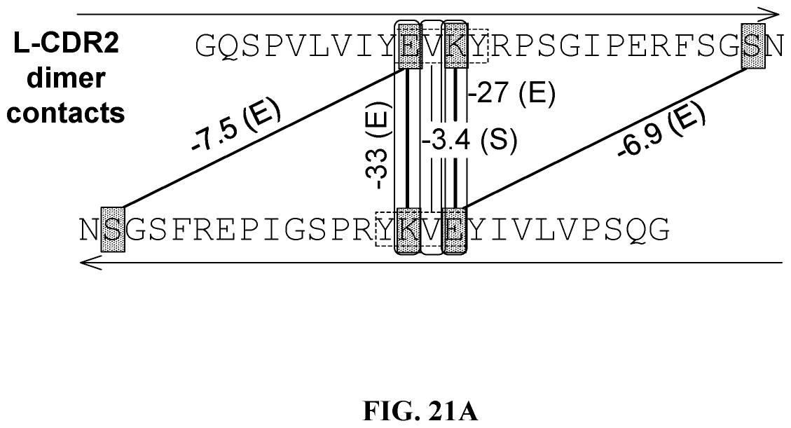

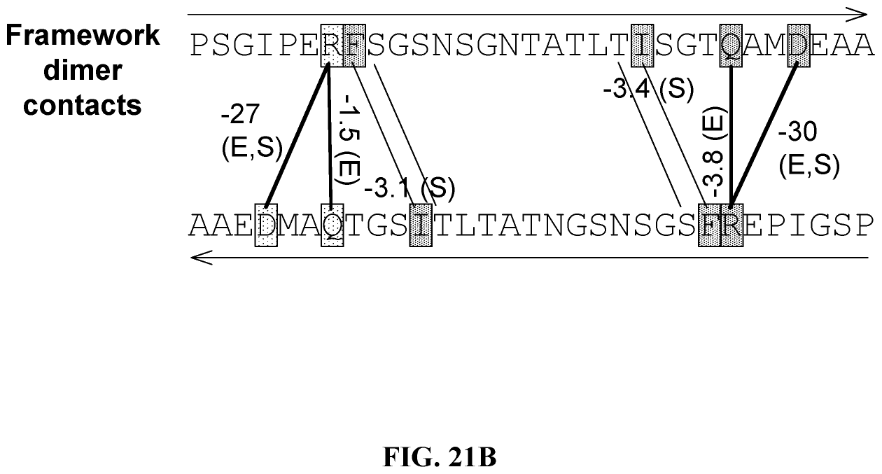

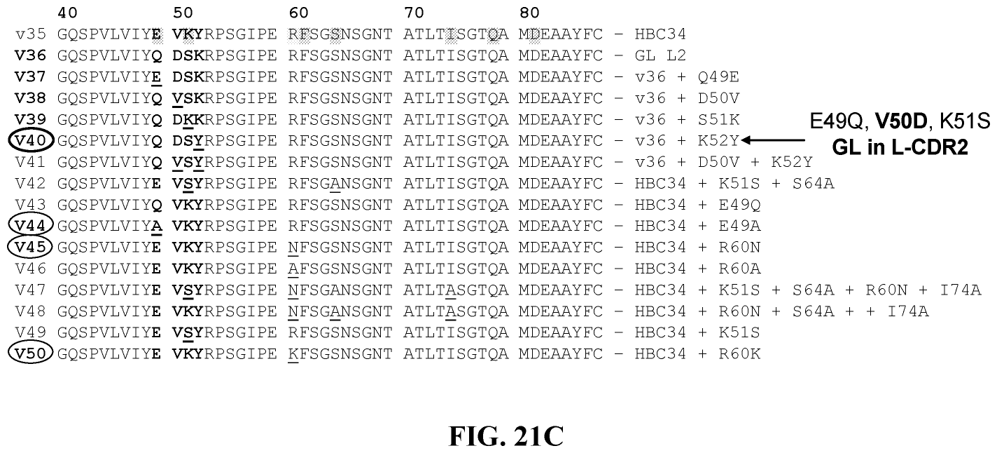

Structural studies identified of number of amino acid residues in the HBC34-v35 VL region that were involved in forming antibody:antibody dimers. Interactions between light chain residues of two HBC34-v35 antibody molecules (herein, “antibody molecule 1” and “antibody molecule 2”) are illustrated in FIGS. 21A, 22A, and 23A, wherein: E49 (antibody molecule 1) interacts with S64 and K51 (antibody molecule 2); V50 (antibody molecule 1) interacts with V50 (antibody molecule 2); K51 (antibody molecule 1) interacts with E49 (antibody molecule 2); and S64 (antibody molecule 1) interacts with E49 (antibody molecule 2). Interactions between other light chain amino acids of two HBC34-v35 antibodies is shown in FIGS. 21B, 22B, and 23B, wherein: R60 (antibody molecule 1) interacts with D81 and Q78 (antibody molecule 2); F61 (antibody 1) interacts with 174 (antibody molecule 2); 174 (antibody molecule 1) interacts with F61 (antibody molecule 2); Q78 (antibody molecule 1) interacts with R60 (antibody molecule 2); and D81 (antibody molecule 1) interacts with R60 (antibody molecule 2).

Four engineered antibodies, HBC34-v40, HBC34-v44, HBC34-v45, and HBC34-v50, were determined to have a low propensity for aggregation while maintaining potent binding.

HBC34-v40 comprises E49Q, V50D, and K51S mutations in L-CDR2 (CCG numbering) compared to parental HBC34-v35, as shown in FIG. 21C. These mutations change from hydrophobic interaction to electrostatic repulsion and the loss of a salt bridge, though loss of the salt bridge alone was not sufficient to reduce aggregation (compare to HBC34-v41 comprising E49Q and K51S mutations, but not V50D, and see FIG. 16D).

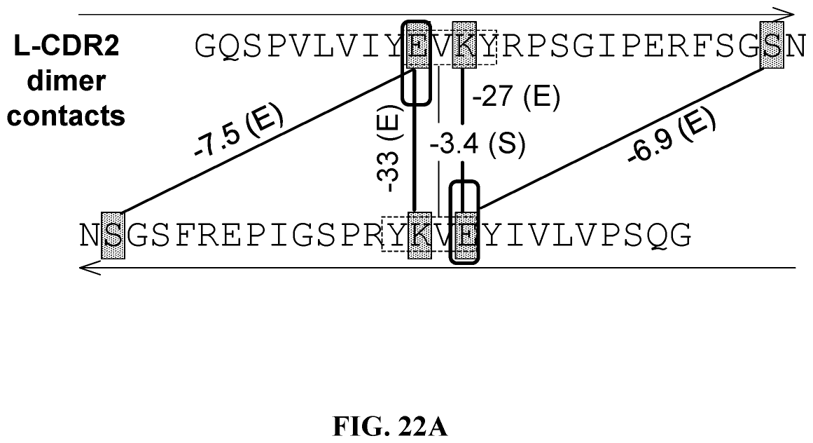

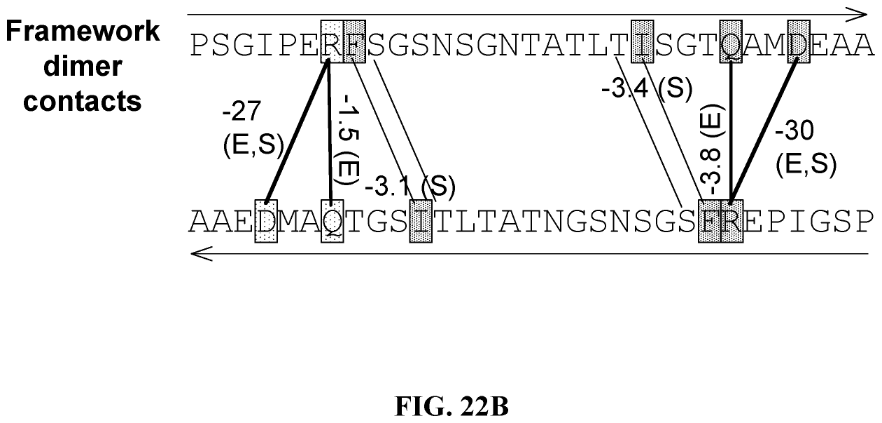

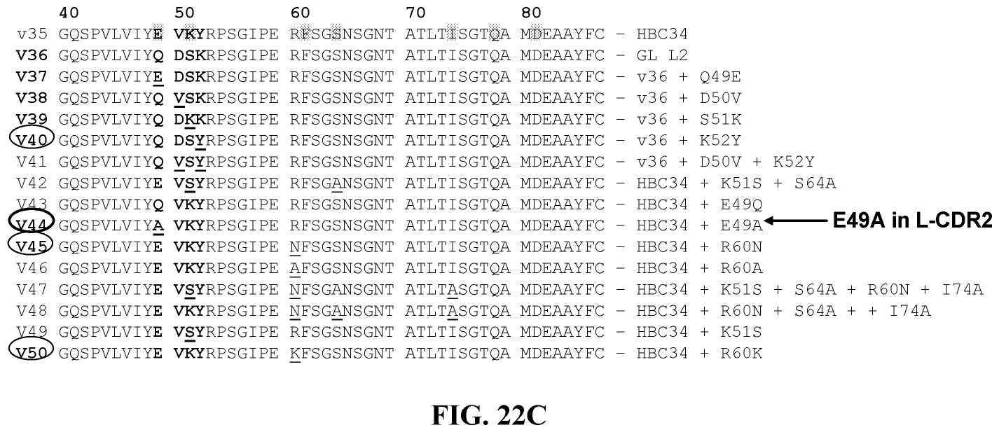

HBC34-v44 comprises an E49A mutation in L-CDR2 as compared to HBC34-v35, as shown in FIG. 22C. This mutation results in loss of a salt bridge.

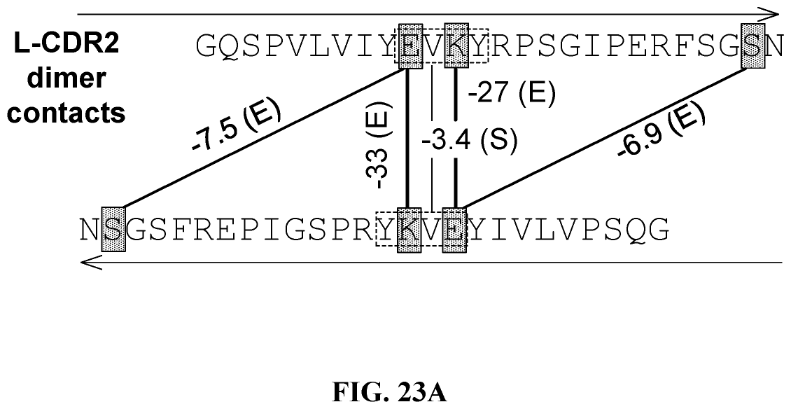

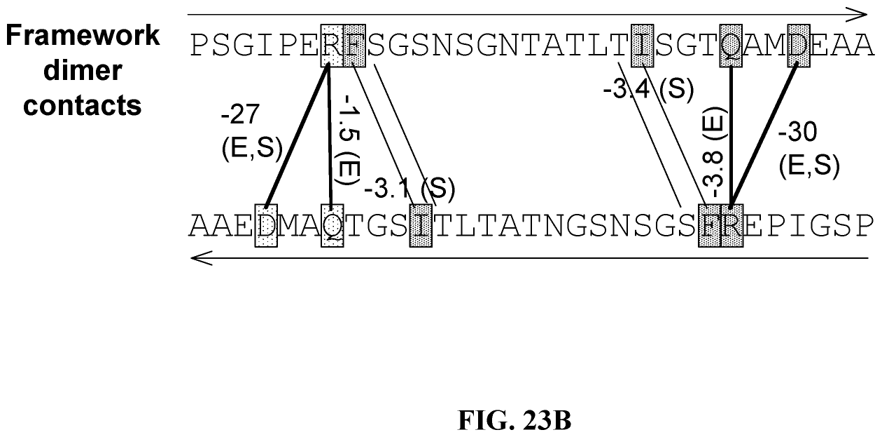

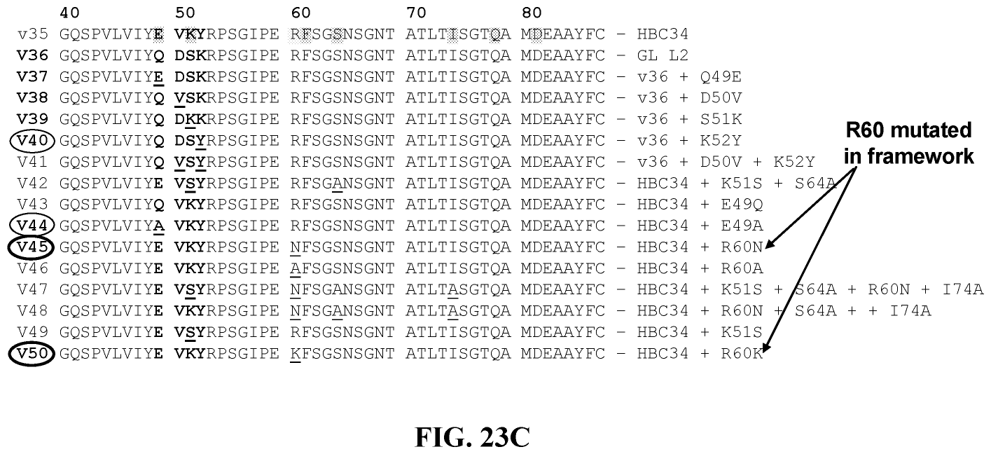

HBC34-v45 and HBC34-v50 comprise framework mutations at R60 relative to HBC34-v35, as shown in FIG. 23C. R60N and R60K mutations in HBC34-v45 and HBC34-v50, respectively, reduced dimer formation. A R60A mutation in HBC34-v46 was less effective in reducing dimer formation (see FIG. 16D).

TABLE OF SEQUENCES AND SEQ ID NUMBERS (SEQUENCE LISTING)

| SEQ | | |
|---|---|---|
| ID | | |
| NO | Sequence | Remarks |
| 1 | X₁ X₂ X₃ TC X₄ X₅ X₆A X₇G | epitope |
| | wherein X₁, X₂, X₃, X₄, X₅, X₆ and X₇ may be any amino acid | |
| 2 | X₁ X, X₃ TC X₄ X₅ X₆A X₇G | |
| | wherein X₁ is P, T or S, | |
| | X₂ is C or S, | |
| | X₃ is R, K, D or I, | |
| | X₄ is M or T, | |
| | X₅ is T, A or I, | |
| | X₆ is T, P or L, and | |
| | X₇ is Q, H or L. | |
| 3 | MENITSGFLGPLLVLQAGFFLLTRILTIPQSLDSWWTSLNF | S domain of HBsAg |
| | LGGTTVCLGQNSQSPTSNHSPTSCPPTCPGYRWMCLRRFI | (GenBank acc. no. |
| | IFLFILLLCLIFLLVLLDYQGMLPVCPLIPGSSTTSTGPCRT | J02203) |
| | CMTTAQGTSMYPSCCCTKPSDGNCTCIPIPSSWAFGKFL | |
| | WEWASARFSWLSLLVPFVQWFVGLSPTVWLSVIWMMW | |
| | YWGPSLYSILSPFLPLLPIFFCLWVYI | |
| 4 | MENVTSGFLGPLLVLQAGFFLLTRILTIPQSLDSWWTSLN | S domain of HBsAg |
| | FLGGTTVCLGQNSQSPTSNHSPTSCPPTCPGYRWMCLRR | (GenBank acc. no. |
| | FIIFLFILLLCLIFLLVLLDYQGMLPVCPLIPGSSTTGTGPCR | FJ899792) |
| | TCTTPAQGTSMYPSCCCTKPSDGNCTCIPIPSSWAFGKFL | |
| | WEWASARFSWLSLLVPFVQWFVGLSPTVWLSVIWMMW | |
| | YWGPSLYSTLSPFLPLLPIFFCLWVYI | |
| 5 | QGMLPVCPLIPGSSTTSTGPCRTCMTTAQGTSMYPSCCCT | J02203 (D, ayw3) |
| | KPSDGNCTCIPIPSSWAFGKFLWEWASARFSW | |
| 6 | QGMLPVCPLIPGSSTTGTGPCRTCTTPAQGTSMYPSCCCT | FJ899792 (D, adw2) |
| | KPSDGNCTCIPIPSSWAFGKFLWEWASARFSW | |
| 7 | QGMLPVCPLIPGTTTTSTGPCKTCTTPAQGNSMFPSCCCT | AM282986 (A) |
| | KPSDGNCTCIPIPSSWAFAKYLWEWASVRFSW | |
| 8 | QGMLPVCPLIPGSSTTSTGPCKTCTTPAQGTSMFPSCCCT | D23678 (B1) |
| | KPTDGNCTCIPIPSSWAFAKYLWEWASVRFSW | |
| 9 | QGMLPVCPLLPGTSTTSTGPCKTCTIPAQGTSMFPSCCCT | AB117758 (C1) |
| | KPSDGNCTCIPIPSSWAFARFLWEWASVRFSW | |
| 10 | QGMLPVCPLIPGSSTTSTGPCRTCTTLAQGTSMFPSCCCS | AB205192 (E) |
| | KPSDGNCTCIPIPSSWAFGKFLWEWASARFSW | |
| 11 | QGMLPVCPLLPGSTTTSTGPCKTCTTLAQGTSMFPSCCCS | X69798 (F4) |
| | KPSDGNCTCIPIPSSWALGKYLWEWASARFSW | |
| 12 | QGMLPVCPLIPGSSTTSTGPCKTCTTPAQGNSMYPSCCCT | AF160501 (G) |
| | KPSDGNCTCIPIPSSWAFAKYLWEWASVRFSW | |
| 13 | QGMLPVCPLLPGSTTTSTGPCKTCTTLAQGTSMFPSCCCT | AY090454 (H) |
| | KPSDGNCTCIPIPSSWAFGKYLWEWASARFSW | |
| 14 | QGMLPVCPLIPGSSTTSTGPCKTCTTPAQGNSMYPSCCCT | AF241409 (I) |
| | KPSDGNCTCIPIPSSWAFAKYLWEWASARFSW | |
| 15 | QGMLPVCPLLPGSTTTSTGPCRTCTITAQGTSMFPSCCCT | AB486012 (J) |
| | KPSDGNCTCIPIPSSWAFAKFLWEWASVRFSW | |
| 16 | CQGMLPVCPLIPGSSTTGTGTCRTCTTPAQGTSMYPSCCC | HBsAg Y100C/P120T |
| | TKPSDGNCTCIPIPSSWAFGKFLWEWASARFSW | |
| 17 | QGMLPVCPLIPGSSTTGTGTCRTCTTPAQGTSMYPSCCCT | HBsAg P120T |
| | KPSDGNCTCIPIPSSWAFGKFLWEWASARFSW | |
| 18 | QGMLPVCPLIPGSSTTGTGTCRTCTTPAQGTSMYPSCCCT | HBsAg P120T/S143L |
| | KPLDGNCTCIPIPSSWAFGKFLWEWASARFSW | |
| 19 | QGMLPVCPLIPGSSTTGTGPSRTCTTPAQGTSMYPSCCCT | HBsAg C121S |
| | KPSDGNCTCIPIPSSWAFGKFLWEWASARFSW | |
| 20 | QGMLPVCPLIPGSSTTGTGPCDTCTTPAQGTSMYPSCCCT | HBsAg R122D |
| | KPSDGNCTCIPIPSSWAFGKFLWEWASARFSW | |
| 21 | QGMLPVCPLIPGSSTTGTGPCITCTTPAQGTSMYPSCCCT | HBsAg R122I |
| | KPSDGNCTCIPIPSSWAFGKFLWEWASARFSW | |
| 22 | QGMLPVCPLIPGSSTTGTGPCRNCTTPAQGTSMYPSCCCT | HBsAg T123N |
| | KPSDGNCTCIPIPSSWAFGKFLWEWASARFSW | |
| 23 | QGMLPVCPLIPGSSTTGTGPCRTCTTPAHGTSMYPSCCCT | HBsAg Q129H |
| | KPSDGNCTCIPIPSSWAFGKFLWEWASARFSW | |
| 24 | QGMLPVCPLIPGSSTTGTGPCRTCTTPALGTSMYPSCCCT | HBsAg Q129L |
| | KPSDGNCTCIPIPSSWAFGKFLWEWASARFSW | |
| 25 | QGMLPVCPLIPGSSTTGTGPCRTCTTPAQGTSHYPSCCCT | HBsAg M133H |
| | KPSDGNCTCIPIPSSWAFGKFLWEWASARFSW | |
| 26 | QGMLPVCPLIPGSSTTGTGPCRTCTTPAQGTSLYPSCCCT | HBsAg M133L |
| | KPSDGNCTCIPIPSSWAFGKFLWEWASARFSW | |
| 27 | QGMLPVCPLIPGSSTTGTGPCRTCTTPAQGTSTYPSCCCT | HBsAg M133T |
| | KPSDGNCTCIPIPSSWAFGKFLWEWASARFSW | |
| 28 | QGMLPVCPLIPGSSTTGTGPCRTCTTPAQGTSMYPSCCCT | HBsAg K14IE |
| | EPSDGNCTCIPIPSSWAFGKFLWEWASARFSW | |
| 29 | QGMLPVCPLIPGSSTTGTGPCRTCTTPAQGTSMYPSCCCT | HBsAg P142S |
| | KSSDGNCTCIPIPSSWAFGKFLWEWASARFSW | |
| 30 | QGMLPVCPLIPGSSTTGTGPCRTCTTPAQGTSMYPSCCCT | HBsAg S143K |
| | KPKDGNCTCIPIPSSWAFGKFLWEWASARFSW | |
| 31 | QGMLPVCPLIPGSSTTGTGPCRTCTTPAQGTSMYPSCCCT | HBsAg D144A |
| | KPSAGNCTCIPIPSSWAFGKFLWEWASARFSW | |
| 32 | QGMLPVCPLIPGSSTTGTGPCRTCTTPAQGTSMYPSCCCT | HBsAg G145R |
| | KPSDRNCTCIPIPSSWAFGKFLWEWASARFSW | |
| 33 | QGMLPVCPLIPGSSTTGTGPCRTCTTPAQGTSMYPSCCCT | HBsAg N146A |
| | KPSDGACTCIPIPSSWAFGKFLWEWASARFSW | |
| 34 | GRIFRSFYMS | HBC34 Ab CDRH1 |
| | | aa (CCG numbering) |
| 35 | TINQDGSEKLYVDSVKG | HBC34 Ab CDRH2 |
| | | aa (CCG numbering) |
| 36 | NINQDGSEKLYVDSVKG | HBC34 Ab CDRH2 2 |
| | | aa (CCG numbering) |
| 37 | WSGNSGGMDV | HBC34 Ab CDRH3 |
| | | aa (CCG numbering) |
| 38 | ELQLVESGGGWVQPGGSQRLSCAASGRIFRSFYMSWVR | HBC34 VH_1 |
| | QAPGKGLEWVATINQDGSEKLYVDSVKGRFTISRDNAK | |
| | NSLFLQMNNLRVEDTAVYYCAAWSGNSGGMDVWGQG | |
| | TTVSVSS | |
| 39 | EVQLVESGGGLVQPGGSLRLSCAASGRIFRSFYMSWVRQ | HBC34 VH_2 |
| | APGKGLEWVANINQDGSEKLYVDSVKGRFTISRDNAKNS | |
| | LFLQMNNLRVEDTAVYYCAAWSGNSGGMDVWGQGTT | |
| | VTVSS | |
| 40 | SGDKLGNKNVC | HBC34, -v7, -v31,- |
| | | v32 CDRL1 aa |
| | | (CCG numbering) |
| 41 | SGDKLGNKNVA | HBC34-v35 - v50 |
| | | CDRL1 aa |
| | | (CCG numbering) |
| 42 | SGDKLGNKNVS | HBC34-v34 CDRL1 |
| | | aa |
| | | (CCG numbering) |
| 43 | SGDKLGNKNAC | HBC34-v23, -v33 |
| | | CDRL1 aa |
| | | (CCG numbering) |
| 44 | EVKYRPS | HBC34, -v7, -v23, - |
| | | v31-v33,-v35,-v45, - |
| | | v46, -v48 CDRL2 aa |
| | | (CCG numbering) |
| 45 | QDSKRPS | HBC34-v36 CDRL2 |
| | | aa |
| | | (CCG numbering) |
| 46 | EDSKRPS | HBC34-v37 CDRL2 |
| | | aa (CCG numbering) |
| 47 | QVSKRPS | HBC34-v38 CDRL2 |
| | | aa (CCG numbering) |
| 48 | QDDKRPS | HBC34-v39 CDRL2 |
| | | aa (CCG numbering) |
| 49 | QDSYRPS | HBC34-v40 CDRL2 |
| | | aa (CCG numbering) |
| 50 | QVSYRPS | HBC34-v41 CDRL2 |
| | | aa (CCG numbering) |
| 51 | EVSYRPS | HBC34-v42, -v47, - |
| | | v49, -v50 CDRL2 aa |
| | | (CCG numbering) |
| 52 | QVKYRPS | HBC34-v43 CDRL2 |
| | | aa (CCG numbering) |
| 53 | AVKYRPS | HBC34-v44 CDRL2 |
| | | aa (CCG numbering) |
| 54 | [reserved] | |
| 55 | QTFDSTTVV | HBC34-v7, -v23, - |
| | | v32,-v33,-v35-v50 |
| | | CDRL3 aa |
| | | (CCG numbering) |
| 56 | QTWDSTTVV | HBC34, -v31 CDRL3 |
| | | aa (CCG numbering) |
| 57 | SYELTQPPSVSVSPGQTVSIPCSGDKLGNKNVAWFQHKP | HBC34-v35 VL aa |
| | GQSPVLVIYEVKYRPSGIPERFSGSNSGNTATLTISGTQAM | |
| | DEAAYFCQTFDSTTVVFGGGTRLTVL | |
| 58 | SYELTQPPSVSVSPGQTVSIPCSGDKLGNKNVAWFQHKP | HBC34-v36 VL aa |
| | GQSPVLVIYQDSKRPSGIPERFSGSNSGNTATLTISGTQAM | |
| | DEAAYFCQTFDSTTVVFGGGTRLTVL | |
| 59 | SYELTQPPSVSVSPGQTVSIPCSGDKLGNKNVAWFQHKP | HBC34-v37 VL aa |
| | GQSPVLVIYEDSKRPSGIPERFSGSNSGNTATLTISGTQAM | |
| | DEAAYFCQTFDSTTVVFGGGTRLTVL | |
| 60 | SYELTQPPSVSVSPGQTVSIPCSGDKLGNKNVAWFQHKP | HBC34-v38 VL aa |
| | GQSPVLVIYQVSKRPSGIPERFSGSNSGNTATLTISGTQAM | |
| | DEAAYFCQTFDSTTVVFGGGTRLTVL | |
| 61 | SYELTQPPSVSVSPGQTVSIPCSGDKLGNKNVAWFQHKP | HBC34-v39 VL aa |
| | GQSPVLVIYQDKKRPSGIPERFSGSNSGNTATLTISGTQA | |
| | MDEAAYFCQTFDSTTVVFGGGTRLTVL | |
| 62 | SYELTQPPSVSVSPGQTVSIPCSGDKLGNKNVAWFQHKP | HBC34-v40 VL aa |
| | GQSPVLVIYQDSYRPSGIPERFSGSNSGNTATLTISGTQAM | |
| | DEAAYFCQTFDSTTVVFGGGTRLTVL | |
| 63 | SYELTQPPSVSVSPGQTVSIPCSGDKLGNKNVAWFQHKP | HBC34-v41 VL aa |
| | GQSPVLVIYQVSYRPSGIPERFSGSNSGNTATLTISGTQAM | |
| | DEAAYFCQTFDSTTVVFGGGTRLTVL | |
| 64 | SYELTQPPSVSVSPGQTVSIPCSGDKLGNKNVAWFQHKP | HBC34-v42 VL aa |
| | GQSPVLVIYEVSYRPSGIPERFSGANSGNTATLTISGTQAM | |
| | DEAAYFCQTFDSTTVVFGGGTRLTVL | |
| 65 | SYELTQPPSVSVSPGQTVSIPCSGDKLGNKNVAWFQHKP | HBC34-v43 VL aa |
| | GQSPVLVIYQVKYRPSGIPERFSGSNSGNTATLTISGTQA | |
| | MDEAAYFCQTFDSTTVVFGGGTRLTVL | |
| 66 | SYELTQPPSVSVSPGQTVSIPCSGDKLGNKNVAWFQHKP | HBC34-v44 VL aa |
| | GQSPVLVIYAVKYRPSGIPERFSGSNSGNTATLTISGTQA | |
| | MDEAAYFCQTFDSTTVVFGGGTRLTVL | |
| 67 | SYELTQPPSVSVSPGQTVSIPCSGDKLGNKNVAWFQHKP | HBC34-v45 VL aa |
| | GQSPVLVIYEVKYRPSGIPENFSGSNSGNTATLTISGTQA | |
| | MDEAAYFCQTFDSTTVVFGGGTRLTVL | |
| 68 | SYELTQPPSVSVSPGQTVSIPCSGDKLGNKNVAWFQHKP | HBC34-v46 VL aa |
| | GQSPVLVIYEVKYRPSGIPEAFSGSNSGNTATLTISGTQA | |
| | MDEAAYFCQTFDSTTVVFGGGTRLTVL | |
| 69 | SYELTQPPSVSVSPGQTVSIPCSGDKLGNKNVAWFQHKP | HBC34-v47 VL aa |
| | GQSPVLVIYEVSYRPSGIPENFSGANSGNTATLTASGTQA | |
| | MDEAAYFCQTFDSTTVVFGGGTRLTVL | |
| 70 | SYELTQPPSVSVSPGQTVSIPCSGDKLGNKNVAWFQHKP | HBC34-v48 VL aa |
| | GQSPVLVIYEVKYRPSGIPENFSGANSGNTATLTASGTQA | |
| | MDEAAYFCQTFDSTTVVFGGGTRLTVL | |
| 71 | SYELTQPPSVSVSPGQTVSIPCSGDKLGNKNVAWFQHKP | HBC34-v49 VL aa |
| | GQSPVLVIYEVSYRPSGIPERFSGSNSGNTATLTISGTQAM | |
| | DEAAYFCQTFDSTTVVFGGGTRLTVL | |
| 72 | SYELTQPPSVSVSPGQTVSIPCSGDKLGNKNVAWFQHKP | HBC34-v50 VL aa |
| | GQSPVLVIYEVKYRPSGIPEKFSGSNSGNTATLTISGTQA | |
| | MDEAAYFCQTFDSTTVVFGGGTRLTVL | |
| 73 | APELLGGPSVFLFPPKPKDTLMISRTPEVTCVVVDVSHED | WT hIgG1 Fc |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPSRDELTKNQVSLTCLVKGFYPSDIAVEWESNGQ | |
| | PENNYKTTPPVLDSDGSFFLYSKLTVDKSRWQQGNVFSC | |
| | SVMHEALHNHYTQKSLSLSPGK | |
| 74 | ESKYGPPCPPCPAPPVAGP | Chimeric hinge |
| | | sequence |
| 75 | ELQLVESGGGWVQPGGSQRLSCAASGRIFRSFYMSWVR | HC of HBC34-v35-50 |
| | QAPGKGLEWVATINQDGSEKLYVDSVKGRFTISRDNAK | MLNS-GAALIE |
| | NSLFLQMNNLRVEDTAVYYCAAWSGNSGGMDVWGQG | (g1M17, 1) |
| | TTVSVSSASTKGPSVFPLAPSSKSTSGGTAALGCLVKDYF | |
| | PEPVTVSWNSGALTSGVHTFPAVLQSSGLYSLSSVVTVPS | |
| | SSLGTQTYICNVNHKPSNTKVDKKVEPKSCDKTHTCPPC | |
| | PAPELLAGPSVFLFPPKPKDTLMISRTPEVTCVVVDVSHE | |
| | DPEVKFNWYVDGVEVHNAKTKPREEQYNSTYRVVSVLT | |
| | VLHQDWLNGKEYKCKVSNKALPLPEEKTISKAKGQPREP | |
| | QVYTLPPSRDELTKNQVSLTCLVKGFYPSDIAVEWESNG | |
| | QPENNYKTTPPVLDSDGSFFLYSKLTVDKSRWQQGNVFS | |
| | CSVLHEALHSHYTQKSLSLSPGK | |
| 76 | ELQLVESGGGWVQPGGSQRLSCAASGRIFRSFYMSWVR | HC of HBC34-v35-50 |
| | QAPGKGLEWVATINQDGSEKLYVDSVKGRFTISRDNAK | MLNS (g1M17, 1) |
| | NSLFLQMNNLRVEDTAVYYCAAWSGNSGGMDVWGQG | |
| | TTVSVSSASTKGPSVFPLAPSSKSTSGGTAALGCLVKDYF | |
| | PEPVTVSWNSGALTSGVHTFPAVLQSSGLYSLSSVVTVPS | |
| | SSLGTQTYICNVNHKPSNTKVDKKVEPKSCDKTHTCPPC | |
| | PAPELLGGPSVFLFPPKPKDTLMISRTPEVTCVVVDVSHE | |
| | DPEVKFNWYVDGVEVHNAKTKPREEQYNSTYRVVSVLT | |
| | VLHQDWLNGKEYKCKVSNKALPAPIEKTISKAKGQPREP | |
| | QVYTLPPSRDELTKNQVSLTCLVKGFYPSDIAVEWESNG | |
| | QPENNYKTTPPVLDSDGSFFLYSKLTVDKSRWQQGNVFS | |
| | CSVLHEALHSHYTQKSLSLSPGK | |
| 77 | ELQLVESGGGWVQPGGSQRLSCAASGRIFRSFYMSWVRQA | HBC34-v35-50 |
| | PGKGLEWVATINQDGSEKLYVDSVKGRFTISRDNAKNSLFL | HC with GAALIE |
| | QMNNLRVEDTAVYYCAAWSGNSGGMDVWGQGTTVSVSS | mutation in hIgG1 Fc |
| | ASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLAGPSVF | |
| | LFPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALPLPEEKTISKAKGQPREPQVYTLPPSRDELTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| 78 | ELQLVESGGGWVQPGGSQRLSCAASGRIFRSFYMSWVR | HBC34-V35-50 |
| | QAPGKGLEWVATINQDGSEKLYVDSVKGRFTISRDNAK | HC (wild-type) |
| | NSLFLQMNNLRVEDTAVYYCAAWSGNSGGMDVWGQG | |
| | TTVSVSSASTKGPSVFPLAPSSKSTSGGTAALGCLVKDYF | |
| | PEPVTVSWNSGALTSGVHTFPAVLQSSGLYSLSSVVTVPS | |
| | SSLGTQTYICNVNHKPSNTKVDKKVEPKSCDKTHTCPPC | |
| | PAPELLGGPSVFLFPPKPKDTLMISRTPEVTCVVVDVSHE | |
| | DPEVKFNWYVDGVEVHNAKTKPREEQYNSTYRVVSVLT | |
| | VLHQDWLNGKEYKCKVSNKALPAPIEKTISKAKGQPREP | |
| | QVYTLPPSRDELTKNQVSLTCLVKGFYPSDIAVEWESNG | |
| | QPENNYKTTPPVLDSDGSFFLYSKLTVDKSRWQQGNVFS | |
| | CSVMHEALHNHYTQKSLSLSPGK | |
| 79 | GQPKAAPSVTLFPPSSEELQANKATLVCLISDFYPGAVTV | HBC antibody light |
| | AWKADSSPVKAGVETTTPSKQSNNKYAASSYLSLTPEQ | chain constant region |
| | WKSHRSYSCQVTHEGSTVEKTVAPTECS | |
| 80 | GAACTGCAGCTGGTGGAGTCTGGGGGAGGCTGGGTCC | HBC34 VH nuc |
| | AGCCGGGGGGGTCCCAGAGACTGTCCTGTGCAGCCTC | |
| | TGGACGCATCTTTAGAAGTTTTTACATGAGCTGGGTCC | |
| | GCCAGGCCCCAGGGAAGGGGCTGGAGTGGGTGGCCAC | |
| | TATAAACCAAGATGGAAGTGAGAAATTATATGTGGAC | |
| | TCTGTGAAGGGCCGATTCACCATCTCCAGAGACAACG | |
| | CCAAGAACTCACTATTTCTGCAAATGAACAACCTGAG | |
| | AGTCGAGGACACGGCCGTTTATTACTGCGCGGCTTGGA | |
| | GCGGCAATAGTGGGGGTATGGACGTCTGGGGCCAGGG | |
| | GACCACGGTCTCCGTCTCCTCA | |
| 81 | GAGGTGCAGCTGGTGGAATCCGGCGGGGGACTGGTGC | HBC34-v35-v50 VH |
| | AGCCTGGCGGCTCACTGAGACTGAGCTGTGCAGCTTCT | (nuc) |
| | GGAAGAATCTTCAGATCTTTTTACATGAGTTGGGTGAG | |
| | ACAGGCTCCTGGGAAGGGACTGGAGTGGGTCGCAAAC | |
| | ATCAATCAGGACGGATCAGAAAAGCTGTATGTGGATA | |
| | GCGTCAAAGGCAGGTTCACTATTTCCCGCGACAACGCC | |
| | AAAAATTCTCTGTTTCTGCAGATGAACAATCTGCGGGT | |
| | GGAGGATACCGCTGTCTACTATTGTGCAGCCTGGTCTG | |
| | GCAACAGTGGAGGCATGGACGTGTGGGGACAGGGAAC | |
| | CACAGTGACAGTCAGCTCC | |
| 82 | GAACTGCAGCTGGTCGAATCAGGAGGAGGGTGGGTCC | HBC34 wt |
| | AGCCCGGAGGGAGCCAGAGACTGTCTTGTGCCGCATC | VH codon optimized |
| | AGGGAGGATCTTCAGGAGCTTCTACATGTCCTGGGTGC | |
| | GCCAGGCACCAGGCAAGGGACTGGAGTGGGTCGCCAC | |
| | CATCAACCAGGACGGATCTGAAAAGCTGTATGTGGAT | |
| | AGTGTCAAAGGCCGGTTCACAATTAGCAGAGACAACG | |
| | CTAAAAATTCTCTGTTTCTGCAGATGAACAATCTGCGA | |
| | GTGGAGGATACCGCCGTCTACTATTGCGCCGCTTGGTC | |
| | TGGCAACAGCGGCGGGATGGATGTCTGGGGGCAGGGC | |
| | ACAACAGTGAGCGTCTCTTCC | |
| 83 | GCCTCCACAAAGGGCCCAAGCGTGTTTCCACTGGCTCCCTCT | HBC34-V35-50 CH1- |
| | TCCAAGTCTACCTCCGGCGGCACAGCCGCTCTGGGATGTCTG | hinge-CH2-CH3 |
| | GTGAAGGATTACTTCCCAGAGCCCGTGACCGTGTCTTGGAA | (codon-optimized) |
| | CTCCGGCGCCCTGACCAGCGGAGTGCATACATTTCCAGCTGT | |
| | GCTGCAGAGCTCTGGCCTGTACTCTCTGTCCAGCGTGGTGAC | |
| | CGTGCCCTCTTCCAGCCTGGGCACCCAGACATATATCTGCAA | |
| | CGTGAATCACAAGCCAAGCAATACAAAGGTGGACAAGAAG | |
| | GTGGAGCCCAAGTCTTGTGATAAGACCCATACATGCCCTCC | |
| | ATGTCCAGCTCCAGAGCTGCTGGGCGGCCCAAGCGTGTTCCT | |
| | GTTTCCACCCAAGCCTAAGGATACCCTGATGATCTCCAGAAC | |
| | CCCCGAGGTGACATGCGTGGTGGTGGACGTGAGCCACGAGG | |
| | ATCCTGAGGTGAAGTTCAACTGGTACGTGGACGGCGTGGAG | |
| | GTGCATAATGCTAAGACCAAGCCCAGGGAGGAGCAGTACAA | |
| | CTCTACCTATCGGGTGGTGTCCGTGCTGACAGTGCTGCACCA | |
| | GGATTGGCTGAACGGCAAGGAGTATAAGTGCAAGGTGTCTA | |
| | ATAAGGCCCTGCCCGCTCCTATCGAGAAGACCATCTCCAAG | |
| | GCCAAGGGCCAGCCTAGAGAGCCACAGGTGTACACACTGCC | |
| | TCCATCTCGCGATGAGCTGACCAAGAACCAGGTGTCCCTGA | |
| | CATGTCTGGTGAAGGGCTTCTATCCTTCCGACATCGCTGTGG | |
| | AGTGGGAGAGCAATGGCCAGCCAGAGAACAATTACAAGAC | |
| | CACACCCCCTGTGCTGGACAGCGATGGCTCTTTCTTTCTGTA | |
| | TAGCAAGCTGACCGTGGACAAGTCTCGCTGGCAGCAGGGCA | |
| | ACGTGTTTAGCTGTTCTGTGATGCATGAGGCCCTGCACAATC | |
| | ATTATACACAGAAGTCCCTGAGCCTGTCTCCTGGCAAG | |
| 84 | GAGCTGCAGCTGGTGGAGTCCGGCGGCGGCTGGGTGCAGCC | HBC34-V35-50 VH- |
| | TGGCGGCTCCCAGAGGCTGAGCTGTGCCGCTTCTGGCAGGA | CH1-hinge-CH2-CH3 |
| | TCTTCCGGTCCTTTTACATGTCTTGGGTGCGGCAGGCTCCAG | (codon-optimized) |
| | GCAAGGGCCTGGAGTGGGTGGCTACCATCAACCAGGACGGC | |
| | TCCGAGAAGCTGTATGTGGATAGCGTGAAGGGCAGATTCAC | |
| | AATCTCTCGCGACAACGCCAAGAACTCCCTGTTTCTGCAGAT | |
| | GAACAATCTGAGGGTGGAGGATACCGCCGTGTACTATTGCG | |
| | CCGCTTGGTCTGGCAATAGCGGCGGCATGGACGTGTGGGGA | |
| | CAGGGCACCACCGTGTCCGTGTCCAGCGCCTCCACAAAGGG | |
| | CCCAAGCGTGTTTCCACTGGCTCCCTCTTCCAAGTCTACCTC | |
| | CGGCGGCACAGCCGCTCTGGGATGTCTGGTGAAGGATTACT | |
| | TCCCAGAGCCCGTGACCGTGTCTTGGAACTCCGGCGCCCTGA | |
| | CCAGCGGAGTGCATACATTTCCAGCTGTGCTGCAGAGCTCTG | |
| | GCCTGTACTCTCTGTCCAGCGTGGTGACCGTGCCCTCTTCCA | |
| | GCCTGGGCACCCAGACATATATCTGCAACGTGAATCACAAG | |
| | CCAAGCAATACAAAGGTGGACAAGAAGGTGGAGCCCAAGT | |
| | CTTGTGATAAGACCCATACATGCCCTCCATGTCCAGCTCCAG | |
| | AGCTGCTGGGCGGCCCAAGCGTGTTCCTGTTTCCACCCAAGC | |
| | CTAAGGATACCCTGATGATCTCCAGAACCCCCGAGGTGACA | |
| | TGCGTGGTGGTGGACGTGAGCCACGAGGATCCTGAGGTGAA | |
| | GTTCAACTGGTACGTGGACGGCGTGGAGGTGCATAATGCTA | |
| | AGACCAAGCCCAGGGAGGAGCAGTACAACTCTACCTATCGG | |
| | GTGGTGTCCGTGCTGACAGTGCTGCACCAGGATTGGCTGAA | |
| | CGGCAAGGAGTATAAGTGCAAGGTGTCTAATAAGGCCCTGC | |
| | CCGCTCCTATCGAGAAGACCATCTCCAAGGCCAAGGGCCAG | |
| | CCTAGAGAGCCACAGGTGTACACACTGCCTCCATCTCGCGA | |
| | TGAGCTGACCAAGAACCAGGTGTCCCTGACATGTCTGGTGA | |
| | AGGGCTTCTATCCTTCCGACATCGCTGTGGAGTGGGAGAGC | |
| | AATGGCCAGCCAGAGAACAATTACAAGACCACACCCCCTGT | |
| | GCTGGACAGCGATGGCTCTTTCTTTCTGTATAGCAAGCTGAC | |
| | CGTGGACAAGTCTCGCTGGCAGCAGGGCAACGTGTTTAGCT | |
| | GTTCTGTGATGCATGAGGCCCTGCACAATCATTATACACAGA | |
| | AGTCCCTGAGCCTGTCTCCTGGCAAGTGATGAGGTACCGTGC | |
| | GACGGCCGGCAAGCCCCCGCTCCCCGGGCTCTCGCGGTCGT | |
| | ACGAGGAAAGCTT | |
| 85 | AGCTGACACAGCCCCCTTCCGTGTCCGTGTCCCCTGGA | HBC34-v36 VL nt |
| | CAGACCGTGTCCATCCCATGCAGCGGCGACAAGCTGG | |
| | GCAACAAGAACGTGGCCTGGTTTCAGCATAAGCCTGG | |
| | CCAGTCCCCCGTGCTGGTCATCTACCAGGACTCCAAGA | |
| | GGCCCAGCGGCATCCCTGAGCGGTTCTCTGGCTCCAAC | |
| | AGCGGCAATACAGCCACCCTGACAATCTCTGGCACAC | |
| | AGGCTATGGACGAGGCCGCTTATTTCTGCCAGACCTTT | |
| | GATTCCACCACAGTGGTGTTCGGCGGCGGCACCAGAC | |
| | TGACAGTGCTGGGTCAGCCCAAGGCTGCCCCCTCGGTC | |
| | ACTCTG | |
| 86 | TCCTACGAGCTGACACAGCCACCTTCCGTGAGCGTGTCTCCA | HBC34-v37 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACGAGGATTCTAAGAGGCCTTCCG | |
| | GCATCCCAGAGCGGTTTTCCGGCAGCAACTCTGGCAATACC | |
| | GCCACACTGACCATCAGCGGCACACAGGCTATGGACGAGGC | |
| | CGCTTATTTCTGTCAGACCTTTGATTCTACCACAGTGGTGTTC | |
| | GGCGGCGGCACAAGGCTGACCGTGCTG | |
| 87 | TCCTACGAGCTGACACAGCCACCTTCCGTGAGCGTGTCTCCA | HBC34-v38 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACCAGGTGTCTAAGAGGCCTTCCG | |
| | GCATCCCAGAGCGGTTTTCCGGCAGCAACTCTGGCAATACC | |
| | GCCACACTGACCATCAGCGGCACACAGGCTATGGACGAGGC | |
| | CGCTTATTTCTGTCAGACCTTTGATTCTACCACAGTGGTGTTC | |
| | GGCGGCGGCACAAGGCTGACCGTGCTG | |
| 88 | TCCTACGAGCTGACACAGCCACCTTCCGTGAGCGTGTCTCCA | HBC34-v39 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACCAGGATAAGAAGAGGCCTTCC | |
| | GGCATCCCAGAGCGGTTTTCCGGCAGCAACTCTGGCAATAC | |
| | CGCCACACTGACCATCAGCGGCACACAGGCTATGGACGAGG | |
| | CCGCTTATTTCTGTCAGACCTTTGATTCTACCACAGTGGTGTT | |
| | CGGCGGCGGCACAAGGCTGACCGTGCTG | |
| 89 | TCCTACGAGCTGACACAGCCACCTTCCGTGAGCGTGTCTCCA | HBC34-v40 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACCAGGATTCTTATAGGCCTTCCG | |
| | GCATCCCAGAGCGGTTTTCCGGCAGCAACTCTGGCAATACC | |
| | GCCACACTGACCATCAGCGGCACACAGGCTATGGACGAGGC | |
| | CGCTTATTTCTGTCAGACCTTTGATTCTACCACAGTGGTGTTC | |
| | GGCGGCGGCACAAGGCTGACCGTGCTG | |
| 90 | TCCTACGAGCTGACACAGCCACCTTCCGTGAGCGTGTCTCCA | HBC34-v41 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACCAGGTGTCTTATAGGCCTTCCG | |
| | GCATCCCAGAGCGGTTTTCCGGCAGCAACTCTGGCAATACC | |
| | GCCACACTGACCATCAGCGGCACACAGGCTATGGACGAGGC | |
| | CGCTTATTTCTGTCAGACCTTTGATTCTACCACAGTGGTGTTC | |
| | GGCGGCGGCACAAGGCTGACCGTGCTG | |
| 91 | TCTTACGAGCTGACACAGCCACCTTCCGTGAGCGTGTCTCCA | HBC34-v42 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACGAGGTGTCTTATAGGCCTTCCG | |
| | GCATCCCAGAGCGGTTTAGCGGCGCCAACTCTGGCAATACC | |
| | GCTACACTGACCATCTCCGGCACACAGGCTATGGACGAGGC | |
| | CGCTTATTTCTGTCAGACCTTTGATAGCACCACAGTGGTGTT | |
| | CGGCGGCGGCACAAGGCTGACCGTGCTG | |
| 92 | TCCTACGAGCTGACACAGCCACCTTCCGTGAGCGTGTCTCCA | HBC34-v43 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACCAGGTGAAGTATAGGCCTTCCG | |
| | GCATCCCAGAGCGGTTTTCCGGCAGCAACTCTGGCAATACC | |
| | GCCACACTGACCATCAGCGGCACACAGGCTATGGACGAGGC | |
| | CGCTTATTTCTGTCAGACCTTTGATTCTACCACAGTGGTGTTC | |
| | GGCGGCGGCACAAGGCTGACCGTGCTG | |
| 93 | TCCTACGAGCTGACACAGCCACCTTCCGTGAGCGTGTCTCCA | HBC34-v44 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACGCTGTGAAGTATAGGCCTTCCG | |
| | GCATCCCAGAGCGGTTTTCCGGCAGCAACTCTGGCAATACC | |
| | GCCACACTGACCATCAGCGGCACACAGGCTATGGACGAGGC | |
| | CGCTTATTTCTGTCAGACCTTTGATTCTACCACAGTGGTGTTC | |
| | GGCGGCGGCACAAGGCTGACCGTGCTG | |
| 94 | TCCTACGAGCTGACACAGCCACCTTCCGTGAGCGTGTCTCCA | HBC34-v45 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACGAGGTGAAGTATAGGCCTTCCG | |
| | GCATCCCAGAGAACTTTTCCGGCAGCAACTCTGGCAATACC | |
| | GCCACACTGACCATCAGCGGCACACAGGCTATGGACGAGGC | |
| | CGCTTATTTCTGTCAGACCTTTGATTCTACCACAGTGGTGTTC | |
| | GGAGGAGGAACAAGGCTGACCGTGCTG | |
| 95 | TCCTACGAGCTGACACAGCCACCTTCCGTGAGCGTGTCTCCA | HBC34-v46 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACGAGGTGAAGTATAGGCCTTCCG | |
| | GCATCCCAGAGGCTTTTTCCGGCAGCAACTCTGGCAATACCG | |
| | CCACACTGACCATCAGCGGCACACAGGCTATGGACGAGGCC | |
| | GCTTATTTCTGTCAGACCTTTGATTCTACCACAGTGGTGTTC | |
| | GGAGGAGGAACAAGGCTGACCGTGCTG | |
| 96 | TCCTACGAGCTGACACAGCCACCTTCCGTGAGCGTGTCTCCA | HBC34-v49 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACGAGGTGTCTTATAGGCCTTCCG | |
| | GCATCCCAGAGCGGTTTTCCGGCAGCAACTCTGGCAATACC | |
| | GCCACACTGACCATCAGCGGCACACAGGCTATGGACGAGGC | |
| | CGCTTATTTCTGTCAGACCTTTGATTCTACCACAGTGGTGTTC | |
| | GGCGGCGGCACAAGGCTGACCGTGCTG | |
| 97 | TCCTACGAGCTGACACAGCCACCTTCCGTGAGCGTGTCTCCA | HBC34-v50 VL nt |
| | GGACAGACCGTGTCCATCCCTTGCAGCGGCGACAAGCTGGG | |
| | CAACAAGAATGTGGCCTGGTTCCAGCACAAGCCAGGCCAGT | |
| | CCCCCGTGCTGGTCATCTACGAGGTGAAGTATAGGCCTTCCG | |
| | GCATCCCAGAGAAGTTTTCCGGCAGCAACTCTGGCAATACC | |
| | GCCACACTGACCATCAGCGGCACACAGGCTATGGACGAGGC | |
| | CGCTTATTTCTGTCAGACCTTTGATTCTACCACAGTGGTGTTC | |
| | GGAGGAGGAACAAGGCTGACCGTGCTG | |
| 98 | GGACAGCCAAAGGCTGCTCCATCTGTGACCCTGTTTCCACCC | HBC34-V35-50 CL |
| | TCTTCCGAGGAGCTGCAGGCCAACAAGGCCACCCTGGTGTG | (codon-optimized)_1 |
| | CCTGATCTCTGACTTCTACCCTGGAGCTGTGACAGTGGCTTG | |
| | GAAGGCTGATAGCTCTCCCGTGAAGGCTGGCGTGGAGACAA | |
| | CAACCCCTAGCAAGCAGTCTAACAATAAGTACGCCGCTTCC | |
| | AGCTATCTGTCTCTGACACCAGAGCAGTGGAAGTCCCACCG | |
| | CTCTTATTCCTGCCAGGTGACCCATGAGGGCAGCACCGTGG | |
| | AGAAGACAGTGGCCCCCACCGAGTGTTCT | |
| 99 | GGACAGCCAAAGGCTGCTCCATCTGTGACCCTGTTTCCACCC | HBC34-V35-50 |
| | TCTTCCGAGGAGCTGCAGGCCAACAAGGCCACCCTGGTGTG | CL (codon- |
| | CCTGATCTCTGACTTCTACCCTGGAGCTGTGACAGTGGCTTG | optimized)_2 |
| | GAAGGCTGATAGCTCTCCCGTGAAGGCTGGCGTGGAGACAA | |
| | CAACCCCTAGCAAGCAGTCTAACAATAAGTACGCCGCTTCC | |
| | AGCTATCTGTCTCTGACACCAGAGCAGTGGAAGTCCCACCG | |
| | CTCTTATTCCTGCCAGGTGACCCATGAGGGCAGCACCGTGG | |
| | AGAAGACAGTGGCCCCCACCGAGTGTTCT | |
| 100 | XGSSTTSTGPCRTCMTXPSDGNATAIPIPSSWX | peptide |
| | wherein the residues coded as X were substituted with Cysteines | |
| 101 | TSTGPCRTCMTTAQG | peptide |
| 102 | GMLPVCPLIPGSSTTSTGPCRTCMTT | peptide |
| 103 | XSMYPSASATKPSDGNXTGPCRTCMTTAQGTSX | peptide |
| | wherein the residues coded as X were substituted with Cysteines | |
| 104 | PCRTCMTTAQG | amino acids 120 - 130 |
| | | of the S domain of |
| | | HBsAg (HBV-D |
| | | J02203 |
| 105 | PCX₁TCX₂X₃X₄AQG, | epitope |
| | wherein X₁ is R or K, | |
| | X₂ is M or T, | |
| | X₃ is T or I, and | |
| | X₄ is T, P or L | |
| 106 | GGSGG | linker |
| 107 | TGPCRTC | epitope |
| 108 | GNCTCIP | epitope |
| 109 | CCIPIPSSWAFGCSTTSTGPCRTCC | discontinuous epitope |
| | wherein in particular the cysteines at positions 2, 21, and 24 are | mimic |
| | coupled to acetamidomethyl. | |
| 110 | CGNCTCIPIPSSWAFCSTTSTGPCRTCC | discontinuous epitope |
| | wherein in particular the cysteines at positions 4, 6, 24, and 27 are | mimic |
| | coupled to acetamidomethyl. | |
| ill | CGGGCSTTSTGPCRTCC | looped epitope mimic |
| | wherein in particular the cysteines at positions 13 and 16 are | |
| | coupled to acetamidomethyl. | |
| 112 | STTSTGPCRTC | epitope |
| 113 | GNCTCIPIPSSWAFC | epitope |
| 114 | GNCTCIPIPSSWAF | epitope |
| 115 | PCRXC | epitope |
| 116 | aattccacaa ccttccacca aactctgcaa gatcccagag tgagaggcct gtatttccct | NC_003977.2 |
| | gctggtggct ccagttcagg aacagtaaac cctgttctga ctactgcctc tcccttatcg | |
| | tcaatcttct cgaggattgg ggaccctgcg ctgaacatgg agaacatcac atcaggattc | |
| | ctaggacccc ttctcgtgtt acaggcgggg tttttcttgt tgacaagaat cctcacaata | |
| | ccgcagagtc tagactcgtg gtggacttct ctcaattttc tagggggaac taccgtgtgt | |
| | cttggccaaa attcgcagtc cccaacctcc aatcactcac caacctcttg tcctccaact | |
| | tgtcctggtt atcgctggat gtgtctgcgg cgttttatca tcttcctctt catcctgctg | |
| | ctatgcctca tcttcttgtt ggttcttctg gactatcaag gtatgttgcc cgtttgtcct | |
| | ctaattccag gatcctcaac aaccagcacg ggaccatgcc ggacctgcat gactactgct | |
| | caaggaacct ctatgtatcc ctcctgttgc tgtaccaaac cttcggacgg aaattgcacc | |
| | tgtattccca tcccatcatc ctgggctttc ggaaaattcc tatgggagtg ggcctcagcc | |
| | cgtttctcct ggctcagttt actagtgcca tttgttcagt ggttcgtagg gctttccccc | |
| | actgtttggc tttcagttat atggatgatg tggtattggg ggccaagtct gtacagcatc | |
| | ttgagtccct ttttaccgct gttaccaatt ttcttttgtc tttgggtata catttaaacc ctaacaaaac | |
| | aaagagatgg ggttactctc taaattttat gggttatgtc attggatgtt atgggtcctt | |
| | gccacaagaa cacatcatac aaaaaatcaa agaatgtttt agaaaacttc ctattaacag | |
| | gcctattgat tggaaagtat gtcaacgaat tgtgggtctt ttgggttttg ctgccccttt | |
| | tacacaatgt ggttatcctg cgttgatgcc tttgtatgca tgtattcaat ctaagcaggc tttcactttc | |
| | tcgccaactt acaaggcctt tctgtgtaaa caatacctga acctttaccc cgttgcccgg | |
| | caacggccag gtctgtgcca agtgtttgct gacgcaaccc ccactggctg gggcttggtc | |
| | atgggccatc agcgcatgcg tggaaccttt tcggctcctc tgccgatcca tactgcggaa | |
| | ctcctagccg cttgttttgc tcgcagcagg tctggagcaa acattatcgg gactgataac | |
| | tctgttgtcc tatcccgcaa atatacatcg tttccatggc tgctaggctg tgctgccaac | |
| | tggatcctgc gcgggacgtc ctttgtttac gtcccgtcgg cgctgaatcc tgcggacgac | |
| | ccttctcggg gtcgcttggg actctctcgt ccccttctcc | |
| | gtctgccgtt ccgaccgacc acggggcgca cctctcttta cgcggactcc ccgtctgtgc | |
| | cttctcatct gccggaccgt gtgcacttcg cttcacctct gcacgtcgca tggagaccac | |
| | cgtgaacgcc caccaaatat tgcccaaggt cttacataag aggactcttg gactctcagc | |
| | aatgtcaacg accgaccttg aggcatactt caaagactgt ttgtttaaag actgggagga | |
| | gttgggggag gagattaggt taaaggtctt tgtactagga ggctgtaggc ataaattggt | |
| | ctgcgcacca gcaccatgca actttttcac ctctgcctaa tcatctcttg ttcatgtcct | |
| | actgttcaag cctccaagct gtgccttggg tggctttggg gcatggacat cgacccttat | |
| | aaagaatttg gagctactgt ggagttactc tcgtttttgc cttctgactt ctttccttca gtacgagatc | |
| | ttctagatac cgcctcagct ctgtatcggg aagccttaga gtctcctgag cattgttcac | |
| | ctcaccatac tgcactcagg caagcaattc tttgctgggg ggaactaatg actctagcta | |
| | cctgggtggg tgttaatttg gaagatccag cgtctagaga cctagtagtc agttatgtca | |
| | acactaatat gggcctaaag ttcaggcaac tcttgtggtt tcacatttct tgtctcactt | |
| | ttggaagaga aacagttata gagtatttgg tgtctttcgg agtgtggatt cgcactcctc | |
| | cagcttatag accaccaaat gcccctatcc tatcaacact tccggagact actgttgtta | |
| | gacgacgagg caggtcccct agaagaagaa ctccctcgcc tcgcagacga | |
| | aggtctcaat cgccgcgtcg cagaagatct caatctcggg aatctcaatg ttagtattcc | |
| | ttggactcat aaggtgggga actttactgg gctttattct tctactgtac ctgtctttaa | |
| | tcctcattgg aaaacaccat cttttcctaa tatacattta caccaagaca ttatcaaaaa | |
| | atgtgaacag tttgtaggcc cactcacagt taatgagaaa agaagattgc aattgattat | |
| | gcctgccagg ttttatccaa aggttaccaa atatttacca ttggataagg gtattaaacc | |
| | ttattatcca gaacatctag ttaatcatta cttccaaact agacactatt tacacactct | |
| | atggaaggcg ggtatattat ataagagaga aacaacacat agcgcctcat tttgtgggtc | |
| | accatattct tgggaacaag atctacagca tggggcagaa tctttccacc agcaatcctc | |
| | tgggattctt tcccgaccac cagttggatc cagccttcag agcaaacacc gcaaatccag | |
| | attgggactt caatcccaac aaggacacct ggccagacgc caacaaggta ggagctggag | |
| | cattcgggct gggtttcacc ccaccgcacg gaggcctttt ggggtggagc | |
| | cctcaggctc agggcatact acaaactttg ccagcaaatc cgcctcctgc | |
| | ctccaccaat cgccagtcag gaaggcagcc taccccgctg tctccacctt | |
| | tgagaaacac tcatcctcag gccatgcagt gg | |
| 117 | GTGTGCACTTCGCTTCAC | 1579-1597 of |
| | | NC_003977.2 |
| 118 | GUGUGCACUUCGCUUCACA | HBV001 sense |
| 119 | UGUGAAGCGAAGUGCACACUU | HBV001 antisense |
| 120 | GGUGGACUUCUCUCAAUUUUA | HBV003 sense |
| 121 | UAAAAUUGAGAGAAGUCCACCAC | HBV003 antisense |
| 122 | gsusguGfcAfCfUfucgcuucacaL96 | HBV002v2 sense |
| 123 | usGfsugaAfgCfGfaaguGfcAfcacsusu | HBV002v2 antisense |
| 124 | gsusguGfcAfCfUfucgcuucacaL96 | HBV002v1 sense |
| 125 | usGfsuga(Agn)gCfGfaaguGfcAfcacsusu | HBV002v1 antisense |
| 126 | gsgsuggaCfuUfCfUfcucaAfUfuuuaL96 | No. 126 sense |
| 127 | usAfsaaaUfuGfAfgagaAfgUfccaccsasc | No. 127 antisense |
| 128 | AAVALLPAVLLALLAP | RFGF |
| 129 | AALLPVLLAAP | RFGF analogue |
| 130 | GRKKRRQRRRPPQ | HIV Tat protein |
| 131 | RQIKIWFQNRRMKWK | Drosophila |
| | | Antennapedia protein |
| 132 | Gly Gln Ser Pro Val Leu Val Ile Tyr Glu Val Lys Tyr Arg Pro Ser | Synthetic sequence |
| | Gly Ile Pro Glu Arg Phe Ser Gly Ser Asn | CDR framework |
| 133 | Pro Ser Gly Ile Pro Glu Arg Phe Ser Gly Ser Asn Ser Gly Asn Thr | Synthetic sequence |
| | Ala Thr Leu Thr Ile Ser Gly Thr Gln Ala Met Asp Glu Ala Ala | CDR framework |
| 134 | Gly Gln Ser Pro Val Leu Val Ile Tyr Glu Val Lys Tyr Arg Pro Ser | Synthetic sequence |
| | Gly Ile Pro Glu Arg Phe Ser Gly Ser Asn Ser Gly Asn Thr Ala Thr | HBC34 |
| | Leu Thr Ile Ser Gly Thr Gln Ala Met Asp Glu Ala Ala Tyr Phe | |
| | Cys | |
| 135 | Gly Gln Ser Pro Val Leu Val Ile Tyr Gln Asp Ser Lys Arg Pro Ser | Synthetic sequence |
| | Gly Ile Pro Glu Arg Phe Ser Gly Ser Asn Ser Gly Asn Thr Ala Thr | GLL2 |
| | Leu Thr Ile Ser Gly Thr Gln Ala Met Asp Glu Ala Ala Tyr Phe | |
| | Cys | |
| 136 | Gly Gln Ser Pro Val Leu Val Ile Tyr Glu Asp Ser Lys Arg Pro Ser | Synthetic sequence |
| | Gly Ile Pro Glu Arg Phe Ser Gly Ser Asn Ser Gly Asn Thr Ala Thr | v36 + Q49E |
| | Leu Thr Ile Ser Gly Thr Gln Ala Met Asp Glu Ala Ala Tyr Phe | |
| | Cys | |
| 137 | Gly Gln Ser Pro Val Leu Val Ile Tyr Gln Val Ser Lys Arg Pro Ser | Synthetic sequence |
| | Gly Ile Pro Glu Arg Phe Ser Gly Ser Asn Ser Gly Asn Thr Ala Thr | v36 + D50V |
| | Leu Thr Ile Ser Gly Thr Gln Ala Met Asp Glu Ala Ala Tyr Phe | |
| | Cys | |
| 138 | Gly Gln Ser Pro Val Leu Val Ile Tyr Gln Asp Lys Lys Arg Pro Ser | Synthetic sequence |
| | Gly Ile Pro Glu Arg Phe Ser Gly Ser Asn Ser Gly Asn Thr Ala | v36 + S51K |
| | Thr Leu Thr Ile Ser Gly Thr Gln Ala Met Asp Glu Ala Ala Tyr | |
| | Phe Cys | |
| 139 | Gly Gln Ser Pro Val Leu Val Ile Tyr Gln Asp Ser Tyr Arg Pro Ser | Synthetic sequence |
| | Gly Ile Pro Glu Arg Phe Ser Gly Ser Asn Ser Gly Asn Thr Ala Thr | v36 + K52Y |
| | Leu Thr Ile Ser Gly Thr Gln Ala Met Asp Glu Ala Ala Tyr Phe | |
| | Cys | |
| 140 | Gly Gln Ser Pro Val Leu Val Ile Tyr Gln Val Ser Tyr Arg Pro Ser | Synthetic sequence |
| | Gly Ile Pro Glu Arg Phe Ser Gly Ser Asn Ser Gly Asn Thr Ala Thr | v36 + D50V + K52Y |
| | Leu Thr Ile Ser Gly Thr Gln Ala Met Asp Glu Ala Ala Tyr Phe | |
| | Cys | |
| 141 | Gly Gln Ser Pro Val Leu Val Ile Tyr Glu Val Ser Tyr Arg Pro Ser | Synthetic sequence |
| | Gly Ile Pro Glu Arg Phe Ser Gly Ala Asn Ser Gly Asn Thr Ala Thr | HBC34 + K51S + |
| | Leu Thr Ile Ser Gly Thr Gln Ala Met Asp Glu Ala Ala Tyr Phe | S64A |
| | Cys | |
| 142 | Gly Gln Ser Pro Val Leu Val Ile Tyr Gln Val Lys Tyr Arg Pro Ser | Synthetic sequence |
| | Gly Ile Pro Glu Arg Phe Ser Gly Ser Asn Ser Gly Asn Thr Ala Thr | HBC34 + E49Q |
| | Leu Thr Ile Ser Gly Thr Gln Ala Met Asp Glu Ala Ala Tyr Phe | |
| | Cys | |
| 143 | Gly Gln Ser Pro Val Leu Val Ile Tyr Ala Val Lys Tyr Arg Pro Ser | Synthetic sequence |
| | Gly Ile Pro Glu Arg Phe Ser Gly Ser Asn Ser Gly Asn Thr Ala Thr | HBC34 + E49A |
| | Leu Thr Ile Ser Gly Thr Gln Ala Met Asp Glu Ala Ala Tyr Phe | |
| | Cys | |
| 144 | Gly Gln Ser Pro Val Leu Val Ile Tyr Glu Val Lys Tyr Arg Pro Ser | Synthetic sequence |
| | Gly Ile Pro Glu Asn Phe Ser Gly Ser Asn Ser Gly Asn Thr Ala Thr | HBC34 + R60N |
| | Leu Thr Ile Ser Gly Thr Gln Ala Met Asp Glu Ala Ala Tyr Phe | |
| | Cys | |
| 145 | Gly Gln Ser Pro Val Leu Val Ile Tyr Glu Val Lys Tyr Arg Pro Ser | Synthetic sequence |
| | Gly Ile Pro Glu Ala Phe Ser Gly Ser Asn Ser Gly Asn Thr Ala Thr | HBC34 + R60A |
| | Leu Thr Ile Ser Gly Thr Gln Ala Met Asp Glu Ala Ala Tyr Phe | |
| | Cys | |
| 146 | Gly Gln Ser Pro Val Leu Val Ile Tyr Glu Val Ser Tyr Arg Pro Ser | Synthetic sequence |
| | Gly Ile Pro Glu Asn Phe Ser Gly Ala Asn Ser Gly Asn Thr Ala | HBC34 + K51S + |
| | Thr Leu Thr Ala Ser Gly Thr Gln Ala Met Asp Glu Ala Ala Tyr | S64A + R60N + |
| | Phe Cys | I74A |
| 147 | Gly Gln Ser Pro Val Leu Val Ile Tyr Glu Val Lys Tyr Arg Pro Ser | Synthetic sequence |
| | Gly Ile Pro Glu Asn Phe Ser Gly Ala Asn Ser Gly Asn Thr Ala | HBC34 + R60N + |
| | Thr Leu Thr Ala Ser Gly Thr Gln Ala Met Asp Glu Ala Ala Tyr | S64A + I74A |
| | Phe Cys | |
| 148 | Gly Gln Ser Pro Val Leu Val Ile Tyr Glu Val Ser Tyr Arg Pro Ser | Synthetic sequence |
| | Gly Ile Pro Glu Arg Phe Ser Gly Ser Asn Ser Gly Asn Thr Ala Thr | HBC34 + K51S |
| | Leu Thr Ile Ser Gly Thr Gln Ala Met Asp Glu Ala Ala Tyr Phe | |
| | Cys | |
| 149 | Gly Gln Ser Pro Val Leu Val Ile Tyr Glu Val Lys Tyr Arg Pro Ser | Synthetic sequence |
| | Gly Ile Pro Glu Lys Phe Ser Gly Ser Asn Ser Gly Asn Thr Ala Thr | HBC34 + R60K |
| | Leu Thr Ile Ser Gly Thr Gln Ala Met Asp Glu Ala Ala Tyr Phe | |
| | Cys | |
| 150 | GRIFRSFY | CDRH1 (IMGT) |
| 151 | QDGSEK | CDRH2 - short |
| | | (IMGT) |
| 152 | INQDGSEK | CDRH2 - long |
| | | (IMGT) |
| 153 | AAWSGNSGGMDV | CDRH3 (IMGT) |
| 154 | KLGNKN | CDRL1 (IMGT) |
| 155 | EVK | HBC34-v35, -v45, |
| | | -v46, -v48, -v50 |
| | | CDRL2 - short |
| 156 | QDS | HBC34-v36, -v40 |
| | | CDRL2 - short |
| | | (IMGT) |
| 157 | EDS | HBC34-v37 CDRL2 - |
| | | short (IMGT) |
| 158 | QVS | HBC34-v38, -v41, |
| | | CDRL2 - short |
| | | (IMGT) |
| 159 | QDK | HBC34-v39 |
| | | CDRL2 - short |
| | | (IMGT) |
| 160 | EVS | HBC34-v42, -v47, |
| | | -v49, -v50 |
| | | CDRL2 - short |
| | | (IMGT) |
| 161 | QVK | HBC34-v43 |
| | | CDRL2 - short |
| | | (IMGT) |
| 162 | AVK | HBC34-v44 |
| | | CDRL2 - short |
| | | (IMGT) |
| 163 | VIYEVKYRPS | HBC34-v35, -v45, |
| | | -v46, -v48, -v50 |
| | | CDRL2 - long |
| 164 | VIYQDSKRPS | HBC34-v36 |
| | | CDRL2 - long |
| | | (IMGT) |
| 165 | VIYEDSKRPS | HBC34-v37 |
| | | CDRL2 - long |
| | | (IMGT) |
| 166 | VIYQVSKRPS | HBC34-v38 |
| | | CDRL2 - long |
| | | (IMGT) |
| 167 | VIYQDKKRPS | HBC34-v39 |
| | | CDRL2 - long |
| | | (IMGT) |
| 168 | VIYQDSYRPS | HBC34-v40 |
| | | CDRL2 - long |
| | | (IMGT) |
| 169 | VIYQVSYRPS | HBC34-v41 |
| | | CDRL2 - long |
| | | (IMGT) |
| 170 | VIYEVSYRPS | HBC34-v42, -v47, - |
| | | v49 |
| | | CDRL2 - long |
| 171 | VIYQVKYRPS | HBC34-v43 |
| | | CDRL2 - long |
| | | (IMGT) |
| 172 | VIYAVKYRPS | HBC34-v44 |
| | | CDRL2 - long |
| | | (IMGT) |
| 173 | QTFDSTTVV | HBC34-v35-v50 |
| | | CDRL3 (IMGT) |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

U.S. Provisional Application 63/043,692, filed Jun. 24, 2020 is incorporated herein by reference, in its entirety.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. (canceled)

2. (canceled)

3. An antibody, or an antigen-binding fragment thereof, comprising
a heavy chain variable region (VH) comprising a CDRH1 amino acid sequence a CDRH2 amino acid sequence, and a CDRH3 amino acid sequence; and
a light chain variable region (VL) comprising a CDRL1 amino acid sequence, a CDRL2 amino acid sequence, and a CDRL3 amino acid sequence, wherein the CDRH1, CDRH2, CDRH3, CDRL1, CDRL2, and CDRL3 amino acid sequences are according to SEQ ID NOs.:
(i) 34, 35, 37, 41, 49, and 55, respectively;
(ii) 34, 35, 37, 41, 46, and 55, respectively;
(iii) 34, 35, 37, 41, 47, and 55, respectively;
(iv) 34, 35, 37, 41, 48, and 55, respectively;
(v) 34, 35, 37, 41, 45, and 55, respectively;
(vi) 34, 35, 37, 41, 50, and 55, respectively;
(vii) 34, 35, 37, 41, 51, and 55, respectively;
(viii) 34, 35, 37, 41, 52, and 55, respectively;
(ix) 34, 35, 37, 41, 53, and 55, respectively; or
(x) 34, 35, 37, 41, 44, and 55, respectively.
wherein CDRs are defined according to the CCG numbering system,
wherein, optionally, the VL comprises a R60N substitution mutation, a R60A substitution mutation, a R60K substitution mutation, a S64A substitution mutation, a I74A substitution mutation, or any combination thereof, relative to SEQ ID NO.:58 and wherein the amino acid numbering of the substitution mutation(s) is according to SEQ ID NO.:58, and still further optionally wherein the VL does not comprise any further mutation(s) relative to SEQ ID NO.:58, and
wherein the antibody or antigen-binding fragment thereof is capable of binding to the antigenic loop region of HBsAg and, optionally, neutralizing infection by a hepatitis B virus (HBV) of genotype D, A, B, C, E, F, G, H, I, or J, or any combination thereof.

4. (canceled)

5. (canceled)

6. (canceled)

7. (canceled)

8. (canceled)

9. The antibody or antigen-binding fragment of claim 3, wherein the VH and the VL comprise or consist of amino acid sequences having at least 90%, 91%, 92%, 93%, 94%, 95%, 96%, 97%, 98%, or 99% identity to the amino acid sequences set forth in SEQ ID NOs.: (i) 38 and 62, respectively; (ii) 38 and 59, respectively; (iii) 38 and 60, respectively; (iv) 38 and 61, respectively; (v) 38 and 58, respectively; (vi) 38 and 63, respectively; (vii) 38 and 64, respectively; (viii) 38 and 65, respectively; (ix) 38 and 66, respectively; (x) 38 and 71, respectively; or (xi) 38 and 72, respectively.

10. (canceled)

11. (canceled)

12. (canceled)

13. The antibody or antigen-binding fragment of claim 3, wherein the VH and the VL comprise or consist of the amino acid sequences set forth in SEQ ID NOs.:
(i) 38 and 62, respectively;
(ii) 38 and 59, respectively;
(iii) 38 and 60, respectively;
(iv) 38 and 61, respectively;
(v) 38 and 58, respectively;
(vi) 38 and 63, respectively;
(vii) 38 and 64, respectively;
(viii) 38 and 65, respectively;
(ix) 38 and 66, respectively;
(x) 38 and 71, respectively; or
(xi) 38 and 72, respectively.

14. An antibody or antigen-binding fragment, comprising a heavy chain variable region (VH) and a light chain variable region (VL), wherein the VH and the VL comprise or consist of the amino acid sequences set forth in SEQ ID NOs.:
(i) 38 and 62, respectively;
(ii) 38 and 66, respectively;
(iii) 38 and 67, respectively;
(iv) 38 and 68, respectively; or
(v) 38 and 72, respectively,
wherein the antibody or antigen-binding fragment thereof is capable of binding to the antigenic loop region of HBsAg and neutralizing infection by a hepatitis B virus (HBV) of genotype D, A, B, C, E, F, G, H, I, or J, or any combination thereof.

15. The antibody or antigen-binding fragment of claim 3, which is capable of neutralizing infection by a hepatitis D virus (HDV).

16. The antibody or antigen-binding fragment of claim 3, wherein, in a sample comprising a plurality of the antibody or antigen-binding fragment, less than 12%, 11% or less, 10% or less, 9% or less, 8% or less, 7% or less, 6% or less, 5% or less, 4% or less, 3% or less, or 2% or less of the plurality is comprised in a dimer when the sample has been incubated for about 120 to about 168 hours at about 40° C., wherein, the presence of dimer is determined by absolute size-exclusion chromatography; and/or wherein incubation of a plurality of the antibody or antigen-binding fragment results in reduced formation of a dimer as compared to incubation of a plurality of a reference antibody or antigen-binding fragment,
wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

17. (canceled)

18. The antibody or antigen-binding fragment of claim 3, which forms a lower amount of dimer, and/or forms dimers at a reduced frequency and/or as a lower percentage of total antibody or antigen-binding fragment molecules in a sample or composition as compared to a reference antibody:
(i) in a 5-day, a 15-day, and/or a 32-day incubation at 4° C.;
(ii) in a 5-day, a 15-day, and/or a 32-day incubation at 25° C.; and/or
(iii) in a 5-day, a 15-day, and/or a 32-day incubation at 40° C.,
wherein the reference antibody or antigen-binding fragment comprises CDRH1, CDRH2, CDRH3, CDRL1, CDRL2, and CDRL3 amino acid sequences according to the amino acid sequences set forth in SEQ ID NOs.:34, 35, 37, 41, 44, and 55, respectively, and optionally comprises the VH amino acid sequence set forth in SEQ ID NO.:38 and the VL amino acid sequence set forth in SEQ ID NO.:57.

19. (canceled)

20. (canceled)

21. (canceled)

22. (canceled)

23. The antibody or antigen-binding fragment of claim 3, wherein the antibody or antigen-binding fragment is capable of binding to a HBsAg (adw) with an EC50 (ng/ml) of about 3.2 or less, less than 3.0, less than 2.5, less than 2.0, less than 1.5, or less than 1.0.

24. (canceled)

25. (canceled)

26. (canceled)

27. (canceled)

28. (canceled)

29. (canceled)

30. The antibody or antigen-binding fragment of claim 3, wherein the antibody, or the antigen-binding fragment thereof, comprises a human antibody, a monoclonal antibody, a purified antibody, a single chain antibody, a Fab, a Fab′, a F(ab′)2, a Fv, or a scFv.

31. The antibody or antigen-binding fragment of claim 3, wherein the antibody or antigen-binding fragment is a multi-specific or bispecific antibody or antigen-binding fragment.

32. (canceled)

33. The antibody, or an antigen-binding fragment thereof of claim 3, wherein the antibody or the antigen-binding fragment comprises a Fc moiety.

34. The antibody or antigen-binding fragment of claim 33, wherein the Fc moiety comprises a mutation that enhances binding to FcRn as compared to a reference Fc moiety that does not comprise the mutation.

35. The antibody or antigen-binding fragment of claim 33, wherein the Fc moiety comprises a mutation that enhances binding to a FcγR, preferably a FcγRIIA and/or a FcγRIIIA, as compared to a reference Fc moiety that does not comprise the mutation.

36. The antibody or antigen-binding fragment of claim 33, wherein the Fc moiety is an IgG isotype, such as IgG1, or is derived from an IgG isotype, such as IgG1.

37. The antibody or antigen-binding fragment of claim 33, which comprises or is derived from Ig G1m17, 1 (IgHG1*01).

38. The antibody or antigen-binding fragment of claim 34, wherein the mutation that enhances binding to FcRn comprises:
(i) M428L/N434S;
(ii) M252Y/S254T/T256E;
(iii) T250Q/M428L;
(iv) P257I/Q311I;
(v) P257I/N434H;
(vi) D376V/N434H;
(vii) T307A/E380A/N434A; or
(viii) any combination of (i)-(vii),
wherein amino acid numbering of the Fc moiety is according to the EU numbering system.

39. The antibody or antigen-binding fragment of claim 38, wherein the mutation that enhances binding to FcRn comprises M428L/N434S.

40. The antibody or antigen-binding fragment of claim 35, wherein the mutation that enhances binding to a FcγR comprises S239D; I332E; A330L; G236A; or any combination thereof, wherein amino acid numbering of the Fc moiety is according to the EU numbering system.

41. The antibody or antigen-binding fragment of claim 40, wherein the mutation that enhances binding to a FcγR comprises:
(i) S239D/I332E;
(ii) S239D/A330L/I332E;
(iii) G236A/S239D/I332E; or
(iv) G236A/A330L/I332E.

42. The antibody or antigen-binding fragment of claim 40, wherein the mutation that enhances binding to a FcγR comprises or consists of G236A/A330L/I332E, and the antibody or antigen-binding fragment does not comprise S239D.

43. The antibody or antigen-binding fragment of claim 33, wherein the Fc moiety comprises the amino acid substitution mutations: M428L; N434S; G236A; A330L; and I332E, and does not comprise S239D.

44. The antibody or antigen-binding fragment of claim 3, comprising a light chain constant region (CL) that comprises or consists of an amino acid sequence having at least 90%, 91%, 92%, 93%, 94%, 95%, 96%, 97%, 98%, 99%, or 100% identity to the amino acid sequence of SEQ ID NO.:79.

45. The antibody or antigen-binding fragment of claim 3, comprising a CH1-CH2-CH3 that comprises or consists of an amino acid sequence having 90%, 91%, 92%, 93%, 94%, 95%, 96%, 97%, 98%, 99%, or 100% identity to the amino acid sequence of SEQ ID NO.:73, or a variant thereof that comprises one or more of the following amino acid substitutions (EU numbering): G236A; A330L; I332E; M428L; N434S.

46. The antibody or antigen-binding fragment of claim 45, wherein the CH1-CH2-CH3 has a C-terminal lysine removed.

47. An antibody comprising: a heavy chain (HC) comprising or consisting of the amino acid sequence set forth in SEQ ID NO.:75, optionally with the C-terminal lysine removed; and a light chain (LC), wherein the LC comprises or consists of (i) the VL amino acid sequence set forth in any one of SEQ ID NOs.:62, 58-61, and 63-72 and (ii) the CL amino acid sequence set forth in SEQ ID NO.:79.

48. The antibody of claim 47, wherein the LC comprises the VL amino acid sequence set forth in any one of SEQ ID NOs.:62, 66, 67, and 72.

49. An antibody comprising: a heavy chain (HC) comprising or consisting of the amino acid sequence set forth in SEQ ID NO.:76, optionally with the C-terminal lysine removed, and a light chain (LC), wherein the LC comprises or consists of (i) the VL amino acid sequence set forth in any one of SEQ ID NOs.:62, 58-61, and 63-72 and (ii) the CL amino acid sequence set forth in SEQ ID NO.:79.

50. The antibody of claim 49, wherein the LC comprises the VL amino acid sequence set forth in any one of SEQ ID NOs.:62, 66, 67, and 72.

51. An antibody comprising: a heavy chain (HC) comprising or consisting of the amino acid sequence set forth in SEQ ID NO.:77, optionally with the C-terminal lysine removed, and a light chain (LC), wherein the LC comprises or consists of (i) the VL amino acid sequence set forth in any one of SEQ ID NOs.:62, 58-61, and 63-72 and (ii) the CL amino acid sequence set forth in SEQ ID NO.:79.

52. The antibody of claim 51, wherein the LC comprises the VL amino acid sequence set forth in any one of SEQ ID NOs.:62, 66, 67, and 72.

53. An antibody comprising: a heavy chain (HC) comprising or consisting of the amino acid sequence set forth in SEQ ID NO.:78, optionally with the C-terminal lysine removed, and a light chain (LC), wherein the LC comprises or consists of (i) the VL amino acid sequence set forth in any one of SEQ ID NOs.:62, 58-61, and 63-72 and (ii) the CL amino acid sequence set forth in SEQ ID NO.:79.

54. The antibody of claim 53, wherein the LC comprises the VL amino acid sequence set forth in any one of SEQ ID NOs.:62, 66, 67, and 72.

55. (canceled)

56. (canceled)

57. (canceled)

58. (canceled)

59. (canceled)

60. A polynucleotide comprising a nucleotide sequence that encodes the antibody, or the antigen-binding fragment, of claim 3.

61. A polynucleotide encoding a light chain variable region (VL) of the antibody, or the antigen-binding fragment, of claim 3.

62. The polynucleotide of claim 60, wherein the nucleotide sequence that encodes the antibody or the antigen-binding fragment is codon optimized for expression in a host cell.

63. The polynucleotide of claim 62, comprising a nucleotide sequence having at least 50% identity to the nucleotide sequence according to any one of SEQ ID Nos.:89, 85-88, and 90-99.

64. The polynucleotide of claim 60, comprising (i) the polynucleotide sequence set forth in SEQ ID NO.:81 or SEQ ID NO.:82, and (ii) the polynucleotide sequence set forth in any one or more of SEQ ID NOs.:89, 85-88, and 90-99.

65. The polynucleotide of claim 60, comprising (i) the polynucleotide sequence set forth in SEQ ID NO.:83, and (ii) the polynucleotide sequence set forth in any one or more of SEQ ID NOs.:89, 85-88, and 90-99.

66. The polynucleotide of claim 60, comprising (i) the polynucleotide sequence set forth in SEQ ID NO.:84, and (ii) the polynucleotide sequence set forth in any one or more of SEQ ID NOs.:89, 85-88, and 90-99.

67. A vector comprising the polynucleotide of claim 60.

68. The vector of claim 67, wherein the vector comprises a lentiviral vector or a retroviral vector.

69. A host cell comprising the polynucleotide of claim 60.

70. A pharmaceutical composition comprising:
the antibody or antigen binding fragment of claim 3,
and a pharmaceutically acceptable excipient, diluent or carrier.

71. A kit comprising:
(a) the antibody or antigen-binding fragment of claim 3; and
(b) (1) instructions for using the component to prevent, treat, attenuate, and/or diagnose a hepatitis B virus infection and/or a hepatitis D virus infection and/or (2) a means for administering the component to the subject, such as a syringe.

72. The composition of claim 70, further comprising:
(i) a polymerase inhibitor, wherein the polymerase inhibitor optionally comprises Lamivudine, Adefovir, Entecavir, Telbivudine, Tenofovir, or any combination thereof;
(ii) an interferon, wherein the interferon optionally comprises IFNbeta and/or IFNalpha;
(iii) a checkpoint inhibitor, wherein the checkpoint inhibitor optionally comprises an anti-PD-1 antibody or antigen binding fragment thereof, an anti-PD-L1 antibody or antigen binding fragment thereof, and/or an anti-CTLA4 antibody or antigen binding fragment thereof;
(iv) an agonist of a stimulatory immune checkpoint molecule; or
(v) any combination of (i)-(iv).

73. (canceled)

74. A method of producing the antibody or antigen binding fragment of, comprising culturing the host cell of claim 69 under conditions and for a time sufficient to produce the antibody or antigen-binding fragment.

75. (canceled)

76. A method of treating, preventing, and/or attenuating a hepatitis B virus and/or hepatitis D virus infection in a subject, comprising administering to the subject an effective amount of the antibody or antigen-binding fragment of claim 3.

77. (canceled)

78. (canceled)

79. (canceled)

80. (canceled)

81. (canceled)

82. (canceled)

83. The method of claim 76, wherein the method comprises administering to the subject a single dose of a pharmaceutical composition comprising the antibody or antigen-binding fragment; and/or the single dose of the pharmaceutical composition comprises the antibody in a range from 2 to 18 mg/kg (subject body weight); and/or the single dose of the pharmaceutical composition comprises the antibody in a range from 2 to 18 mg/kg (subject body weight); and/or the single dose of the pharmaceutical composition comprises up to 6 mg, up to 10 mg, up to 15 mg, up to 18 mg, up to 25 mg, up to 30 mg, up to 35 mg, up to 40 mg, up to 45 mg, up to 50 mg, up to 55 mg, up to 60 mg, up to 75 mg, up to 90 mg, up to 300 mg, up to 900 mg, or up to 3000 mg of the antibody,
or wherein the single dose of the pharmaceutical composition comprises the antibody in an amount that is in a range from 1 mg to 3000 mg, or in a range from 5 mg to 3000 mg, or in a range from 10 mg to 3000 mg, or in a range from 25 mg to 3000 mg, or in a range from 30 mg to 3000 mg, or in a range from 50 mg to 3000 mg, or in a range from 60 mg to 3000 mg, or in a range from 75 mg to 3000 mg, or in a range from 90 mg to 3000 mg, or in a range from 100 mg to 3000 mg, or in a range from 150 mg to 3000 mg, or in a range from 200 mg to 3000 mg, or in a range from 300 mg to 3000 mg, or in a range from 500 mg to 3000 mg, or in a range from 750 mg to 3000 mg, or in a range from 900 mg to 3000 mg, or in a range from 1500 mg to 3000 mg, or in a range from 2000 mg to 3000 mg,
or wherein the single dose of the pharmaceutical composition comprises the antibody in an amount that is in a range from 1 mg to 900 mg, or in a range from 5 mg to 900 mg, or in a range from 10 mg to 900 mg, or in a range from 25 mg to 900 mg, or in a range from 30 mg to 900 mg, or in a range from 50 mg to 900 mg, or in a range from 60 mg to 900 mg, or in a range from 75 mg to 900 mg, or in a range from 90 mg to 900 mg, or in a range from 100 mg to 900 mg, or in a range from 150 mg to 900 mg, or in a range from 200 mg to 900 mg, or in a range from 300 mg to 900 mg, or in a range from 500 mg to 900 mg, or in a range from 750 mg to 900 mg,
or wherein the single dose of the pharmaceutical composition comprises the antibody in an amount that is wherein the single dose of the pharmaceutical composition comprises the antibody in an amount that is in a range from 1 mg to 500 mg, or in a range from 5 mg to 500 mg, or in a range from 10 mg to 500 mg, or in a range from 25 mg to 500 mg, or in a range from 30 mg to 500 mg, or in a range from 50 mg to 500 mg, or in a range from 60 mg to 500 mg, or in a range from 75 mg to 500 mg, or in a range from 90 mg to 500 mg, or in a range from 100 mg to 500 mg, or in a range from 150 mg to 500 mg, or in a range from 200 mg to 500 mg, or in a range from 300 mg to 500 mg, or in a range from 400 mg to 500 mg,
or wherein the single dose of the pharmaceutical composition comprises the antibody in an amount that is in a range from 1 mg to 300 mg, or in a range from 5 mg to 300 mg, or in a range from 10 mg to 300 mg, or in a range from 25 mg to 300 mg, or in a range from 30 mg to 300 mg, or in a range from 50 mg to 300 mg, or in a range from 60 mg to 300 mg, or in a range from 75 mg to 300 mg, or in a range from 90 mg to 300 mg, or in a range from 100 mg to 300 mg, or in a range from 150 mg to 300 mg, or in a range from 200 mg to 300 mg,
or wherein the single dose of the pharmaceutical composition comprises the antibody in an amount that is in a range from 1 mg to 200 mg, or in a range from 5 mg to 200 mg, or in a range from 10 mg to 200 mg, or in a range from 25 mg to 200 mg, or in a range from 30 mg to 200 mg, or in a range from 50 mg to 200 mg, or in a range from 60 mg to 200 mg, or in a range from 75 mg to 200 mg, or in a range from 90 mg to 200 mg, or in a range from 100 mg to 200 mg, or in a range from 150 mg to 200 mg,
or wherein the single dose of the pharmaceutical composition comprises the antibody in an amount that is in a range from 1 mg to 100 mg, or in a range from 5 mg to 100 mg, or in a range from 10 mg to 100 mg, or in a range from 25 mg to 100 mg, or in a range from 30 mg to 100 mg, or in a range from 50 mg to 100 mg, or in a range from 60 mg to 100 mg, or in a range from 75 mg to 100 mg, or in a range from 75 mg to 100 mg, or in a range from 90 mg to 100 mg,
or wherein the single dose of the pharmaceutical composition comprises the antibody in an amount that is in a range from 1 mg to 25 mg, or in a range from 5 mg to 25 mg, or in a range from 10 mg to 25 mg, or in a range from 15 mg to 25 mg, or in a range from 20 mg to 25 mg,
or wherein the single dose of the pharmaceutical composition comprises the antibody in an amount that is in a range from 1 mg to 50 mg, or in a range from 1 mg to 25 mg, or in a range from 5 mg to 50 mg, or in a range from 5 mg to 25 mg, or in a range from 10 to 50 mg, or in a range from 10 to 25 mg, or in a range from 1 to 15 mg, or in a range from 5 mg to 15 mg, or in a range from 10 mg to 15 mg, or wherein the single dose of the pharmaceutical composition comprises 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 mg, or more, of the antibody,
or wherein the single dose of the pharmaceutical composition comprises the antibody in an amount that is less than 3000 mg, less than 2500 mg, less than 2000 mg, less than 1500 mg, less than 1000 mg, less than 900 mg, less than 500 mg, less than 300 mg, less than 200 mg, less than 100 mg, less than 90 mg, less than 75 mg, less than 50 mg, less than 25 mg, or less than 10 mg, but is more than 1 mg, more than 2 mg, more than 3 mg, more than 4 mg, or more than 5 mg; and/or the single dose of the pharmaceutical composition comprises the antibody at a concentration in a range from 100 mg/mL to 200 mg/mL, such as 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, preferably 150 mg/mL.

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. A pharmaceutical composition comprising the antibody or antigen-binding fragment of claim 3 at a concentration ranging from 100 mg/mL to 200 mg/mL, such as 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, preferably 150 mg/mL,
and a pharmaceutically acceptable carrier, excipient, or diluent.

119. (canceled)

120. (canceled)

121. (canceled)

122. (canceled)

123. (canceled)

124. (canceled)

125. The pharmaceutical composition of claim 118, wherein the pharmaceutical composition comprises water, optionally USP water; and/or pharmaceutical composition comprises histidine, optionally at a concentration from 10 mM to 40 mM, such as 20 mM, in the pharmaceutical composition; and/or the pharmaceutical composition comprises a disaccharide, such as sucrose, optionally at 5%, 6%, 7%, 8%, or 9%, preferably about 7% (w/v); and/or the pharmaceutical composition comprises a surfactant, optionally a polysorbate, preferably polysorbate 80 (PS80), wherein, optionally, the polysorbate is present in a range from 0.01% to 0.05% (w/v), preferably 0.02% (w/v); and/or the pharmaceutical composition has a pH ranging from 5.8 to 6.2, ranging from 5.9 to 6.1, or of 5.8, of 5.9, of 6.0, of 6.1, or of 6.2.

126. (canceled)

127. (canceled)

128. (canceled)

129. (canceled)

130. The pharmaceutical composition of claim 118, wherein the pharmaceutical composition comprises:
(i) the antibody at 150 mg/mL;
(ii) USP water;
(iii) 20 mM histidine;
(iv) 7% sucrose; and
(v) 0.02% PS80,
wherein the pharmaceutical composition comprises a pH of 6.

131. (canceled)

132. (canceled)

133. A method for in vitro diagnosis of a hepatitis B virus infection, the method comprising:
(i) contacting a sample from a subject with an antibody or antigen-binding fragment of claim 3; and
(ii) detecting a complex comprising an antigen and the antibody, or comprising an antigen and the antigen-binding fragment.

134. (canceled)

135. A method for detecting the presence or absence of an epitope in a correct conformation in an anti-hepatitis-B and/or an anti-hepatitis-D vaccine, the method comprising:
(i) contacting the vaccine with an antibody or antigen-binding fragment of claim 3; and
(ii) determining whether a complex comprising an antigen and the antibody, or comprising an antigen and the antigen binding fragment, has been formed.

136. (canceled)

137. A method of treating chronic HBV infection in a subject in need thereof, comprising:
administering to the subject an agent that reduces HBV antigenic load and/or an inhibitor of HBV gene expression; and
administering to the subject an anti-HBV antibody from of claim 3; wherein the agent that reduces HBV antigenic load and/or the inhibitor of HBV gene expression is an RNAi agent that inhibits expression of an HBV transcript.

138. (canceled)

139. The method according to claim 137, wherein the RNAi agent comprises a sense strand and an antisense strand forming a double-stranded region, wherein the sense strand comprises at least 15 contiguous nucleotides differing by no more than 3 nucleotides from nucleotides 1579-1597 of SEQ ID NO:116.

140. (canceled)

141. (canceled)

142. (canceled)

143. The method according to claim 137, wherein the double-stranded region of the RNAi agent is 15-30 nucleotide pairs in length or wherein each strand of the RNAi agent has 15-30 nucleotides.

144. (canceled)

145. (canceled)

146. (canceled)

147. (canceled)

148. (canceled)

149. (canceled)

150. (canceled)

151. The method according to claim 137, wherein the RNAi agent is an siRNA.

152. The method according to claim 151, wherein (a) the siRNA inhibits expression of an HBV transcript that encodes an HBsAg protein, an HBcAg protein, and HBx protein, or an HBV DNA polymerase protein; (b) the siRNA binds to at least 15 contiguous nucleotides of a target encoded by: P gene, nucleotides 2309-3182 and 1-1625 of NC_003977.2; S gene (encoding L, M, and S proteins), nucleotides 2850-3182 and 1-837 of NC_003977.2; HBx, nucleotides 1376-1840 of NC_003977.2; or C gene, nucleotides 1816-2454 of NC_003977.2; (c) siRNA that comprises an antisense strand, wherein the antisense strand of the siRNA comprises at least 15 contiguous nucleotides of the nucleotide sequence of 5′-UGUGAAGCGAAGUGCACACUU-3′ (SEQ ID NO:119); and/or (d) siRNA that comprises an antisense strand, wherein the antisense strand of the siRNA comprises at least 15 contiguous nucleotides of the nucleotide sequence of 5′-UAAAAUUGAGAGAAGUCCACCAC-3′ (SEQ ID NO:121).

153. (canceled)

154. (canceled)

155. (canceled)

156. (canceled)

157. (canceled)

158. (canceled)

159. (canceled)

160. (canceled)

161. (canceled)

162. (canceled)

163. (canceled)

164. (canceled)

165. (canceled)

166. The method according to claim 151, wherein substantially all of the nucleotides of said sense strand and substantially all of the nucleotides of said antisense strand are modified nucleotides, and
wherein said sense strand is conjugated to a ligand attached at the 3′-terminus.

167. The method according to claim 166, wherein fa) the ligand is one or more GalNAc derivatives attached through a monovalent linker, bivalent branched linker, or trivalent branched linker; or (b) the ligand is 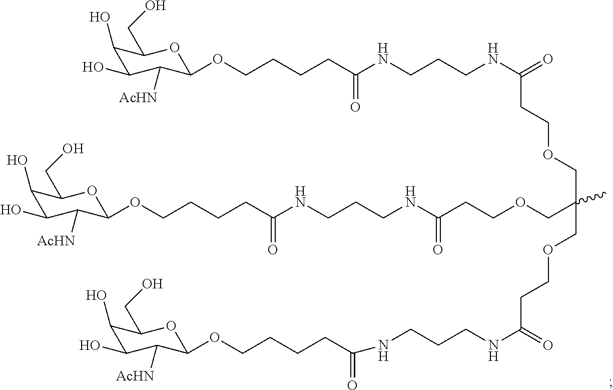 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]
or the siRNA is conjugated to the ligand as shown in the following structure: 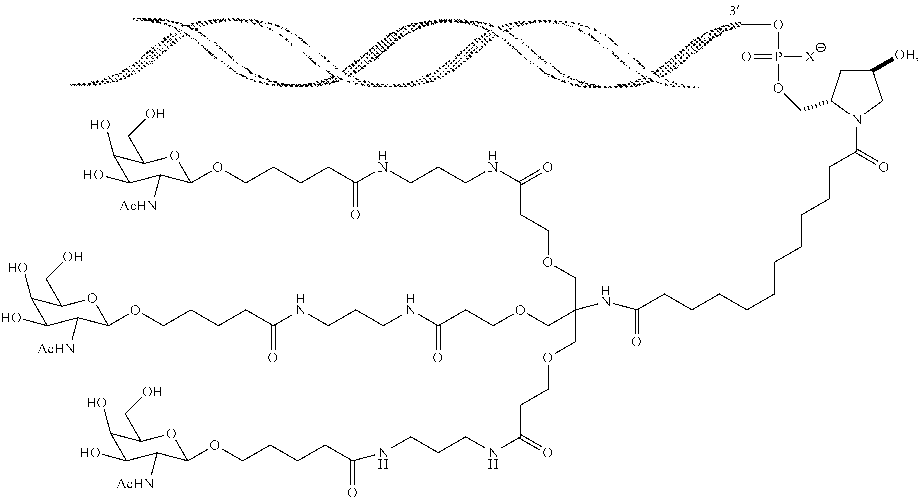
wherein X is O or S.

168. (canceled)

169. (canceled)

170. The method according to claim 151, wherein at least one nucleotide of the siRNA is a modified nucleotide comprising a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, an adenosine-glycol nucleic acid, or a nucleotide comprising a 5′-phosphate mimic; and/or the siRNA comprises a phosphate backbone modification, a 2′ ribose modification, 5′ triphosphate modification, or a GalNAc conjugation modification; and/or the phosphate backbone modification comprises a phosphorothioate bond; and/or the 2′ ribose modification comprises a fluoro or —O-methyl substitution.

171. (canceled)

172. (canceled)

173. (canceled)

174. The method according to claim 151, wherein
(a) the siRNA has a sense strand comprising 5′-gsusguGfcAfCfUfucgcuucacaL96-3′ (SEQ ID NO:122) and an antisense strand comprising 5′-usGfsugaAfgCfGfaaguGfcAfcacsusu-3′ (SEQ ID NO:123),
(b) the siRNA has a sense strand comprising 5′-gsusguGfcAfCfUfucgcuucacaL96-3′ (SEQ ID NO:124) and an antisense strand comprising 5′-usGfsuga(Agn)gCfGfaaguGfcAfcacsusu-3′ (SEQ ID NO:125), and/or
(c) the siRNA has a sense strand comprising 5′-gsgsuggaCfuUfCfUfcucaAfUfuuuaL96-3′ (SEQ ID NO:126) and an antisense strand comprising 5′-usAfsaaaUfuGfAfgagaAfgUfccaccsasc-3′ (SEQ ID NO:127); and
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
s is a phosphorothioate linkage; and
L96 is N-[tris(GalNAc-alkyl)-amidodecanoyl)]-4-hydroxyprolinol.

175. (canceled)

176. (canceled)

177. (canceled)

178. (canceled)

179. (canceled)

180. (canceled)

181. (canceled)

182. (canceled)

183. (canceled)

184. (canceled)

185. (canceled)

186. (canceled)

187. An antibody or antigen-binding fragment, comprising a heavy chain variable region (VH) and a light chain variable region (VL), wherein the VH and the VL comprise or consist of the amino acid sequences set forth in SEQ ID NOs.: 38 and 62, respectively;
wherein the antibody or antigen-binding fragment thereof is capable of binding to the antigenic loop region of HBsAg and neutralizing infection by a hepatitis B virus (HBV) of genotype D, A, B, C, E, F, G, H, I, or J, or any combination thereof and/or capable of neutralizing infection by a hepatitis D virus (HDV).

188. The antibody of claim 187, wherein the antibody comprises:
a heavy chain (HC) comprising or consisting of the amino acid sequence set forth in SEQ ID NO.:76, and
a light chain constant region (CL) that comprises or consists of an amino acid sequence set forth in SEQ ID NO.:79.

189. The antibody of claim 187, wherein the antibody comprises:
a heavy chain (HC) comprising or consisting of the amino acid sequence set forth in SEQ ID NO.:77, and
a light chain constant region (CL) that comprises or consists of an amino acid sequence set forth in SEQ ID NO.:79.

190. The antibody of claim 187, wherein the antibody comprises:
a heavy chain (HC) comprising or consisting of the amino acid sequence set forth in SEQ ID NO.:78, and
a light chain constant region (CL) that comprises or consists of an amino acid sequence set forth in SEQ ID NO.:79.

191. An antibody or antigen-binding fragment, comprising a heavy chain variable region (VH) and a light chain variable region (VL), wherein the VH and the VL comprise or consist of the amino acid sequences set forth in SEQ ID NOs.: 38 and 66, respectively;
wherein the antibody or antigen-binding fragment thereof is capable of binding to the antigenic loop region of HBsAg and neutralizing infection by a hepatitis B virus (HBV) of genotype D, A, B, C, E, F, G, H, I, or J, or any combination thereof and/or capable of neutralizing infection by a hepatitis D virus (HDV).

192. A method of treating chronic HBV infection and/or HDV infection in a subject in need thereof, comprising administering to the subject an anti-HBV antibody from claim 187.

193. A method of treating chronic HBV infection and/or HDV infection in a subject in need thereof, comprising:
administering to the subject an anti-HBV antibody from claim 187; and
administering to the subject an siRNA, wherein the siRNA has a sense strand comprising 5′-gsusguGfcAfCfUfucgcuucacaL96-3′ (SEQ ID NO:124) and an antisense strand comprising 5′-usGfsuga(Agn)gCfGfaaguGfcAfcacsusu-3′ (SEQ ID NO:125);
wherein a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively;
Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively;
s is a phosphorothioate linkage; and
L96 is N-[tris(GalNAc-alkyl)-amidodecanoyl)]-4-hydroxyprolinol.
